SEQUENCE LISTING (69 sequences)

SEQ ID NO: 1 - DNA, 1683 bp, Unknown
  Clostridium saccharogumia
cagaattaagaaacacacgtcaaagaaggatccggaaacggatagaagagaaaaagaagagctaaacaaacgaaagattgtaaagataaagagacaatggagagtttgatcctggctcaggatgaacgctggcggcgtgcctaatacatgcaagtcgaacgcgggcagcaatgcccgagtggcgaacgggtgagtaatacataagtaacctgccctttacagggggataactattggaaacgatagctaagaccgcataggtaaagataccgcatggtaagtttattaaaagtgccaaggcactggtagaggatggacttatggcgcattagctagttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaggaaggtcttcggactgtaaacttctgttataaaggaagaaaggcggatacagggaatggtatccgagtgacggtactttatgaggaagccacggctaactacgtgccagcagccgcggtaatacgtaggtggcaagcgttatccggaattattgggcgtaaagagggagcaggcggcagcaagggtctgtggtgaaagactgaagcttaacttcagtaagccatagaaaccgggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttgggagtcaaatctcagtgctgcagttaacgcagtaagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagatatatgggatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtggagcgaaacccagaaaaccattctcagttcggattgtagtctgcaactcgactacatgaagttggaatcgctagtaatcgcgaatcagcatgtcgcggtgaatacgttctcgggccttgtacacaccgcccgtcacaccatgagagttgataacacccgaagccggtggcctaaccgcaaggagggagctgtctaaggtgggattgatgattggggtgaagtcgtaacaaggtatccctacgggaacgtggggatggatcacctcctttctaaggagaaagaagagaagtgaagtttcattaatactgtttagttttgagtgatgctcaggagg

SEQ ID NO: 2 - DNA, 1685 bp, Unknown
  Flavonifractor plautii
ttaagcttcgataagatgatttgaggaacggagacgttctttagataccattttattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctcctttctaaggagacttcaacggaagtagttttctgttgtaacgtcctaaggtcagctttctggagcgaatggctttcacgttgtttaatttagagggtacactctc

SEQ ID NO: 3 - DNA, 1757 bp, Unknown
  Clostridium hathewayi
attcagaacgaatacaattattgtatacgaaccaaacaacaagtaaaacgggaaataaattagctagttagttgattttgaccccggatcaaacgaatcatttaacatgagagttcgatcctggctcaggatgaacgctggcggcgtgcttaacacatgcaagtcgagcgaagcggtttcaatgaagttttcggatggaattgaaattgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaacgtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccgtgatccctcttcggagcaatggagacaggtggtgcatggttgtcgtcagctcgtgtcgtgagatgttgggttaagtcccgcaacgagcgcaacccttatccttagtagccagcacatgatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaaggtgcggctggatcacctcctttctaaggaagaagaagtaagggttttatatactgttgagtcttgtgtttcaaaggaaggcgaaagccgaagaaaaatagaataaaagaatagaaacatcagagacaaagagattctggtgccga

SEQ ID NO: 4 - DNA, 2512 bp, Unknown
  Blautia producta
cgaagtctggtgtggaatattcagacaccccaaattttttcatacactgcgatccgggaagcatatgaggaagcgcggcagcagagcatggaggatattacagatgatgccatggtcatggagcgctttggaaacttgaaaattaagttggtggaaggttcctatgagaacattaaaatcactacgccggaggacattcttgtcgctgagaaaattttggaaaaaaattagagaaatctattgacaacttccgacataagtgataagataccacttgtcgctgaccgagtgacaaacatggagagatatcgaagtggtcataacgaggcggtcttgaaaaccgtttgtccgcaagggcgcgtgggttcgaatcccactctctccgttcccgaaagcagatatcgagaacgaatcagagtgacgagttcaagattagttatgatgcttttggatatatagagaagcagttatttgaaagcttactttcaaactgttgttttattcagcctggagaagtacccaagcggctgaaggggctcccctggaaagggagtaggtcgttaatagcggcgcgagggttcaaatccctccttctccgttttctcttaaaaaaagagaaaaaagttgttgacaagcgatcggatatatgataatctattatagctgtcgcaaaaacgacaacgatttatgacattgaaaatgagctttgcttaaaagctttgtgagtaaatgcaaaggtttagcgtagttgaaagaagtttcgacacaacgtaaaaaaacttttgaaaaacaatacaattaaaaataaatttttaattgtcataagttcttgacaagcacgaaacgatatgataaaatatcaaagctgtcaaaaactgacaggaaccttgataactgaacagtgaaacacatgaatcgaaaattcttttacattcattagtaacaaaacgaacggttctaaacgaaccaaaacagtaaaagggatatcttattgagcctagctcaataagccagaattagccaaagttaattctgttcctggacaaacacttgatcagagagtttgatcctggctcaggatgaacgctggcggcgtgcttaacacatgcaagtcgagcgaagcattaagacagatttcttcggattgaagtctttgtgactgagcggcggacgggtgagtaacgcgtgggtaacctgccttggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacctcctttctaaggaagaagaagtagagaaaagtgtttcactgttgagttaccaaga

SEQ ID NO: 5 - DNA, 1702 bp, Unknown
  Clostridium bolteae
gaaaattcagaacaaaaccaagtaatgaaggttaaagataactagccaagctaagtagtctgaaacctggaatcaacttttaaacatgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctcctttctaaggaagaagaagtaggggtttatgtattattgagtccttgattaaaaggtgcttaaaattttccggtggcgatgcgcttagggg

SEQ ID NO: 6 - DNA, 1703 bp, Unknown
  Clostridium indolis
aaaaaagatacacgaacgttctttataaaaggacacagtaaaacaaggataaaaaagctagcgctttttagatccaagattgaacatgagagtttgatcctggctcaggatgaacgctggcggcgtgcttaacacatgcaagtcgaacgaagcattttggaaggaagttttcggatggaattccttaatgactgagtggcggacgggtgagtaacgcgtggggaacctgccctatacagggggataacagctggaaacggctgctaataccgcataagcgcacagaatcgcatgattcggtgtgaaaagctccggcagtataggatggtcccgcgtctgattagctggttggcggggtaacggcccaccaaggcgacgatcagtagccggcttgagagagtggacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaaaagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatctaactgaccggttcgtaatgggacctttccttcgggacagttaagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccttataggagggagctgccgaaggtgggaccgataactggggtgaagtcgtaacaaggtagccgtatcggaaggtgcggctggatcacctcctttctaaggaagaagaagtaggggttgtcttattgtttagttatcaatgataacaaaaagtatcaagcaaggaagttctattgcactaaa

SEQ ID NO: 7 - DNA, 1211 bp, Unknown
  Anaerotruncus colihominis
atcaaagagtttgatcctggctcaggacgaacgctggcggcgcgcctaacacatgcaagtgtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccgaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgggcatccagacaggtggtgcatggttgtcgtcagctcgtgtcgtgagatgttgggttaagtcccgcaacgagcgcaacccttattattagttgctacgcaagagcactctaatgagactgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgctgtcgaaggtgggattgatgactggggtgaagtcgtaacaaggtagccgtatcggaaggtgcggctggatcacctcctttctaaggagcgaagccggcggagagccggcggactctggtcagacaggg

SEQ ID NO: 8 - DNA, 1700 bp, Unknown
  Drancourtella massiliensis
aacaggaactttaagttccacaaaccagacaacagtaacgggatagattagcgagagttgatcttgaccagaacaagttcttattacgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctcctttctaagggaagaagtaagggacgttgtctattgttgagcgatcgaagaggggagacccgaagaggagctcagaagcagttggtggtga

SEQ ID NO: 9 - DNA, 1691 bp, Clostridium lavalense
gaaccttgaaaatcaaagattgaacagtatgtaaaaccctgaaaattctaaaaaataaaggtctggtttagacctttggatttgagaaaattcagaacaaaaccaagtaatgaaggttaaagataactagccaagctaagtagtctaagaccaagaatcaacttttaatttgagagtttgatcctggctcaggatgaacgctggcggcgtgcctaacacatgcaagtcgaacgaagcattttagatgaagttttcggatggattctgagatgactgagtggcggacgggtgagtaacacgtggataacctgcctcacactgggggacaacagttagaaatgactgctaataccgcataaggattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagagagacaggtggtgcatggttgtcgtcagctcgtgtcgtgagatgttgggttaagtcccgcaacgagcgcaacccttatccttagtagccagcattaagatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctggatcacctcctttctaaggaagaagaagtaagggttttatatactgttgaatcttggatttgccaaggt

SEQ ID NO: 10 - DNA, 2435 bp, Unknown
  Clostridium symbiosum
caaaacaagattgatgaaaatatataaaaatatctggaaaaacaggcttgacaacccaatcttaatagttttaagtaataactgcggagaaatactcaagaggccgaagaggcgcccctgctaagggtgtaggtcgggcaaccggcgcgagggttcaaatccctctttctccgttttcttttgctgctgagaacaaacgacagcgttcaacaacaaagaaccttgaaaactgaacagtatgtaaaaccttgaaaattcttaaagaacaaaggcttttggttaagcctaaaagttcgagagttttgaacggaatcaaaacttaaacatgagagtttgatcctagctcaggatgaacgctggcggcgtgcctaacacatgcaagtcgaacgaagcaatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctcctttctaaggaagaagaagtagaggttttatatactgttgagttttcaagctcagacacttccggtgtcgatgcgctcaggggtcacacccgttcccatcccgaacacgatggttaagacctgagcggccgatgatactatattggagacgatatgggaaagcaggtggatgccggatttttctttaaaaaactcactcgtaagagtgttttatatagaacggacacatctgaatggatgaattcgtagtttctttagatgaaaaagacatctgtgacgacgatgtgttaacccactccagcagggaagtgctgttccatataactggtttttaccagtgattagtgaatttgagtttatcgagtttgctaatgactgataaagttcagtcagatatgtaccttgaaaactgcatattgaaataatatctagatagagtttcgaaactcttccgagtggcttgccacaagggagaaaagagagacaaaatcaagacatccgaggtgttacacgaaagtgtaaccaaaacaaaccaaaaattttattgtaaacctaaaccaaagattcaacgctatgaatcttagact

SEQ ID NO: 11 - DNA, 1644 bp, Clostridium ramosum
attgaaaactaaacagaattaagaaacacacgtcaatatccagaaggataaaaaagaaaaaagagctaaacaaacgaagagttgtttaaataaacaatggagagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaaggaaggagctgtctaaggtgggattgatgctaaggagaaacgcatgatcgaagatgatgcacaagagaagtgatgtttcgaagatactgtttagttttgagtgatactcaggt

SEQ ID NO: 12 - DNA, 1525 bp, Eubacterium contortum
actgagattaatctcggtggtaagaacagtaaaaagggatagaattagctagtagttaattctgacccggatacaaacacttttaacgagagtttgatcctggctcaggatgaacgctggcggcgtgcttaacacatgcaagtcgagcgaagcgctttacttagatttcttcggattgaaaagttttgcgactgagcggcggacgggtgagtaacgcgtgggtaacctgcctcatacagggggataacagttagaaatgactgctaataccgcataagaccacagtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggttatgtaagtctgatgtgaaaacccggggctcaaccccgggactgcattggaaactatgtaactagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgcatactaggtgtcgggtggcaaagccattcggtgccgcagcaaacgcaataagtatgccacctggggagtacgttcgcaagaatgaaactcaaaggaattgacggggacccgcacaagcggtggagcatgtggtttaattcgaagcaacgcgaagaaccttacctgctcttgacatccccctgaccggcgcgtaatggtgcctttccttcgggacaggggagacaggtggtgcatggttgtcgtcagctcgtgtcgtgagatgttgggttaagtcccgcaacgagcgcaacccttatctttagtagccagcggtttggccgggcactctagagagactgccagggataacctggaggaaggtggggatgacgtcaaatcatcatgccccttatgagcagggctacacacgtgctacaatggcgtaaacaaagggaggcgaagccgcgaggtggagcaaatcccaaaaataacgtctcagttcggattgtagtctgcaactcgactacatgaagctggaatcgctagtaatcgcgaatcagaatgtcggtgaagtcgtaacaaggtagccgtatcggaaggtgcggctggatcacctcctttctaaggaagaaagaagtaaagagtatgttgt

SEQ ID NO: 13 - DNA, 1440 bp, Clostridium scindens
tcaaccacagaaccttgataattgaacaatagacaacaaccctgaaaatttcttgtaaagagagaatttcagaacagaacgggacatcgagagatgtccgaccttaaaaacagtaataacgggaaagaattagccaagagttgattctgaccgcgatcacaacttttaacgagagtttgagcgtgggcaacctgccttgcactgggggataacagccagaaatggctgctaataccgcataagaccgaagcgccgcatggcgcagcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattccggatgggcactctggagagactgccagggacaacctggaggaaggtggggatgacgtcaaatcatcatgccccttatgaccagggccaaaaataacgtctcagttcggattgtagtctgcaactcgactacatgaagctggaatcgctagtaatcgcgaatcagaatgtcgcggtgaatacgttcccgggtcttgtacacaccgccccgtcgaaggtgggaccgataactggggtgaagtcgtaacaaggtagccgtatcggaagg

SEQ ID NO: 14 - DNA, 1440 bp, Unknown
  Lachnospiraceae bacterium
tagagttaatcttaactgggttaaacacttaaacaagagagtttgatcctggctcaggatgaacgctggcggcgtgcctaacacatgcaagtcgaacggagttatgcagaggaagttttcggatggaatcggcgtaacttagtggcggacgggtgagtaacgcgtgggaaacctgccctgtaccgggggataacacttagaaataggtgctaataccgcataagcgcacagcttcacatgaggcagtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgtgtccgtaacggggcattctcttcggagcattggagacaggtggtgcatggttgtcgtcagctcgtgtcgtgagatgttgggttaagtcccgcaacgagcgcaacccttatccttagtagccagcaggtaaagctgggcactctagggagactgccggggataacccggaggaaggcggggatgacgtcaaatcatcatgccccttatgatttgggctacacacgtgctacaatggcgtaaacaaagggaagcgagacagtgatgttgagcaaatcccagaaataacgtctcagttcggattgtagtctgcaactcgactacatgaagctggaatcgctagtaatcgcgaatcataactggggtgaagtcgtaacaaggtagccgtatcggaaggtgcggctggatcacctcct

SEQ ID NO: 15 - DNA, 1320 bp, Unknown
  Clostridium sp.
aatttgagagtttgatcctggctcaggatgaacgctggcggcgtgcctaacacatgcaaggggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaggagactgccagggataacctggaggaaggtggggatgacgtcaaatcatcatgcccctttgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgt

SEQ ID NO: 16 - DNA, 1451 bp, Unknown
  Lachnospiraceae bacterium
tgactggcagaacaacatgtgataagatatcaactgttgcggctgagaaagcagcacacagtgagtaacgcgtgggaaacctgccctgtaccgggggataacacttagaaataggtgctaggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgcacctgtaaagaggtgttcccttcggggcattggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaacaaggtagccgtatcggaaggtgcggctggatcacctcctttctaaggcgaagaagtaggggttgtattactgtttagatgtt

SEQ ID NO: 17 - DNA, 25 bp, Unknown
  Clostridium saccharogumia
ttttggcgttatcgggcaaacatat

SEQ ID NO: 18 - DNA, 25 bp, Unknown
  Flavonifractor plautii
acccggatctaattttggtcgaaaa

SEQ ID NO: 19 - DNA, 25 bp, Unknown
  Clostridium hathewayi
acagaaatattgaaagctttgtgat

SEQ ID NO: 20 - DNA, 25 bp, Unknown
  Blautia coccoides
tccgtggccccgaaggaggcttctg

SEQ ID NO: 21 - DNA, 25 bp, Clostridium bolteae
gaaatcttacacacctattcagacc

SEQ ID NO: 22 - DNA, 25 bp, Unknown
  Clostridium indolis
caagggagctttggtgagtttacac

SEQ ID NO: 23 - DNA, 25 bp, Unknown
  Anaerotruncus colihominis
ttgtactgttcccaatgagtatcaa

SEQ ID NO: 24 - DNA, 25 bp, Unknown
  Ruminococcus sp.
ctgatagacgaagaaggcgcatact

SEQ ID NO: 25 - DNA, 25 bp, Unknown
  Clostridium lavalense
gttgcttcgggaatcaatcaaatat

SEQ ID NO: 26 - DNA, 25 bp, Clostridium symbiosum
atcatccgctactccaaacccgatt

SEQ ID NO: 27 - DNA, 25 bp, Clostridium ramosum
ttacatttattcctttaatccccat

SEQ ID NO: 28 - DNA, 25 bp, Eubacterium contortum
cggaaggatcaaagtatatatggat

SEQ ID NO: 29 - DNA, 25 bp, Clostridium scindens
ttcgccacttaatatacggatgtta

SEQ ID NO: 30 - DNA, 25 bp, Unknown
  Lachnospiraceae bacterium
tcgataccaatttgtaataatccag

SEQ ID NO: 31 - DNA, 25 bp, Unknown
  Clostridium sp.
catccaccaaaagcagtccccctac

SEQ ID NO: 32 - DNA, 25 bp, Unknown
  Lachnospiraceae bacterium
gatcgtccgggtgctgcaaaaaatg

SEQ ID NO: 33 - DNA, 25 bp, Unknown
  Clostridium saccharogumia
gaaaaatcatcagctatagcattaa

SEQ ID NO: 34 - DNA, 25 bp, Unknown
  Flavonifractor plautii
ctccgtcacttaaatacatatcaac

SEQ ID NO: 35 - DNA, 25 bp, Unknown
  Clostridium hathewayi
ttatcagcatctaatatgtgcgtat

SEQ ID NO: 36 - DNA, 25 bp, Unknown
  Blautia coccoides
tgccccaaagacagaatgtatacct

SEQ ID NO: 37 - DNA, 25 bp, Unknown
  Clostridium bolteae
atcatccatctgctgttccaggctc

SEQ ID NO: 38 - DNA, 25 bp, Unknown
  Clostridium indolis
tgagccatcctgagatgaaatattg

SEQ ID NO: 39 - DNA, 25 bp, Unknown
  Anaerotruncus colihominis
gaatataatcaagatcctccagcgg

SEQ ID NO: 40 - DNA, 25 bp, Unknown
  Ruminococcus sp.
ttctccattttccatcatcctttca

SEQ ID NO: 41 - DNA, 25 bp, Unknown
  Clostridium lavalense
ctccatggctctggcggaattaaag

SEQ ID NO: 42 - DNA, 25 bp, Clostridium symbiosum
gtgcgatggggaggacgattatgcc

SEQ ID NO: 43 - DNA, 25 bp, Clostridium ramosum
tcacagtcggcattgggatataccg

SEQ ID NO: 44 - DNA, 25 bp, Eubacterium contortum
tcaaatatttctaactcgttggctt

SEQ ID NO: 45 - DNA, 25 bp, Clostridium scindens
ggacacgaattatccaaaaaagccg

SEQ ID NO: 46 - DNA, 25 bp, Unknown
  Lachnospiraceae bacterium
aatgaagcggcagcagcgtccaata

SEQ ID NO: 47 - DNA, 25 bp, Clostridium sp.
ggttaagattggttggcaggccata

SEQ ID NO: 48 - DNA, 25 bp, Unknown
  Lachnospiraceae bacterium
tccttcagtgctccatagtatcctt

SEQ ID NO: 49 - DNA, 150 bp, Clostridium saccharogumia
ttttggcgttatcgggcaaacatatgatgaatatgtaaaaccggcatttgataaaataaagcagggattaacaagtattgtaagcaccgtacttgatgcctataactctaatatcgctccagtagttaatgctatagctgatgatttttc

SEQ ID NO: 50 - DNA, 150 bp, Unknown
  Flavonifractor plautii
acccggatctaattttggtcgaaaacagcgaaggaattcttggatatgttaaatcagaggatttagatggttccaattatctgacgccggaggaggcgatattgaaccaatcacaagggtacagcgttgatatgtatttaagtgacggag

SEQ ID NO: 51 - DNA, 150 bp, Unknown
  Clostridium hathewayi
acagaaatattgaaagctttgtgattcctatagtcaaagttatcggtagaaaaggaacgattttataaggattgcaggctatgttcaaacgccagctttaaggagatgcacgaaaatacgcgtaaatacgcacatattagatgctgataa

SEQ ID NO: 52 - DNA, 150 bp, Unknown
  Blautia coccoides
tccgtggccccgaaggaggcttctgcccgcagagccacgccgtccatagcgcttgccgcataatggggtgcacagataacagcataaacagcctttgcagtgaccctcccgcaggcctcctgaacaggtatacattctgtctttggggca

SEQ ID NO: 53 - DNA, 150 bp, Unknown
  Clostridium bolteae
gaaatcttacacacctattcagaccagatgccgaagccgccgccgctttccaagcagaaagaattcaaacaaatcaagaacataattatccgcgtggccatggagttggaaccggccggaacatggagcctggaacagcagatggatgat

SEQ ID NO: 54 - DNA, 150 bp, Unknown
  Clostridium indolis
caagggagctttggtgagtttacaccgacagatggagattctggatcttctacacaaaagaagcgaagaatcaccgtccgaaaagtcacggaggaggaacttagaaagtttgcagagaatagcagcaatatttcatctcaggatggctca

SEQ ID NO: 55 - DNA, 150 bp, Unknown
  Anaerotruncus colihominis
ttgtactgttcccaatgagtatcaaagatgggcgaatcgatttttcccggttggaacaatatattacacccaaaacaaaaatatttgggttatgcaacccgcataacccgctgggaatgtgttatccgctggaggatcttgattatattc

SEQ ID NO: 56 - DNA, 150 bp, Unknown
  Ruminococcus sp.
ctgatagacgaagaaggcgcatactatcggtggagctgtccgaaaacagctgtatggaagggctgttctgtctcaatgcacccggagatggagaaaatcacagatccagtccgttgcagacaatgtgaaaggatgatggaaaatggagaa

SEQ ID NO: 57 - DNA, 150 bp, Unknown
  Clostridium lavalense
gttgcttcgggaatcaatcaaatatcatcagccattcaaggattttccgagcggactaagaccgccgattttagccgggttgttactggaatgaataagctggcccaggtagattcgcaaggagtctttaattccgccagagccatggag

SEQ ID NO: 58 - DNA, 150 bp, Clostridium symbiosum
atcatccgctactccaaacccgattccttttctcctcagcgtttcccagaatggctcaaaggtaatcatatcctcatcttgtccattgcccgcataattgcctcaaaagcatcatcccagcctctggcataatcgtcctccccatcgcac

SEQ ID NO: 59 - DNA, 150 bp, Clostridium ramosum
ttacatttattcctttaatccccatatgtccagtactcctatcctctgtcttttaagtcccagtcttgataattcactttttttaataaacagccctccaccgggtacaagatatgttcctgaagcggtatatcccaatgccgactgtga

SEQ ID NO: 60 - DNA, 150 bp, Eubacterium contortum
cgkaaggatcaaagtatatatggatgatgccttctgtggtgatatcactatcgcggacggtttacaaagcggaggaaaattgaacgctgatgccggaaaatatgagctgcggttgtcctttgaggaagccaacgagttagaaatatttga

SEQ ID NO: 61 - DNA, 150 bp, Clostridium scindens
ttcgccacttaatatacggatgttaggccgtatatggctattatctaaaatattgccaatgtcgtagtttaagccttcggcagaaataaccatgggatagtctgcaagatcctgtgccagtattgcggcttttttggataattcgtgtcc

SEQ ID NO: 62 - DNA, 150 bp, Unknown
  Lachnospiraceae bacterium
tcgataccaatttgtaataatccagttcacacgcttaatctgccccatcttgccgctatccacgatttccttcatcttacgatacacacaattggtccgctggttaaacataattgcaaacgtcttattggacgctgctgccgcttcatt

SEQ ID NO: 63 - DNA, 150 bp, Unknown
  Clostridium sp.
yatccaccaaaagcagtccccctacccacttcccagcaaaaaccactaaactccacatcccaggcctgccagcccctcatattcaccaaaccatggcatggccaatctaaaccattaaactatggtatggcctgccaaccaatctwaacc

SEQ ID NO: 64 - DNA, 150 bp, Unknown
  Lachnospiraceae bacterium
gatcgtccgggtgctgcaaaaaatgataagtatcggggactttacgatgtacttatctgcaataaacagcttcagcagttcaatgaatgatttaatgaaaagctgtgttgaaatcaaacagtttgaaggatactatggagcactgaagga

SEQ ID NO: 65 - DNA, 25 bp, Unknown
  Dielma fastidiosa
tgtctacggaaagatgcaaaaagtg

SEQ ID NO: 66 - DNA, 25 bp, Unknown
  Dielma fastidiosa
attgctgctgcatcagcattcaata

SEQ ID NO: 67 - DNA, 150 bp, Unknown
  Dielma fastidiosa
tgtctacggaaagatgcaaaaagtgtatacaattgaatcacaattwaatacagtcggtaacacaccttcattcagcgttataaaaatgtccaatgaagatatgaaaaaagcttcctttgctgcgttattgaatgctgatgcagcagcaat

SEQ ID NO: 68 - DNA, 150 bp, Unknown
  Dielma fastidiosa
tgtctacggaaagatgcaaaaagtgtatacaattgaatcacaattaaatacagtcggtaacacaccttcattcagcgttataaaaatgtccaatgaagatatgaaaaaagcttcctttgctgcgttattgaatgctgatgcagcagcaat

SEQ ID NO: 69 - DNA, 150 bp, Unknown
  Clostridium indolis
yaakggagctttggtgagtttacaccgacatatggagattctggatcttctacacaaaagaagcgaagaatcaccgtccgaaaagtcacggaggaggaacttagaaagtttgcagagaatagcagcaatatttcatctcaggatggctca

(19) United States
(12) Patent Application Publication
(10) Pub. No.: US 2024/0173365 A1
(43) Pub. Date: May 30, 2024

(54) METHODS OF COLONIZING A MICROBIOME, TREATING AND/OR PREVENTING INFLAMMATORY BOWEL DISEASE AND GRAFT VERSUS HOST DISEASE

(71) Applicant: Vedanta Biosciences, Inc., Cambridge, MA (US)

(72) Inventors: Emily Crossette, Somerville, MA (US); Bernat Olle, Cambridge, MA (US)

(73) Assignee: Vedanta Biosciences, Inc., Cambridge, MA (US)

(21) Appl. No.: 18/574,232

(22) PCT Filed: Jun. 28, 2022
(86) PCT No.: PCT/US2022/035356

§ 371 Date: Dec. 26, 2023

Related U.S. Application Data
(60) Provisional application No. 63/215,954, filed on Jun. 28, 2021.

(51) Int. Cl. A61K 35/742; A61K 31/606; A61K 35/741; A61K 38/14; A61P 1/04; C12Q 1/6851; C12Q 1/689; A61K 35/00

(52) U.S. Cl. CPC A61K 35/742; A61K 31/606; A61K 35/741; A61K 38/14; A61P 1/04; C12Q 1/6851; C12Q 1/689; A61K 2035/115

(57) ABSTRACT

The disclosure relates to methods for colonizing a microbiome, by administering pharmaceutical compositions comprising a purified bacterial mixture to the subject. Also provided are methods for treating and/or preventing inflammatory bowel disease, methods for reducing the risk of inflammatory bowel disease and/or reducing the occurrence of inflammatory bowel disease in a subject by administering pharmaceutical compositions comprising a purified bacterial mixture to the subject. Also provided are methods for treating and/or preventing graft versus host disease in a subject by administering pharmaceutical compositions comprising a purified bacterial mixture to the subject.

### RELATED APPLICATION

This application claims the benefit under 35 U.S.C. § 119(c) of U.S. provisional application No. 63/215,954, filed Jun. 28, 2021, the entire contents of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 28, 2022, is named P074570030WO00-SEQ-NTJ, and is 49,845 bytes in size.

### BACKGROUND

The human intestinal microbiome comprises tens of trillions of bacteria from over 1000 identified species. The composition of an individual's microbiome is as unique as a fingerprint, with wide variety existing between even close relatives. The vast majority of species present in the human microbiome are commensals, which neither harm nor hurt the host. Numerous bacterial species which inhabit the human intestinal tract, including members of the taxa Lactobacillus, Firmicutes, and Bacteriodetes are symbionts which perform functions which benefit the human, such as the metabolism of food by-products into nutrients which can be absorbed. However, pathogenic bacterial species, such as strains of Escherichia coli, may also inhabit the human intestinal tract and may cause disease if allowed to overpopulate the human microbiome. Thus, the balance of bacterial species within the human microbiome is critical to maintaining overall human health.

### SUMMARY

Aspects of the present disclosure relate to methods for colonizing a microbiome in a subject, the method comprising administering to the subject a therapeutically effective amount of a pharmaceutical composition comprising a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, he purified bacterial mixture comprises at least three, four, five, six, seven, eight, nine, ten, eleven, twelve, thirteen, fourteen, fifteen, or sixteen, bacterial strains.

In some embodiments, the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the subject has, is suspected of having, or is at risk of having inflammatory bowel disorder (IBD).

In some embodiments, the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium. In some embodiments, the subject has, is suspected of having, or is at risk of having graft versus host disease (GvHD).

Aspects of the present disclosure relate to methods for colonizing a microbiome in a subject, the method comprising administering to the subject a therapeutically effective of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16. In some embodiments, the purified bacterial mixture comprises at least three, four, five, six, seven, eight, nine, ten, eleven, twelve, thirteen, fourteen, fifteen, or sixteen bacterial strains.

In some embodiments, he pharmaceutical composition comprises a purified bacterial mixture consisting of bacterial strains that comprise 16S rDNA sequences of at least 97% sequence identity to the nucleic acid sequences set forth as SEQ ID NOs: 1-16. In some embodiments, the subject has, is suspected of having, or is at risk of having inflammatory bowel disorder (IBD). In some embodiments, the IBD is ulcerative colitis or Crohn's disease. In some embodiments, the subject has one or more risk factors associated with IBD.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs:3, 5-10, 12, and 14-16. In some embodiments, the subject has, is suspected of having, or is at risk of having graft versus host disease (GvHD).

In some embodiments, each of the bacterial strains of the pharmaceutical composition colonizes the microbiome. In some embodiments, the bacterial strains of the pharmaceutical composition colonize the microbiome over an extended period of time.

In some embodiments, the pharmaceutical composition reduces the amount of one or more primary bile acids in the subject. In some embodiments, the primary bile acid is chenodeoxycholic acid, cholic acid, glycochenodeoxycholic acid, glycocholic acid, taurochenodeoxycholic acid, or taurocholic acid. In some embodiments, the pharmaceutical composition reduces levels of primary bile acids in the subject by 2-fold to 100,000-fold.

In some embodiments, the pharmaceutical composition increases the amount of one or more secondary bile acids in the subject. In some embodiments, the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid. In some embodiments, the pharmaceutical composition increases levels of secondary bile acids in the subject by 2-fold to 100,000-fold.

In some embodiments, the pharmaceutical composition increases the amount of one or more short-chain fatty acids (SCFAs) in the subject. In some embodiments, the SCFA is 2-methylbutyric acid, acetic acid, butyric acid, hexanoic acid, isobutyric acid, isovaleric acid, propionic acid, or valeric acid. In some embodiments, the pharmaceutical composition increases levels of short-chain fatty acids (SCFAs) in the subject by 2-fold to 100,000-fold.

In some embodiments, the pharmaceutical composition increases the amount of one or more indoles in the subject. In some embodiments, the indole is kynurenic acid, serotonin, nicotinic acid, indole, indole 3-acetic, or indole 3-propionic acid. In some embodiments, the pharmaceutical composition increases levels of indoles in the subject by 2-fold to 100,000-fold. In some embodiments, the method further comprises administering to the subject an antibiotic. In some embodiments, in the subject was administered an antibiotic prior to administration of the pharmaceutical composition. In some embodiments, administration of the pharmaceutical composition is not preceded by administration of an antibiotic. In some embodiments, the antibiotic is vancomycin, fidaxomycin, or ridinilazole. In some embodiments, the antibiotic is vancomycin.

In some embodiments, the vancomycin is administered at a dose sufficient to allow for colonization of one or more of the bacterial strains of the pharmaceutical composition. In some embodiments, the vancomycin is administered in 4 doses of 125 mg per day. In some embodiments, the vancomycin is administered for five consecutive days. In some embodiments, the vancomycin is administered on five consecutive days immediately prior to the day of the administration of the pharmaceutical composition. In some embodiments, the vancomycin is administered on five consecutive days up to two days prior to the day of the administration of the pharmaceutical composition, and wherein the method includes a washout day one day prior to the day of the administration of the pharmaceutical composition.

In some embodiments, the pharmaceutical composition is administered as a single dose. In some embodiments, the pharmaceutical composition is administered in multiple doses. In some embodiments, a dose of the pharmaceutical composition comprises between 10⁸ to 10¹¹ total colony forming units (CFUs). In some embodiments, a dose of the pharmaceutical composition comprises about 10⁹ total CFUs. In some embodiments, a dose of the pharmaceutical composition comprises about 10¹⁰ total CFUs. In some embodiments, each dose comprises the administration of multiple capsules. In some embodiments, each capsule comprises about 10⁹ total CFUs. In some embodiments, each dose comprises administration of 10 capsules each comprising about 10⁹ total CFUs.

In some embodiments, the multiple doses are administered on consecutive days. In some embodiments, the multiple doses are administered on 7-14 consecutive days. In some embodiments, the method further comprises administering a therapeutic agent. In some embodiments, the therapeutic agent is a therapeutic agent for treating IBD or GvHD.

In some embodiments, the two or more bacterial strains are lyophilized. In some embodiments, the two or more bacterial strains are spray-dried. In some embodiments, one or more of the two or more bacterial strains are in spore form. In some embodiments, each of the two or more bacterial strains are in spore form. In some embodiments, one or more of the two or more bacterial strains are in vegetative form. In some embodiments, each of the two or more bacterial strains are in vegetative form.

In some embodiments, the pharmaceutical composition further comprises one or more enteric polymers. In some embodiments, the administration is oral administration. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

Aspects of the present disclosure relate to methods for treating and/or preventing inflammatory bowel disease (IBD) in a subject, the method comprising administering to the subject a therapeutically effective amount of a pharmaceutical composition comprising a purified bacterial mixture comprising two or more bacterial strains of species elected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture comprises at least three, four, five, six, seven, eight, nine, ten, eleven, twelve, thirteen, fourteen, fifteen, or sixteen bacterial strains.

In some embodiments, the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

Aspects of the present disclosure relate to methods for treating and/or preventing inflammatory bowel disease (IBD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16. In some embodiments, the purified bacterial mixture comprises at least three, four, five, six, seven, eight, nine, ten, eleven, twelve, thirteen, fourteen, fifteen, or sixteen bacterial strains.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 1-16.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

In some embodiments, the subject is at risk of developing IBD. In some embodiments, the IBD is ulcerative colitis or Crohn's disease. In some embodiments, the pharmaceutical composition is administered after a first therapeutic agent for treating IBD. In some embodiments, the first therapeutic for treating IBD is an antibiotic or aminosalicylate (5-ASA) agent. In some embodiments, the subject has one or more risk factors associated with IBD.

In some embodiments, each of the bacterial strains of the pharmaceutical composition colonizes the microbiome. In some embodiments, the bacterial strains of the pharmaceutical composition colonize the microbiome over an extended period of time.

In some embodiments, the pharmaceutical composition reduces the amount of one or more primary bile acids in the subject. In some embodiments, the primary bile acid is chenodeoxycholic acid, cholic acid, glycochenodeoxycholic acid, glycocholic acid, taurochenodeoxycholic acid, or taurocholic acid. In some embodiments, the pharmaceutical composition reduces levels of primary bile acids in the subject by 2-fold to 100,000-fold.

In some embodiments, the pharmaceutical composition increases the amount of one or more secondary bile acids in the subject. In some embodiments, the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid. In some embodiments, the pharmaceutical composition increases levels of secondary bile acids in the subject by 2-fold to 100,000-fold.

In some embodiments, the pharmaceutical composition increases the amount of one or more short-chain fatty acids (SCFAs) in the subject. In some embodiments, the SCFA is 2-methylbutyric acid, acetic acid, butyric acid, hexanoic acid, isobutyric acid, isovaleric acid, propionic acid, or valeric acid. In some embodiments, the pharmaceutical composition increases levels of short-chain fatty acids (SCFAs) in the subject by 2-fold to 100,000-fold.

In some embodiments, the pharmaceutical composition increases the amount of one or more indoles in the subject. In some embodiments, the indole is kynurenic acid, serotonin, nicotinic acid, indole, indole 3-acetic, or indole 3-propionic acid. In some embodiments, the pharmaceutical composition increases levels of indoles in the subject by 2-fold to 100,000-fold. In some embodiments, the method further comprises administering to the subject an antibiotic. In some embodiments, in the subject was administered an antibiotic prior to administration of the pharmaceutical composition. In some embodiments, administration of the pharmaceutical composition is not preceded by administration of an antibiotic. In some embodiments, the antibiotic is vancomycin, fidaxomycin, or ridinilazole. In some embodiments, the antibiotic is vancomycin.

In some embodiments, the vancomycin is administered at a dose sufficient to allow for colonization of one or more of the bacterial strains of the pharmaceutical composition. In some embodiments, the vancomycin is administered in 4 doses of 125 mg per day. In some embodiments, the vancomycin is administered for five consecutive days. In some embodiments, the vancomycin is administered on five consecutive days immediately prior to the day of the administration of the pharmaceutical composition. In some embodiments, the vancomycin is administered on five consecutive days up to two days prior to the day of the administration of the pharmaceutical composition, and wherein the method includes a washout day one day prior to the day of the administration of the pharmaceutical composition.

In some embodiments, the pharmaceutical composition is administered as a single dose. In some embodiments, the pharmaceutical composition is administered in multiple doses. In some embodiments, a dose of the pharmaceutical composition comprises between 10⁸ to 10¹¹ total colony forming units (CFUs). In some embodiments, a dose of the pharmaceutical composition comprises about 10⁹ total CFUs. In some embodiments, a dose of the pharmaceutical composition comprises about 10¹⁰ total CFUs. In some embodiments, each dose comprises the administration of multiple capsules. In some embodiments, each capsule comprises about 10⁹ total CFUs. In some embodiments, each dose comprises administration of 10 capsules each comprising about 10⁹ total CFUs.

In some embodiments, the multiple doses are administered on consecutive days. In some embodiments, the multiple doses are administered on 7-14 consecutive days. In some embodiments, the method further comprises administering a therapeutic agent. In some embodiments, the therapeutic agent is a therapeutic agent for treating IBD or GvHD.

In some embodiments, the two or more bacterial strains are lyophilized. In some embodiments, the two or more bacterial strains are spray-dried. In some embodiments, one or more of the two or more bacterial strains are in spore form. In some embodiments, each of the two or more bacterial strains are in spore form. In some embodiments, one or more of the two or more bacterial strains are in vegetative form. In some embodiments, each of the two or more bacterial strains are in vegetative form.

In some embodiments, the pharmaceutical composition further comprises one or more enteric polymers. In some embodiments, the administration is oral administration. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

Aspects of the present disclosure relate to methods for reducing the risk and/or occurrence of inflammatory bowel disease (IBD) in a subject, the method comprising administering to the subject a therapeutically effective amount of a pharmaceutical composition comprising a purified bacterial mixture comprising two or more bacterial strains of species elected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture comprises at least three, four, five, six, seven, eight, nine, ten, eleven, twelve, thirteen, fourteen, fifteen, or sixteen bacterial strains.

In some embodiments, the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

Aspects of the present disclosure provide methods for reducing the risk and/or occurrence of inflammatory bowel disease (IBD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16. In some embodiments, the purified bacterial mixture comprises at least three, four, five, six, seven, eight, nine, ten, eleven, twelve, thirteen, fourteen, fifteen, or sixteen bacterial strains.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 1-16. In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

In some embodiments, the IBD is ulcerative colitis or Crohn's disease. In some embodiments, the pharmaceutical composition is administered after a first therapeutic agent for treating IBD. In some embodiments, the first therapeutic for treating IBD is an antibiotic or aminosalicylate (5-ASA) agent. In some embodiments, the subject has one or more risk factors associated with IBD.

In some embodiments, each of the bacterial strains of the pharmaceutical composition colonizes the microbiome. In some embodiments, the bacterial strains of the pharmaceutical composition colonize the microbiome over an extended period of time.

In some embodiments, the pharmaceutical composition reduces the amount of one or more primary bile acids in the subject. In some embodiments, the primary bile acid is chenodeoxycholic acid, cholic acid, glycochenodeoxycholic acid, glycocholic acid, taurochenodeoxycholic acid, or taurocholic acid. In some embodiments, the pharmaceutical composition reduces levels of primary bile acids in the subject by 2-fold to 100,000-fold.

In some embodiments, the pharmaceutical composition increases the amount of one or more secondary bile acids in the subject. In some embodiments, the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid. In some embodiments, the pharmaceutical composition increases levels of secondary bile acids in the subject by 2-fold to 100,000-fold.

In some embodiments, the pharmaceutical composition increases the amount of one or more short-chain fatty acids (SCFAs) in the subject. In some embodiments, the SCFA is 2-methylbutyric acid, acetic acid, butyric acid, hexanoic acid, isobutyric acid, isovaleric acid, propionic acid, or valeric acid. In some embodiments, the pharmaceutical composition increases levels of short-chain fatty acids (SCFAs) in the subject by 2-fold to 100,000-fold.

In some embodiments, the pharmaceutical composition increases the amount of one or more indoles in the subject. In some embodiments, the indole is kynurenic acid, serotonin, nicotinic acid, indole, indole 3-acetic, or indole 3-propionic acid. In some embodiments, the pharmaceutical composition increases levels of indoles in the subject by 2-fold to 100,000-fold. In some embodiments, the method further comprises administering to the subject an antibiotic. In some embodiments, in the subject was administered an antibiotic prior to administration of the pharmaceutical composition. In some embodiments, administration of the pharmaceutical composition is not preceded by administration of an antibiotic. In some embodiments, the antibiotic is vancomycin, fidaxomycin, or ridinilazole. In some embodiments, the antibiotic is vancomycin.

In some embodiments, the vancomycin is administered at a dose sufficient to allow for colonization of one or more of the bacterial strains of the pharmaceutical composition. In some embodiments, the vancomycin is administered in 4 doses of 125 mg per day. In some embodiments, the vancomycin is administered for five consecutive days. In some embodiments, the vancomycin is administered on five consecutive days immediately prior to the day of the administration of the pharmaceutical composition. In some embodiments, the vancomycin is administered on five consecutive days up to two days prior to the day of the administration of the pharmaceutical composition, and wherein the method includes a washout day one day prior to the day of the administration of the pharmaceutical composition.

In some embodiments, the pharmaceutical composition is administered as a single dose. In some embodiments, the pharmaceutical composition is administered in multiple doses. In some embodiments, a dose of the pharmaceutical composition comprises between 10⁸ to 10¹¹ total colony forming units (CFUs). In some embodiments, a dose of the pharmaceutical composition comprises about 10⁹ total CFUs. In some embodiments, a dose of the pharmaceutical composition comprises about 10¹⁰ total CFUs. In some embodiments, each dose comprises the administration of multiple capsules. In some embodiments, each capsule comprises about 10⁹ total CFUs. In some embodiments, each dose comprises administration of 10 capsules each comprising about 10⁹ total CFUs.

In some embodiments, the multiple doses are administered on consecutive days. In some embodiments, the multiple doses are administered on 7-14 consecutive days. In some embodiments, the method further comprises administering a therapeutic agent. In some embodiments, the therapeutic agent is a therapeutic agent for treating IBD or GvHD.

In some embodiments, the two or more bacterial strains are lyophilized. In some embodiments, the two or more bacterial strains are spray-dried. In some embodiments, one or more of the two or more bacterial strains are in spore form. In some embodiments, each of the two or more bacterial strains are in spore form. In some embodiments, one or more of the two or more bacterial strains are in vegetative form. In some embodiments, each of the two or more bacterial strains are in vegetative form.

In some embodiments, the pharmaceutical composition further comprises one or more enteric polymers. In some embodiments, the administration is oral administration. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

Aspects of the present disclosure provide methods for treating and/or preventing graft versus host disease (GvHD) in a subject, the method comprising administering to the subject a therapeutically effective amount of a pharmaceutical composition comprising a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture comprises at least three, four, five, six, seven, eight, nine, ten, eleven, twelve, thirteen, fourteen, fifteen, or sixteen bacterial strains.

In some embodiments, the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

Aspects of the present disclosure provide methods for treating and/or preventing graft versus host disease (GvHD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16. In some embodiments, the purified bacterial mixture comprises at least three, four, five, six, seven, eight, nine, ten, eleven, twelve, thirteen, fourteen, fifteen, or sixteen bacterial strains.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 1-16. In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

In some embodiments, the subject is at risk of developing GvHD. In some embodiments, the pharmaceutical composition is administered after a first therapeutic agent for treating GvHD.

In some embodiments, each of the bacterial strains of the pharmaceutical composition colonizes the microbiome. In some embodiments, the bacterial strains of the pharmaceutical composition colonize the microbiome over an extended period of time.

In some embodiments, the pharmaceutical composition reduces the amount of one or more primary bile acids in the subject. In some embodiments, the primary bile acid is chenodeoxycholic acid, cholic acid, glycochenodeoxycholic acid, glycocholic acid, taurochenodeoxycholic acid, or taurocholic acid. In some embodiments, the pharmaceutical composition reduces levels of primary bile acids in the subject by 2-fold to 100,000-fold.

In some embodiments, the pharmaceutical composition increases the amount of one or more secondary bile acids in the subject. In some embodiments, the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid. In some embodiments, the pharmaceutical composition increases levels of secondary bile acids in the subject by 2-fold to 100,000-fold.

In some embodiments, the pharmaceutical composition increases the amount of one or more short-chain fatty acids (SCFAs) in the subject. In some embodiments, the SCFA is 2-methylbutyric acid, acetic acid, butyric acid, hexanoic acid, isobutyric acid, isovaleric acid, propionic acid, or valeric acid. In some embodiments, the pharmaceutical composition increases levels of short-chain fatty acids (SCFAs) in the subject by 2-fold to 100,000-fold. In some embodiments, the pharmaceutical composition increases the amount of one or more indoles in the subject. In some embodiments, the indole is kynurenic acid, serotonin, nicotinic acid, indole, indole 3-acetic, or indole 3-propionic acid. In some embodiments, the pharmaceutical composition increases levels of indoles in the subject by 2-fold to 100,000-fold. In some embodiments, the method further comprises administering to the subject an antibiotic. In some embodiments, in the subject was administered an antibiotic prior to administration of the pharmaceutical composition. In some embodiments, administration of the pharmaceutical composition is not preceded by administration of an antibiotic. In some embodiments, the antibiotic is vancomycin, fidaxomycin, or ridinilazole. In some embodiments, the antibiotic is vancomycin.

In some embodiments, the vancomycin is administered at a dose sufficient to allow for colonization of one or more of the bacterial strains of the pharmaceutical composition. In some embodiments, the vancomycin is administered in 4 doses of 125 mg per day. In some embodiments, the vancomycin is administered for five consecutive days. In some embodiments, the vancomycin is administered on five consecutive days immediately prior to the day of the administration of the pharmaceutical composition. In some embodiments, the vancomycin is administered on five consecutive days up to two days prior to the day of the administration of the pharmaceutical composition, and wherein the method includes a washout day one day prior to the day of the administration of the pharmaceutical composition.

In some embodiments, the pharmaceutical composition is administered as a single dose. In some embodiments, the pharmaceutical composition is administered in multiple doses. In some embodiments, a dose of the pharmaceutical composition comprises between 10⁸ to 10¹¹ total colony forming units (CFUs). In some embodiments, a dose of the pharmaceutical composition comprises about 10⁹ total CFUs. In some embodiments, a dose of the pharmaceutical composition comprises about 10¹⁰ total CFUs. In some embodiments, each dose comprises the administration of multiple capsules. In some embodiments, each capsule comprises about 10⁹ total CFUs. In some embodiments, each dose comprises administration of 10 capsules each comprising about 10⁹ total CFUs.

In some embodiments, the multiple doses are administered on consecutive days. In some embodiments, the multiple doses are administered on 7-14 consecutive days. In some embodiments, the method further comprises administering a therapeutic agent. In some embodiments, the therapeutic agent is a therapeutic agent for treating IBD or GvHD.

In some embodiments, the two or more bacterial strains are lyophilized. In some embodiments, the two or more bacterial strains are spray-dried. In some embodiments, one or more of the two or more bacterial strains are in spore form. In some embodiments, each of the two or more bacterial strains are in spore form. In some embodiments, one or more of the two or more bacterial strains are in vegetative form. In some embodiments, each of the two or more bacterial strains are in vegetative form.

In some embodiments, the pharmaceutical composition further comprises one or more enteric polymers. In some embodiments, the administration is oral administration. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

Aspects of the present disclosure provide methods comprising administering to a subject a therapeutically effective amount of a pharmaceutical composition in a loading period followed by administering to the subject a therapeutically effective amount of the pharmaceutical composition a maintenance period, wherein the pharmaceutical composition comprises a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture comprises at least three, four, five, six, seven, eight, nine, ten, eleven, twelve, thirteen, fourteen, fifteen, or sixteen bacterial strains.

In some embodiments, the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium. In some embodiments, the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

Aspects of the present disclosure provide methods comprising administering to a subject a therapeutically effective amount of a pharmaceutical composition in a loading period followed by administering to the subject a therapeutically effective amount of the pharmaceutical composition a maintenance period, wherein the pharmaceutical composition comprises a purified bacterial mixture comprising two or more bacterial strains that comprise 16S rDNA sequences of at least 97% sequence identity to the nucleic acid sequences set forth as SEQ ID NOs: 1-16. In some embodiments, the purified bacterial mixture comprises at least three, four, five, six, seven, eight, nine, ten, eleven, twelve, thirteen, fourteen, fifteen, or sixteen bacterial strains.

In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 1-16. In some embodiments, the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

In some embodiments, the pharmaceutical composition reduces the amount of one or more primary bile acids in the subject. In some embodiments, the primary bile acid is chenodeoxycholic acid, cholic acid, glycochenodeoxycholic acid, glycocholic acid, taurochenodeoxycholic acid, or taurocholic acid. In some embodiments, the pharmaceutical composition reduces levels of primary bile acids in the subject by 2-fold to 100,000-fold.

In some embodiments, the pharmaceutical composition increases the amount of one or more secondary bile acids in the subject. In some embodiments, the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid. In some embodiments, the pharmaceutical composition increases levels of secondary bile acids in the subject by 2-fold to 100,000-fold.

In some embodiments, the pharmaceutical composition increases the amount of one or more short-chain fatty acids (SCFAs) in the subject. In some embodiments, the SCFA is 2-methylbutyric acid, acetic acid, butyric acid, hexanoic acid, isobutyric acid, isovaleric acid, propionic acid, or valeric acid. In some embodiments, the pharmaceutical composition increases levels of short-chain fatty acids (SCFAs) in the subject by 2-fold to 100,000-fold. In some embodiments, the pharmaceutical composition increases the amount of one or more indoles in the subject. In some embodiments, the indole is kynurenic acid, serotonin, nicotinic acid, indole, indole 3-acetic, or indole 3-propionic acid. In some embodiments, the pharmaceutical composition increases levels of indoles in the subject by 2-fold to 100,000-fold.

In some embodiments, the method further comprises administering to the subject an antibiotic prior to the loading period. In some embodiments, the loading period is not preceded by administration of an antibiotic. In some embodiments, the antibiotic is vancomycin, fidaxomycin, or ridinilazole. In some embodiments, the antibiotic is vancomycin.

In some embodiments, the loading period is at least 7 days and a loading dose of the pharmaceutical composition is administered to the subject at least every 3 days for the loading period. In some embodiments, the loading period is between 7 and 14 days. In some embodiments, the loading dose of the pharmaceutical composition is administered to the subject daily for the loading period. In some embodiments, the maintenance period is 6 weeks, and a maintenance dose of the pharmaceutical composition is administered to the subject daily for the maintenance period. In some embodiments, the maintenance dose comprises fewer total colony forming units as compared to the loading dose. In some embodiments, the loading period and maintenance period are repeated every 2 months, 3 months, 4 months, 5 months, 6 months, 12 months, 18 months, or 24 months.

In some embodiments, the two or more bacterial strains are lyophilized. In some embodiments, the two or more bacterial strains are spray-dried. In some embodiments, one or more of the two or more bacterial strains are in spore form. In some embodiments, each of the two or more bacterial strains are in spore form. In some embodiments, one or more of the two or more bacterial strains are in vegetative form. In some embodiments, each of the two or more bacterial strains are in vegetative form.

In some embodiments, the pharmaceutical composition further comprises one or more enteric polymers. In some embodiments, the administration is oral administration. In some embodiments, the pharmaceutical composition is formulated for oral delivery. In some embodiments, the pharmaceutical composition is formulated for rectal delivery. In some embodiments, the pharmaceutical composition is formulated for delivery to the intestine. In some embodiments, the pharmaceutical composition is formulated for delivery to the colon.

Aspects of the present disclosure relate to methods for assessing colonization of one or more bacterial strains of a bacterial composition in a microbiome of a subject, the method comprising isolating nucleic acid from a sample of the microbiome of the subject; and determining the presence of at least one bacterial strain of the bacterial composition by amplifying a nucleotide sequence of a genomic marker for the at least one the bacterial strains in the isolated nucleic acid; wherein if a genomic marker for a bacterial strain is present in the amplified nucleotide sequences, the microbiome is colonized with the bacterial strain.

In some embodiments, amplifying comprises performing one or more quantitative polymerase chain reactions (qPCR). In some embodiments, the qPCR is performed using one or more pair of primers, wherein each pair of primers comprises a forward primer and a reverse primer for amplifying the nucleotide sequence of the genomic marker of bacterial strain. In some embodiments, the pair of primers for amplifying the nucleotide sequence of the genomic marker of comprises the forward primer set forth in any one of SEQ ID NOS: 17-32 and the reverse primer set forth in any one of SEQ ID NOs: 33-48.

In some embodiments, the qPCR reaction further comprises a DNA probe. In some embodiments, the DNA probe comprises a fluorophore and at least one quencher. In some embodiments, the DNA probe comprises a sequence that is present in any one of the sequences set forth in SEQ ID NOs: 49-64.

In some embodiments, if a genomic marker for a bacterial strain is absent in the amplified nucleotide sequences, the method further comprises administering one or more additional doses of the bacterial composition to the subject.

These and other aspects of the disclosure, as well as various embodiments thereof, will become more apparent in reference to the drawings and detailed description of the disclosure.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 shows the design and results of a study evaluating use of a live bacterial product containing 11 bacterial strains (11-mix) for treating inflammatory bowel disease (IBD). Healthy subjects were treated with a single or multiple doses of the 11-mix with or without pretreatment with vancomycin for 5 days. “SD” refers to a single day of administration of the 11-mix; “MD” refers to multiple days (i.e., 14 days) of administration of the 11-mix. “Dose 1” refers to administration of one capsule containing approximately 1×10⁹ colony forming units (CFUs); “Dose 2” refers to administration of ten capsules each containing approximately 1×10⁹ colony forming units (CFUs) (i.e. ˜1×10¹⁰ CFU total).

FIG. 2 shows microbial colonization results from subjects of the study shown in FIG. 1. The left panel shows microbial density of subjects that were administered a single dose of 1×10⁹ CFU of the 11-mix (or control subjects) (cohort 1), and the right panel shows microbial density of subjects that were administered a single dose of 1×10¹⁰ CFU of the 11-mix (or control subjects) (cohort 2). The single dose of the 11-mix was administered on day 1, and stool samples were collected for analysis on the indicated days. The line indicating subjects that were administered the placebo control is shown with an asterisks, and the line indicating control subjects that administered the 11-mix is shown with a #symbol. Data is shown as the mean+/−standard deviation.

FIG. 3 shows microbial colonization results from subjects of the study shown in FIG. 1. The left panel shows microbial density of subjects that were administered multiple doses (i.e., 14 days) of 1×10⁹ CFU of the 11-mix (or control subjects) (cohort 4), and the right panel shows microbial density of subjects that were administered a multiple doses (i.e., 14 days) of 1×10¹⁰ CFU of the 11-mix (or control subjects) (cohort 6). The 5-day period of vancomycin pretreatment is indicated in with a “1,” the 14-day period of administration of the 11-mix is indicated with a “2,” and stool samples were collected for analysis on the indicated days. “*” indicates subjects that were administered the placebo control, and “#” indicates control subjects that were administered the 11-mix. Data is shown as the mean+/−standard deviation.

FIGS. 4A and 4B show the change in strain detection and strain abundance in subjects of the study shown in FIG. 1. FIG. 4A shows the number of strains of the 11-mix detected at the indicated time points. FIG. 4B shows the abundance of the strains of the 11-mix detected at the indicated time points. The left panel shows microbial density of subjects that were administered a single dose of 1×10⁹ CFU of the 11-mix (or control subjects) (cohort 1), and the right panel shows microbial density of subjects that were administered a single dose of 1×10¹⁰ CFU of the 11-mix (or control subjects) (cohort 2). The single dose of the 11-mix was administered on day 1 (indicated by the vertical dashed line), and stool samples were collected for analysis on the indicated days. The line showing subjects that were administered the placebo control is indicated with an asterisks, and the line showing control subjects that were administered the 11-mix is indicated with a #symbol. Data is shown as the mean+/−standard deviation.

FIGS. 5A and 5B show the change in strain detection and strain abundance in subjects of the study shown in FIG. 1. FIG. 5A shows the number of strains of the 11-mix detected at the indicated time points. FIG. 5B shows the abundance of the strains of the 11-mix detected at the indicated time points. The top graphs show microbial density of subjects that were administered multiple doses of 1×10⁹ CFU of the 11-mix (or control subjects) without vancomycin pretreatment (cohort 3, left panel) or with vancomycin pretreatment (cohort 4, right panel). The bottom graphs show microbial density of subjects that were administered multiple doses of 1×10¹⁰ CFU of the 11-mix (or control subjects) without vancomycin pretreatment (cohort 5, left panel) or with vancomycin pretreatment (cohort 6, right panel). The 5-day period of vancomycin pretreatment is indicated in light shading, the 14-day period of administration of the 11-mix are shown in dark shading, and stool samples were collected for analysis on the indicated days. In each graph, bottom line indicates subjects that were administered the placebo control, and the top line indicates control subjects that were administered the 11-mix. Data is shown as the mean+/−standard deviation.

FIGS. 6A-6F show the relative abundance of each of the strains of the 11-mix in subjects of the study shown in FIG. 1. Each panel corresponds to an individual subject with stool samples collected for analysis on the indicated days. Each line corresponds to a bacterial strain as detected with the indicated primer. FIG. 6A shows subjects that were administered a single dose of 1×10⁹ CFU of the 11-mix (or control subjects, “placebo”) without vancomycin pretreatment (cohort 1). FIG. 6B shows subjects that were administered a single dose of 1×10¹⁰ CFU of the 11-mix (or control subjects, “placebo”) without vancomycin pretreatment (cohort 2). FIG. 6C shows subjects that were administered multiple doses of 1×10⁹ CFU of the 11-mix (or control subjects, “placebo”) without vancomycin pretreatment (cohort 3). FIG. 6D shows subjects that were administered multiple doses of 1×10⁹ CFU of the 11-mix (or control subjects, “placebo”) with vancomycin pretreatment (cohort 4). FIG. 6E shows subjects that were administered multiple doses of 1×10¹⁰ CFU of the 11-mix (or control subjects, “placebo”) without vancomycin pretreatment (cohort 5). FIG. 6F shows subjects that were administered multiple doses of 1×10¹⁰ CFU of the 11-mix (or control subjects, “placebo”) with vancomycin pretreatment (cohort 6).

FIG. 7 shows the design and results of a study evaluating use of a live bacterial product containing 16-bacterial strains (16-mix) for treating inflammatory bowel disease (IBD). Healthy subjects were treated with a single dose or multiple doses of the 16-mix after pretreatment with vancomycin pretreatment for 5 days. “SD” refers to a single day of administration of the 16-mix; “MD” refers to multiple days (i.e., 14 days) of administration of the 16-mix. “Dose 1” refers to administration of one capsule containing approximately 1×10⁹ colony forming units (CFUs); “Dose 2” refers to administration of ten capsules each containing approximately 1×10⁹ colony forming units (CFUs) (i.e. ˜ 1×10¹⁰ CFU total).

FIGS. 8A and 8B show the change in strain detection and strain abundance of in subjects of the study shown in FIG. 7. FIG. 8A shows the number of strains of the 16-mix detected at the indicated time points. FIG. 8B shows the abundance of the strains of the 16-mix detected at the indicated time points. After vancomycin pretreatment, subjects received either a single dose of 1×10⁹ CFU (cohort 1, left panels), a single dose of 1×10¹⁰ CFU (cohort 2, middle panels), or multiple doses of 1×10⁹ CFU (cohort 3, right panels). Stool samples were collected for analysis on the indicated days. In each graph, the bottom line indicates subjects that were administered the placebo control, and the top line indicates control subjects that were administered the 16-mix. Data is shown as the mean+/−standard deviation.

FIG. 9 shows the total detected strains of the 16-mix in subjects of that received multiple doses of 1×10⁹ (cohort 3) following vancomycin pretreatment in the study shown in FIG. 7. Stool samples were collected for analysis on the indicated days. Data are shown as stacked columns, with the presence of a strain in the column indicating that it was detected in the sample. The total number of strains observed in each sample is shown above the column.

FIGS. 10A and 10B show the change in the relative abundances of individual bacterial strains of the 16-mix in subjects of the study shown in FIG. 7. FIG. 10A shows the relative abundance over time of individual strains of the 16-mix in subjects that received multiple doses of 1×10⁹ (cohort 3) following vancomycin pretreatment (cohort 3). Each panel corresponds to an individual bacterial strain detected with the indicated primer, with stool samples collected for analysis on the indicated days. Individual time courses are shown as thin lines, while thick lines show the mean+/−standard deviation of a strain's abundance over time. Dark lines represent subjects of cohort 3, while light lines represent control subjects (placebo-treated). FIG. 10B shows a plot of the estimated pharmacokinetic parameters based on the relative abundance data shown in FIGS. 2, 4B, 5B, 6A-6F, 8B, 10A, and 11A-11C. Cₘₐₓ=maximum abundance; tₘₐₓ=time at which maximum abundance is observed; Cₗₐₛₜ=last observed abundance; tₗₐₛₜ=time of last observation. The pretreatment period with vancomycin (“vanco”), expansion phase, and persistence phase are each indicated.

FIGS. 11A-11C show the relative abundance of each of the strains of the 16-mix bacterial strains in subjects of the study shown in FIG. 7. Each panel corresponds to an individual subject with stool samples collected for analysis on the indicated days. Each line corresponds to a bacterial strain as detected with the indicated primer. FIG. 11A shows subjects that were administered a single dose of 1×10⁹ CFU of the 16-mix (or control subjects, “placebo”) after vancomycin pretreatment (cohort 1). FIG. 11B shows subjects that were administered a single dose of 1×10¹⁰ CFU of the 16-mix (or control subjects, “placebo”) after vancomycin pretreatment (cohort 2). FIG. 11C shows subjects that were administered multiple doses of 1×10⁹ CFU of the 16-mix (or control subjects, “placebo”) after vancomycin pretreatment (cohort 3).

FIG. 12 shows the design and results of a study evaluating use of a live bacterial product containing 16-bacterial strains (16-mix) for treating mild-to-moderate ulcerative colitis. In Part 1 of the study, a first group of subjects (Group A) are pretreated with vancomycin, followed by multiple doses of the 16-mix (5 capsules per day, each capsule containing 1×10⁹ colony forming units (CFUs) (i.e., 5×10⁹ CFU total)) for a loading period of 14 days. From day 15 to day 56, subjects are administered multiple doses of the 16-mix (1 capsule per day containing 1×10⁹ colony forming units (CFUs)) for a maintenance period. In Part 2 of the study, subjects of Group A receive placebo during the pretreatment period, followed by administration of multiple doses of a placebo (5 capsules per day) for a 14 day loading period, followed by no therapy from days 15-56. In Part 1 of the study, a second group of subjects (Group B) are pretreated with vancomycin, followed by multiple doses of a placebo (5 capsules per day) for a loading period of 14 days. From day 15 to day 56, subjects are administered multiple doses of a placebo for a maintenance period. In Part 2 of the study, subjects of Group B are pretreated with vancomycin, followed by multiple doses of the 16-mix (5 capsules per day, each capsule containing 1×10⁹ colony forming units (CFUs) (i.e., 5×10⁹ CFU total)) for a 14 day loading period, followed by no therapy from days 15-56. In Part 3, both groups are subjected to long-term follow-up over the course of a year.

FIGS. 13A-13D show the concentration of metabolites quantified in subjects pre-treated with vancomycin then administered 1×10⁹ CFU per day of 16-mix (or control subjects, “placebo”) over 14 days and subjects pre-treated with vancomycin then administered 1×10¹⁰ CFU per day of the 11-mix (or control subjects, “placebo”). FIG. 13A shows primary bile acid concentrations. FIG. 13B shows secondary bile acid concentrations. FIG. 13C shows short-chain fatty acid (SCFA) concentrations. FIG. 13D shows indole concentrations. Each panel contains data of a single metabolite quantified across subjects, with each point representing the concentration of the metabolite in a single subject's stool sample, and the solid line representing the median concentration across each cohort and pooled placebo subjects.

### DETAILED DESCRIPTION

Provided herein are methods for colonizing a microbiome in a subject involving administering pharmaceutical compositions comprising two or more purified bacterial strains. Provided herein are methods for treating and/or preventing inflammatory bowel disease (IBD) in a subject involving administering pharmaceutical compositions comprising two or more purified bacterial strains. Also provided herein are methods for reducing risk and/or occurrence of inflammatory bowel disease (IBD) in a subject involving administering pharmaceutical compositions comprising two or more purified bacterial strains to a subject. Also provided herein are methods for treating and/or preventing graft versus host disease (GvHD) a subject involving administering pharmaceutical compositions comprising two or more purified bacterial strains to a subject. Also provided herein are methods involving administering pharmaceutical compositions to a subject in a loading period followed by administering to the subject a therapeutically effective amount of the pharmaceutical composition a maintenance period. Also provided herein are methods for assessing colonization of one or more bacterial strains of a bacterial composition in a microbiome of a subject.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including.” “comprising,” or “having,” “containing,” “involving.” and variations thereof herein, is meant to encompass the items listed thereafter and equivalents thereof as well as additional items.

Microbiomes are present, for example in mammalian subjects, on the skin, within the gastrointestinal tract (i.e., the gut), within the oral cavity, and within the vaginal tract of female subjects, and comprise bacteria, archaca, protists, fungi, and viruses. In some instances, the species present in a microbiome benefit the subject by performing useful or necessary functions, such as aiding in the digestion of food in the intestinal tract of the subject, protecting the body from penetration by pathogenic microbes, and promoting immunological development. Organisms within the microbiota that perform these functions may be referred to as symbiotic or commensal organisms because they exist in the subject without harming, and, in some cases, actually benefit the host. In dysbiosis, a state of imbalance of the microbiome of a subject, the normal microbiome of the subject is perturbed or damaged, which may lead to a variety of diseases and/or disorders. In some embodiments, the species that normally dominate the microbiome become underrepresented (e.g., commensal or symbiotic species) and species which are normally underrepresented (e.g., opportunistic species) become overrepresented. See also Petersen et al., “Defining dysbiosis and its influence on host immunity and disease.” Cell Microbiol (2014) 16 (7), 1024-1033.

Aspects of the present disclosure relate to administration of pharmaceutical composition comprising a purified bacterial mixture comprising two or more bacterial strains.

In general, bacterial strains may be classified phylogenetically with other closely related strains and species based on their 16S rRNA (or 16S rDNA) nucleic acid sequence. Methods for determining the identity of specific bacterial species based on their 16S rRNA (or 16S rDNA) nucleic acid sequence are well known in the art (See, e.g., Jumpstart Consortium Human Microbiome Project Data Generation Working, G. PLOS One (2012) 7, e39315).

The present disclosure encompasses compositions comprising bacterial strains having close sequence identity or homology to and/or fall within the species of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp, Lachnospiraceae bacterium, Clostridium ramosum; Erysipelatoclostridium saccharogumia, Erysipelatoclostridium sp000508865, Pseudoflavinofractor capillolus, Lachnospiraceae bacterium, Clostridium orbiscindens, Flavonifractor sp000508885, Clostridium saccharolyticum, Hungatella hathewayi, Hungatella effluvia, Lachnospiraceae bacterium; Blautia producta, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Erysipelatoclostridium ramosum, Eubacterium fissicatena, Ruminococcus fissicatena, Faecalicatena contorta, Muricomes sp000509105, Faecalicatena sp000509105, Lachnospiraceae bacterium, Dorea scindens, Dorea sp000509125, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16 or more bacterial strains (e.g., purified bacterial strains).

It should be appreciated that the terms “bacteria” and “bacterial strains” as used herein are interchangeable. The compositions described herein containing multiple purified bacterial strains may also be referred to as “live bacterial products.”

In one aspect, the disclosure provides a live bacterial product referred to as “11-mix.” As shown in Table 1, the composition referred to as 11-mix contains 11 bacterial strains related to the following species: Clostridium hathewayi Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp, Lachnospiraceae bacterium, Clostridium saccharolyticum, Hungatella hathewayi, Hungatella effluvia, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Eubacterium fissicatena, Ruminococcus fissicatena, Faecalicatena contorta, Muricomes sp000509105, Faecalicatena sp000509105, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In one aspect, the disclosure provides a live bacterial product referred to as “16-mix.” As shown in Table 1, the composition referred to as 16-mix contains 16 bacterial strains related to the following species: Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp, Lachnospiraceae bacterium, Clostridium ramosum; Erysipelatoclostridium saccharogumia, Erysipelatoclostridium sp000508865, Pseudoflavinofractor capillolus, Lachnospiraceae bacterium, Clostridium orbiscindens, Flavonifractor sp000508885, Clostridium saccharolyticum, Hungatella hathewayi, Hungatella effluvia, Lachnospiraceae bacterium; Blautia producta, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Erysipelatoclostridium ramosum, Eubacterium fissicatena, Ruminococcus fissicatena, Faecalicatena contorta, Muricomes sp000509105, Faecalicatena sp000509105, Lachnospiraceae bacterium, Dorea scindens, Dorea sp000509125, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In some embodiments, the compositions comprise two or more (e.g., 2, 3, 4, 5, or more) purified bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp, Lachnospiraceae bacterium, Clostridium ramosum; Erysipelatoclostridium saccharogumia, Erysipelatoclostridium sp000508865, Pseudoflavinofractor capillolus, Lachnospiraceae bacterium, Clostridium orbiscindens, Flavonifractor sp000508885, Clostridium saccharolyticum, Hungatella hathewayi, Hungatella effluvia, Lachnospiraceae bacterium; Blautia producta, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Erysipelatoclostridium ramosum, Eubacterium fissicatena, Ruminococcus fissicatena, Faecalicatena contorta, Muricomes sp000509105, Faecalicatena sp000509105, Lachnospiraceae bacterium, Dorea scindens, Dorea sp000509125, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In some embodiments, the compositions consist of two or more (e.g., 2, 3, 4, 5, or more) bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp, Lachnospiraceae bacterium, Clostridium ramosum; Erysipelatoclostridium saccharogumia, Erysipelatoclostridium sp000508865, Pseudoflavinofractor capillolus, Lachnospiraceae bacterium, Clostridium orbiscindens, Flavonifractor sp000508885, Clostridium saccharolyticum, Hungatella hathewayi, Hungatella effluvia, Lachnospiraceae bacterium; Blautia producta, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Erysipelatoclostridium ramosum, Eubacterium fissicatena, Ruminococcus fissicatena, Faecalicatena contorta, Muricomes sp000509105, Faecalicatena sp000509105, Lachnospiraceae bacterium, Dorea scindens, Dorea sp000509125, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In some embodiments, the compositions consist essentially of two or more (e.g., 2, 3, 4, 5, or more) bacterial strains of species selected from the group consisting Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp, Lachnospiraceae bacterium, Clostridium ramosum, Erysipelatoclostridium saccharogumia, Erysipelatoclostridium sp000508865, Pseudoflavinofractor capillolus, Lachnospiraceae bacterium, Clostridium orbiscindens, Flavonifractor sp000508885, Clostridium saccharolyticum, Hungatella hathewayi, Hungatella effluvia, Lachnospiraceae bacterium; Blautia producta, Lachnoclostridium bolteae, Enterocloster bolteae, Anaerostipes caccae, Anaerostipes sp000508985, Lachnospiraceae bacterium, Sellimonas intestinalis, Eubacterium fissicatena, Drancourtella massiliensis, Ruminococcus torques, Sellimonas intestinalis, Lachnoclostridium lavalense, Clostridium asparagiforme, Lachnoclostridium asparagiforme, Enterocloster asparagiforme, Lachnoclostridium symbiosum, Clostridium_Q symbiosum, Erysipelatoclostridium ramosum, Eubacterium fissicatena, Ruminococcus fissicatena, Faecalicatena contorta, Muricomes sp000509105, Faecalicatena sp000509105, Lachnospiraceae bacterium, Dorea scindens, Dorea sp000509125, Eisenbergiella massiliensis, Eisenbergiella tayi, Clostridium aldenense, Clostridium symbiosum, Lachnoclostridium symbiosum, Enterocloster sp000155435, Lachnoclostridium pacaense, Eubacterium fissicatena, Ruminococcus fissicatena, Faecalicatena contorta, Eubacterium contortum, and Eisenbergiella tayi.

In some embodiments, the compositions comprise bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

In some embodiments, the compositions consist of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

In some embodiments, the compositions consist essentially of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

In some embodiments, the compositions comprise bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

In some embodiments, the compositions consist of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

In some embodiments, the compositions consist essentially of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

Aspects of the disclosure relate to bacterial strains with 16S rDNA sequences that have sequence identity to a nucleic acid sequence of any one of the sequences of the bacterial strains or species described herein. As will be appreciated by one of ordinary skill in the art, the 16S rDNA sequences represent DNA sequences corresponding to the 16S rRNA sequence. The terms “identical,” or percent “identity,” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. Sec, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. WI), or by manual alignment and visual inspection (sec. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

In some embodiments, a bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the strains or bacterial species described herein over a specified region or over the entire sequence. It would be appreciated by one of skill in the art that the term “sequence identity” or “percent sequence identity,” in the context of two or more nucleic acid sequences or amino acid sequences, refers to a measure of similarity between two or more sequences or portion(s) thereof.

In some aspects, the bacterial composition comprises two or more (e.g., 2, 3, 4, 5, 6, 7, 8, 9, 10, 11, 12, 13, 14, 15, or 16) bacterial strains, wherein the two or more bacterial strains contain 16S rDNA sequences having at least 97% sequence identity with nucleic acid sequences selected from SEQ ID NOs: 1-16. In some aspects, the bacterial composition consists of two or more (e.g., 2, 3, 4, 5, 6, 7, 8, 9, 10, 11, 12, 13, 14, 15, or 16) bacterial strains, wherein the two or more bacterial strains contain 16S rDNA sequences having at least 97% sequence identity with nucleic acid sequences selected from SEQ ID NOs: 1-16. In some aspects, the bacterial composition consists essentially of two or more (e.g., 2, 3, 4, 5, 6, 7, 8, 9, 10, 11, 12, 13, 14, 15, or 16) bacterial strains, wherein the two or more bacterial strains contain 16S rDNA sequences having at least 97% sequence identity with nucleic acid sequences selected from SEQ ID NOs: 1-16.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, or more bacterial strains (e.g., purified bacterial strains).

In some embodiments, the pharmaceutical composition includes three or more bacterial strains, wherein the three or more bacterial strains contain 16S rDNA sequences having at least 97% sequence identity with nucleic acid sequences selected from SEQ ID NOs: 1-16. In some embodiments, the pharmaceutical composition includes four or more bacterial strains, wherein the four or more bacterial strains contain 16S rDNA sequences having at least 97% sequence identity with nucleic acid sequences selected from SEQ ID NOs: 1-16. In some embodiments, the pharmaceutical composition includes five or more bacterial strains, wherein the five or more bacterial strains contain 16S rDNA sequences having at least 97% sequence identity with nucleic acid sequences selected from SEQ ID NOs: 1-16. In some embodiments, the pharmaceutical composition includes six or more bacterial strains, wherein the six or more bacterial strains contain 16S rDNA sequences having at least 97% sequence identity with nucleic acid sequences selected from SEQ ID NOs: 1-16. In some embodiments, the pharmaceutical composition includes seven or more bacterial strains, wherein the seven or more bacterial strains contain 16S rDNA sequences having at least 97% sequence identity with nucleic acid sequences selected from SEQ ID NOs: 1-16. In some embodiments, the pharmaceutical composition includes eight or more bacterial strains, wherein the eight or more bacterial strains contain 16S rDNA sequences having at least 97% sequence identity with nucleic acid sequences selected from SEQ ID NOs: 1-16. In some embodiments, the pharmaceutical composition includes nine or more bacterial strains, wherein the nine or more bacterial strains contain 16S rDNA sequences having at least 97% sequence identity with nucleic acid sequences selected from SEQ ID NOs: 1-16. In some embodiments, the pharmaceutical composition includes ten or more bacterial strains, wherein the ten or more bacterial strains contain 16S rDNA sequences having at least 97% sequence identity with nucleic acid sequences selected from SEQ ID NOs: 1-16. In some embodiments, the pharmaceutical composition includes eleven or more bacterial strains, wherein the eleven or more bacterial strains contain 16S rDNA sequences having at least 97% sequence identity with nucleic acid sequences selected from SEQ ID NOs: 1-16. In some embodiments, the pharmaceutical composition includes twelve or more bacterial strains, wherein the twelve or more bacterial strains contain 16S rDNA sequences having at least 97% sequence identity with nucleic acid sequences selected from SEQ ID NOs: 1-16. In some embodiments, the pharmaceutical composition includes thirteen or more bacterial strains, wherein the thirteen or more bacterial strains contain 16S rDNA sequences having at least 97% sequence identity with nucleic acid sequences selected from SEQ ID NOs: 1-16. In some embodiments, the pharmaceutical composition includes fourteen or more bacterial strains, wherein the fourteen or more bacterial strains contain 16S rDNA sequences having at least 97% sequence identity with nucleic acid sequences selected from SEQ ID NOs: 1-16. In some embodiments, the pharmaceutical composition includes fifteen or more bacterial strains, wherein the fifteen or more bacterial strains contain 16S rDNA sequences having at least 97% sequence identity with nucleic acid sequences selected from SEQ ID NOs: 1-16. In some embodiments, the pharmaceutical composition includes sixteen or more bacterial strains, wherein the sixteen or more bacterial strains contain 16S rDNA sequences having at least 97% sequence identity with nucleic acid sequences selected from SEQ ID NOs: 1-16.

In some embodiments, the pharmaceutical composition comprises a purified bacterial mixture comprising eleven bacterial strains, wherein the eleven bacterial strains contain 16S rDNA sequences having at least 97% sequence identity with the nucleic acid sequences of SEQ ID NOs: 3, 5-10, 12, and 14-16. In some embodiments, each of the eleven bacterial strains comprises a 16S rDNA sequence having at least 97% sequence identity to any one of the nucleic acid sequences of SEQ ID NOs: SEQ ID NOs: 3, 5-10, 12, and 14-16, wherein each of the nucleic acid sequences of SEQ ID NOs: 3, 5-10, 12, and 14-16 is present in at least one bacterial strain.

In some embodiments, the pharmaceutical composition comprises a purified bacterial mixture consisting of eleven bacterial strains, wherein the eleven bacterial strains contain 16S rDNA sequences having at least 97% sequence identity with the nucleic acid sequences of SEQ ID NOs: 3, 5-10, 12, and 14-16. In some embodiments, the pharmaceutical composition comprises a purified bacterial mixture consisting essentially of eleven bacterial strains, wherein the eleven bacterial strains contain 16S rDNA sequences having at least 97% sequence identity with the nucleic acid sequences of SEQ ID NOs: 3, 5-10, 12, and 14-16.

In some embodiments, the pharmaceutical composition comprises a purified bacterial mixture comprising sixteen bacterial strains, wherein the sixteen bacterial strains contain 16S rDNA sequences having at least 97% sequence identity with the nucleic acid sequences of SEQ ID NOs: 1-16. In some embodiments, each of the sixteen bacterial strains comprises a 16S rDNA sequence having at least 97% sequence identity to any one of the nucleic acid sequences of SEQ ID NOs: 1-16, wherein each of the nucleic acid sequences of SEQ ID NOs: 1-16 is present in at least one bacterial strain.

In some embodiments, the pharmaceutical composition comprises a purified bacterial mixture consisting of sixteen bacterial strains, wherein the sixteen bacterial strains contain 16S rDNA sequences having at least 97% sequence identity with the nucleic acid sequences of SEQ ID NOs: 1-16. In some embodiments, the pharmaceutical composition comprises a purified bacterial mixture consisting essentially of sixteen bacterial strains, wherein the sixteen bacterial strains contain 16S rDNA sequences having at least 97% sequence identity with the nucleic acid sequences of SEQ ID NOs: 1-16.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

It should be appreciated that the terms “bacteria” and “bacterial strains” as used herein are interchangeable. The compositions described herein containing multiple purified bacterial strains may also be referred to as “live bacterial products.”

In one aspect, the 16S rDNA sequences of purified bacterial strains of the compositions were compared to 16S rDNA sequences of known bacterial species/strains in a bacterial sequence database to identify the closest known related bacterial species to the bacterial stains disclosed herein (sec, Table 1).

Homologies based on 16S sequence analysis are presented in Table 1. Table 1 provides the closest known species by homology when the 16S rDNA sequences, or portion thereof, comprising SEQ ID NOs: 1-16 are compared to 16S rDNA sequences of bacterial species available in public databases. Throughout the instant application the bacterial strains associated with 16S rDNA sequences comprising SEQ ID NOs: 1-16 may alternatively or in addition be referred to by any of the additional nucleic acid sequences provided by SEQ ID NOs: 1-16, as shown in Table 1. It should be appreciated that multiple bacterial strains of the compositions described herein may have the same closest related bacterial species.

It should be appreciated that the bacterial strains described herein have a 16S rDNA sequence selected from SEQ ID NOs: 1-16 may also be homologous to other strains based on their whole genome sequence, or subset of their whole genome sequence.

As will be appreciated by one of skill in the art, the bacterial strains disclosed herein may identified as having a high level of homology with a bacterial species but may be referred to by an alternative species name. It should further be appreciated that the bacterial strains disclosed herein that have a 16S rDNA sequence with a nucleic acid sequence selected from the group consisting of SEQ ID NOs: 1-16 are also homologous to other strains based on additional sequences, e.g., their whole genome sequence, or subset of their whole genome sequence. By way of an example, the bacterial strain comprising a 16S rDNA sequence of SEQ ID NO: 1 may be referred to as a bacterial strain of species Clostridium saccharogumia but, in addition or alternatively, may be referred to as Clostridium ramosum, Erysipelatoclostridium saccharogumia, and/or Erysipelatoclostridium sp000508865.

TABLE 1
Bacterial strains

| | | | | | SEQ |
|---|---|---|---|---|---|
| Strain | Original | | 11- | 16- | ID |
| No. | Consensus | Alternative Species | mix | mix | NO |
| 1 | Clostridium | Clostridium ramosum; | | X | 1 |
| | saccharogumia | Erysipelatoclostridium | | | |
| | | saccharogumia; | | | |
| | | Erysipelatoclostridium | | | |
| | | sp000508865 | | | |
| 2 | Flavonifractor | Pseudoflavinofractor | | X | 2 |
| | plautii | capillolus; | | | |
| | | Lachnospiraceae | | | |
| | | bacterium; Clostridium | | | |
| | | orbiscindens; | | | |
| | | Flavonifractor | | | |
| | | sp000508885 | | | |
| 3 | Clostridium | Clostridium | X | X | 3 |
| | hathewayi | saccharolyticum; | | | |
| | | Hungatella hathewayi; | | | |
| | | Hungatella effluvii | | | |
| 4 | Blautia | Lachnospiraceae | | X | 4 |
| | coccoides | bacterium; | | | |
| | | Blautia producta | | | |
| 5 | Clostridium | Lachnoclostridium | X | X | 5 |
| | bolteae | bolteae; | | | |
| | | Enterocloster bolteae | | | |
| 6 | Clostridium | Anaerostipes caccae; | X | X | 6 |
| | indolis | Anaerostipes | | | |
| | | sp000508985 | | | |
| 7 | Anaerotruncus | | X | X | 7 |
| | colihominis | | | | |
| 8 | Ruminococcus | Lachnospiraceae | X | X | 8 |
| | sp. | bacterium; | | | |
| | | Sellimonas intestinales; | | | |
| | | Eubacterium fissicatena; | | | |
| | | Drancourtella | | | |
| | | massiliensis; | | | |
| | | Ruminococcus torques; | | | |
| | | Sellimonas intestinalis | | | |
| 9 | Clostridium | Lachnoclostridium | X | X | 9 |
| | lavalense | lavalense; | | | |
| | | Clostridium | | | |
| | | asparagiforme; | | | |
| | | Lachnoclostridium | | | |
| | | asparagiforme; | | | |
| | | Enterocloster | | | |
| | | asparagiforme | | | |
| 10 | Clostridium | Lachnoclostridium | X | X | 10 |
| | symbiosum | symbiosum; | | | |
| | | Clostridium_Q | | | |
| | | symbiosum | | | |
| 11 | Clostridium | Erysipelatoclostridium | | X | 11 |
| | ramosum | ramosum | | | |
| 12 | Eubacterium | Eubacterium fissicatena; | X | X | 12 |
| | contortum | Ruminococcus | | | |
| | | fissicatena; | | | |
| | | Faecalicatena contorta; | | | |
| | | Muricomes | | | |
| | | sp000509105; | | | |
| | | Faecalicatena | | | |
| | | sp000509105 | | | |
| 13 | Clostridium | Lachnospiraceae | | X | 13 |
| | scindens | bacterium; | | | |
| | | Dorea scindens; Dorea | | | |
| | | sp000509125 | | | |
| 14 | Lachnospiraceae | Eisenbergiella | X | X | 14 |
| | bacterium | massiliensis; | | | |
| | | Eisenbergiella tayi | | | |
| 15 | Clostridium sp. | Clostridium aldenense; | X | X | 15 |
| | | Clostridium symbiosum; | | | |
| | | Lachnoclostridium | | | |
| | | symbiosum; | | | |
| | | Enterocloster | | | |
| | | sp000155435; | | | |
| | | Lachnoclostridium | | | |
| | | pacaense | | | |
| 16 | Lachnospiraceae | Eubacterium | X | X | 16 |
| | bacterium | fissicatena; | | | |
| | | Ruminococcus | | | |
| | | fissicatena; | | | |
| | | Faecalicatena contorta; | | | |
| | | Eubacterium contortum; | | | |
| | | Eisenbergiella tayi | | | |

In some embodiments, one or more of the bacterial strains are human-derived bacteria, meaning the one or more bacterial strains were obtained from or identified from a human or a sample therefrom (e.g., a human donor). In some embodiments, all of the bacterial strains are human-derived bacteria. In some embodiments, the bacterial strains are derived from more than one human donor.

The bacterial strains used in the pharmaceutical compositions provided herein generally are isolated from the microbiome of healthy individuals. In some embodiments, the pharmaceutical compositions include strains originating from a single individual. In some embodiments, the pharmaceutical compositions include strains originating from multiple individuals. In some embodiments, the pharmaceutical compositions are obtained from multiple individuals, isolated, and grown up individually. The bacterial compositions that are grown up individually may subsequently be combined to provide the pharmaceutical compositions of the disclosure. It should be appreciated that the origin of the bacterial strains of the pharmaceutical compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments, the pharmaceutical composition includes one or more anaerobic bacteria. In some embodiments, the pharmaceutical composition includes only anaerobic bacteria. In some embodiments, the pharmaceutical composition includes one or more facultative anaerobic bacteria. In some embodiments, the pharmaceutical composition includes only facultative anaerobic bacteria. In some embodiments, the pharmaceutical composition includes one or more obligate anaerobic bacteria. In some embodiments, the pharmaceutical composition includes only obligate anaerobic bacteria.

In some embodiments, at least one (e.g., 1, 2, 3, 4, 5, 6, 7, 8, 9, 10, 11, 12, 13, 14, 15, or 16) of the bacterial strains in the pharmaceutical composition is a spore former. In some embodiments, at least one (e.g., 1, 2, 3, 4, 5, 6, 7, 8, 9, 10, 11, 12, 13, 14, 15, or 16) of the bacterial strains in the pharmaceutical composition is in spore form. In some embodiments, at least one (e.g., 1, 2, 3, 4, 5, 6, 7, 8, 9, 10, 11, 12, 13, 14, 15, or 16) of the bacterial strains in the pharmaceutical composition is a non-spore former. In some embodiments, at least one (e.g., 1, 2, 3, 4, 5, 6, 7, 8, 9, 10, 11, 12, 13, 14, 15, or 16) of the bacterial strains in the pharmaceutical composition is in vegetative form. As discussed above, spore forming bacteria can also be in vegetative form. In some embodiments, at least one (e.g., 1, 2, 3, 4, 5, 6, 7, 8, 9, 10, 11, 12, 13, 14, 15, or 16) of the bacterial strains in the pharmaceutical composition is in spore form and at least one (e.g., 1, 2, 3, 4, 5, 6, 7, 8, 9, 10, 11, 12, 13, 14, 15, or 16) of the bacterial strains in the pharmaceutical composition is in vegetative form. In some embodiments, at least one bacterial strain that is considered able to form spores (i.e., a spore-former) but is present in the composition in vegetative form. In some embodiments, at least one bacterial strain that is considered able to form spores is present in the pharmaceutical composition both in spore form and in vegetative form. In some embodiments, each of the bacterial strains are in vegetative form.

It is envisioned that the bacterial strains of the pharmaceutical compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the live bacterial products provided herein, in some embodiments, the bacterial strains are purified. In any of the live bacterial products provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals. As used herein, the term “isolated” in the bacteria refers to bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected (e.g., below the level of detection). As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that are grown under appropriate conditions for bacterial replication can subsequently be isolated/purified from the culture in which they are grown.

Aspects of the present disclosure relate to methods for colonizing the microbiome of a subject comprising administering a therapeutically effective amount of a pharmaceutical composition comprising two or more purified bacterial strains. In some embodiments, the method further comprises administering one or more additional doses or amounts of the pharmaceutical compositions described herein. In some embodiments, the method further comprises administering an antibiotic to the subject prior to administration of the pharmaceutical compositions. In some embodiments, the method further comprises administering vancomycin to the subject prior to administration of any of the pharmaceutical compositions described herein.

In some embodiments, the two or more of the bacterial strains of the pharmaceutical compositions provided herein colonize or recolonize the gastrointestinal tract or parts thereof (e.g., the colon or the cecum) of the subject. Such colonization may also be referred to as grafting or engraftment. In some embodiments, two or more of the bacterial strains of the compositions recolonize the intestinal tract (e.g., the colon or the cecum) of the subject after the naturally present microbiome has been partially or completely removed, e.g., due to administration of an antibiotic. In some embodiments, the two or more of the bacterial strains of the compositions recolonize the intestinal tract (e.g., the colon or the cecum) of the subject after the naturally present microbiome has been partially or completely removed by antibiotic (e.g., vancomycin) treatment. In some embodiments, the two or more of the bacterial strains of the compositions colonize a dysbiotic gastrointestinal tract (e.g., a gastrointestinal tract that has undergone antibiotic treatment). In some embodiments, all of the bacterial strains of the composition colonize the gastrointestinal tract. In some embodiments, at least one (e.g., 1, 2, 3, 4, 5, 6, 7, 8, 9, 10, 11, 12, 13, 14, 15, or 16) of the bacterial strains of the compositions colonize the gastrointestinal tract. In some embodiments, all of the bacterial strains of the compositions colonize a dysbiotic gastrointestinal tract. In some embodiments, multiple doses of the bacterial compositions are administered to allow for all of the bacterial strains of the composition colonize the gastrointestinal tract. In some embodiments, multiple doses of the bacterial compositions are administered to allow for all of the bacterial strains of the compositions colonize a dysbiotic gastrointestinal tract. In some embodiments, colonization of the microbiome using the compositions and methods provide for an increase in the abundance of bacterial species beneficial to the microbiome.

In some embodiments, colonization of a microbiome with the bacterial strains of the compositions described herein results in a healthy microbiome. As used herein, a “healthy microbiome,” refers to a microbiome from a subject who does not have overt disease (e.g., a healthy subject). Although the microbial composition of healthy microbiomes can vary widely, several trends have emerged which characterize healthy microbiomes. For example, the gastrointestinal microbiome may perform a number of metabolic and/or other molecular functions, including the metabolism of carbohydrates, lipids, and other nutrients which are performed by healthy microbiomes, regardless of the specific species composition. In some instances, the metabolic and molecular functions carried out by a healthy microbiome cannot be performed by the host subject, resulting in a symbiotic host-microbial relationship. Additionally, healthy microbiomes tend to be resilient to external (e.g., dietary or pharmaceutical) and/or internal (e.g., age, disease-state, stress, inflammation) changes in the subject. The resilience of a healthy microbiome can also be characterized by the ability and the rate at which a healthy state is restored after occurrence of a perturbation. Alternatively, or in addition, a healthy microbiome may be characterized by a high (e.g., greater than 75%) relative abundance of bacterial species from the phylum Firmicutes and genus Bacteroides relative to species from the phylum Proteobacteria.

In some embodiments, administration of the pharmaceutical compositions described herein results in a decrease in the abundance of microorganisms associated with inflammation. In some embodiments, administration of the pharmaceutical compositions described herein results in a decrease in the abundance of one or more microorganisms associated with inflammation. As used, herein, the term “microorganisms associated with inflammation” refers to microorganisms that induce pro-inflammatory responses upon colonization or infection of a subject. Microorganisms associated with inflammation are described for instance in Zechner: “Inflammatory disease caused by intestinal pathobionts,” Current Opinion in Microbiology (2017) 35: 64-69; and in numerous publications describing the role of the microorganisms in IBD (See eg., Hoffmann et al., ISME J. 2016 February; 10(2): 460-477.) In some embodiments, the microorganisms associated with inflammation induce acute inflammation characterized, for example, by the presence of pro-inflammatory cytokines and/or infiltration of inflammatory immune cells to the site of colonization or infection. In some embodiments, the microorganisms associated with inflammation induce chronic inflammation. In some embodiments, the microorganisms associated with inflammation are Proteobacteria. In some embodiments, decreasing dysbiosis results in a decrease in the abundance of one or more bacterial species that are associated with inflammation. In some embodiments, decreasing dysbiosis results in a decrease in the abundance overall of bacterial species that are associated with inflammation. In some embodiments, the abundance of bacterial species that are associated with inflammation (e.g., Proteobacteria) in the subject prior to the administration was increased because of treatment with an antibiotic.

In some embodiments, administration of the pharmaceutical compositions described herein results in a decrease in the abundance of Proteobacteria. In some embodiments, administration of the pharmaceutical compositions described herein results in a decrease in the abundance of one or more bacterial strain that belongs to the phylum Proteobacteria. Proteobacteria is a phylum of Gram-negative bacteria that includes a number of pathogens, such as Escherichia coli, Klebsiella sp., Salmonella sp., Campylobacter sp., and Pseudomonas sp. In some embodiments, decreasing dysbiosis results in a decrease in the abundance of one or more bacterial species belonging to the phylum Proteobacteria. In some embodiments, decreasing dysbiosis results in a decrease in the abundance overall of bacterial species belonging to the phylum Proteobacteria.

In some embodiments, administration of the pharmaceutical compositions described herein results in a decrease in abundance of Proteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold. 104-fold, 105-fold or more, as compared to the abundance of Proteobacteria in the subject (or microbiome thereof) prior to administering the pharmaceutical compositions. In some embodiments, the abundance of Proteobacteria in the subject prior to the administration of the pharmaceutical compositions was higher because of treatment with an antibiotic. In some embodiments, the abundance of Proteobacteria in the subject prior to the administration of the pharmaceutical compositions was higher because of treatment with an antibiotic. In some embodiments, administration of the pharmaceutical compositions described herein results in a decrease in abundance of Proteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the abundance of Proteobacteria in another subject (e.g., a reference subject) (or microbiome thereof) who did not receive the pharmaceutical compositions.

In some embodiments, administration of the pharmaceutical compositions described herein results in a decrease in abundance of Proteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Proteobacteria in the subject (or microbiome thereof) prior to administering the pharmaceutical compositions. In some embodiments, administration of the pharmaceutical compositions described herein results in decrease in the abundance of Proteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Proteobacteria in a subject (e.g., a reference subject) (or microbiome thereof) who did not receive the compositions.

The abundance of bacteria, including the abundance of specific species or strains of bacteria and abundance of a population of bacteria (e.g., bacteria belonging to a particular phylum) may be assessed using any method known in the art. In general, the abundance of bacteria may be assessed directly or indirectly. Examples of methods for directly assessing the abundance of bacteria in a sample (e.g., a microbiome or sample thereof) include identifying and quantifying bacterial strains in a fecal sample from the subject. Examples of methods for indirectly assessing the abundance of bacteria in a sample (e.g., a microbiome or sample thereof) include sequencing of nucleic acid samples (e.g., 16S rRNA gene for a given bacterial species or other bacterial genes) obtained from a fecal or biopsy sample, and detecting and quantifying metabolites associated with specific bacteria (e.g., phospholipid fatty acid metabolism, microbial biomass carbon analysis) in a fecal sample from the subject.

In some embodiments, the one or more bacterial strains of the pharmaceutical compositions colonize the microbiome because they can “outgrow” other bacterial strains (e.g., pathogens). In some embodiments, the subject has been treated with an antibiotic resulting in a removal of most of the microbiome, providing a “clean slate” environment for both the one or more bacterial strains of compositions and any other bacterial strains (e.g., pathogens, strains associated with inflammation or undesired immune responses). Thus, without being limited to a specific mechanism, if a pathogen and bacterial strains of the compositions provided herein are both present in the intestinal tract (e.g., the colon or the cecum), the bacterial strains of compositions provided herein grow faster (e.g., have a shorter doubling time) than the pathogen, thereby preventing the pathogen from accumulating in the intestinal tract (e.g., the colon or the cecum) and allowing the bacterial strains of the compositions to colonize. In some embodiments, the faster growth results because the bacterial strains of the compositions provided herein are better at grafting in the intestinal tract (e.g., the colon or the cecum). In some embodiments, the faster growth results because the bacterial strains of the compositions provided herein are better at metabolizing nutrients present in the intestinal tract (e.g., the colon or the cecum). In some embodiments, the compositions of bacterial strains provided herein prevent or inhibit production of bacterial toxins by an infectious agent, or prevent or inhibit the cytopathic or cytotoxic effects of such toxins. In some embodiments, the bacterial strains of the compositions provided herein can treat pathogenic infections, because of the synergy between the bacterial strains. Thus, without being limiting, in some embodiments, the combination of the bacterial strains of the compositions provided herein act synergistically because the combination of the strains is particularly well-suited to use nutrients in the intestinal tract (e.g., the colon or the cecum), or instance through metabolic interactions, and/or because the combination is superior in grafting (e.g., by providing a favorable microenvironment). In some embodiments, the bacterial strains of the compositions described herein are able to colonize specific niches in the intestinal tract (e.g., the colon or the cecum). In some embodiments, the bacterial strains of the compositions described herein are able to colonize specific niches in the intestinal tract (e.g., the colon or the cecum) that became available after antibiotic treatment.

In some embodiments, the pharmaceutical composition contains eleven bacterial strains and at least two bacterial strains colonize the microbiome of the subject. In some embodiments, the pharmaceutical composition contains eleven bacterial strains and at least three bacterial strains colonize the microbiome of the subject. In some embodiments, the pharmaceutical composition contains eleven bacterial strains and at least four bacterial strains colonize the microbiome of the subject. In some embodiments, the pharmaceutical composition contains eleven bacterial strains and at least five bacterial strains colonize the microbiome of the subject. In some embodiments, the pharmaceutical composition contains eleven bacterial strains and at least six bacterial strains colonize the microbiome of the subject. In some embodiments, the pharmaceutical composition contains eleven bacterial strains and at least seven bacterial strains colonize the microbiome of the subject. In some embodiments, the pharmaceutical composition contains eleven bacterial strains and at least eight bacterial strains colonize the microbiome of the subject. In some embodiments, the pharmaceutical composition contains eleven bacterial strains and at least nine bacterial strains colonize the microbiome of the subject. In some embodiments, the pharmaceutical composition contains eleven bacterial strains and at least ten bacterial strains colonize the microbiome of the subject. In some embodiments, the pharmaceutical composition contains eleven bacterial strains and each of the eleven bacterial strains colonize the microbiome of the subject.

In some embodiments, the pharmaceutical composition contains sixteen bacterial strains and at least two bacterial strains colonize the microbiome of the subject. In some embodiments, the pharmaceutical composition contains sixteen bacterial strains and at least three bacterial strains colonize the microbiome of the subject. In some embodiments, the pharmaceutical composition contains sixteen bacterial strains and at least four bacterial strains colonize the microbiome of the subject. In some embodiments, the pharmaceutical composition contains sixteen bacterial strains and at least five bacterial strains colonize the microbiome of the subject. In some embodiments, the pharmaceutical composition contains sixteen bacterial strains and at least six bacterial strains colonize the microbiome of the subject. In some embodiments, the pharmaceutical composition contains sixteen bacterial strains and at least seven bacterial strains colonize the microbiome of the subject. In some embodiments, the pharmaceutical composition contains sixteen bacterial strains and at least eight bacterial strains colonize the microbiome of the subject. In some embodiments, the pharmaceutical composition contains sixteen bacterial strains and at least nine bacterial strains colonize the microbiome of the subject. In some embodiments, the pharmaceutical composition contains sixteen bacterial strains and at least ten bacterial strains colonize the microbiome of the subject. In some embodiments, the pharmaceutical composition contains sixteen bacterial strains and at least eleven bacterial strains colonize the microbiome of the subject. In some embodiments, the pharmaceutical composition contains sixteen bacterial strains and at least twelve bacterial strains colonize the microbiome of the subject. In some embodiments, the pharmaceutical composition contains sixteen bacterial strains and at least thirteen bacterial strains colonize the microbiome of the subject. In some embodiments, the pharmaceutical composition contains sixteen bacterial strains and at least fourteen bacterial strains colonize the microbiome of the subject. In some embodiments, the pharmaceutical composition contains sixteen bacterial strains and at least fifteen bacterial strains colonize the microbiome of the subject. In some embodiments, the pharmaceutical composition contains sixteen bacterial strains and each of the sixteen bacterial strains colonize the microbiome of the subject.

The extent of colonization of any of the bacterial strains may be determined, for example by detecting the presence of one or more bacterial strains and/or by quantifying the abundance of the one or more bacterial strains. In some embodiments, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the bacterial strains of the pharmaceutical compositions colonize the microbiome of the subject. In some embodiments, at least 25% of the bacterial strains of the pharmaceutical compositions colonize the microbiome of the subject. In some embodiments, at least 50% of the bacterial strains of the pharmaceutical compositions colonize the microbiome of the subject. In some embodiments, 100% of the bacterial strains of the pharmaceutical compositions colonize the microbiome of the subject. In some embodiments, the percentage of the bacterial strains of the pharmaceutical compositions that colonize the microbiome of the subject is increased by administering additional doses of the pharmaceutical compositions.

It should be appreciated that in one aspect, the methods provided herein provide better colonization (e.g., engraftment of a higher number/percentage and/or higher abundance) of the bacterial strains of the pharmaceutical compositions when compared to the colonization of the same pharmaceutical compositions administered according to different methods. Thus, in one aspect, the colonization is better because the pharmaceutical compositions are administered according to the specific dose regimens, amounts, and/or antibiotic treatment regimens provided herein.

The extent of colonization of the microbiome by the one or more bacterial strains of the pharmaceutical compositions described herein may be based on the relative abundance of the bacterial strains of the pharmaceutical compositions in the microbiome. For example, in some embodiments, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%. 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the bacterial strains of the microbiome of the subject are the bacterial strains of the pharmaceutical compositions. In some embodiments, at least 25% of the bacterial strains detected in the microbiome of the subject are the bacterial strains of the pharmaceutical compositions. In some embodiments, at least 50% of the bacterial strains detected in the microbiome of the subject are the bacterial strains of the pharmaceutical compositions. In some embodiments, the percentage of the bacterial strains of the bacterial compositions in the microbiome of the subject is increased by administering additional doses of the pharmaceutical compositions. In some embodiments, the percentage of the bacterial strains of the bacterial compositions in the microbiome of the subject is increased by administering an antibiotic (e.g., vancomycin) to the subject prior to administration of the pharmaceutical compositions.

In some embodiments, the pharmaceutical compositions described herein result in durable colonization by two or more bacterial strains in the composition. In some embodiments, two or more bacterial strains of the pharmaceutical compositions described herein are detected in the microbiome of the subject for an extended period of time. In some embodiments, two or more bacterial strains of the pharmaceutical compositions described herein colonize the microbiome of the subject for an extended period of time. In some embodiments, two or more bacterial strains of the pharmaceutical compositions described herein are detected in the microbiome of the subject at least one week, two weeks, three weeks, four weeks, five weeks, or six weeks, seven weeks, eight weeks, nine weeks, ten weeks, eleven weeks, twelve weeks, three months, four months, five months, six months, seven months, eight months, nine months, ten months, eleven months, or one year following administration of the pharmaceutical composition.

It should be appreciated that in one aspect, the methods provided herein provide better colonization (e.g., engraftment of a higher number/percentage and/or higher abundance) and more durable colonization of the bacterial strains of the pharmaceutical compositions when compared to the colonization of the same pharmaceutical compositions administered according to different methods. Thus, in one aspect, the colonization is better and more durable because the pharmaceutical compositions are administered according to the specific dose regimens, amounts and/or antibiotic treatment regimens provided herein.

It should be appreciated that in one aspect, the methods provided herein provide more durable colonization of the bacterial strains of the pharmaceutical compositions when compared to the colonization of the same pharmaceutical compositions administered according to different methods. Thus, in one aspect, the colonization is more durable because the pharmaceutical compositions are administered according to the specific dose regimens, amounts, and/or antibiotic treatment regimens provided herein.

The methods described herein involve administering any of the pharmaceutical compositions described herein to a subject in need thereof. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human. In some embodiments, the human subject is a neonatal subject, a pediatric subject, an adolescent subject, an adult subject, or a geriatric subject.

Any of the compositions described herein may be administered to a subject in a therapeutically effective amount or a dose of a therapeutically effective amount. In some embodiments, the therapeutically effective amount is an amount sufficient to treat or prevent a disease or disorder. The terms “treat” or “treatment” refer to reducing or alleviating one or more of the symptoms associated with a disease or disorder (e.g., inflammatory bowel disease (IBD), graft versus host disease (GvHD)).

In some embodiments, a therapeutically effective amount of any of the compositions described herein may be administered to prevent a disease or disorder (e.g., inflammatory bowel disease (IBD), graft versus host disease (GvHD)). The terms “prevent” or “prevention” encompass prophylactic administration and may reduce the incidence or likelihood of the occurrence, for example, of the disease or disorder. In some embodiments, the composition reduces the incidence or likelihood of the occurrence of perturbation of the microbiome. In some embodiments, the composition reduces the incidence or likelihood of the occurrence of a disease or disorder (e.g., inflammatory bowel disease (IBD), graft versus host disease (GvHD)). In some embodiments, the composition reduces the risk of a disease or disorder (e.g., inflammatory bowel disease (IBD), graft versus host disease (GvHD)). For instance, in some embodiments, administration of any of the compositions provided herein results in a healthy microbiome in the subject that provides an effect in a subject that reduces the incidence or likelihood of a disease or disorder. In some embodiments, administration of any of the compositions provided herein results in a reduction or alleviation of one or more symptom associated with disease or disorder.

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein, including but not limited to delay the manifestation, arrest the progression, relieve or alleviate at least one symptom of a disease or disorder (e.g., inflammatory bowel disease (IBD), graft versus host disease (GvHD)), reduce the incidence or likelihood of the occurrence of a disease or disorder (e.g., inflammatory bowel disease (IBD), graft versus host disease (GvHD), reduces the risk of a disease or disorder (e.g., inflammatory bowel disease (IBD), graft versus host disease (GvHD), and/or colonization of the microbiome by bacterial strains of the pharmaceutical compositions described herein. In some embodiments, the therapeutically effective amount is an amount sufficient to colonize the microbiome of a subject. In some embodiments, the therapeutically effective amount is an amount sufficient to restore the microbiome following a state of disease. In some embodiments, the therapeutically effective amount is an amount sufficient to reduce or eliminate at least one symptom associated with IBD, or reduce the severity of at least one symptom associated with IBD. In some embodiments, the therapeutically effective amount is an amount sufficient to reduce or eliminate at least one symptom associated with GvHD, or reduce the severity of at least one symptom associated with GvHD.

It should be appreciated that the term “effective amount,” in reference to a composition comprising bacterial strains, may be expressed as the number of bacteria or CFUs to be administered. It should further be appreciated that the bacteria can multiply once administered. Thus, administration of even a relatively small amount of bacteria may have therapeutic effects.

Some aspects of the present disclosure provide compositions and methods for treating and/or preventing inflammatory bowel disease (IBD) comprising administering any of the compositions described herein, for example, to a subject having, suspected of having, or at risk of having or developing IBD. IBD is generally characterized by mucosal immune dysregulation and an altered microbiome (dysbiosis). The gut microbiome of patients with Inflammatory Bowel Disease (IBD) is characterized by decreased α-diversity, decreased levels of Clostridium clusters IV and XIVa, and increased levels of Enterobacteriacea and Ruminococcus gnavus.

In some embodiments, the IBD is ulcerative colitis or Crohn's disease. In some embodiments, the IBD is ulcerative colitis, including chronic or recurring ulcerative colitis. In some embodiments, the IBD is Crohn's disease, such as ilcal Crohn's disease or colonic Crohn's disease. The inflammation of the gastrointestinal tract associated with IBD may lead to the formation of ulcers, fistulas, strictures, and/or scarring of the intestinal wall.

Symptoms associated with IBD, such as ulcerative colitis and Crohn's disease may include urgent and frequent bowel movements, irregular bowel movements, persistent diarrhea, incontinence, rectal bleeding and pain, bloody stool, abdominal pain, and cramping, constipation, loss of appetite, unexpected weight loss, fatigue, skin rashes, and arthritis.

In some embodiments, a therapeutically effective amount of any of the compositions described herein is administered to a subject having or suspected of having IBD such that one or more symptoms of IBD are improved (e.g., as compared to the one or more symptom prior to the administration). In some embodiments, a therapeutically effective amount of any of the compositions described herein is administered to a subject at risk of having IBD (e.g., a subject who has recently undergone a transplantation or will undergo a transplantation) such that the subject does not experience one or more symptoms of IBD or the subject experiences one or more symptoms of IBD to a lesser degree (e.g., as compared to subjects that do not receive the compositions).

In some embodiments, the subject is at risk of having or developing IBD, or at risk of having a recurrence of IBD. Non-limiting examples of risk factors for IBD include smoking, family history of IBD, Jewish ancestry, prior appendectomy, diet, breastfeeding, use of nonsteroidal anti-inflammatory drugs (NSAIDs), use of oral contraceptives, and use of antibiotics. See, e.g., Molodecky and Kaplan. Gastroenterol Hepatol (NY). 2010. 6(5):339-346.

In some embodiments, the risk of a subject developing IBD is evaluated, and a bacterial composition is administered to the subject (in one or more doses) if they are at risk of developing IBD to colonize the microbiome of the subject with the strains of the pharmaceutical compositions described herein and reduce the risk of developing IBD. In some embodiments, evaluating the risk of a subject developing IBD comprises determining whether the subject has one or more risk factors for IBD. In some embodiments, the subject has a history of smoking. In some embodiments, the subject has one or more blood relatives that have or previously had IBD. In some embodiments, the subject is of Jewish ancestry. In some embodiments, the subject has previously had an appendectomy. In some embodiments, the subject uses or has used oral contraceptives. In some embodiments, the subject has used or is using antibiotics.

Any of the methods described herein may further involve administering to the subject one or more additional therapeutics, such as one or more additional therapeutics of the treatment of IBD. Conventional therapy for the treatment of IBD typically involves a first line of therapy involving an aminosalicylate agent (5-ASA) and/or an antibiotic. For more moderate to severe instances of IBD or if the first line of therapy is not effective, the subject may be administered a corticosteroid, such as prednisone or budesonide, followed by immunomodulators, such as 6-mercaptopurine (6-MP), or methotrexate (MTX) for even more severe cases. In the most severe cases or instances in which other therapies have not been effective, the subject may receive a biologic agent (e.g., anti-TNF agents (e.g., infliximab, adalimumab, golimumab, certolizumab, pegol), anti-integrin agents (e.g., natalizumab, vedolizumab) or undergo a surgical procedure (e.g., partial colectomy), JAK inhibitors (e.g., tofacitinib, filgotinib, ruxollitinib), S1P modulators (e.g., ozanimod), IL-12/IL-23 blockers (e.g., ustekinumab).

In some embodiments, any of the pharmaceutical compositions described herein may be administered in combination with an additional therapeutic for IBD, such as an antibiotic and/or 5-ASA.

Examples of aminosalicylate agents (5-ASA) include, without limitation, 4-aminosalicylic acid (4-ASA), balsalazide, olsalazine, sulfasalazine, and mesalazine (5-aminosalicylic acid, or 5-ASA). In some embodiments, the pharmaceutical composition is administered to the subject immediately after a steroid or aminosalicylate agent.

The pharmaceutical compositions described herein have been found to be safe and well tolerated when administered to subject. Therefore, the pharmaceutical compositions described herein may be administered to improve the efficacy of additional therapeutic for IBD without introducing additional safety or tolerability risks. In some embodiments, any of the pharmaceutical compositions described herein may be administered following administration of an additional therapeutic for IBD, such as an antibiotic and/or 5-ASA. In some embodiments, the pharmaceutical compositions described herein are administered to the subject as an alternative to administering a steroid. Although steroid treatment may be used to treat or reduce symptoms associated with IBD, steroid administration may be associated with undesired side effects, such as immunosuppression. In such instances, the pharmaceutical composition may be referred to as a “steroid-sparing” agent and be administered in effort to avoid administration of a steroid (e.g., prednisone, budesonide), or more significant therapeutic intervention.

In some embodiments, any of the pharmaceutical compositions described herein may be administered in combination with a steroid, such as prednisone, budesonide. In some embodiments, any of the pharmaceutical compositions described herein may be administered following administration of steroid, such as prednisone or budesonide. In some embodiments, the pharmaceutical composition is administered to the subject immediately after a steroid.

Some aspects of the present disclosure provide compositions and methods for treating and/or preventing graft versus host disease (GvHD) comprising administering any of the compositions described herein, for example to a subject having, suspected of having, or at risk of having GvHD. In some embodiments, the methods involve administering a therapeutically effective amount of any of the compositions described herein to colonize the microbiome of the subject.

In general, a subject may experience or be at risk of experiencing GvHD after receiving a transplant, e.g., a bone marrow transplant, stem cell transplant. GvHD may occur when cells from the donor recognize histocompatibility antigens of the transplant recipient as foreign, leading to inflammatory responses, including T cell activation and cytokine production, targeting tissues of the subject and may lead to multi-organ dysfunction and destruction.

In some embodiments, a therapeutically effective amount of any of the compositions described herein is administered to a subject having or suspected of having GvHD such that one or more symptoms of GvHD are improved (e.g., as compared to the one or more symptom prior to the administration). In some embodiments, a therapeutically effective amount of any of the compositions described herein is administered to a subject at risk of having GvHD (e.g., a subject who has recently undergone a transplantation or will undergo a transplantation) such that the subject does not experience one or more symptoms of GvHD or the subject experiences one or more symptoms of GvHD to a lesser degree (e.g., as compared to subjects that do not receive the compositions).

In some embodiments, any of the pharmaceutical compositions described herein may be administered in combination with one or more additional therapeutic for treating GvHD. Examples of therapeutics used to treat GvHD include, without limitation, ethotrexate, cyclosporine, tacrolimus, mycophenolate mofetil, sirolimus, corticosteroids (methylprednisolone or prednisone), antithymocyte globulin, alemtuzumab, cyclophosphamide, and ruxolitinib (Jakafi®). In some embodiments, any of the pharmaceutical compositions described herein may be administered following administration of one or more additional therapeutic for treating GvHD. In some embodiments, the pharmaceutical composition is administered to the subject immediately after administration of one or more additional therapeutic for treating GvHD.

Also within the scope of the present disclosure are methods involving determining whether a subject has or is at risk of having IBD or GvHD or in need of administration of any of the pharmaceutical compositions described herein.

In some embodiments, the specific combination of one or more bacterial strains of the compositions described herein provides a synergistic effect that promotes treating and/or preventing IBD and/or reducing the risk and/or occurrence of IBD. In some embodiments, the specific combination of one or more bacterial strains of the compositions described herein provides a synergistic effect that promotes treating and/or preventing GvHD in a subject. In some embodiments, the synergistic effect is provided by the capacity of the combination to metabolize specific nutrients. In some embodiments, the synergistic effect is provided by the capacity of the combination to provide specific metabolites to the environment. Such specific metabolites may suppress growth of the pathogen and/or stimulate growth of non-pathogens.

In some embodiments, the synergistic effect is provided by the capacity of the combination to provide short-chain fatty acids to the environment. In some embodiments, the synergistic effect is provided by the capacity of the combination to provide specific short-chain fatty acids to the environment. In some embodiments, the synergistic effect is provided by the capacity of the combination to produce butyrate. In some embodiments, the synergistic effect is provided by the capacity of the combination to produce acetate. In some embodiments, the synergistic effect is provided by the capacity of the combination to produce lactate. In some embodiments, the synergistic effect is provided by the capacity of the combination to produce propionate. In some embodiments, the synergistic effect is provided by the capacity of the combination to produce succinate. In some embodiments, the synergistic effect is provided by the capacity of the combination to produce multiple metabolites. In some embodiments, the synergistic effect is provided by the capacity of the combination to produce multiple short-chain fatty acids. In some embodiments, the synergistic effect is provided by the capacity of the combination to produce both butyrate and acetate. In some embodiments, the synergistic effect is provided by the capacity of the combination to produce both butyrate and lactate. In some embodiments, the synergistic effect is provided by the capacity of the combination to produce both butyrate and propionate. In some embodiments, the synergistic effect is provided by the capacity of the combination to produce both butyrate and succinate. In some embodiments, the synergistic effect is provided by the capacity of the combination to produce butyrate, acetate, and additional short-chain fatty acids.

In some embodiments, the specific combination of two or more bacterial strains of the compositions provided herein is superior in the use of nutrients and in grafting when compared to other strains (e.g., pathogens), thereby protecting and or restoring the microbiome, for instance through suppressing the growth of the pathogen. In some embodiments, the specific combination of one or more bacterial strains of the compositions provided herein induces an immune response in the subject that promotes colonizing the microbiome of the subject with one of or of the bacterial strains of the pharmaceutical compositions. In some embodiments, the specific combination of one or more bacterial strains of the compositions provided herein induces an immune response in the subject that reduces IBD or risk of IBD. In some embodiments, the specific combination of one or more bacterial strains of the compositions provided herein induces an immune response in the subject that reduces GvHD.

Also within the scope of the present disclosure are compositions, e.g., compositions for administering to a subject, such as pharmaceutical compositions. In some embodiments, the composition comprises any of the bacterial strains described herein.

In one aspect, the disclosure provides pharmaceutical compositions comprising any of the bacterial strains described herein. In some embodiments, the pharmaceutical composition comprises a pharmaceutical acceptable excipient. In some embodiments, the pharmaceutical composition is formulated for oral administration. In some embodiments, the pharmaceutical composition is formulated for rectal administration. In some embodiments, the pharmaceutical composition is formulated for delivery to the intestine. In some embodiments, the pharmaceutical composition is formulated for delivery to the colon.

In some embodiments, the pharmaceutical compositions described herein contain one or more bacterial strains. In some embodiments, the pharmaceutical compositions may be lyophilized. In some embodiments, the pharmaceutical composition is in the form of a capsule. In some embodiments, the pharmaceutical composition further comprises a pH sensitive composition comprising one or more enteric polymers. In some embodiments, the pharmaceutical composition comprises one or more enteric polymers.

In some embodiments, one of or more of the bacterial strains of the pharmaceutical compositions has been spray-dried. The process of spray-drying refers to production of a dry powder from a liquid comprising bacterial compositions. (See e.g., Ledet et al., Spray-Drying of Pharmaceuticals in “Lyophilized Biologics and Vaccines” pages 273-194, Springer). In general, the process involves rapidly drying the bacterial compositions with a hot gas.

Any of the compositions described herein, including the pharmaceutical compositions and food products comprising bacterial strains, the bacterial strains in any form, for example in an aqueous form, such as a solution or a suspension, embedded in a semi-solid form, in a powdered form, or freeze-dried form. In some embodiments, the composition or the bacterial strains are lyophilized. In some embodiments, a subset of the bacterial strains is lyophilized. Methods of lyophilizing compositions, specifically compositions comprising bacteria, are well known in the art. Sec, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (See, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains can be manufactured using fermentation techniques well known in the art. In some embodiments, the bacteria are propagated or manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the live bacterial product may be formulated for administration as a pharmaceutical composition. The term “pharmaceutical composition” as used herein means a product that results from the mixing or combining of at least one active ingredient, such as any of the bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

In some embodiments, the compositions comprising bacterial strains are formulated for oral delivery. In some embodiments, the compositions comprising bacterial strains are formulated for delivery to the intestines (e.g., the small intestine and/or the colon). In some embodiments, the composition comprising bacterial strains may be formulated with an enteric coating that increases the survival of the bacteria through the harsh environment in the stomach. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of polymers and copolymers well known in the art, such as commercially available EUDRAGIT (Evonik Industries). (See e.g., Zhang, AAPS PharmSciTech (2016) 17 (1), 56-67).

The compositions comprising bacterial strains may also be formulated for rectal delivery to the intestine (e.g., the colon). Thus, in some embodiments, compositions comprising bacterial strains may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J Pharm 2015, 487 (1-2): 314-9).

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, a pharmaceutical composition may include an additional component that enables efficient delivery of the compositions of the disclosure to a desired site, such as the gastrointestinal tract (e.g., the colon).

In some embodiments, the pharmaceutical composition includes an adjuvant associated with providing a benefit in the treatment of allergy. In some embodiments, the pharmaceutical composition includes one or more components of an oral immunotherapeutic, an epicutancous immunotherapeutic, or a sublingual immunotherapeutic.

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition comprises bacterial strains and is formulated such that the bacteria, or a portion thereof, remain viable after passage through the stomach of the subject. In some embodiments, the pharmaceutical composition is formulated for rectal administration, e.g., as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions described herein can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., colonization of a microbiome, treatment of IBD, treatment of GvHD) is achieved. In general, effective doses of the compositions described herein, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of a single dose of any of the compositions described herein. In some embodiments, the dosing regimen entails oral administration of multiple doses of any of the compositions described herein. In some embodiments, any of the compositions described herein are administered the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered the subject in multiple doses at a regular interval, such as every day, every 2 days, every 3 days, every 4 days, every 5 days, every 6 days, every week, every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In one aspect, the disclosure provides methods comprising administration of multiple doses of the pharmaceutical compositions. In some embodiments, the disclosure provides methods comprising administration of antibiotic (e.g., vancomycin) followed by multiple doses of the pharmaceutical compositions. In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. In some embodiments, administration of multiple doses of the pharmaceutical compositions described herein provides enhanced recovery of one or more bacterial strains of the pharmaceutical compositions as compared to administration of a single dose of the pharmaceutical composition. In some embodiments, administration of multiple doses of the pharmaceutical compositions described herein provides increased abundance of one or more bacterial strains of the pharmaceutical compositions as compared to administration of a single dose of the pharmaceutical composition. In some embodiments, administration of multiple doses of the pharmaceutical compositions described herein provides an increase in the number of subjects that were colonized with of all of bacterial strains of the pharmaceutical compositions as compared to administration of a single dose of the pharmaceutical composition. In some embodiments, administration of multiple doses of the pharmaceutical compositions described herein provides enhanced durability of colonization with one or more bacterial strains of the pharmaceutical compositions as compared to administration of a single dose of the pharmaceutical composition. In some embodiments, administration of multiple doses of the pharmaceutical compositions described herein provides durable colonization of some or all of the bacterial strains of the pharmaceutical compositions described herein over an extended period of time as compared to administration of a single dose of the pharmaceutical composition. In some embodiments, administration of multiple doses of the pharmaceutical compositions described herein provides durable colonization (e.g., up to 6 months) of one or more bacterial strains of the pharmaceutical compositions as compared to administration of a single dose of the pharmaceutical composition. In some embodiments, administration of multiple doses of the pharmaceutical compositions described herein provides durable colonization (e.g., up to 6 months or longer) of some or all of the bacterial strains of the pharmaceutical compositions as compared to administration of a single dose of the pharmaceutical composition. It should further be appreciated that administration of multiple doses may result in a combination of the results described. Thus, for example, in some embodiments, administration of multiple doses of the pharmaceutical compositions described herein provides enhanced colonization (engraftment) and increased rate of recovery of one or more bacterial strains of the pharmaceutical compositions as compared to administration of a single dose of the pharmaceutical composition.

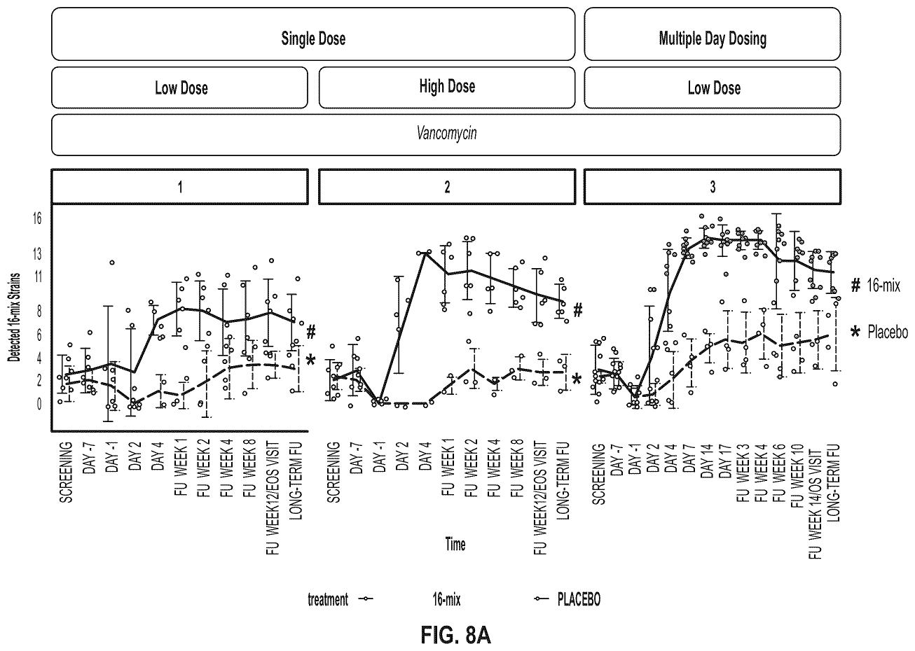

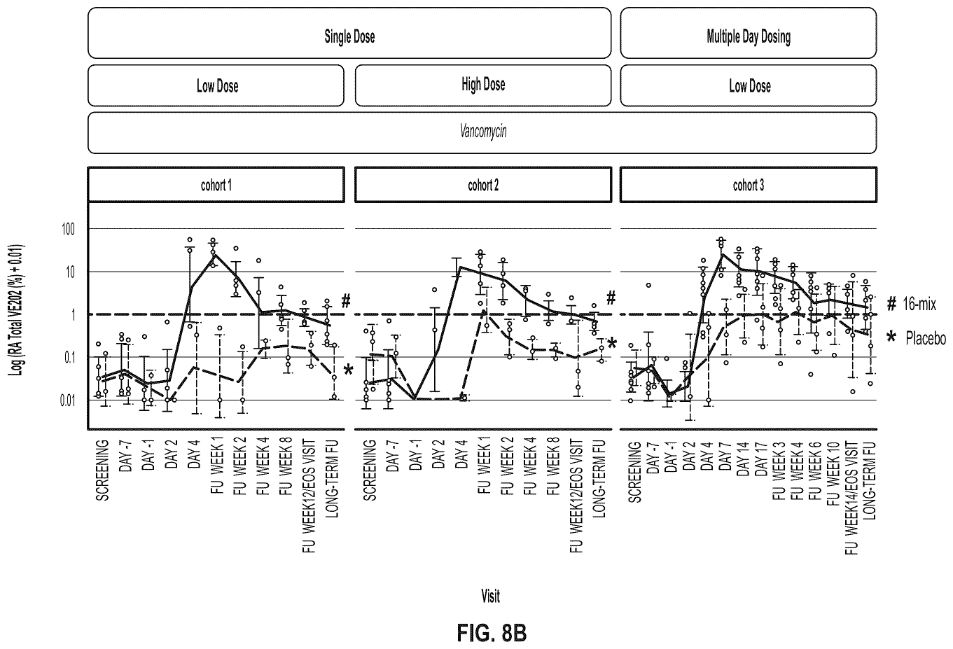

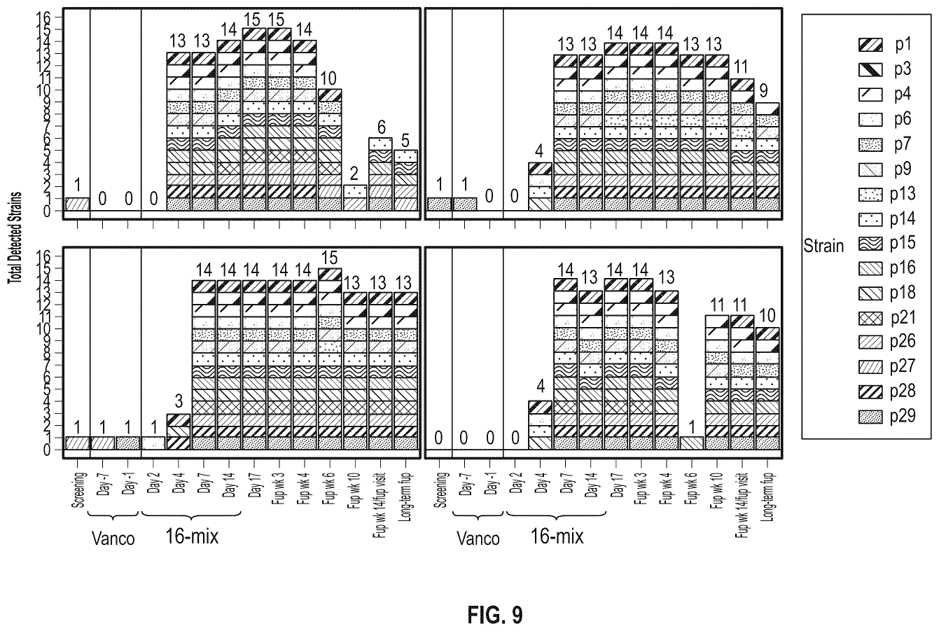

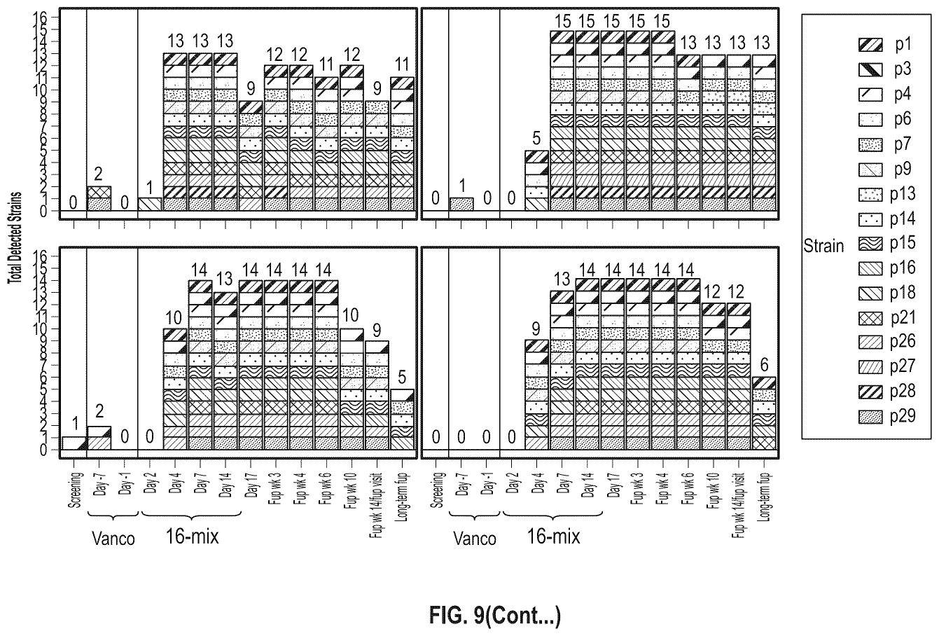

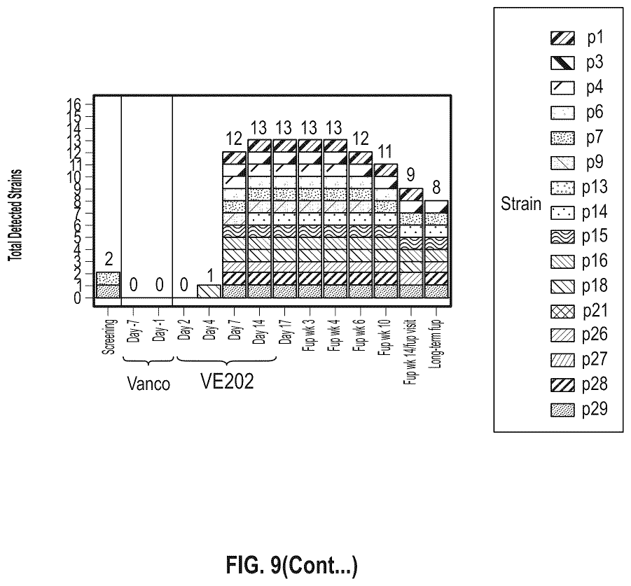

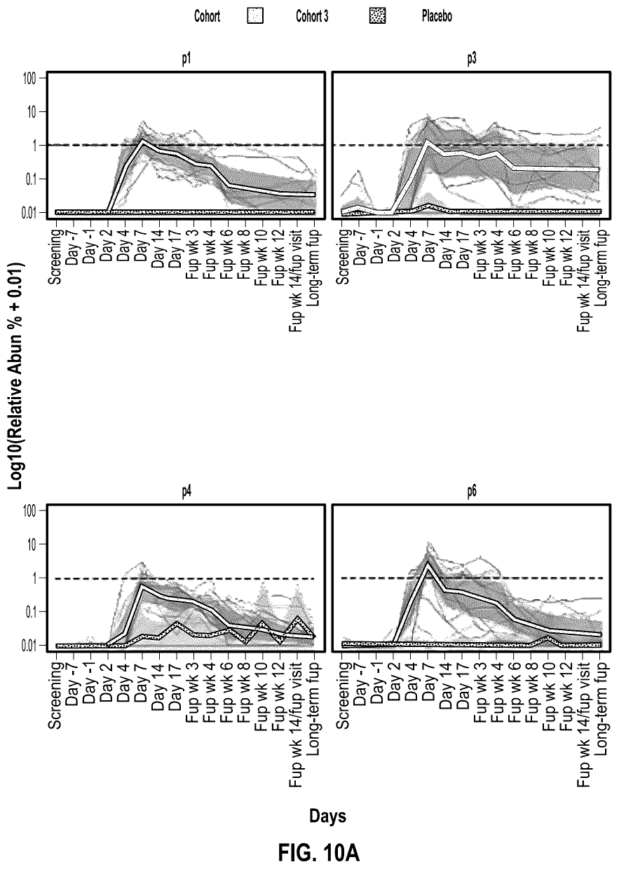

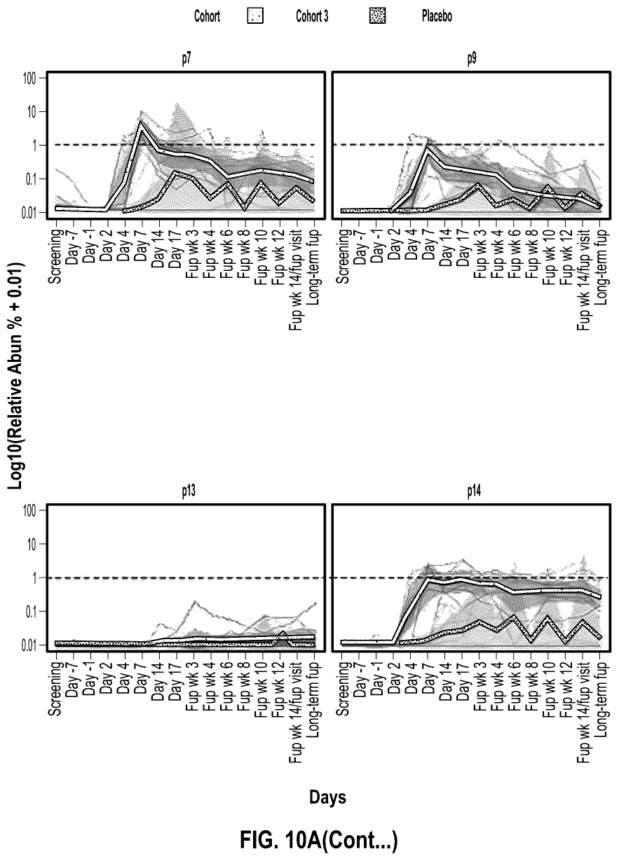

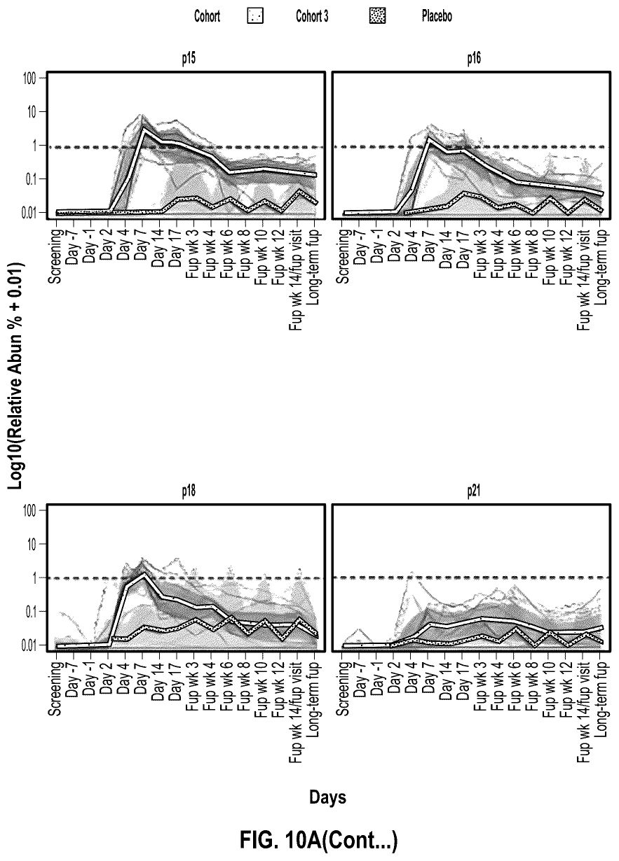

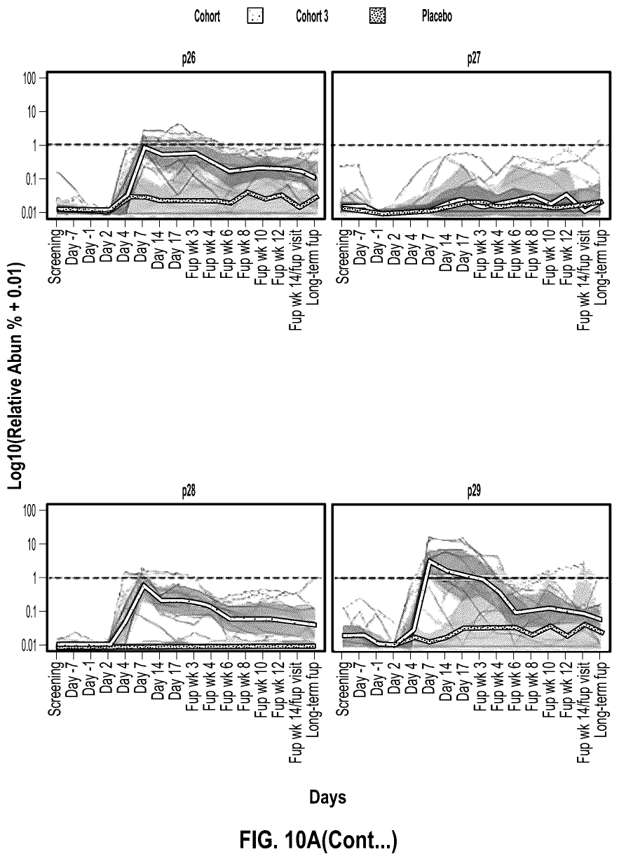

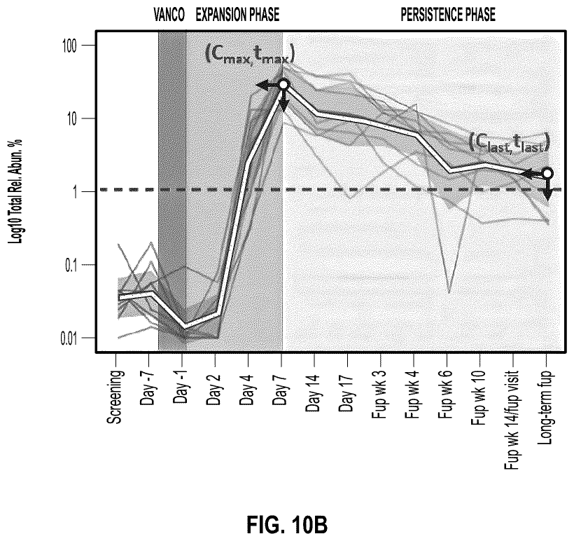

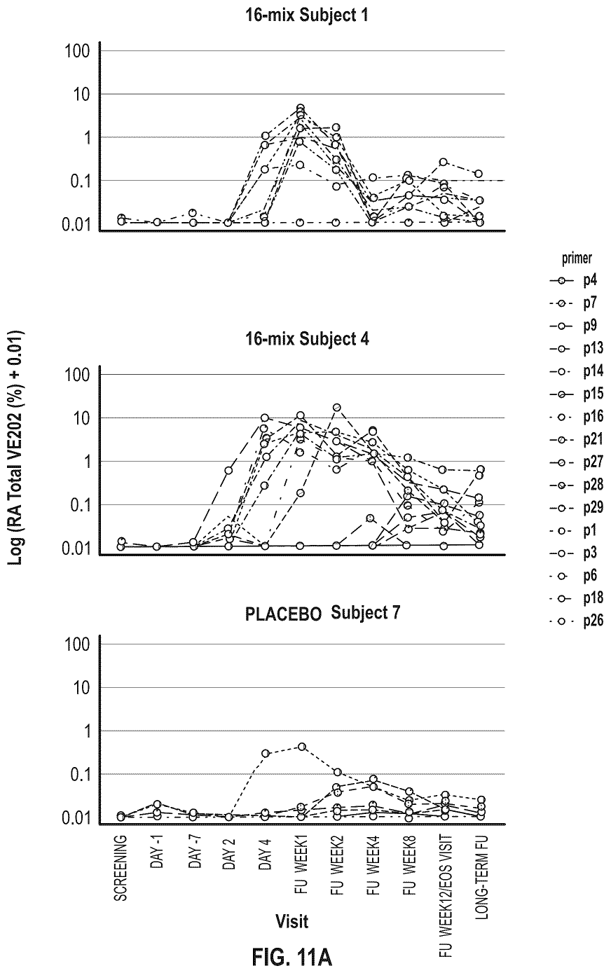

In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. As shown in FIGS. 8A and 8B, administration of multiple doses of the pharmaceutical composition results in enhanced colonization (engraftment) and an increased abundance of each of the bacterial strains of the pharmaceutical composition. In some embodiments, administration of a single dose of the pharmaceutical composition results in the same or a similar level of engraftment (e.g., total bacteria) as administration of multiple doses of the pharmaceutical composition, however the engraftment may be dominated by one bacterial strain or only a subset of the bacterial strains of the pharmaceutical compositions.

Any of the methods described herein may further comprise administering an antibiotic to the subject prior to administration of the pharmaceutical compositions described herein. In some embodiments, the subject was previously administered an antibiotic prior to administration of the pharmaceutical compositions described herein. In some embodiments, administration of any of the pharmaceutical compositions described herein is not preceded by administration of an antibiotic.

In some embodiments, the antibiotic is vancomycin, fidaxomycin or ridinilazole. Non-limiting examples of antibiotics that may be used in any of the methods provided herein include cephalosporin antibiotics cephalexin, cefuroxime, cefadroxil, cefazolin, cephalothin, cefaclor, cefamandole, cefoxitin, cefprozil, ceftobiprole, clindamycin, ceftriaxone, cefotaxime, cefazolin, cefoperazone, cefuroxime, cefmetazole, fluoroquinolone, ciprofloxacin, Levaquin, floxin, tequin, avelox, norflox, tetracycline, minocycline, oxytetracycline, doxycycline, amoxicillin, ampicillin, penicillin V, dicloxacillin, benzylpenicillin, carbenicillin, vancomycin, and methicillin), ertapenem, doripenem, imipenem/cilastatin, meropenem, clavulanate, tazobactam, piperacillin, ceftriaxone, cefotaxime, cefazolin, fluoroquinolone, imipenem, meropenem, metronidazole, fidaxomyxin, or ridinilazole. In some embodiments, any of the methods described herein may further comprise administering vancomycin to the subject prior to administration of the pharmaceutical compositions described herein. In some embodiments, the method does not comprise administering vancomycin to the subject prior to administration of the pharmaceutical compositions described herein. Vancomycin administration has been found to alter the composition of human gut microbiota. See, e.g., Reijnders et al. Cell Metabolism (2016) 24(1): 63-72. Without wishing to be bound by any particular theory, it is thought that administration of vancomycin may aid engraftment of the bacterial strain(s) of the pharmaceutical compositions described herein, for example by removing other microbes present in the gastrointestinal tract.

In some embodiments, the vancomycin is administered at a dose (a single dose or multiple doses) in a sufficient amount to allow for colonization of one or more of the bacterial strains of the pharmaceutical compositions described herein. In some embodiments, the vancomycin is administered to the subject once, as a single dose. In some embodiments, the vancomycin is administered to the subject in multiple doses. In some embodiments, the vancomycin is administered to the subject in at least 2, 3, 4, 5, 6, 7, 8, 9, 10, 11, 12, 13, 14, 15 or more doses. The multiple doses of vancomycin may be administered to the subject at regular intervals prior to administering any of the pharmaceutical compositions described herein. In some embodiments, each of the multiple doses of vancomycin are administered on consecutive days (e.g., first dose on day 1, second dose of day 2, third dose on day 3, etc.). In some embodiments, the vancomycin is administered to the subject for 2, 3, 4, 5, 6, 7, 8, 9, 10, 11, 12, 13, 14, 15, or more consecutive days. In some embodiments, vancomycin is administered to the subject for one day. In some embodiments, vancomycin is administered to the subject each day for three consecutive days. In some embodiments, vancomycin is administered to the subject each day for five consecutive days. In some embodiments, the vancomycin is administered to the subject each day for seven consecutive days. In any of the embodiments described herein, a subject may be administered one or more doses of a first antibiotic followed by one or more doses of a second antibiotic.

In some embodiments, a single dose of any of the pharmaceutical compositions described herein, or the first dose in a treatment regimen of multiple doses, is administered, the same day as the administration of the final dose of vancomycin. In some embodiments, a single dose of any of the pharmaceutical compositions described herein, or the first dose in a treatment regimen of multiple doses, is administered, immediately following the administration of the final dose of vancomycin. In some embodiments, a single dose of any of the pharmaceutical compositions described herein, or the first dose in a treatment regimen of multiple doses, is administered, the day after administration of the final dose of vancomycin. In some embodiments, a single dose of any of the pharmaceutical compositions described herein, or the first dose in a treatment regimen of multiple doses, is administered, two days after administration of the final dose of vancomycin. In some embodiments, the methods provided herein allow for a wash out day between the final dose of vancomycin and the first dose of the pharmaceutical composition. In some embodiments, a single dose of any of the pharmaceutical compositions described herein, or the first dose in a treatment regimen of multiple doses, is administered, three days, four days, five days, six days, ten days or more, after administration of the final dose of vancomycin. In some embodiments, the methods provided herein allow for multiple wash out days between the final dose of vancomycin and the first dose of the pharmaceutical composition. In some embodiments, the methods provided herein allow for two wash out days between the final dose of vancomycin and the first dose of the pharmaceutical composition.

Each dose of the vancomycin may be the same amount of vancomycin or may be a different amount of vancomycin. In some embodiments, the vancomycin is administered in an amount sufficient to allow for colonization of one or more of the bacterial strains of the pharmaceutical compositions described herein. In some embodiments, the subject is administered between about 50 mg and 1 g. 100 mg and 750 mg, 100 mg and 500 mg, 200 mg and 750 mg. 200 mg and 500 mg, 300 mg and 750 mg, 300 mg and 500 mg, 100 mg and 400 mg, 100 mg and 300 mg, 100 mg and 200 mg, 200 mg and 400 mg, 200 mg and 300 mg, or 450 mg to 550 mg vancomycin per day. As will be appreciated by one of skill in the art, the total amount of vancomycin administered to the subject per day may be administered in a single dose or between multiple doses, which in sum results in the total amount of vancomycin per day.

In some embodiments, the subject is administered about 500 mg vancomycin per day prior to administration of any of the pharmaceutical compositions described herein. In some embodiments, 500 mg vancomycin per day is administered in a single dose (e.g., 500 mg). In some embodiments, 500 mg vancomycin per day is administered in multiple doses (e.g., 2, 3, 4, 5 or more), which in sum results in 500 mg vancomycin per day. In some embodiments, 500 mg vancomycin is administered in 4 doses of 125 mg vancomycin per day. In some embodiments, 500 mg vancomycin is administered to the subject for one day. In some embodiments, 500 mg vancomycin is administered to the subject per day for two days. In some embodiments, 500 mg vancomycin is administered to the subject per day for three days. In some embodiments, 500 mg vancomycin is administered to the subject per day for four days. In some embodiments, 500 mg vancomycin is administered to the subject per day for five days. In some embodiments, 500 mg vancomycin is administered to the subject per day for six days. In some embodiments, 500 mg vancomycin is administered to the subject per day for seven days. In some embodiments, 500 mg vancomycin is administered to the subject per day for eight days. In some embodiments, 500 mg vancomycin is administered to the subject per day for nine days. In some embodiments, 500 mg vancomycin is administered to the subject per day for ten days.

In some embodiments, 500 mg vancomycin is administered in 4 doses of 125 mg vancomycin per day for one day. In some embodiments, 500 mg vancomycin is administered in 4 doses of 125 mg vancomycin per day for two days. In some embodiments, 500 mg vancomycin is administered in 4 doses of 125 mg vancomycin per day for three days. In some embodiments, 500 mg vancomycin is administered in 4 doses of 125 mg vancomycin per day for four days. In some embodiments, 500 mg vancomycin is administered in 4 doses of 125 mg vancomycin per day for five days. In some embodiments, 500 mg vancomycin is administered in 4 doses of 125 mg vancomycin per day for six days. In some embodiments, 500 mg vancomycin is administered in 4 doses of 125 mg vancomycin per day for seven days. In some embodiments, 500 mg vancomycin is administered in 4 doses of 125 mg vancomycin per day for eight days. In some embodiments, 500 mg vancomycin is administered in 4 doses of 125 mg vancomycin per day for nine days. In some embodiments, 500 mg vancomycin is administered in 4 doses of 125 mg vancomycin per day for ten days.

In some embodiments, the vancomycin is administered according to a pulse tapered-regime. See e.g., Sirbu et al., Clinical Infectious Diseases (2017) 65: 1396-1399.

In some embodiments, the vancomycin is administered to the subject at least 1, 2, 3, 4, 5, 6, 7 days or more prior to administration of the pharmaceutical compositions described herein. In some embodiments, administration of vancomycin is terminated at least one day (e.g., 1, 2, 3, 4, 5, or more) prior to administration of any of the pharmaceutical compositions described herein. In some embodiments, administration of vancomycin is terminated two days prior to administration of any of the pharmaceutical compositions described herein.

In some embodiments, additional antibiotics are administered in combination with the vancomycin regimes provided herein.

It should be appreciated, in some embodiments, that any of the vancomycin doses or administration regimens may be combined with any of the pharmaceutical composition doses or administration regimens provided herein.

In some embodiments, the disclosure provides methods comprising administering one or more antibiotics to the subject and subsequently administering any of the bacterial compositions to the subject once, twice, 3 times, 4 times, 5 times, 6 times, 7 times, 8 times, 9 times, 10 times, 11 times, 12 times, 13 times, at least 14 times or more. In some embodiments, the disclosure provides methods comprising administering one or more antibiotics to the subject and subsequently administering any of the bacterial compositions described herein to the subject in multiple doses at a regular interval, such as every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose on day 2, third dose on day 3, etc.).

In some embodiments, the subject is treated by multiple cycles, each of which may involve a period of administering multiple doses of any of the compositions described herein, followed by a period during which the compositions are not administered. In some embodiments, each cycle further involves administering antibiotics prior to administering any of the compositions described herein. In some embodiments, the subject is treated by multiple cycles, each of which may involve a period of administering multiple doses of any of the compositions described herein for at least 7 days, followed by a period during which the compositions are not administered. In some embodiments, the subject is treated by multiple cycles, each of which may involve a period of administering multiple doses of any of the compositions described herein for at least 14 days, followed by a period during which the compositions are not administered. In some embodiments, a cycle further comprises determining whether the subject is colonized by one or more of the bacterial strains of the composition, and administering the composition if the one or more of the bacteria of the composition are not detected. In some embodiments, the cycle is repeated after a period of time, such as after 2, 3, 4, 5, 6, 7, 8, 9, 10, 11, or 12 months.

In one aspect, the disclosure provides methods comprising administering one or more antibiotics to the subject and subsequently administering any of the bacterial compositions as multiple daily doses of the pharmaceutical compositions. In some embodiments, the pharmaceutical compositions are administered on a daily basis for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, or more.

In some embodiments, the pharmaceutical compositions are administered on a daily basis for a period of time, followed by a period of time during which the pharmaceutical compositions are not administered. In some embodiments, the methods may involve administering the pharmaceutical compositions to the subject on a daily basis for a second period of time (e.g., a cycle), which may be followed a second period of time during which the pharmaceutical compositions are not administered.

In one aspect the disclosure provides methods comprising the administration of an antibiotic (e.g., vancomycin) followed by the administration of a pharmaceutical composition provided herein, wherein the administration of an antibiotic (e.g., vancomycin) is followed by the administration of a single dose or multiple doses of the pharmaceutical composition. In some embodiments, administration of an antibiotic (e.g., vancomycin) followed by the administration of a single dose or multiple doses of the pharmaceutical composition results in an increase in the abundance of bacterial strains of the pharmaceutical compositions in the microbiome of the subject (engraftment) compared to the administration of a pharmaceutical composition without the administration of the antibiotic. In some embodiments, administration of an antibiotic (e.g., vancomycin) followed by the administration of a single dose or multiple doses of the pharmaceutical composition results in an increase in the duration of the colonization of bacterial strains of the pharmaceutical composition in the microbiome of the subject (e.g., up to 6 months) compared to the administration of a pharmaceutical composition without the administration of the antibiotic.

In some embodiments, administration of an antibiotic (e.g., vancomycin) followed by the administration of a single dose or multiple doses of the pharmaceutical composition results in an increase in the rate of engraftment of the initial amount of the bacterial strains of the pharmaceutical composition in the microbiome of the subject by between ten- to one hundred-fold (e.g., within the first 48 hours) compared to the administration of a pharmaceutical composition without the administration of the antibiotic.

In some embodiments, administration of an antibiotic (e.g., vancomycin) followed by the administration of a single dose or multiple doses of the pharmaceutical composition results in a greater number (amount) of subjects having all of the bacterial strains of the pharmaceutical composition present in their microbiome as compared to compared to the administration of a pharmaceutical composition without the administration of the antibiotic.

In some embodiments, administration of an antibiotic (e.g., vancomycin) followed by the administration of multiple doses of the pharmaceutical composition results in a higher abundance of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition results in higher abundance of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a greater number (amount) of subjects having all of the bacterial strains of the pharmaceutical composition present in their microbiome as compared to the administration of a single dose of the pharmaceutical composition. In some embodiments, the disclosure provides methods comprising the administration of a pharmaceutical composition provided herein, wherein the administration of multiple doses of the pharmaceutical results in a greater number (amount) of subject having all of the bacterial strains of the pharmaceutical composition in their microbiome as compared to the administration of a single dose of the pharmaceutical composition.

The compositions, including the pharmaceutical compositions disclosed herein, include compositions that contain selected bacterial strains. The amount of bacteria, including the amount of bacteria of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that bacteria of each of the bacterial strains may be present in different amounts. Thus, for instance, as a non-limiting example, composition may include 10³ of bacteria A, 10⁴ of bacteria B and 10⁶ of bacteria C. In some embodiments, compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacterial strains per dosage amount. In some embodiments, compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 105, about 10⁻⁴, about 10⁻³, about 10⁻², about 10⁻¹ or more grams of bacteria of each of the bacterial strains in the composition per dosage amount. In some embodiments, the compositions, including pharmaceutical compositions, contain about 10⁻⁷, about 106, about 10⁻⁵, about 10⁻⁴, about 10⁻³, about 10⁻², about 10⁻¹ or more grams of bacteria in total for all of the bacterial strains combined per dosage amount. In some embodiments, compositions, including pharmaceutical compositions, comprise about 10⁹ CFUs in total for all of the bacterial strains combined per dosage amount. In some embodiments, compositions, including pharmaceutical compositions, comprise about 10¹⁰ CFUs in total for all of the bacterial strains combined per dosage amount.

In some embodiments, the dosage amount is one administration device (e.g., one table, pill or capsule). In some embodiments, the dosage amount is the amount administered at one time, which may be in the form of more than one administration device (e.g., more than one table, pill or capsule). In some embodiment, the dosage amount is the amount that is administered in a particular period (e.g., one day or one week).

As described herein, any of the pharmaceutical compositions described herein may be administered once, as a single dose. In some embodiments, the pharmaceutical compositions described herein are administered in multiple doses. In some embodiments, each dose is administered in the form of one or more capsules. In some embodiments, each dose comprises administration of multiple capsules. In some embodiments, each dose is administered in the form of 1, 2, 3, 4, 5, 6, 7, 8, 9, 10, 11, 12, 13, 14, 15, or more capsules. In some embodiments, each dose is administered in the form of 5 capsules. In some embodiments, each dose is administered in the form of 10 capsules.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacterial strains per capsule.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per capsule. In some embodiments, each capsule contains between 10⁷ and 10⁹, between 10⁷ and 10⁸, or between 10⁸ and 10⁹ total bacteria. In some embodiments, each capsule contains about 1.0×10⁷, 2.0×10⁷, 3.0×10⁷, 4.0×10⁷, 5.0×10⁷, 6.0×10⁷, 7.0×10⁷, 8.0×10⁷, 9.0×10⁷, 1.0×10⁸, 2.0×10⁸, 3.0×10⁸, 4.0×10⁸, 5.0×10⁸, 6.0×10⁸, 7.0×10⁸, 8.0×10⁸, 9.0×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹ total bacteria. In some embodiments, each capsule contains about 1×10⁹ total bacteria.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each bacterial strain per capsule.

In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacterial strains per dosage amount. In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the pharmaceutical compositions contain at least about 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6.0×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7.0×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8.0×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9.0×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹, 5.1×10⁹, 5.2×10⁹, 5.3×10⁹, 5.4×10⁹, 5.5×10⁹, 5.6×10⁹, 5.7×10⁹, 5.8×10⁹, 5.9×10⁹, 6.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 7.1×10⁹, 7.2×10⁹, 7.3×10⁹, 7.4×10⁹, 7.5×10⁹, 7.6×10⁹, 7.7×10⁹, 7.8×10⁹, 7.9×10⁹, 8.0×10⁹, 8.1×10⁹, 8.2×10⁹, 8.3×10⁹, 8.4×10⁹, 8.5×10⁹, 8.6×10⁹, 8.7×10⁹, 8.8×10⁹, 8.9×10⁹, 9.0×10⁹, 9.1×10⁹, 9.2×10⁹, 9.3×10⁹, 9.4×10⁹, 9.5×10⁹, 9.6×10⁹, 9.7×10⁹, 9.8×10⁹, 9.9×10⁹, 1.0×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2.0×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3.0×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4.0×10¹⁰, 4.1×10¹⁰, 4.2×10¹⁰, 4.3×10¹⁰, 4.4×10¹⁰, 4.5×10¹⁰, 4.6×10¹⁰, 4.7×10¹⁰, 4.8×10¹⁰, 4.9×10¹⁰, 5.0×10¹⁰, 5.1×10¹⁰, 5.2×10¹⁰, 5.3×10¹⁰, 5.4×10¹⁰, 5.5×10¹⁰, 5.6×10¹⁰, 5.7×10¹⁰, 5.8×10¹⁰, 5.9×10¹⁰, 6.0×10¹⁰, 6.1×10¹⁰, 6.2×10¹⁰, 6.3×10¹⁰, 6.4×10¹⁰, 6.5×10¹⁰, 6.6×10¹⁰, 6.7×10¹⁰, 6.8×10¹⁰, 6.9×10¹⁰, 7.0×10¹⁰, 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, 7.9×10¹⁰, 8.0×10¹⁰, 8.1×10¹⁰, 8.2×10¹⁰, 8.3×10¹⁰, 8.4×10¹⁰, 8.5×10¹⁰, 8.6×10¹⁰, 8.7×10¹⁰, 8.8×10¹⁰, 8.9×10¹⁰, 9.0×10¹⁰, 9.1×10¹⁰, 9.2×10¹⁰, 9.3× 10¹⁰, 9.4×10¹⁰, 9.5×10¹⁰, 9.6×10¹⁰, 9.7×10¹⁰, 9.8×10¹⁰, 9.9×10¹⁰, 1.0×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2.0×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3.0×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4.0×10¹¹, 4.1×10¹¹, 4.2×10¹¹, 4.3×10¹¹, 4.4×10¹¹, 4.5×10¹¹, 4.6×10¹¹, 4.7×10¹¹, 4.8×10¹¹, 4.9×10¹¹, 5.0×10¹¹, 5.1×10¹¹, 5.2×10¹¹, 5.3×10¹¹, 5.4×10¹¹, 5.5×10¹¹, 5.6×10¹¹, 5.7×10¹¹, 5.8×10¹¹, 5.9×10¹¹, 6.0×10¹¹, 6.1×10¹¹, 6.2×10¹¹, 6.3×10¹¹, 6.4×10¹¹, 6.5×10¹¹, 6.6×10¹¹, 6.7×10¹¹, 6.8×10¹¹, 6.9×10¹¹, 7.0×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8.0×10¹¹, 8.1×10¹¹, 8.2×10¹¹, 8.3×10¹¹, 8.4×10¹¹, 8.5×10¹¹, 8.6×10¹¹, 8.7×10¹¹, 8.8×10¹¹, 8.9×10¹¹, 9.0×10¹¹, 9.1×10¹¹, 9.2×10¹¹, 9.3×10¹¹, 9.4×10¹¹, 9.5×10¹¹, 9.6×10¹¹, 9.7×10¹¹, 9.8×10¹¹, 9.9×10¹¹, or 1.0×10¹² total CFUs.

In some embodiments, the pharmaceutical composition comprises about 1×10⁹ total CFUs. In some embodiments, the pharmaceutical composition comprises about 1×10⁹ total CFUs and is administered as a single dose. In some embodiments, the pharmaceutical composition comprises about 1×10⁹ total CFUs and is administered as multiple (e.g., 2, 3, 4, 5, or more) doses. In some embodiments, the pharmaceutical composition comprises about 1×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises about 1×10¹⁰ total CFUs. In some embodiments, the pharmaceutical composition comprises about 1×10¹⁰ total CFUs and is administered as a single dose. In some embodiments, the pharmaceutical composition comprises about 1×10¹⁰ total CFUs and is administered as multiple (e.g., 2, 3, 4, 5, or more) doses. In some embodiments, the pharmaceutical composition comprises about 1×10¹⁰ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises about 5×10⁹ total CFUs. In some embodiments, the pharmaceutical composition comprises about 5×10⁹ total CFUs and is administered as a single dose. In some embodiments, the pharmaceutical composition comprises about 5×10⁹ total CFUs and is administered as multiple (e.g., 2, 3, 4, 5, or more) doses. In some embodiments, the pharmaceutical composition comprises about 5×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

As described herein, any of the pharmaceutical compositions described herein may be administered to a subject in one dose or in multiple doses (e.g., initial administration), which may be followed by one or more additional doses of any of the pharmaceutical compositions described herein. In some embodiments, any of pharmaceutical composition described herein may be administered to a subject in one dose or in multiple doses in an initial administration, followed by one or more additional doses of a pharmaceutical composition comprising the same one or more bacterial strains as the pharmaceutical composition of the initial administration. In some embodiments, any of pharmaceutical composition described herein may be administered to a subject in one dose or in multiple doses in an initial administration, followed by one or more additional doses of a pharmaceutical composition comprising more total bacteria (colony-forming units) relative to the initial administration of the pharmaceutical composition. In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In some embodiments, the initial administration includes at least 1, 2, 3, 4, 5, 6, 7, 8, 9, 10, 11, 12, 13, 14, 15 or more doses of any of the pharmaceutical compositions described herein. In some embodiments, the additional administration includes at least 1, 2, 3, 4, 5, 6, 7, 8, 9, 10, 11, 12, 13, 14, 15 or more doses of any of the pharmaceutical compositions described herein. In some embodiments, the initial administration comprises two doses of any of the pharmaceutical composition and the additional administration comprises three doses of any of the pharmaceutical compositions described herein.

In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition.

Aspects of the present disclosure provide methods involving administering a subject a therapeutically effective amount of a pharmaceutical composition in a loading period followed by administering to the subject a therapeutically effective amount of the pharmaceutical composition a maintenance period. As will be appreciated by one of ordinary skill in the art, the dosing amount and/or dosing frequency of the composition during the loading period and the maintenance period may be the same or different. In some embodiments, the loading period involves administration of any of the compositions described herein in an increased dosing amount and/or dosing frequency as compared to the dosing amount and/or dosing frequency of the maintenance period.

In some embodiments, the method further comprises administering to the subject an antibiotic (e.g., vancomycin) prior to the loading period. In some embodiments, the loading period is not preceded by administration of an antibiotic.

In some embodiments, the one or more additional administrations is performed on the day following the initial administration (e.g., consecutive days). In some embodiments, the one or more additional administrations is performed at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks or longer following the initial administration. In some embodiments, the one or more additional administrations is performed at least 6 weeks after the initial administration (e.g., at least 6 weeks after the final dose of the initial administration). In some embodiments, the one or more additional administrations is performed at least 12 weeks after the initial administration (e.g., at least 6 weeks after the final dose of the initial administration).

In some embodiments, the compositions, including pharmaceutical compositions, contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 105 and 10⁻², between 104 and 10⁻², between 10⁻³ and 10⁻², between 10⁻⁷ and 10⁻³, between 106 and 10⁻³, between 105 and 10⁻³, between 10⁻⁴ and 10⁻³, between 10⁻⁷ and 10⁻⁴, between 106 and 10⁻⁴, between 105 and 104, between 10⁻⁷ and 10⁻⁵, between 106 and 10-5, or between 10⁻⁷ and 10⁻⁶ grams of bacteria of each of the bacterial strains in the composition per dosage amount. In some embodiments, the compositions, including pharmaceutical compositions, disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 104 and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of all of the bacteria combined (total) per dosage amount.

In one aspect the disclosure provides methods comprising the administration of an antibiotic (e.g., vancomycin) followed by the administration of a pharmaceutical composition provided herein, wherein the administration of an antibiotic (e.g., vancomycin) is followed by the administration of a single dose or multiple doses of the pharmaceutical composition. In some embodiments, administration of an antibiotic (e.g., vancomycin) followed by the administration of a single dose or multiple doses of the pharmaceutical composition results in an increase in the abundance of bacterial strains of the pharmaceutical compositions in the microbiome of the subject (engraftment) compared to the administration of a pharmaceutical composition without the administration of the antibiotic. In some embodiments, administration of an antibiotic (e.g., vancomycin) followed by the administration of a single dose or multiple doses of the pharmaceutical composition results in an increase in the duration of the colonization of bacterial strains of the pharmaceutical composition in the microbiome of the subject (e.g., up to 6 months) compared to the administration of a pharmaceutical composition without the administration of the antibiotic.

In some embodiments, administration of an antibiotic (e.g., vancomycin) followed by the administration of a single dose or multiple doses of the pharmaceutical composition results in an increase in the rate of engraftment of the initial amount of the bacterial strains of the pharmaceutical composition in the microbiome of the subject by between ten- to one hundred-fold (e.g., within the first 48 hours) compared to the administration of a pharmaceutical composition without the administration of the antibiotic.

In some embodiments, administration of an antibiotic (e.g., vancomycin) followed by the administration of a single dose or multiple doses of the pharmaceutical composition results in a greater number (amount) of subjects having all of the bacterial strains of the pharmaceutical composition present in their microbiome as compared to compared to the administration of a pharmaceutical composition without the administration of the antibiotic.

In some embodiments, administration of an antibiotic (e.g., vancomycin) followed by the administration of multiple doses of the pharmaceutical composition results in an increase in the abundance of bacterial strains of the pharmaceutical composition in the microbiome of the subject (engraftment) compared to the administration of a single dose of the pharmaceutical composition. In some embodiments, the disclosure provides methods comprising the administration of a pharmaceutical composition provided herein, wherein the administration of multiple doses of the pharmaceutical composition increases the abundance of bacterial strains in the microbiota of the subject (engraftment) of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in an increase in the rate of engraftment of the initial amount of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition increases the rate of engraftment of the initial amount of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a higher abundance of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition results in higher abundance of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a greater number (amount) of subjects having all of the bacterial strains of the pharmaceutical composition present in their microbiome as compared to the administration of a single dose of the pharmaceutical composition. In some embodiments, the disclosure provides methods comprising the administration of a pharmaceutical composition provided herein, wherein the administration of multiple doses of the pharmaceutical results in a greater number (amount) of subject having all of the bacterial strains of the pharmaceutical composition in their microbiome as compared to the administration of a single dose of the pharmaceutical composition.

In some embodiments, administration of an antibiotic (e.g., vancomycin) followed by the administration of multiple doses of the pharmaceutical composition results in an accelerated recovery of the microbiome (e.g., increase in bacterial species of Bacteroidetes and/or Firmicutes, and/or decrease in Proteobacteria) as compared to the administration of a single dose of the pharmaceutical composition. In some embodiments, the disclosure provides methods comprising the administration of a pharmaceutical composition provided herein, wherein the administration of multiple doses of the pharmaceutical results in an accelerated recovery of the microbiome (e.g., increase in bacterial species of Bacteroidetes and/or Firmicutes, and/or decrease in Proteobacteria) as compared the administration of a single dose of the pharmaceutical composition.

In some embodiments, the methods described herein may involve subjecting the subject to a bowel lavage (bowel irrigation, whole bowel irrigation, gastrointestinal lavage, gastric lavage) prior to administration of the compositions described herein. In some embodiments, a bowel lavage may remove or aid in removing microbiota from the gastrointestinal tract of the subject, creating a niche for the bacterial strains of the compositions described herein. In some embodiments, the bowel lavage may be an oral bowel lavage or a rectal bowel lavage.

Methods of performing a bowel lavage are known in the art, and generally involve the rapid administration of large volumes of a solution, such as polyethylene glycol or a balanced electrolyte solution. A rectal bowel lavage can involve the administration of a solution or a suppository containing the pharmaceutical composition. A bowel lavage may be performed under doctor supervision, hospitalization, or at home.

In some embodiments, the compositions provided herein induce the proliferation and/or accumulation of regulatory T cells in the subject. As will be evident to one of ordinary skill in the art, regulatory T cells, also referred to as “Tregs,” are a subset of T lymphocytes that are generally thought to suppress an abnormal or excessive immune response and play a role in immune tolerance. Regulatory T cells may be identified based expression of the markers Foxp3 and CD4 (Foxp3+CD4+). The term regulatory T cells may also include Foxp3-negative regulatory T cells that are IL-10-producing CD4-positive T cells.

In some embodiments, the therapeutically effective amount is an amount sufficient to induce the proliferation and/or accumulation of Tregs in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of Tregs in a subject (e.g., a subject having or at risk of IBD or GvHD) that has not received any of the compositions described herein or as compared to a fecal sample from the same subject that was collected prior to administration of any of the compositions.

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, administration of the compositions described herein results in an increase the proliferation and/or accumulation of regulatory T cells (e.g., total Tregs or a specific subset of Treg) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or a specific subset of Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the quantity of regulatory T cells in another subject (e.g., a reference subject) who did not receive the compositions.

In some embodiments, administration of the compositions described herein results in an increase in the proliferation and/or accumulation of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

In some embodiments, administration of the compositions described herein results in an increase in activity of regulatory T cells (e.g., total Tregs or a specific subset of Tregs) at a particular site (e.g., the gastrointestinal tract) in the subject. In some embodiments, administration of the compositions described herein results in an increase in activity of regulatory T cells (e.g., total Tregs or a specific subset of Tregs) by at least 1.1-fold, 1.2-fold, 1.3-fold. 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the activity of regulatory T cells in the subject (or particular site in the subject) prior to administration of the compositions. In some embodiments, administration of the compositions described herein results in an increase in activity of regulatory T cells (e.g., total Tregs or a specific subset of Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the activity of regulatory T cells in another subject (e.g., a reference subject) who did not receive the compositions.

In some embodiments, administration of the compositions described herein results in an increase in the activity of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject (or particular site in the subject) prior to administration of the compositions. In some embodiments, administration of the compositions described herein results in an increase in the activity of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total Tregs or a specific subset of Tregs) can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

Aspects of the present disclosure also provide food products comprising any of the compositions provided herein and a nutrient. Also within the scope of the present disclosure are food products comprising any of the bacterial strains described herein and a nutrient. Food products are, in general, intended for the consumption of a human or an animal. Any of the compositions described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing the bacterial strains described herein may be produced using methods known in the art and may contain the same amount of bacteria (e.g., by weight, amount, or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

Some aspects of the compositions and methods described herein decrease the abundance of primary bile acids, increase the abundance of secondary bile acids, and/or promote conversion of primary bile acids into secondary bile acids. Bile acids are steroid acids that allow the digestion of dietary fats and oils by acting as surfactants that turn the fats and oils into micelles. Bile acids also act as hormones utilizing the farnesoid X receptor and GBPAR1. Primary bile acids are synthesized in the liver from cholesterol and a conjugated with either taurine or glycine prior to secretion. When the primary bile acids are secreted into the lumen of the intestine, bacteria partially dehydroxylate and remove the glycine or taurine groups, forming secondary bile acids.

Non-limiting examples of primary bile acids are cholic acid (CA), chenodeoxycholic acid (CDCA), glycocholic acid (GCA), glycochenodeoxycholic acid (GCDCA), glycodeoxycholic acid (GDCA), taurocholic acid (TCA), and taurochenodeoxycholic acid (TCDCA). Non-limiting examples of secondary bile acids are deoxycholic acid (DCA), lithocholic acid (LCA), ursodeoxycholic acid (UDCA), taurodeoxycholic acid (TDCA), taurolithocholic acid (TLCA), and tauroursodeoxycholic acid (TUDCA).

Several diseases and disorders, such as inflammatory bowel disease, cancer (e.g., colorectal carcinoma), and pathogenic organism infections (e.g., Clostridium difficile infection), are associated with increased primary bile acids and reduced secondary bile acids. The primary bile acids are reduced, and the secondary bile acids are increased following fecal matter transplant (FMT) (Seckatz, et al., Anderobe (2018) 53: 64-73). In some embodiments, administration of the bacterial strains or a pharmaceutical composition as described herein reduces primary bile acids and/or increases secondary bile acids.

In some embodiments, the levels of primary bile acids are reduced by 10-fold to 100,000-fold following administration of the bacterial strains or any of the pharmaceutical compositions described herein. In some embodiments, the levels of primary bile acids are reduced by 2-fold to 1,000-fold following administration of the bacterial strains or any of the pharmaceutical compositions described herein. In some embodiments, the levels of primary bile acids are reduced by 10-fold to 1,000-fold following administration of the bacterial strains or any of the pharmaceutical compositions described herein. In some embodiments, the levels of primary bile acids are reduced 20-fold to 10,000-fold following administration of the bacterial strains or any of the pharmaceutical compositions described herein. In some embodiments, the levels of primary bile acids are reduced by 2-fold, 5-fold, 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 40,000-fold, 50,000-fold, 60,000-fold, 70,000-fold, 80,000-fold, 90,000-fold, or 100,000-fold following administration of the bacterial strains or any of the pharmaceutical compositions described herein.

In some embodiments, the levels of secondary bile acids are increased by 2-fold to 10,000-fold following administration of the bacterial strains or any of the pharmaceutical compositions described herein. In some embodiments, the levels of secondary bile acids are increased by 10-fold to 10,000-fold following administration of the bacterial strains or any of the pharmaceutical compositions described herein. In some embodiments, the levels of secondary bile acids are increased by 10-fold to 1.000-fold following administration of the bacterial strains or any of the pharmaceutical compositions described herein. In some embodiments, the levels of secondary bile acids are increased by 20-fold to 100-fold following administration of the bacterial strains or any of the pharmaceutical compositions described herein. In some embodiments, the levels of secondary bile acids are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 1,000-fold following administration of the bacterial strains or any of the pharmaceutical compositions described herein.

Some aspects of the compositions and methods described herein increase production of regulatory metabolites, such as short chain fatty acids (e.g., in the gastrointestinal tract of the subject). In some embodiments, the methods involve administering to a subject one or more compositions containing bacterial strains that produce short chain fatty acids. SCFAs are abundant in healthy subjects and decreased in subjects having particular diseases and disorders.

SCFA produced in the gastrointestinal tract are thought to function as signaling molecules between the gut microbiota and the host organism, with the SCFA playing a role in local, intermediary and peripheral metabolism of the host. Sec, e.g., Morrison, et al. Gut Microbes (2016) 7(3): 189-200.

Short chain fatty acids (SCFAs) are fatty acids containing six or less carbon atoms. They are produced when dietary fiber is fermented in the intestine. They are primarily absorbed in the portal vein following lipid digestion. SCFAs can affect the production of lipids, energy, and vitamins, as well as playing a critical role in maintaining intestinal epithelial cell membrane integrity. Examples of SCFA include, without limitation, formic acid, acetic acid, butyric acid, isobutyric acid, valeric acid, or isovaleric acid. In some embodiments, the SCFA is butyric acid (butyrate).

In some embodiments, SCFAs are increased by 2-fold to 10,000-fold following administration of the bacterial strains or any of the pharmaceutical compositions described herein. In some embodiments, SCFAs are increased by 10-fold to 500-fold following administration of the bacterial strains or any of the pharmaceutical compositions described herein. In some embodiments, SCFAs are increased by 2-fold to 250-fold following administration of the bacterial strains or any of the pharmaceutical compositions described herein. In some embodiments, SCFAs are increased by 100-fold to 500-fold following administration of the bacterial strains or any of the pharmaceutical compositions described herein. In some embodiments, SCFAs are increased by 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, or 500-fold following administration of the bacterial strains or any of the pharmaceutical compositions described herein.

Some aspects of the compositions and methods described herein increase production of regulatory metabolites, such as indoles (e.g., in the gastrointestinal tract of the subject). In some embodiments, the methods involve administering to a subject one or more of the compositions described herein containing bacterial strains that produce indoles. Indoles are abundant in healthy subjects and decreased in subjects having particular diseases and disorders.

Indoles are tryptophan derivatives, and are produced when bacteria catabolize tryptophan in the intestine. Indoles produced in the gastrointestinal tract are thought to contribute to intestinal and systemic homeostasis, and may hinder the development of hepatic pathologies such as alcoholic steatohepatitis and nonalcoholic fatty liver disease. Sec, e.g., Hendrikx et al., J Intern Med. 2019. 286(1):32-40. Examples of indoles include, without limitation, kynurenic acid, serotonin, nicotinic acid, indole, indole 3-acetic, and indole 3-propionic acid.

In some embodiments, the levels of indoles are increased by 2-fold to 10,000-fold following administration of the bacterial strains or any of the pharmaceutical compositions described herein. In some embodiments, the levels of indoles are increased by 10-fold to 10,000-fold following administration of the bacterial strains or any of the pharmaceutical compositions described herein. In some embodiments, the levels of indoles are increased by 10-fold to 1,000-fold following administration of the bacterial strains or any of the pharmaceutical compositions described herein. In some embodiments, the levels of indoles are increased by 20-fold to 100-fold following administration of the bacterial strains or any of the pharmaceutical compositions described herein. In some embodiments, the levels of indoles are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 1,000-fold following administration of the bacterial strains or any of the pharmaceutical compositions described herein.

Any of the methods described herein may further comprise administering one or more additional therapeutic agents, which may be administered before, at about the same time, or after the administration of the pharmaceutical composition. The one or more therapeutic agents may be administered in combination, for example as part of the pharmaceutical composition, or administered in a separate composition. In some embodiments, the therapeutic agent is a tumor necrosis factor α (TNF-α) inhibitor, T cell chemoattractant, Janus kinase (JAK) inhibitor, IL23/12 blocker, or sphingosine-1-phosphate (S1P) receptor modulator.

Non-limiting examples of TNF-α inhibitors include anti-TNF-α antibodies, soluble TNF-α receptors (e.g., etanercept), thalidomide, lenalidomide, pomalidomide, xanthine derivatives (e.g., pentoxifylline), and bupropion.

T cell chemoattractants include cytokines and chemokines that are capable of interacting with receptors on T cells, thereby attracting T cells to the location of the chemoattractant.

Non-limiting examples of cytokines include TNFα, IFN-γ, IFN-α, TGF-β, IL-1β, IL-2, IL-4. IL-10, IL-13, IL-17, and IL-18. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, and CXCL10.

Non-limiting examples of JAK inhibitors include Tofacitinib, CYT387, Baricitinib, Ruxolitinib, TG101348, Lestaurtinib, AZD1480, R348, VX-509, GLPG0634, GSK2586184, AC-430, Pacritinib, BMS-911543. See, e.g., Furumoto and Gadina. BioDrugs. 2013. 27(5):431-438.

Non-limiting examples of S1P receptor modulators include Fingolimod, Siponimod, Ozanimod, Ceralifimod, GSK2018682, Ponesimod, and MT-1303. See, e.g., Chaudhry et al. Neurotherapeutics. (2017) 14(4):859-873.

Also provided herein are methods for assessing colonization of one or more bacterial strains of a bacterial composition in a microbiome of a subject comprising isolating nucleic acid from a sample of the microbiome of the subject and determining the presence of each least one bacterial strain of the bacterial composition by detecting one or more genomic markers for bacterial strains of the bacterial composition. In some embodiments, provided herein are methods for assessing recovery or restoration of a microbiome of a subject, for example following a dysbiosis inducing event. In some embodiments, if a genomic marker for a bacterial strain is present, the microbiome is colonized with the bacterial strain. In some embodiments, if a genomic marker for a bacterial strain is absent, the microbiome is not colonized with the bacterial strain. In some embodiments, a genomic marker for a bacterial strain that is absent indicates that the subject should be administered one or more further doses of the bacterial composition.

In some embodiments, detection of a genomic marker of one bacterial strain is used as a proxy for the presence of each of the bacterial strains of the bacterial composition.

Also provided herein are methods for assessing colonization of one or more bacterial strains of a bacterial composition in a microbiome of a subject comprising isolating nucleic acid from a sample of the microbiome of the subject; amplifying one or more nucleotide sequence of the bacterial strains in the isolated nucleic acid; and determining the presence of each bacterial strain of the bacterial composition by amplifying a nucleotide sequence of a genomic marker for the bacterial strains in the isolated nucleic acid; wherein if a genomic marker for a bacterial strain is present in the plurality of nucleotide sequences, the microbiome is colonized with the bacterial strain.

In some embodiments, the one or more of the bacterial strains of the bacterial compositions colonize the microbiome of the gastrointestinal tract or parts thereof (e.g., the colon or the cecum) of the subject. Such colonization may also be referred to as grafting or engraftment. The methods described herein allow for the determination of the presence of each bacterial strain of the bacterial composition within the microbiome, which indicates whether the bacterial strain has colonized the microbiome.

In some embodiments, the methods described herein may be used as a companion diagnostic for use with a bacterial composition comprising a mixture of bacterial strains. For example, the described herein may be used for identifying subjects in need of one or more (e.g., 1, 2, 3, 4, 5 or more) additional doses of a bacterial composition. In some embodiments, if one or more of the bacterial strains of the bacterial composition is not present in the plurality of nucleotides sequences, the method further comprising administering one or more additional doses of the bacterial composition to the subject.

In some embodiments, the subject was previously administered one or more doses of the bacterial composition and the methods involve determining whether the subject is in need of one or more additional doses of a bacterial composition.

In some embodiments, the methods described herein include collecting a sample that comprises the microbiome from a subject that has previously been administered one or more doses of a bacterial composition. A sample may be any biological sample that may contain the bacterial strains from the bacterial composition. In some embodiments, a sample is a fecal sample, a urine sample, a blood sample, a serum sample, a plasma sample, a lymph sample, a swab sample, a sputum sample, an aspirate sample, a saliva sample, a lavage sample, a brushing sample, and a biopsy sample. In some embodiments, the sample is a fecal sample.

In some embodiments, the bacterial compositions described herein are administered to the subject such that the bacterial strains may engraft into the gastrointestinal tract of the subject. Accordingly, in some embodiments, collecting and analyzing a biological sample of the gastrointestinal tract of the subject may indicate whether one or more of the bacterial strains of the composition engrafted in the gastrointestinal tract. In some embodiments, the sample is a sample representative of the gastrointestinal tract, or region thereof (e.g., small intestine, colon) of the subject. In some embodiments, the microbiota of the sample is representative of the microbiota of the gastrointestinal tract, or region thereof (e.g., small intestine, colon) of the subject.

In some embodiments, the methods described herein include removal of microorganism and/or cells of the subject (e.g., host cells) from the sample collected from the subject. Microbes may include bacteria, yeast, protozoa, and viruses. Microbes may be removed by any method known in the art including, but not limited to, selective lysis, centrifugation, size-based exclusion, and specific binding and removal of microorganism or cells of the subject (e.g., host cells).

In some embodiments, the methods described herein include lysing cells in the sample prior to isolating nucleic acid from the sample. Methods of lysing cells will be evident to one ordinary skill in the art and may depend on the type of cells present in the sample or type of cells for which lysis is desired. Examples of for cell lysis, include but are not limited to, contacting the cells with anionic detergents, cationic detergents, non-ionic detergents, guanadinium chloride, urea, alcohols, ethers, chloroforms, sonication, freeze-thaw cycles, electroporation, French press, manual grinding, and extrusion.

In some embodiments, specific nucleic acids or types of nucleic acids are removed from lysed cells prior to selective amplification. In some embodiments, RNA is selectively removed from the sample. RNA may be removed from lysed cells by any method known in the art including, but not limited to, addition of RNAse (RNAse A, RNAse H, etc.), centrifugation, and precipitation. In some embodiments, DNA is selectively removed from the sample. DNA may be removed from lysed cells by any method known in the art including, but not limited to, addition of DNAse, centrifugation, and precipitation.

In some embodiments, one or more genomic markers of a bacterial strain are determined to be present in the sample if the genomic marker is detected in a plurality of nucleotide sequences sequenced from the sample. In some embodiments, a bacterial strain is determined to be present in the sample if one or more genomic markers associated with the bacterial strain is detected in a plurality of nucleotide sequences sequenced from the sample. In some embodiments, the one or more genomic markers of a bacterial strain are determined to be present in the sample if the abundance of the genomic marker associated with the bacterial strain represents greater than a particular percentage of the plurality of nucleotide sequences sequenced from the sample. In some embodiments, the one or more genomic markers of a bacterial strain are determined to be present in the sample if the abundance of the genomic marker associated with the bacterial strain represents greater than 0.0001%, 0.0005%. 0.001%, 0.005%, 0.01%, 0.05%, 0.1% 0.5%, 1.0%, 1.5%, 2%, 2.5%, 3%, 3.5%, 4.0%, 4.5%, or 5% of the plurality of nucleotide sequences sequenced from the sample.

As used herein, the term “genomic marker” is used to refer to a sequence that is associated with a target bacterial strain, whereby detection of the genomic marker indicates presence of the target bacterial strain. In some embodiments, the genomic marker is unique to given genome (e.g., present in the genome of target bacterial strain but not present in the genome of other non-target bacterial strains or host genome). The target genomic marker is the genomic marker for a given genome (e.g., bacterial strain 16S rDNA).

In some embodiments, the genomic marker is about 50 nucleotides-about 900 nucleotides. In some embodiments, the target amplified piece of DNA is about 500 nucleotides-about 1000 nucleotides. In some embodiments, the target amplified piece of DNA is about 50 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 1000 nucleotides, 1050 nucleotides, 1100 nucleotides, 1150 nucleotides, or 1200 nucleotides.

In some embodiments, the presence of one or more genomic markers of a plurality of genomic markers of a bacterial strain indicates that the bacterial strain has colonized the microbiome. In some embodiments, the plurality of genomic markers comprises between 200 to 1000 nucleotide sequences for each bacterial strain of the bacterial composition. In some embodiments, each of the genomic markers is a nucleotide sequence that is generally not present in a healthy microbiome. In some embodiments, each of the genomic markers of a bacterial strain is a nucleotide sequence that is not present in the genome of other microorganisms, including other bacterial strains of the bacterial composition. Therefore, in some embodiments, the presence of a genomic marker of a bacterial strain uniquely identifies the presence of a specific bacterial strain. In some embodiments, each genomic marker of the plurality of genomic markers comprises between 25 and 75 nucleotides. In some embodiments, each genomic marker of the plurality of genomic markers comprises about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 73, 74, or 75 nucleotides. In some embodiments, each genomic marker of the plurality of genomic markers comprises about 50 nucleotides.

In some embodiments, the colonization of one or more bacterial strains from the bacterial composition is assessed in the subject by selectively amplifying the nucleotide sequence of a genomic marker for one or more of the bacterial strains. As used herein, the term “selectively amplifying” refers to amplifying a nucleotide sequence of genome marker for at least one of the bacterial strains in isolated nucleic acid. In some embodiments, selective amplification involves providing one or more DNA primers (e.g., a pair of DNA primers) that hybridize (bind) to a region of the isolated nucleic acid that is associated with the target bacterial strain. In some embodiments, the pair of DNA primers are used to amplify a genomic marker from the one or more bacterial strains in the isolated nucleic acids. Selective amplification may be by any method known in the art including, but not limited to, quantitative polymerase chain reaction (qPCR), quantitative real time polymerase chain reaction (qRT-PCR), real time polymerase chain reaction (RT-PCR), and polymerase chain reaction (PCR).

In some embodiments, selectively amplifying one or more nucleotide sequences of the bacterial strains in the isolated nucleic acid involves performing quantitative polymerase chain reactions (qPCR). In some embodiments, the qPCR comprises a pair of DNA primers that specifically hybridize to a genomic marker for a bacterial strains of the bacterial composition, thereby amplifying the genomic marker or portion thereof. In some embodiments, the method further involves selecting a pair of primers (e.g., qPCR primers) for amplifying the nucleotide sequence of the genomic marker of bacterial strain.

In some embodiments, a qPCR reaction mixture contains all components necessary to perform a qPCR reaction. In some embodiments, the qPCR reaction mixture contains isolated DNA, a pair of forward and reverse primers, a DNA probe and/or fluorophore that fluoresces in the presence of double-stranded DNA, an enzyme (e.g., polymerase), mixture of deoxyribonucleotide triphosphates (e.g., dCTP, dATP, dTTP, dGTP), one or more buffers, and water.

In some embodiments, the qPCR reaction mixture contains about 10% isolated DNA. It should be understood that the terms “isolated DNA” and “extracted DNA” are interchangeable herein. In some embodiments, the qPCR reaction mixture contains at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 49%, or 50% isolated DNA. In some embodiments, the qPCR reaction mixture contains 10% isolated DNA.

The qPCR reaction contains a forward primer and a reverse primer that hybridize (bind) to and selectively amplify a genomic marker for a target bacterial strain. The sequences of the forward and reverse primers may be designed to specifically recognize a genomic marker for a given bacterial strain (e.g., of the bacterial composition). The sequences of the forward and reverse primers may be designed to preferentially recognize a genomic marker for a given bacterial strain (e.g., of the bacterial composition) (e.g., better than the primer pair recognizes one or more other sequences). The sequence composition and length of the forward and reverse primers are designed to bind and selectively amplify a target genomic marker. In some embodiments, the qPCR reaction contains more than one pair of primers. In some embodiments, the qPCR reaction contains at least one pair, at least two pairs, at least three pairs, at least four pairs, at least five pairs, at least six pairs, at least seven pairs, or at least eight pairs of qPCR primers. In some embodiments, the qPCR reaction contains at least eight pairs of qPCR primers.

Without wishing to be bound by any particular theory, it is generally thought that longer qPCR primers (e.g., >25 nucleotides) may have greater binding specificity for a target genomic marker as compared to short qPCR primers (e.g., <25 nucleotides). However, longer qPCR primers (e.g., >25 nucleotides) may have lower amplification of the target genomic marker as compared to shorter qPCR primers due to formation of secondary structures.

In some embodiments, the forward primer is 25-45 nucleotides in length. In some embodiments, the forward primer is 10-35 nucleotides in length. In some embodiments, the forward primer is 15-40 nucleotides in length. In some embodiments, the forward primer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the reverse primer is 25-45 nucleotides in length. In some embodiments, the reverse primer is 10-35 nucleotides in length. In some embodiments, the reverse primer is 15-40 nucleotides in length. In some embodiments, the reverse primer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. The forward primer and the reverse primer need not be the same length. In some embodiments, the forward primer and the reverse primer are the same length.

In general, the primers of a primer pair may not be 100% complementary to a target genomic marker in order to hybridize and selectively amplify target genomic markers in the isolated nucleic acid. In some embodiments, the forward primer is 100% complementary to a region of the target genomic marker. In some embodiments, the reverse primer is 100% complementary to a region of the target genomic marker. In some embodiments, the forward primer is at least 75%, 76%, 77%, 78%, 79%, 80%, 81%, 82%, 83%, 84%, 85%, 86,%, 87%, 88%, 89%, 90%, 91%, 92%, 93%, 94%, 95%, 96%, 97%, 98%, 99%, 99.5%, or 99.9% complementary to a region of the target genomic marker. In some embodiments, the reverse primer is at least 75%, 76%, 77%, 78%, 79%, 80%, 81%, 82%, 83%, 84%, 85%, 86,%, 87%, 88%, 89%, 90%, 91%, 92%, 93%, 94%, 95%, 96%, 97%, 98%, 99%, 99.5%, or 99.9% complementary to a region of the target genomic marker. In some embodiments, the forward primer and the reverse primer are sufficiently complementary to a region of the target genomic marker such that the genomic marker is amplified in the qPCR reaction.

In some embodiments, the qPCR reaction mixture contains about 3% primer DNA. Primer DNA may include forward primers and/or reverse primers. In some embodiments, the qPCR reaction mixture contains at least 2%, 3%, 4%, 5%, 6%, 7%, 8%, 9%, 10%, 11%, 12%, 13%, 14%, 15%, 16%, 17%, 18%, 19%, or 20% primer DNA. In some embodiments, the qPCR reaction mixture contains 3% primer DNA.

In some embodiments, the qPCR reaction mixture contains a DNA probe. A DNA probe is single-stranded DNA molecule that is complementary to a sequence on the target genomic marker. Binding of the DNA probe to the amplified genomic marker in a qPCR reaction produces a measurable signal that can be used to quantify the bacterial genomic marker present in the qPCR reaction. In some embodiments, the DNA probe contains a fluorescent molecule (e.g., fluorophore) and the measurable signal is fluorescence. In some embodiments, the qPCR reaction mixture contains about 2% DNA probe. In some embodiments the qPCR reaction mixture contains at least 1%, 2%, 3%, 4%, 5%, 6%, 7%, 8%, 9%, 10%, 11%, 12%, 13%, 14%, 15%, 16%, 17%, 18%, 19%, or 20% DNA probe. In some embodiments, the qPCR reaction mixture contains 2% DNA probe.

In some embodiments, the DNA probe is 100% complementary to a region of the target genomic marker. In some embodiments, the DNA probe is at least 75%, 76%, 77%, 78%. 79%, 80%, 81%, 82%, 83%, 84%, 85%, 86,%, 87%, 88%, 89%, 90%, 91%, 92%, 93%, 94%, 95%, 96%, 97%, 98%, 99%, 99.5%, or 99.9% complementary to a region of the target genomic marker.

In some embodiments, the DNA probe contains a fluorophore and a quencher, wherein the fluorophore produces a fluorescent signal that can be detected at a particular wavelength of light and the quencher diminishes the fluorescent signal. In some embodiments, the quencher diminishes the fluorescent signal by absorbing the energy produced by the fluorophore (e.g., fluorescence) and converting that energy to another form (e.g., heat). In some embodiments, the DNA probe contains more than one quencher. The presence of more than one quencher in a DNA probe may decrease the number of false positives in a qPCR reaction by decreasing the likelihood that a fluorescent signal will be detected on a non-target amplified DNA.

In some embodiments, the DNA probe contains one fluorophore and one quencher. In some embodiments, the DNA probe contains one fluorophore and two quenchers. In some embodiments, the DNA probe contains one fluorophore and two, three, four, five, six, seven, eight, nine, or ten quenchers.

In some embodiments, the fluorophore is present at the 5′ end of the DNA probe and one quencher is present at the 3′ end of the DNA probe. In some embodiments, the fluorophore is present at the 3′ end of the DNA probe and one quencher is present at the 5′ end of the DNA probe. In some embodiments, the fluorophore is present at the 5′ end of the DNA probe, one quencher is present at the 3′ end of the DNA probe, and at least one quencher is internal in the DNA probe (e.g., not at the 5′ end or 3′ end). In some embodiments, the fluorophore is present at the 3′ end of the DNA probe, one quencher is present at the 5′ end of the DNA probe, and at least one quencher is internal in the DNA probe (e.g., not at the 5′ end or 3′ end).

The fluorophore may be any fluorophore known in the art. Selection of the fluorophore may be based, for example, on the excitation and emission wavelengths of the fluorophore, as well as the chemical modifications required to incorporate the fluorophore into the DNA probe. Non-limiting examples of fluorophores that may be present in DNA probes of the present disclosure include: fluorescein (FAM), fluorescein dT, cyanine 3 (Cy3™), TAMRA™, 6-carboxy-4′,5′-dichloro-2′,7′-dimethoxyfluorescein, JOE, cyanine 5 (Cy5™), MAX, tetrachlorofluorescein (TET™), cyanine 5.5 (Cy5.5™), carboxy-X-rhodamine (ROX), TYE™ 563, Yakima Yellow®, hexachlorofluorescein (HEX), TEX 615, TYE™ 665, TYE 705, Alexa Fluor® 488, Alexa Fluor® 532, Alexa Fluor® 546, Alexa Fluor® 594, Alexa Fluor® 647, Alexa Fluor® 660, Alexa Fluor® 750, IRDye® 700, IRDye® 800, IRDye® 800 CW, ATTO™ 488, ATTO™ 532, ATTO™ 550, ATTO™ 565, ATTO™ Rho101, ATTO™ 590, ATTO™ 633, ATTO™ 674N, Rhodamine Green™-X, Rhodamine Red™-X, WellRED D4, WellRED D3, WellRED D2, Texas Red®-X, Lightcycler® 640, and Dy750. In some embodiments, the fluorophore is FAM.

The quencher(s) may be any quencher(s) known in the art. Selection of one or more quenchers may be based, for example, on the excitation and emission wavelengths of the fluorophore that the quencher is quenching, as well as the chemical modifications required to incorporate the quencher into the DNA probe. In some embodiments, when more than one quencher is present in the DNA probe, the quenchers are different. In some embodiments, when more than one quencher is present in the DNA probe, the quenchers are the same. Non-limiting examples of quenchers that may be present in DNA probes of the present disclosure include: ZEN™, TAO™, Iowa Black™, Iowa Black FQ™ (IABKFQ), Eclipse Dark Quencher, IQ4, Black Hole Quencher 1, Black Hole Quencher 2, and Black Hole Quencher 3. In some embodiments, the 3′ quencher is Iowa Black FQ™. In some embodiments, the internal quencher is ZEN™. In some embodiments, the 3′ quencher is Iowa Black FQ™ and the internal quencher is ZEN™.

In general, a qPCR reactions involve cycles that may comprise a denaturing step, an annealing step that allows the forward primer and reverse primer to hybridize (bind) to a region of the target genomic marker, followed by an amplification/extension step that allows the enzyme (e.g., polymerase), to synthesize a complementary strand of DNA. In some embodiments, the annealing step and the amplification/extension step are performed as a single step (e.g., at one temperature). The length and temperature of the annealing step is determined, for example, by the length and sequence composition of the primer pair and the target genomic marker. In some embodiments, primer sequences greater than 60 base pairs and/or primer sequences with a high concentration of adenine-thymine base pairs (e.g., >50%) may require a longer annealing step and/or a higher annealing temperature as compared to primer sequences less than 60 base pairs and/or primer sequences with a low concentration of adenine-thymine base pairs (e.g., <50%).

In some embodiments, the qPCR cycle(s) include a denaturing step. As will be evident to one of ordinary skill in the art, a denaturing step involves increasing the temperature of the qPCR reaction to a sufficient temperature such that the DNA is melted (e.g., separate double stranded DNA to single stranded DNA). In some embodiments, the temperature of the denaturing step is between 75° C.-115° ° C. In some embodiments, the denaturing step temperature is about 75° C., 76° C. 77° C., 78° C., 79° C., 80° C., 81° C., 82° C., 83° C. 84° C., 85° ° C., 86° ° C., 87° C., 88° C., 89° C., 90° ° C., 91° C., 92° ° C., 93° C., 94° ° C., 95° C., 96° C., 97° C., 98° C. 99° ° C. 100° ° C., 101° C., 102° C., 103° C., 104° C., 105° C., 106° C., 107° C., 108° C., 109° C. 110° C., 111° C., 112° C., 113° C., 114° C., or 115° C. In some embodiments, the temperature of the denaturing step is about 95° C.

In some embodiments, the length of time of the denaturing step is between 0.5 seconds and 9.0 seconds. In some embodiments, the length of time of the denaturing step is about 0.5 seconds. 1.0 seconds, 1.5 seconds, 2.0 seconds, 2.5 seconds, 3.0 seconds, 3.5 seconds, 4.0 seconds, 4.5 seconds, 5.0 seconds, 5.5 seconds, 6.0 seconds, 6.5 seconds, 7.0 seconds, 7.5 seconds, 8.0 seconds, 8.5 seconds, or 9.0 seconds. In some embodiments, the denaturing step length is 3 seconds.

The length and temperature of the amplification/extension step of a qPCR reaction cycle may be depend, for example, on the length of the target genomic marker and/or the activity of the enzyme.

In some embodiments, the amplification step is performed at a temperature between 45° C.-75° C. In some embodiments, the amplification step is performed at a temperature of about 45° ° C., 46° ° C., 47° C., 48° C., 49° ° C., 50° ° C., 51° ° C., 52° ° C., 53° C., 54° C., 55° C., 56° ° C., 57° C., 58° ° C., 59° C., 60° ° C. 61° C., 62° ° C., 63° C., 64° C., 65° ° C., 66° ° C. 67° C., 68° C., 69° ° C., 70° C. 71° C., 72° ° C., 73° C., 74° C., or 75° C. In some embodiments, the amplification step is performed at about 60° C.

In some embodiments, the length of time of the amplification step is between 15 seconds and 1 minute. In some embodiments, the length of time of the amplification step is about 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, or 45 seconds. In some embodiments, the length of time of the amplification step is about 30 seconds.

The number of cycles (e.g., denaturing, annealing, amplifying/extension) may vary based, for example, on the detection of a target amplified piece of DNA (e.g., by fluorescence of the DNA probe). In some embodiments, the number of cycles is selected for robust detection of positive sample. In some embodiments, the number of cycles is selected to minimize the number of false positives.

In some embodiments, the qPCR reaction comprises 20 cycles, 21 cycles, 22 cycles, 23 cycles, 24 cycles, 25 cycles, 26 cycles, 27 cycles, 28 cycles, 29 cycles, 30 cycles, 31 cycles, 32 cycles, 33 cycles, 34 cycles, 35 cycles, 36 cycles, 37 cycles, 38 cycles, 39 cycles, or 40 cycles, 41 cycles, 42 cycles, 43 cycles, 44 cycles, 45 cycles, 46 cycles, 47 cycles, 48 cycles, 49 cycles, or 50 cycles. In some embodiments, the qPCR reaction comprises 40 cycles

An amplified piece of DNA is the DNA that is produced during a qPCR reaction after the sets of primers bind target genomic markers and are extended by enzyme (e.g., polymerase). The number of cycles in the qPCR reaction lead to amplification of the DNA. In some embodiments, the amplified nucleotide sequence of the genomic marker is between about 50 nucleotides-about 1200 nucleotides. In some embodiments, the amplified nucleotide sequence of the genomic marker is about 50 nucleotides-about 900 nucleotides. In some embodiments, the target amplified piece of DNA is about 500 nucleotides-about 1000 nucleotides. In some embodiments, the amplified nucleotide sequence of the genomic marker is about 50 nucleotides, 100 nucleotides, 150 nucleotides, 200 nucleotides, 250 nucleotides, 300 nucleotides, 350 nucleotides, 400 nucleotides, 450 nucleotides, 500 nucleotides, 550 nucleotides, 600 nucleotides, 650 nucleotides, 700 nucleotides, 750 nucleotides, 800 nucleotides, 850 nucleotides, 900 nucleotides, 1000 nucleotides, 1050 nucleotides, 1100 nucleotides, 1150 nucleotides, or 1200 nucleotides. In some embodiments, the genomic markers are about 150 nucleotides long. In some embodiments, the genomic markers are about 100 nucleotides long. In some embodiments, the genomic markers are about 200 nucleotides long. In some embodiments, the genomic markers are about 100, 105, 110, 115, 120, 125, 130, 135, 140, 145, 150, 155, 160, 165, 170, 175, 180, 185, 190, 195, or 200 nucleotides long.

In some embodiments, determining whether a target genomic marker is present is assessed at a particular time point during the qPCR reaction. In some embodiments, determining whether a target genomic marker is present is assessed at a particular cycle number during the qPCR reaction. In some embodiments, determining whether a target genomic marker is present is assessed at a particular cycle number during the qPCR reaction, for example by analyzing the amplification plot. In some embodiments, determining whether a target genomic marker is present is assessed at cycle 25, 26, 27, 28, 29, 30, 31, 32, 33, 34, or 35. In some embodiments, determining whether a target genomic marker is present is assessed at cycle 25. In some embodiments, determining whether a target genomic marker is present is assessed at cycle 30. In some embodiments, determining whether a target genomic marker is present is assessed at cycle 35.

In some embodiments, a target genomic marker is determined to be present in a sample if a detectable amount of an amplified product corresponding to the genomic marker for the strain is detected at a particular time point during the qPCR reaction. In some embodiments, a target genomic marker is determined to be present in a sample if a fluorescent signal corresponding to the amount of an amplified product is detected at a particular time point during the qPCR reaction. In some embodiments, a target genomic marker is determined to be present in a sample if the amplification peak of the qPCR reaction crosses a threshold particular time point during the qPCR reaction. In some embodiments, the threshold cycle is cycle 25 and a target genomic marker is determined to be present in a sample if the amplification peak of the qPCR reaction crosses a threshold at cycle 25. In some embodiments, the threshold cycle is cycle 30 and a target genomic marker is determined to be present in a sample if the amplification peak of the qPCR reaction crosses a threshold at cycle 35.

As will be evident to one of ordinary skill in the art, selection of a threshold cycle at which to determine whether a genomic marker has been amplified and therefore indicating that the corresponding bacterial strain is present in the sample, depends on balancing one or more factors. For example, use of a higher cycle number for the threshold cycle may result a higher rate of false positives as increased cycles of qPCR increase the likelihood of non-specific (off-target) amplification. Alternatively, use of a higher cycle number for the threshold cycle may result aa higher rate of false negatives as fewer cycles of qPCR may not allow sufficient amplification of genomic markers that are present albeit at low abundance.

In some embodiments, the genomic marker sequence is a protein coding sequence. In some embodiments, the genomic marker sequence is a non-protein coding sequence.

In some embodiments, the target bacterial strain is Clostridium saccharogumia. Any nucleotide sequence that is capable of identifying a nucleotide sequence as belonging to Clostridium saccharogumia may be used in the methods described herein. In some embodiments, the genomic marker is a Clostridium saccharogumia 16S rDNA sequence. In some embodiments, the genomic marker is unique to Clostridium saccharogumia. In some embodiments, the genomic marker identifying Clostridium saccharogumia is a protein coding sequence, or portion thereof. In some embodiments, the genomic marker identifying Clostridium saccharogumia is a non-coding sequence. In some embodiments, the genomic marker identifying Clostridium saccharogumia is a nucleotide sequence encoding a transmembrane protein, or portion thereof. In some embodiments, the genomic marker identifying Clostridium saccharogumia is a nucleotide sequence encoding glutaconyl-CoA decarboxylase subunit beta (gcdB), or portion thereof.

In some embodiments, the presence of Clostridium saccharogumia is determined by amplifying a nucleotide sequence of a genomic marker for Clostridium saccharogumia. In some embodiments, one or more genomic marker for Clostridium saccharogumia is amplified by qPCR. In some embodiments, the genomic marker for determining the presence of Clostridium saccharogumia godB, or a portion thereof. In some embodiments, the genomic marker for determining the presence of Clostridium saccharogumia is amplified using a forward primer having a nucleotide sequence provided by SEQ ID NO: 17. In some embodiments, the genomic marker for determining the presence of Clostridium saccharogumia is amplified using a reverse primer having a nucleotide sequence provided by SEQ ID NO: 33. In some embodiments, the genomic marker for determining the presence of Clostridium saccharogumia is amplified using a forward primer having a nucleotide sequence provided by SEQ ID NO: 17 and a reverse primer having a nucleotide sequence provided by SEQ ID NO: 33.

In some embodiments, the genomic marker for determining the presence of Clostridium saccharogumia is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 17. In some embodiments, the genomic marker for determining the presence of Clostridium saccharogumia is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 33. In some embodiments, the genomic marker for determining the presence of Clostridium saccharogumia is amplified using a forward primer having a nucleotide sequence provided by SEQ ID NO: 17 and a reverse primer having a nucleotide sequence provided by SEQ ID NO: 33. In some embodiments, the genomic marker for determining the presence of Clostridium saccharogumia comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 49.

In some embodiments, amplification of the Clostridium saccharogumia genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 49. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 49.

In some embodiments, the target bacterial strain is Flavonifractor plautii. Any nucleotide sequence that is capable of identifying a nucleotide sequence as belonging to Flavonifractor plautii may be used in the methods described herein. In some embodiments, the genomic marker is a Flavonifractor plautii 16S rDNA sequence. In some embodiments, the genomic marker is unique to Flavonifractor plautii. In some embodiments, the genomic marker identifying Flavonifractor plautii is a protein coding sequence, or portion thereof. In some embodiments, the genomic marker identifying Flavonifractor plautii is a non-coding sequence. In some embodiments, the genomic marker identifying Flavonifractor plautii is a nucleotide sequence encoding a transmembrane protein, or portion thereof. In some embodiments, the genomic marker identifying Flavonifractor plautii is a nucleotide sequence encoding glutaconyl-CoA decarboxylase subunit beta (gcdB), or portion thereof.

In some embodiments, the presence of Flavonifractor plautii is determined by amplifying a nucleotide sequence of a genomic marker for Flavonifractor plautii. In some embodiments, one or more genomic marker for Flavonifractor plautii is amplified by qPCR. In some embodiments, the genomic marker for determining the presence of Flavonifractor plautii gcdB, or a portion thereof. In some embodiments, the genomic marker for determining the presence of Flavonifractor plautii is amplified using a forward primer having a nucleotide sequence provided by SEQ ID NO: 18. In some embodiments, the genomic marker for determining the presence of Flavonifractor plautii is amplified using a reverse primer having a nucleotide sequence provided by SEQ ID NO: 34. In some embodiments, the genomic marker for determining the presence of Flavonifractor plautii is amplified using a forward primer having a nucleotide sequence provided by SEQ ID NO: 18 and a reverse primer having a nucleotide sequence provided by SEQ ID NO: 34.

In some embodiments, the genomic marker for determining the presence of Flavonifractor plautii is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 18. In some embodiments, the genomic marker for determining the presence of Flavonifractor plautii is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 34. In some embodiments, the genomic marker for determining the presence of Flavonifractor plautii is amplified using a forward primer having a nucleotide sequence provided by SEQ ID NO: 18 and a reverse primer having a nucleotide sequence provided by SEQ ID NO: 34. In some embodiments, the genomic marker for determining the presence of Flavonifractor plautii comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 50.

In some embodiments, amplification of the Flavonifractor plautii genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 50. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 50.

In some embodiments, the target bacterial strain is Clostridium hathewayi. Any nucleotide sequence that is capable of identifying a nucleotide sequence as belonging to Clostridium hathewayi may be used in the methods described herein. In some embodiments, the genomic marker is a Clostridium hathewayi 16S rDNA sequence. In some embodiments, the genomic marker is unique to Clostridium hathewayi. In some embodiments, the genomic marker identifying Clostridium hathewayi is a protein coding sequence, or portion thereof. In some embodiments, the genomic marker identifying Clostridium hathewayi is a non-coding sequence. In some embodiments, the genomic marker identifying Clostridium hathewayi is a nucleotide sequence encoding a transmembrane protein, or portion thereof. In some embodiments, the genomic marker identifying Clostridium hathewayi is a nucleotide sequence encoding glutaconyl-CoA decarboxylase subunit beta (gcdB), or portion thereof.

In some embodiments, the presence of Clostridium hathewayi is determined by amplifying a nucleotide sequence of a genomic marker for Clostridium hathewayi. In some embodiments, one or more genomic marker for Clostridium hathewayi is amplified by qPCR. In some embodiments, the genomic marker for determining the presence of Clostridium hathewayi gcdB, or a portion thereof. In some embodiments, the genomic marker for determining the presence of Clostridium hathewayi is amplified using a forward primer having a nucleotide sequence provided by SEQ ID NO: 19. In some embodiments, the genomic marker for determining the presence of Clostridium hathewayi is amplified using a reverse primer having a nucleotide sequence provided by SEQ ID NO: 35. In some embodiments, the genomic marker for determining the presence of Clostridium hathewayi is amplified using a forward primer having a nucleotide sequence provided by SEQ ID NO: 19 and a reverse primer having a nucleotide sequence provided by SEQ ID NO: 35.

In some embodiments, the genomic marker for determining the presence of Clostridium hathewayi is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 19. In some embodiments, the genomic marker for determining the presence of Clostridium hathewayi is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 35. In some embodiments, the genomic marker for determining the presence of Clostridium hathewayi is amplified using a forward primer having a nucleotide sequence provided by SEQ ID NO: 19 and a reverse primer having a nucleotide sequence provided by SEQ ID NO: 35. In some embodiments, the genomic marker for determining the presence of Clostridium hathewayi comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 51.

In some embodiments, amplification of the Clostridium hathewayi genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 51. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 51.

In some embodiments, the target bacterial strain is Blautia coccoides. Any nucleotide sequence that is capable of identifying a nucleotide sequence as belonging to Blautia coccoides may be used in the methods described herein. In some embodiments, the genomic marker is a Blautia coccoides 16S rDNA sequence. In some embodiments, the genomic marker is unique to Blautia coccoides. In some embodiments, the genomic marker identifying Blautia coccoides is a protein coding sequence, or portion thereof. In some embodiments, the genomic marker identifying Blautia coccoides is a non-coding sequence. In some embodiments, the genomic marker identifying Blautia coccoides is a nucleotide sequence encoding a transmembrane protein, or portion thereof. In some embodiments, the genomic marker identifying Blautia coccoides is a nucleotide sequence encoding glutaconyl-CoA decarboxylase subunit beta (gcdB), or portion thereof.

In some embodiments, the presence of Blautia coccoides is determined by amplifying a nucleotide sequence of a genomic marker for Blautia coccoides. In some embodiments, one or more genomic marker for Blautia coccoides is amplified by qPCR. In some embodiments, the genomic marker for determining the presence of Blautia coccoides gcdB, or a portion thereof. In some embodiments, the genomic marker for determining the presence of Blautia coccoides is amplified using a forward primer having a nucleotide sequence provided by SEQ ID NO: 20. In some embodiments, the genomic marker for determining the presence of Blautia coccoides is amplified using a reverse primer having a nucleotide sequence provided by SEQ ID NO: 36. In some embodiments, the genomic marker for determining the presence of Blautia coccoides is amplified using a forward primer having a nucleotide sequence provided by SEQ ID NO: 20 and a reverse primer having a nucleotide sequence provided by SEQ ID NO: 36.

In some embodiments, the genomic marker for determining the presence of Blautia coccoides is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 20. In some embodiments, the genomic marker for determining the presence of Blautia coccoides is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 36. In some embodiments, the genomic marker for determining the presence of Blautia coccoides is amplified using a forward primer having a nucleotide sequence provided by SEQ ID NO: 20 and a reverse primer having a nucleotide sequence provided by SEQ ID NO: 36. In some embodiments, the genomic marker for determining the presence of Blautia coccoides comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 52.

In some embodiments, amplification of the Blautia coccoides genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 52. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 52.

In some embodiments, the target bacterial strain is Clostridium bolteae. Any nucleotide sequence that is capable of identifying a nucleotide sequence as belonging to Clostridium bolteae may be used in the methods described herein. In some embodiments, the genomic marker is a Clostridium bolteae 16S rDNA sequence. In some embodiments, the genomic marker is unique to Clostridium bolteae. In some embodiments, the genomic marker identifying Clostridium bolteae is a protein coding sequence, or portion thereof. In some embodiments, the genomic marker identifying Clostridium bolteae is a non-coding sequence. In some embodiments, the genomic marker identifying Clostridium bolteae is a nucleotide sequence encoding a transmembrane protein, or portion thereof. In some embodiments, the genomic marker identifying Clostridium bolteae is a nucleotide sequence encoding glutaconyl-CoA decarboxylase subunit beta (gcdB), or portion thereof.

In some embodiments, the presence of Clostridium bolteae is determined by amplifying a nucleotide sequence of a genomic marker for Clostridium bolteae. In some embodiments, one or more genomic marker for Clostridium bolteae is amplified by qPCR. In some embodiments, the genomic marker for determining the presence of Clostridium bolteae gcdB, or a portion thereof. In some embodiments, the genomic marker for determining the presence of Clostridium bolteae is amplified using a forward primer having a nucleotide sequence provided by SEQ ID NO: 21. In some embodiments, the genomic marker for determining the presence of Clostridium bolteae is amplified using a reverse primer having a nucleotide sequence provided by SEQ ID NO: 37. In some embodiments, the genomic marker for determining the presence of Clostridium bolteae is amplified using a forward primer having a nucleotide sequence provided by SEQ ID NO: 21 and a reverse primer having a nucleotide sequence provided by SEQ ID NO: 37.

In some embodiments, the genomic marker for determining the presence of Clostridium bolteae is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 21. In some embodiments, the genomic marker for determining the presence of Clostridium bolteae is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 37. In some embodiments, the genomic marker for determining the presence of Clostridium bolteae is amplified using a forward primer having a nucleotide sequence provided by SEQ ID NO: 21 and a reverse primer having a nucleotide sequence provided by SEQ ID NO: 37. In some embodiments, the genomic marker for determining the presence of Clostridium bolteae comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 53.

In some embodiments, amplification of the Clostridium bolteae genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 53. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 53.

In some embodiments, the target bacterial strain is Clostridium indolis. Any nucleotide sequence that is capable of identifying a nucleotide sequence as belonging to Clostridium indolis may be used in the methods described herein. In some embodiments, the genomic marker is a Clostridium indolis 16S rDNA sequence. In some embodiments, the genomic marker is unique to Clostridium indolis. In some embodiments, the genomic marker identifying Clostridium indolis is a protein coding sequence, or portion thereof. In some embodiments, the genomic marker identifying Clostridium indolis is a non-coding sequence. In some embodiments, the genomic marker identifying Clostridium indolis is a nucleotide sequence encoding a transmembrane protein, or portion thereof. In some embodiments, the genomic marker identifying Clostridium indolis is a nucleotide sequence encoding glutaconyl-CoA decarboxylase subunit beta (gcdB), or portion thereof.

In some embodiments, the presence of Clostridium indolis is determined by amplifying a nucleotide sequence of a genomic marker for Clostridium indolis. In some embodiments, one or more genomic marker for Clostridium indolis is amplified by qPCR. In some embodiments, the genomic marker for determining the presence of Clostridium indolis gcdB, or a portion thereof. In some embodiments, the genomic marker for determining the presence of Clostridium indolis is amplified using a forward primer having a nucleotide sequence provided by SEQ ID NO: 22. In some embodiments, the genomic marker for determining the presence of Clostridium indolis is amplified using a reverse primer having a nucleotide sequence provided by SEQ ID NO: 38. In some embodiments, the genomic marker for determining the presence of Clostridium indolis is amplified using a forward primer having a nucleotide sequence provided by SEQ ID NO: 22 and a reverse primer having a nucleotide sequence provided by SEQ ID NO: 38.

In some embodiments, the genomic marker for determining the presence of Clostridium indolis is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 22. In some embodiments, the genomic marker for determining the presence of Clostridium indolis is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 38. In some embodiments, the genomic marker for determining the presence of Clostridium indolis is amplified using a forward primer having a nucleotide sequence provided by SEQ ID NO: 22 and a reverse primer having a nucleotide sequence provided by SEQ ID NO: 38. In some embodiments, the genomic marker for determining the presence of Clostridium indolis comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 54.

In some embodiments, amplification of the Clostridium indolis genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 54. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 54.

In some embodiments, the target bacterial strain is Anaerotruncus colihominis. Any nucleotide sequence that is capable of identifying a nucleotide sequence as belonging to Anaerotruncus colihominis may be used in the methods described herein. In some embodiments, the genomic marker is a Anaerotruncus colihominis 16S rDNA sequence. In some embodiments, the genomic marker is unique to Anaerotruncus colihominis. In some embodiments, the genomic marker identifying Anaerotruncus colihominis is a protein coding sequence, or portion thereof. In some embodiments, the genomic marker identifying Anaerotruncus colihominis is a non-coding sequence. In some embodiments, the genomic marker identifying Anaerotruncus colihominis is a nucleotide sequence encoding a transmembrane protein, or portion thereof. In some embodiments, the genomic marker identifying Anaerotruncus colihominis is a nucleotide sequence encoding glutaconyl-CoA decarboxylase subunit beta (gcdB), or portion thereof.

In some embodiments, the presence of Anaerotruncus colihominis is determined by amplifying a nucleotide sequence of a genomic marker for Anaerotruncus colihominis. In some embodiments, one or more genomic marker for Anaerotruncus colihominis is amplified by qPCR. In some embodiments, the genomic marker for determining the presence of Anaerotruncus colihominis gcdB, or a portion thereof. In some embodiments, the genomic marker for determining the presence of Anaerotruncus colihominis is amplified using a forward primer having a nucleotide sequence provided by SEQ ID NO: 23. In some embodiments, the genomic marker for determining the presence of Anaerotruncus colihominis is amplified using a reverse primer having a nucleotide sequence provided by SEQ ID NO: 39. In some embodiments, the genomic marker for determining the presence of Anaerotruncus colihominis is amplified using a forward primer having a nucleotide sequence provided by SEQ ID NO: 23 and a reverse primer having a nucleotide sequence provided by SEQ ID NO: 39.

In some embodiments, the genomic marker for determining the presence of Anaerotruncus colihominis is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 23. In some embodiments, the genomic marker for determining the presence of Anaerotruncus colihominis is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 39. In some embodiments, the genomic marker for determining the presence of Anaerotruncus colihominis is amplified using a forward primer having a nucleotide sequence provided by SEQ ID NO: 23 and a reverse primer having a nucleotide sequence provided by SEQ ID NO: 39. In some embodiments, the genomic marker for determining the presence of Anaerotruncus colihominis comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 55.

In some embodiments, amplification of the Anaerotruncus colihominis genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 55. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 55.

In some embodiments, the target bacterial strain is Ruminococcus sp. Any nucleotide sequence that is capable of identifying a nucleotide sequence as belonging to Ruminococcus sp. may be used in the methods described herein. In some embodiments, the genomic marker is a Ruminococcus sp. 16S rDNA sequence. In some embodiments, the genomic marker is unique to Ruminococcus sp. In some embodiments, the genomic marker identifying Ruminococcus sp. is a protein coding sequence, or portion thereof. In some embodiments, the genomic marker identifying Ruminococcus sp. is a non-coding sequence. In some embodiments, the genomic marker identifying Ruminococcus sp. is a nucleotide sequence encoding a transmembrane protein, or portion thereof. In some embodiments, the genomic marker identifying Ruminococcus sp. is a nucleotide sequence encoding glutaconyl-CoA decarboxylase subunit beta (gcdB), or portion thereof.

In some embodiments, the presence of Ruminococcus sp. is determined by amplifying a nucleotide sequence of a genomic marker for Ruminococcus sp. In some embodiments, one or more genomic marker for Ruminococcus sp. is amplified by qPCR. In some embodiments, the genomic marker for determining the presence of Ruminococcus sp. gcdB, or a portion thereof. In some embodiments, the genomic marker for determining the presence of Ruminococcus sp. is amplified using a forward primer having a nucleotide sequence provided by SEQ ID NO: 24. In some embodiments, the genomic marker for determining the presence of Ruminococcus sp. is amplified using a reverse primer having a nucleotide sequence provided by SEQ ID NO: 40. In some embodiments, the genomic marker for determining the presence of Ruminococcus sp. is amplified using a forward primer having a nucleotide sequence provided by SEQ ID NO: 24 and a reverse primer having a nucleotide sequence provided by SEQ ID NO: 40.

In some embodiments, the genomic marker for determining the presence of Ruminococcus sp. is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 24. In some embodiments, the genomic marker for determining the presence of Ruminococcus sp. is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 40. In some embodiments, the genomic marker for determining the presence of Ruminococcus sp. is amplified using a forward primer having a nucleotide sequence provided by SEQ ID NO: 24 and a reverse primer having a nucleotide sequence provided by SEQ ID NO: 40. In some embodiments, the genomic marker for determining the presence of Ruminococcus sp. comprises a nucleotide sequence having at least 85%, 86%, 87%, 88%. 89%, 90%, 91%, 92%, 93%, 94%, 95%, 96%, 97%, 98%, or 99% identical to the sequence provided by SEQ ID NO: 56.

In some embodiments, amplification of the Ruminococcus sp. genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 56. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 56.

In some embodiments, the target bacterial strain is Clostridium lavalense. Any nucleotide sequence that is capable of identifying a nucleotide sequence as belonging to Clostridium lavalense may be used in the methods described herein. In some embodiments, the genomic marker is a Clostridium lavalense 16S rDNA sequence. In some embodiments, the genomic marker is unique to Clostridium lavalense. In some embodiments, the genomic marker identifying Clostridium lavalense is a protein coding sequence, or portion thereof. In some embodiments, the genomic marker identifying Clostridium lavalense is a non-coding sequence. In some embodiments, the genomic marker identifying Clostridium lavalense is a nucleotide sequence encoding a transmembrane protein, or portion thereof. In some embodiments, the genomic marker identifying Clostridium lavalense is a nucleotide sequence encoding glutaconyl-CoA decarboxylase subunit beta (gcdB), or portion thereof.

In some embodiments, the presence of Clostridium lavalense is determined by amplifying a nucleotide sequence of a genomic marker for Clostridium lavalense. In some embodiments, one or more genomic marker for Clostridium lavalense is amplified by qPCR. In some embodiments, the genomic marker for determining the presence of Clostridium lavalense gcdB, or a portion thereof. In some embodiments, the genomic marker for determining the presence of Clostridium lavalense is amplified using a forward primer having a nucleotide sequence provided by SEQ ID NO: 25. In some embodiments, the genomic marker for determining the presence of Clostridium lavalense is amplified using a reverse primer having a nucleotide sequence provided by SEQ ID NO: 41. In some embodiments, the genomic marker for determining the presence of Clostridium lavalense is amplified using a forward primer having a nucleotide sequence provided by SEQ ID NO: 25 and a reverse primer having a nucleotide sequence provided by SEQ ID NO: 41.

In some embodiments, the genomic marker for determining the presence of Clostridium lavalense is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 25. In some embodiments, the genomic marker for determining the presence of Clostridium lavalense is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 41. In some embodiments, the genomic marker for determining the presence of Clostridium lavalense is amplified using a forward primer having a nucleotide sequence provided by SEQ ID NO: 25 and a reverse primer having a nucleotide sequence provided by SEQ ID NO: 41. In some embodiments, the genomic marker for determining the presence of Clostridium lavalense comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 57.

In some embodiments, amplification of the Clostridium lavalense genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 57. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 57.

In some embodiments, the target bacterial strain is Clostridium symbiosum. Any nucleotide sequence that is capable of identifying a nucleotide sequence as belonging to Clostridium symbiosum may be used in the methods described herein. In some embodiments, the genomic marker is a Clostridium symbiosum 16S rDNA sequence. In some embodiments, the genomic marker is unique to Clostridium symbiosum. In some embodiments, the genomic marker identifying Clostridium symbiosum is a protein coding sequence, or portion thereof. In some embodiments, the genomic marker identifying Clostridium symbiosum is a non-coding sequence. In some embodiments, the genomic marker identifying Clostridium symbiosum is a nucleotide sequence encoding a transmembrane protein, or portion thereof. In some embodiments, the genomic marker identifying Clostridium symbiosum is a nucleotide sequence encoding glutaconyl-CoA decarboxylase subunit beta (gcdB), or portion thereof.

In some embodiments, the presence of Clostridium symbiosum is determined by amplifying a nucleotide sequence of a genomic marker for Clostridium symbiosum. In some embodiments, one or more genomic marker for Clostridium symbiosum is amplified by qPCR. In some embodiments, the genomic marker for determining the presence of Clostridium symbiosum gcdB, or a portion thereof. In some embodiments, the genomic marker for determining the presence of Clostridium symbiosum is amplified using a forward primer having a nucleotide sequence provided by SEQ ID NO: 26. In some embodiments, the genomic marker for determining the presence of Clostridium symbiosum is amplified using a reverse primer having a nucleotide sequence provided by SEQ ID NO: 42. In some embodiments, the genomic marker for determining the presence of Clostridium symbiosum is amplified using a forward primer having a nucleotide sequence provided by SEQ ID NO: 26 and a reverse primer having a nucleotide sequence provided by SEQ ID NO: 42.

In some embodiments, the genomic marker for determining the presence of Clostridium symbiosum is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 26. In some embodiments, the genomic marker for determining the presence of Clostridium symbiosum is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 42. In some embodiments, the genomic marker for determining the presence of Clostridium symbiosum is amplified using a forward primer having a nucleotide sequence provided by SEQ ID NO: 26 and a reverse primer having a nucleotide sequence provided by SEQ ID NO: 42. In some embodiments, the genomic marker for determining the presence of Clostridium symbiosum comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 58.

In some embodiments, amplification of the Clostridium symbiosum genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 58. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 58.

In some embodiments, the target bacterial strain is Clostridium ramosum. Any nucleotide sequence that is capable of identifying a nucleotide sequence as belonging to Clostridium ramosum may be used in the methods described herein. In some embodiments, the genomic marker is a Clostridium ramosum 16S rDNA sequence. In some embodiments, the genomic marker is unique to Clostridium ramosum. In some embodiments, the genomic marker identifying Clostridium ramosum is a protein coding sequence, or portion thereof. In some embodiments, the genomic marker identifying Clostridium ramosum is a non-coding sequence. In some embodiments, the genomic marker identifying Clostridium ramosum is a nucleotide sequence encoding a transmembrane protein, or portion thereof. In some embodiments, the genomic marker identifying Clostridium ramosum is a nucleotide sequence encoding glutaconyl-CoA decarboxylase subunit beta (gcdB), or portion thereof.

In some embodiments, the presence of Clostridium ramosum is determined by amplifying a nucleotide sequence of a genomic marker for Clostridium ramosum. In some embodiments, one or more genomic marker for Clostridium ramosum is amplified by qPCR. In some embodiments, the genomic marker for determining the presence of Clostridium ramosum gcdB, or a portion thereof. In some embodiments, the genomic marker for determining the presence of Clostridium ramosum is amplified using a forward primer having a nucleotide sequence provided by SEQ ID NO: 27. In some embodiments, the genomic marker for determining the presence of Clostridium ramosum is amplified using a reverse primer having a nucleotide sequence provided by SEQ ID NO: 43. In some embodiments, the genomic marker for determining the presence of Clostridium ramosum is amplified using a forward primer having a nucleotide sequence provided by SEQ ID NO: 27 and a reverse primer having a nucleotide sequence provided by SEQ ID NO: 43.

In some embodiments, the genomic marker for determining the presence of Clostridium ramosum is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 27. In some embodiments, the genomic marker for determining the presence of Clostridium ramosum is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 43. In some embodiments, the genomic marker for determining the presence of Clostridium ramosum is amplified using a forward primer having a nucleotide sequence provided by SEQ ID NO: 27 and a reverse primer having a nucleotide sequence provided by SEQ ID NO: 43. In some embodiments, the genomic marker for determining the presence of Clostridium ramosum comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 59.

In some embodiments, amplification of the Clostridium ramosum genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 59. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 59.

In some embodiments, the target bacterial strain is Eubacterium contortum. Any nucleotide sequence that is capable of identifying a nucleotide sequence as belonging to Eubacterium contortum may be used in the methods described herein. In some embodiments, the genomic marker is a Eubacterium contortum 16S rDNA sequence. In some embodiments, the genomic marker is unique to Eubacterium contortum. In some embodiments, the genomic marker identifying Eubacterium contortum is a protein coding sequence, or portion thereof. In some embodiments, the genomic marker identifying Eubacterium contortum is a non-coding sequence. In some embodiments, the genomic marker identifying Eubacterium contortum is a nucleotide sequence encoding a transmembrane protein, or portion thereof. In some embodiments, the genomic marker identifying Eubacterium contortum is a nucleotide sequence encoding glutaconyl-CoA decarboxylase subunit beta (gcdB), or portion thereof.

In some embodiments, the presence of Eubacterium contortum is determined by amplifying a nucleotide sequence of a genomic marker for Eubacterium contortum. In some embodiments, one or more genomic marker for Eubacterium contortum is amplified by qPCR. In some embodiments, the genomic marker for determining the presence of Eubacterium contortum gcdB, or a portion thereof. In some embodiments, the genomic marker for determining the presence of Eubacterium contortum is amplified using a forward primer having a nucleotide sequence provided by SEQ ID NO: 28. In some embodiments, the genomic marker for determining the presence of Eubacterium contortum is amplified using a reverse primer having a nucleotide sequence provided by SEQ ID NO: 44. In some embodiments, the genomic marker for determining the presence of Eubacterium contortum is amplified using a forward primer having a nucleotide sequence provided by SEQ ID NO: 28 and a reverse primer having a nucleotide sequence provided by SEQ ID NO: 44.

In some embodiments, the genomic marker for determining the presence of Eubacterium contortum is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 28. In some embodiments, the genomic marker for determining the presence of Eubacterium contortum is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 44. In some embodiments, the genomic marker for determining the presence of Eubacterium contortum is amplified using a forward primer having a nucleotide sequence provided by SEQ ID NO: 28 and a reverse primer having a nucleotide sequence provided by SEQ ID NO: 44. In some embodiments, the genomic marker for determining the presence of Eubacterium contortum comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 60.

In some embodiments, amplification of the Eubacterium contortum genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 60. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 60.

In some embodiments, the target bacterial strain is Clostridium scindens. Any nucleotide sequence that is capable of identifying a nucleotide sequence as belonging to Clostridium scindens may be used in the methods described herein. In some embodiments, the genomic marker is a Clostridium scindens 16S rDNA sequence. In some embodiments, the genomic marker is unique to Clostridium scindens. In some embodiments, the genomic marker identifying Clostridium scindens is a protein coding sequence, or portion thereof. In some embodiments, the genomic marker identifying Clostridium scindens is a non-coding sequence. In some embodiments, the genomic marker identifying Clostridium scindens is a nucleotide sequence encoding a transmembrane protein, or portion thereof. In some embodiments, the genomic marker identifying Clostridium scindens is a nucleotide sequence encoding glutaconyl-CoA decarboxylase subunit beta (gcdB), or portion thereof.

In some embodiments, the presence of Clostridium scindens is determined by amplifying a nucleotide sequence of a genomic marker for Clostridium scindens. In some embodiments, one or more genomic marker for Clostridium scindens is amplified by qPCR. In some embodiments, the genomic marker for determining the presence of Clostridium scindens gcdB, or a portion thereof. In some embodiments, the genomic marker for determining the presence of Clostridium scindens is amplified using a forward primer having a nucleotide sequence provided by SEQ ID NO: 29. In some embodiments, the genomic marker for determining the presence of Clostridium scindens is amplified using a reverse primer having a nucleotide sequence provided by SEQ ID NO: 45. In some embodiments, the genomic marker for determining the presence of Clostridium scindens is amplified using a forward primer having a nucleotide sequence provided by SEQ ID NO: 29 and a reverse primer having a nucleotide sequence provided by SEQ ID NO: 45.

In some embodiments, the genomic marker for determining the presence of Clostridium scindens is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 29. In some embodiments, the genomic marker for determining the presence of Clostridium scindens is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 45. In some embodiments, the genomic marker for determining the presence of Clostridium scindens is amplified using a forward primer having a nucleotide sequence provided by SEQ ID NO: 29 and a reverse primer having a nucleotide sequence provided by SEQ ID NO: 45. In some embodiments, the genomic marker for determining the presence of Clostridium scindens comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 61.

In some embodiments, amplification of the Clostridium scindens genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 61. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 61.

In some embodiments, the target bacterial strain is Lachnospiraceae bacterium. Any nucleotide sequence that is capable of identifying a nucleotide sequence as belonging to Lachnospiraceae bacterium may be used in the methods described herein. In some embodiments, the genomic marker is a Lachnospiraceae bacterium 16S rDNA sequence. In some embodiments, the genomic marker is unique to Lachnospiraceae bacterium. In some embodiments, the genomic marker identifying Lachnospiraceae bacterium is a protein coding sequence, or portion thereof. In some embodiments, the genomic marker identifying Lachnospiraceae bacterium is a non-coding sequence. In some embodiments, the genomic marker identifying Lachnospiraceae bacterium is a nucleotide sequence encoding a transmembrane protein, or portion thereof. In some embodiments, the genomic marker identifying Lachnospiraceae bacterium is a nucleotide sequence encoding glutaconyl-CoA decarboxylase subunit beta (gcdB), or portion thereof.

In some embodiments, the presence of Lachnospiraceae bacterium is determined by amplifying a nucleotide sequence of a genomic marker for Lachnospiraceae bacterium. In some embodiments, one or more genomic marker for Lachnospiraceae bacterium is amplified by qPCR. In some embodiments, the genomic marker for determining the presence of Lachnospiraceae bacterium gcdB, or a portion thereof. In some embodiments, the genomic marker for determining the presence of Lachnospiraceae bacterium is amplified using a forward primer having a nucleotide sequence provided by SEQ ID NO: 30. In some embodiments, the genomic marker for determining the presence of Lachnospiraceae bacterium is amplified using a reverse primer having a nucleotide sequence provided by SEQ ID NO: 46. In some embodiments, the genomic marker for determining the presence of Lachnospiraceae bacterium is amplified using a forward primer having a nucleotide sequence provided by SEQ ID NO: 30 and a reverse primer having a nucleotide sequence provided by SEQ ID NO: 46.

In some embodiments, the genomic marker for determining the presence of Lachnospiraceae bacterium is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 30. In some embodiments, the genomic marker for determining the presence of Lachnospiraceae bacterium is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 46. In some embodiments, the genomic marker for determining the presence of Lachnospiraceae bacterium is amplified using a forward primer having a nucleotide sequence provided by SEQ ID NO: 30 and a reverse primer having a nucleotide sequence provided by SEQ ID NO: 46. In some embodiments, the genomic marker for determining the presence of Lachnospiraceae bacterium comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 62.

In some embodiments, amplification of the Lachnospiraceae bacterium genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 62. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 62.

In some embodiments, the target bacterial strain is Clostridium sp. Any nucleotide sequence that is capable of identifying a nucleotide sequence as belonging to Clostridium sp. may be used in the methods described herein. In some embodiments, the genomic marker is a Clostridium sp. 16S rDNA sequence. In some embodiments, the genomic marker is unique to Clostridium sp. In some embodiments, the genomic marker identifying Clostridium sp. is a protein coding sequence, or portion thereof. In some embodiments, the genomic marker identifying Clostridium sp. is a non-coding sequence. In some embodiments, the genomic marker identifying Clostridium sp. is a nucleotide sequence encoding a transmembrane protein, or portion thereof. In some embodiments, the genomic marker identifying Clostridium sp. is a nucleotide sequence encoding glutaconyl-CoA decarboxylase subunit beta (gcdB), or portion thereof.

In some embodiments, the presence of Clostridium sp. is determined by amplifying a nucleotide sequence of a genomic marker for Clostridium sp. In some embodiments, one or more genomic marker for Clostridium sp. is amplified by qPCR. In some embodiments, the genomic marker for determining the presence of Clostridium sp. gcdB, or a portion thereof. In some embodiments, the genomic marker for determining the presence of Clostridium sp. is amplified using a forward primer having a nucleotide sequence provided by SEQ ID NO: 31. In some embodiments, the genomic marker for determining the presence of Clostridium sp. is amplified using a reverse primer having a nucleotide sequence provided by SEQ ID NO: 47. In some embodiments, the genomic marker for determining the presence of Clostridium sp. is amplified using a forward primer having a nucleotide sequence provided by SEQ ID NO: 31 and a reverse primer having a nucleotide sequence provided by SEQ ID NO: 47.

In some embodiments, the genomic marker for determining the presence of Clostridium sp. is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 31. In some embodiments, the genomic marker for determining the presence of Clostridium sp. is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 47. In some embodiments, the genomic marker for determining the presence of Clostridium sp. is amplified using a forward primer having a nucleotide sequence provided by SEQ ID NO: 31 and a reverse primer having a nucleotide sequence provided by SEQ ID NO: 47. In some embodiments, the genomic marker for determining the presence of Clostridium sp. comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 63.

In some embodiments, amplification of the Clostridium sp. genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 63. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 63.

In some embodiments, the target bacterial strain is Lachnospiraceae bacterium. Any nucleotide sequence that is capable of identifying a nucleotide sequence as belonging to Lachnospiraceae bacterium may be used in the methods described herein. In some embodiments, the genomic marker is a Lachnospiraceae bacterium 16S rDNA sequence. In some embodiments, the genomic marker is unique to Lachnospiraceae bacterium. In some embodiments, the genomic marker identifying Lachnospiraceae bacterium is a protein coding sequence, or portion thereof. In some embodiments, the genomic marker identifying Lachnospiraceae bacterium is a non-coding sequence. In some embodiments, the genomic marker identifying Lachnospiraceae bacterium is a nucleotide sequence encoding a transmembrane protein, or portion thereof. In some embodiments, the genomic marker identifying Lachnospiraceae bacterium is a nucleotide sequence encoding glutaconyl-CoA decarboxylase subunit beta (gcdB), or portion thereof.

In some embodiments, the presence of Lachnospiraceae bacterium is determined by amplifying a nucleotide sequence of a genomic marker for Lachnospiraceae bacterium. In some embodiments, one or more genomic marker for Lachnospiraceae bacterium is amplified by qPCR. In some embodiments, the genomic marker for determining the presence of Lachnospiraceae bacterium gcdB, or a portion thereof. In some embodiments, the genomic marker for determining the presence of Lachnospiraceae bacterium is amplified using a forward primer having a nucleotide sequence provided by SEQ ID NO: 32. In some embodiments, the genomic marker for determining the presence of Lachnospiraceae bacterium is amplified using a reverse primer having a nucleotide sequence provided by SEQ ID NO: 48. In some embodiments, the genomic marker for determining the presence of Lachnospiraceae bacterium is amplified using a forward primer having a nucleotide sequence provided by SEQ ID NO: 32 and a reverse primer having a nucleotide sequence provided by SEQ ID NO: 48.

In some embodiments, the genomic marker for determining the presence of Lachnospiraceae bacterium is amplified using a forward primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 32. In some embodiments, the genomic marker for determining the presence of Lachnospiraceae bacterium is amplified using a reverse primer having a nucleotide sequence that is at least 85%, 86%, 87%, 88%, 89%, 90%, 91%, 92%, 93%, 94%, 95%, 96%, 97%, 98%, or 99% identical to the sequence provided by SEQ ID NO: 48. In some embodiments, the genomic marker for determining the presence of Lachnospiraceae bacterium is amplified using a forward primer having a nucleotide sequence provided by SEQ ID NO: 32 and a reverse primer having a nucleotide sequence provided by SEQ ID NO: 48. In some embodiments, the genomic marker for determining the presence of Lachnospiraceae bacterium comprises a nucleotide sequence having at least 85%, 86%, 87%, 88%, 89%, 90%, 91%, 92%, 93%, 94%, 95%, 96%, 97%, 98%, or 99% identical to the sequence provided by SEQ ID NO: 64.

In some embodiments, amplification of the Lachnospiraceae bacterium genomic marker GCDB, or portion thereof, is detected by a DNA probe, e.g., a DNA probe that is included in the qPCR reaction. In some embodiments, the DNA probe has the sequence that is present in the nucleotide sequence of SEQ ID NO: 64. In some embodiments, the DNA probe has a sequence that is at least 85%, 86%, 87%, 88%, 89%, 90%, 91%, 92%, 93%, 94%, 95%, 96%, 97%, 98%, or 99% identical to a nucleotide sequence that is present in SEQ ID NO: 64.

Strain 1 16S ribosomal RNA Clostridium saccharogumia
SEQ ID NO: 1
CAGAATTAAGAAACACACGTCAAAGAAGGATCCGGAAACGGATAGAAGAGAAAAAGAAGAGCTAAACA
AACGAAAGATTGTAAAGATAAAGAGACAATGGAGAGTTTGATCCTGGCTCAGGATGAACGCTGGCGGC
GTGCCTAATACATGCAAGTCGAACGCGGGCAGCAATGCCCGAGTGGCGAACGGGTGAGTAATACATAA
GTAACCTGCCCTTTACAGGGGGATAACTATTGGAAACGATAGCTAAGACCGCATAGGTAAAGATACCG
CATGGTAAGTTTATTAAAAGTGCCAAGGCACTGGTAGAGGATGGACTTATGGCGCATTAGCTAGTTGG
TGAGGTAACGGCTCACCAAGGCGACGATGCGTAGCCGACCTGAGAGGGTGACCGGCCACACTGGGACT
GAGACACGGCCCAGACTCCTACGGGAGGCAGCAGTAGGGAATTTTCGGCAATGGGGGGAACCCTGACC
GAGCAACGCCGCGTGAAGGAGGAAGGTCTTCGGACTGTAAACTTCTGTTATAAAGGAAGAAAGGCGGA
TACAGGGAATGGTATCCGAGTGACGGTACTTTATGAGGAAGCCACGGCTAACTACGTGCCAGCAGCCG
CGGTAATACGTAGGTGGCAAGCGTTATCCGGAATTATTGGGCGTAAAGAGGGAGCAGGCGGCAGCAAG
GGTCTGTGGTGAAAGACTGAAGCTTAACTTCAGTAAGCCATAGAAACCGGGCAGCTAGAGTGCAGGAG
AGGATCGTGGAATTCCATGTGTAGCGGTGAAATGCGTAGATATATGGAGGAACACCAGTGGCGAAGGC
GACGATCTGGCCTGCAACTGACGCTCAGTCCCGAAAGCGTGGGGAGCAAATAGGATTAGATACCCTAG
TAGTCCACGCCGTAAACGATGAGTACTAAGTGTTGGGAGTCAAATCTCAGTGCTGCAGTTAACGCAGT
AAGTACTCCGCCTGAGTAGTACGTTCGCAAGAATGAAACTCAAAGGAATTGACGGGGGCCCGCACAAG
CGGTGGAGCATGTGGTTTAATTCGAAGCAACGCGAAGAACCTTACCAGGTCTTGACATACTCATAAAG
GCTCCAGAGATGGAGAGATAGATATATGGGATACAGGTGGTGCATGGTTGTCGTCAGCTCGTGTCGTG
AGATGTTGGGTTAAGTCCCGCAACGAGCGCAACCCTTATCGTTAGTTACCATCATTAAGTTGGGGACT
CTAGCGAGACTGCCAGTGACAAGCTGGAGGAAGGCGGGGATGACGTCAAATCATCATGCCCCTTATGA
CCTGGGCTACACACGTGCTACAATGGATGGAGCAGAGGGAAGCGAAGCCGCGAGGTGGAGCGAAACCC
AGAAAACCATTCTCAGTTCGGATTGTAGTCTGCAACTCGACTACATGAAGTTGGAATCGCTAGTAATC
GCGAATCAGCATGTCGCGGTGAATACGTTCTCGGGCCTTGTACACACCGCCCGTCACACCATGAGAGT
TGATAACACCCGAAGCCGGTGGCCTAACCGCAAGGAGGGAGCTGTCTAAGGTGGGATTGATGATTGGG
GTGAAGTCGTAACAAGGTATCCCTACGGGAACGTGGGGATGGATCACCTCCTTTCTAAGGAGAAAGAA
GAGAAGTGAAGTTTCATTAATACTGTTTAGTTTTGAGTGATGCTCAGGAGG
Strain 2 16S ribosomal RNA Flavonifractor plautii
SEQ ID NO: 2
TTAAGCTTCGATAAGATGATTTGAGGAACGGAGACGTTCTTTAGATACCATTTTATTGAGAGTTTGAT
CCTGGCTCAGGATGAACGCTGGCGGCGTGCTTAACACATGCAAGTCGAACGGGGTGCTCATGACGGAG
GATTCGTCCAACGGATTGAGTTACCTAGTGGCGGACGGGTGAGTAACGCGTGAGGAACCTGCCTTGGA
GAGGGGAATAACACTCCGAAAGGAGTGCTAATACCGCATGATGCAGTTGGGTCGCATGGCTCTGACTG
CCAAAGATTTATCGCTCTGAGATGGCCTCGCGTCTGATTAGCTAGTAGGCGGGGTAACGGCCCACCTA
GGCGACGATCAGTAGCCGGACTGAGAGGTTGACCGGCCACATTGGGACTGAGACACGGCCCAGACTCC
TACGGGAGGCAGCAGTGGGGAATATTGGGCAATGGGCGCAAGCCTGACCCAGCAACGCCGCGTGAAGG
AAGAAGGCTTTCGGGTTGTAAACTTCTTTTGTCGGGGACGAAACAAATGACGGTACCCGACGAATAAG
CCACGGCTAACTACGTGCCAGCAGCCGCGGTAATACGTAGGTGGCAAGCGTTATCCGGATTTACTGGG
TGTAAAGGGCGTGTAGGCGGGATTGCAAGTCAGATGTGAAAACTGGGGGCTCAACCTCCAGCCTGCAT
TTGAAACTGTAGTTCTTGAGTGCTGGAGAGGCAATCGGAATTCCGTGTGTAGCGGTGAAATGCGTAGA
TATACGGAGGAACACCAGTGGCGAAGGCGGATTGCTGGACAGTAACTGACGCTGAGGCGCGAAAGCGT
GGGGAGCAAACAGGATTAGATACCCTGGTAGTCCACGCCGTAAACGATGGATACTAGGTGTGGGGGGT
CTGACCCCCTCCGTGCCGCAGTTAACACAATAAGTATCCCACCTGGGGAGTACGATCGCAAGGTTGAA
ACTCAAAGGAATTGACGGGGGCCCGCACAAGCGGTGGAGTATGTGGTTTAATTCGAAGCAACGCGAAG
AACCTTACCAGGGCTTGACATCCCACTAACGAAGCAGAGATGCATTAGGTGCCCTTCGGGGAAAGTGG
AGACAGGTGGTGCATGGTTGTCGTCAGCTCGTGTCGTGAGATGTTGGGTTAAGTCCCGCAACGAGCGC
AACCCTTATTGTTAGTTGCTACGCAAGAGCACTCTAGCGAGACTGCCGTTGACAAAACGGAGGAAGGT
GGGGACGACGTCAAATCATCATGCCCCTTATGTCCTGGGCCACACACGTACTACAATGGTGGTTAACA
GAGGGAGGCAATACCGCGAGGTGGAGCAAATCCCTAAAAGCCATCCCAGTTCGGATTGCAGGCTGAAA
CCCGCCTGTATGAAGTTGGAATCGCTAGTAATCGCGGATCAGCATGCCGCGGTGAATACGTTCCCGGG
CCTTGTACACACCGCCCGTCACACCATGAGAGTCGGGAACACCCGAAGTCCGTAGCCTAACCGCAAGG
AGGGCGCGGCCGAAGGTGGGTTCGATAATTGGGGTGAAGTCGTAACAAGGTAGCCGTATCGGAAGGTG
CGGCTGGATCACCTCCTTTCTAAGGAGACTTCAACGGAAGTAGTTTTCTGTTGTAACGTCCTAAGGTC
AGCTTTCTGGAGCGAATGGCTTTCACGTIGTTTAATTTAGAGGGTACACTCTC
Strain 3 16S ribosomal RNA Clostridium hathewayi
SEQ ID NO: 3
ATTCAGAACGAATACAATTATTGTATACGAACCAAACAACAAGTAAAACGGGAAATAAATTAGCTAGT
TAGTTGATTTTGACCCCGGATCAAACGAATCATTTAACATGAGAGTTCGATCCTGGCTCAGGATGAAC
GCTGGCGGCGTGCTTAACACATGCAAGTCGAGCGAAGCGGITTCAATGAAGTTTTCGGATGGAATTGA
AATTGACTTAGCGGCGGACGGGTGAGTAACGCGTGGGTAACCTGCCTTACACTGGGGGATAACAGTTA
GAAATGACTGCTAATACCGCATAAGCGCACAGGGCCGCATGGTCTGGTGTGAAAAACTCCGGTGGTGT
AAGATGGACCCGCGTCTGATTAGGTAGTTGGTGGGGTAACGGCCCACCAAGCCGACGATCAGTAGCCG
ACCTGAGAGGGTGACCGGCCACATTGGGACTGAGACACGGCCCAAACTCCTACGGGAGGCAGCAGTGG
GGAATATTGGACAATGGGCGAAAGCCTGATCCAGCGACGCCGCGTGAGTGAAGAAGTATTTCGGTATG
TAAAGCTCTATCAGCAGGGAAGAAAATGACGGTACCTGACTAAGAAGCCCCGGCTAACTACGTGCCAG
CAGCCGCGGTAATACGTAGGGGGCAAGCGTTATCCGGATTTACTGGGTGTAAAGGGAGCGTAGACGGT
TAAGCAAGTCTGAAGTGAAAGCCCGGGGCTCAACCCCGGTACTGCTTTGGAAACTGTTTGACTTGAGT
GCAGGAGAGGTAAGTGGAATTCCTAGTGTAGCGGTGAAATGCGTAGATATTAGGAGGAACACCAGTGG
CGAAGGCGGCTTACTGGACTGTAACTGACGTTGAGGCTCGAAAGCGTGGGGAGCAAACAGGATTAGAT
ACCCTGGTAGTCCACGCCGTAAACGATGAATACTAGGTGTCGGGGGACAACGTCCTTCGGTGCCGCCG
CTAACGCAATAAGTATTCCACCTGGGGAGTACGTTCGCAAGAATGAAACTCAAAGGAATTGACGGGGA
CCCGCACAAGCGGTGGAGCATGTGGTTTAATTCGAAGCAACGCGAAGAACCTTACCAAGTCTTGACAT
CCCATTGAAAATCATTTAACCGTGATCCCTCTTCGGAGCAATGGAGACAGGTGGTGCATGGTTGTCGT
CAGCTCGTGTCGTGAGATGTTGGGTTAAGTCCCGCAACGAGCGCAACCCTTATCCTTAGTAGCCAGCA
CATGATGGTGGGCACTCTGGGGAGACTGCCAGGGATAACCTGGAGGAAGGTGGGGATGACGTCAAATC
ATCATGCCCCTTATGATTTGGGCTACACACGTGCTACAATGGCGTAAACAAAGGGAAGCAAAGGAGCG
ATCTGGAGCAAACCCCAAAAATAACGTCTCAGTTCGGATTGCAGGCTGCAACTCGCCTGCATGAAGCT
GGAATCGCTAGTAATCGCGAATCAGAATGTCGCGGTGAATACGTTCCCGGGTCTTGTACACACCGCCC
GTCACACCATGGGAGTTGGTAACGCCCGAAGTCAGTGACCCAACCGTAAGGAGGGAGCTGCCGAAGGC
GGGACTGATAACTGGGGTGAAGTCGTAACAAGGTAGCCGTATCGGAAGGTGCGGCTGGATCACCTCCT
TTCTAAGGAAGAAGAAGTAAGGGTTTTATATACTGTTGAGTCTTGTGTTTCAAAGGAAGGCGAAAGCC
GAAGAAAAATAGAATAAAAGAATAGAAACATCAGAGACAAAGAGATTCTGGTGCCGA
Strain 4 16S ribosomal RNA Blautia producta
SEQ ID NO: 4
CGAAGTCTGGTGTGGAATATTCAGACACCCCAAATTTTTTCATACACTGCGATCCGGGAAGCATATGA
GGAAGCGCGGCAGCAGAGCATGGAGGATATTACAGATGATGCCATGGTCATGGAGCGCTTTGGAAACT
TGAAAATTAAGTTGGTGGAAGGTTCCTATGAGAACATTAAAATCACTACGCCGGAGGACATTCTTGTC
GCTGAGAAAATTTTGGAAAAAAATTAGAGAAATCTATTGACAACTTCCGACATAAGTGATAAGATACC
ACTTGTCGCTGACCGAGTGACAAACATGGAGAGATATCGAAGTGGTCATAACGAGGCGGTCTTGAAAA
CCGTTTGTCCGCAAGGGCGCGTGGGTTCGAATCCCACTCTCTCCGTTCCCGAAAGCAGATATCGAGAA
CGAATCAGAGTGACGAGTTCAAGATTAGTTATGATGCTTTTGGATATATAGAGAAGCAGTTATTTGAA
AGCTTACTTTCAAACTGTTGTTTTATTCAGCCTGGAGAAGTACCCAAGCGGCTGAAGGGGCTCCCCTG
GAAAGGGAGTAGGTCGTTAATAGCGGCGCGAGGGTTCAAATCCCTCCTTCTCCGTTTTCTCTTAAAAA
AAGAGAAAAAAGTTGTTGACAAGCGATCGGATATATGATAATCTATTATAGCTGTCGCAAAAACGACA
ACGATTTATGACATTGAAAATGAGCTTTGCTTAAAAGCTTTGTGAGTAAATGCAAAGGTTTAGCGTAG
TTGAAAGAAGTTTCGACACAACGTAAAAAAACTTTTGAAAAACAATACAATTAAAAATAAATTTTTAA
TTGTCATAAGTTCTTGACAAGCACGAAACGATATGATAAAATATCAAAGCTGTCAAAAACTGACAGGA
ACCTTGATAACTGAACAGTGAAACACATGAATCGAAAATTCTTTTACATTCATTAGTAACAAAACGAA
CGGTTCTAAACGAACCAAAACAGTAAAAGGGATATCTTATTGAGCCTAGCTCAATAAGCCAGAATTAG
CCAAAGTTAATTCTGTTCCTGGACAAACACTTGATCAGAGAGTTTGATCCTGGCTCAGGATGAACGCT
GGCGGCGTGCTTAACACATGCAAGTCGAGCGAAGCATTAAGACAGATTTCTTCGGATTGAAGTCTTTG
TGACTGAGCGGCGGACGGGTGAGTAACGCGTGGGTAACCTGCCTTGGTCTGGTGTGAAAAACTCCGGT
GGTATGAGATGGACCCGCGTCTGATTAGCTAGTTGGAGGGGTAACGGCCCACCAAGGCGACGATCAGT
AGCCGGCCTGAGAGGGTGAACGGCCACATTGGGACTGAGACACGGCCCAGACTCCTACGGGAGGCAGC
AGTGGGGAATATTGCACAATGGGGGAAACCCTGATGCAGCGACGCCGCGTGAAGGAAGAAGTATCTCG
GTATGTAAACTTCTATCAGCAGGGAAGAAAATGACGGTACCTGACTAAGAAGCCCCGGCTAACTACGT
GCCAGCAGCCGCGGTAATACGTAGGGGGCAAGCGTTATCCGGATTTACTGGGTGTAAAGGGAGCGTAG
ACGGAAGAGCAAGTCTGATGTGAAAGGCTGGGGCTTAACCCCAGGACTGCATTGGAAACTGTTGTTCT
AGAGTGCCGGAGAGGTAAGCGGAATTCCTAGTGTAGCGGTGAAATGCGTAGATATTAGGAGGAACACC
AGTGGCGAAGGCGGCTTACTGGACGGTAACTGACGTTGAGGCTCGAAAGCGTGGGGAGCAAACAGGAT
TAGATACCCTGGTAGTCCACGCCGTAAACGATGAATACTAGGTGTCGGGTGGCAAAGCCATTCGGTGC
CGCAGCAAACGCAATAAGTATTCCACCTGGGGAGTACGTTCGCAAGAATGAAACTCAAAGGAATTGAC
GGGGACCCGCACAAGCGGTGGAGCATGTGGTTTAATTCGAAGCAACGCGAAGAACCTTACCAAGTCTT
GACATCCCGTCGTCAGCTCGTGTCGTGAGATGTTGGGTTAAGTCCCGCAACGAGCGCAACCCTTATCC
TTAGTAGCCAGCACATGATGGTGGGCACTCTAGGGAGACTGCCGGGGATAACCCGGAGGAAGGCGGGG
ACGACGTCAAATCATCATGCCCCTTATGATTTGGGCTACACACGTGCTACAATGGCGTAAACAAAGGG
AAGCGAGACAGCGATGTTGAGCGAATCCCAAAAATAACGTCCCAGTTCGGACTGCAGTCTGCAACTCG
ACTGCACGAAGCTGGAATCGCTAGTAATCGCGGATCAGAATGCCGCGGTGAATACGTTCCCGGGTCTT
GTACACACCGCCCGTCACACCATGGGAGTCAGTAACGCCCGAAGTCAGTGACCTAACCGAAAGGAAGG
AGCTGCCGAAGGCGGGACCGATAACTGGGGTGAAGTCGTAACAAGGTAGCCGTATCGGAAGGTGCGGC
TGGATCACCTCCTTTCTAAGGAAGAAGAAGTAGAGAAAAGTGTTTCACTGTTGAGTTACCAAGA
Strain 5 16S ribosomal RNA Clostridium bolteae
SEQ ID NO: 5
GAAAATTCAGAACAAAACCAAGTAATGAAGGTTAAAGATAACTAGCCAAGCTAAGTAGTCTGAAACCT
GGAATCAACTTTTAAACATGAGAGTTTGATCCTGGCTCAGGATGAACGCTGGCGGCGTGCCTAACACA
TGCAAGTCGAACGAAGCAATTAAAATGAAGTTTTCGGATGGATTTTTGATTGACTGAGTGGCGGACGG
GTGAGTAACGCGTGGATAACCTGCCTCACACTGGGGGATAACAGTTAGAAATGACTGCTAATACCGCA
TAAGCGCACAGTACCGCATGGTACGGTGTGAAAAACTCCGGTGGTGTGAGATGGATCCGCGTCTGATT
AGCCAGTTGGCGGGGTAACGGCCCACCAAAGCGACGATCAGTAGCCGACCTGAGAGGGTGACCGGCCA
CATTGGGACTGAGACACGGCCCAAACTCCTACGGGAGGCAGCAGTGGGGAATATTGCACAATGGGCGA
AAGCCTGATGCAGCGACGCCGCGTGAGTGAAGAAGTATTTCGGTATGTAAAGCTCTATCAGCAGGGAA
GAAAATGACGGTACCTGACTAAGAAGCCCCGGCTAACTACGTGCCAGCAGCCGCGGTAATACGTAGGG
GGCAAGCGTTATCCGGATTTACTGGGTGTAAAGGGAGCGTAGACGGCGAAGCAAGTCTGAAGTGAAAA
CCCAGGGCTCAACCCTGGGACTGCTTTGGAAACTGTTTTGCTAGAGTGTCGGAGAGGTAAGTGGAATT
CCTAGTGTAGCGGTGAAATGCGTAGATATTAGGAGGAACACCAGTGGCGAAGGCGGCTTACTGGACGA
TAACTGACGTTGAGGCTCGAAAGCGTGGGGAGCAAACAGGATTAGATACCCTGGTAGTCCACGCCGTA
AACGATGAATGCTAGGTGTTGGGGGGCAAAGCCCTTCGGTGCCGTCGCAAACGCAGTAAGCATTCCAC
CTGGGGAGTACGTTCGCAAGAATGAAACTCAAAGGAATTGACGGGGACCCGCACAAGCGGTGGAGCAT
GTGGTTTAATTCGAAGCAACGCGAAGAACCTTACCAAGTCTTGACATCCTCTTGACCGGCGTGTAACG
GCGCCTTCCCTTCGGGGCAAGAGAGACAGGTGGTGCATGGTTGTCGTCAGCTCGTGTCGTGAGATGTT
GGGTTAAGTCCCGCAACGAGCGCAACCCTTATCCTTAGTAGCCAGCAGGTAAAGCTGGGCACTCTAGG
GAGACTGCCAGGGATAACCTGGAGGAAGGTGGGGATGACGTCAAATCATCATGCCCCTTATGATTTGG
GCTACACACGTGCTACAATGGCGTAAACAAAGGGAAGCAAGACAGTGATGTGGAGCAAATCCCAAAAA
TAACGTCCCAGTTCGGACTGTAGTCTGCAACCCGACTACACGAAGCTGGAATCGCTAGTAATCGCGAA
TCAGAATGTCGCGGTGAATACGTTCCCGGGTCTTGTACACACCGCCCGTCACACCATGGGAGTCAGCA
ACGCCCGAAGTCAGTGACCCAACTCGCAAGAGAGGGAGCTGCCGAAGGCGGGGCAGGTAACTGGGGTG
AAGTCGTAACAAGGTAGCCGTATCGGAAGGTGCGGCTGGATCACCTCCTTTCTAAGGAAGAAGAAGTA
GGGGTTTATGTATTATTGAGTCCTTGATTAAAAGGTGCTTAAAATTTTCCGGTGGCGATGCGCTTAGG
GG
Strain 6 16S ribosomal RNA Clostridium indolis
SEQ ID NO: 6
AAAAAAGATACACGAACGTTCTTTATAAAAGGACACAGTAAAACAAGGATAAAAAAGCTAGCGCTTTT
TAGATCCAAGATTGAACATGAGAGTTTGATCCTGGCTCAGGATGAACGCTGGCGGCGTGCTTAACACA
TGCAAGTCGAACGAAGCATTTTGGAAGGAAGTTTTCGGATGGAATTCCTTAATGACTGAGTGGCGGAC
GGGTGAGTAACGCGTGGGGAACCTGCCCTATACAGGGGGATAACAGCTGGAAACGGCTGCTAATACCG
CATAAGCGCACAGAATCGCATGATTCGGTGTGAAAAGCTCCGGCAGTATAGGATGGTCCCGCGTCTGA
TTAGCTGGTTGGCGGGGTAACGGCCCACCAAGGCGACGATCAGTAGCCGGCTTGAGAGAGTGGACGGC
CACATTGGGACTGAGACACGGCCCAAACTCCTACGGGAGGCAGCAGTGGGGAATATTGCACAATGGGG
GAAACCCTGATGCAGCGACGCCGCGTGAGTGAAGAAGTATTTCGGTATGTAAAGCTCTATCAGCAGGG
AAGAAAAAAGACGGTACCTGACTAAGAAGCCCCGGCTAACTACGTGCCAGCAGCCGCGGTAATACGTA
GGGGGCAAGCGTTATCCGGAATTACTGGGTGTAAAGGGTGCGTAGGTGGCATGGTAAGTCAGAAGTGA
AAGCCCGGGGCTTAACCCCGGGACTGCTTTTGAAACTGTCATGCTGGAGTGCAGGAGAGGTAAGCGGA
ATTCCTAGTGTAGCGGTGAAATGCGTAGATATTAGGAGGAACACCAGTGGCGAAGGCGGCTTACTGGA
CTGTCACTGACACTGATGCACGAAAGCGTGGGGAGCAAACAGGATTAGATACCCTGGTAGTCCACGCC
GTAAACGATGAATACTAGGTGTCGGGGCCGTAGAGGCTTCGGTGCCGCAGCAAACGCAGTAAGTATTC
CACCTGGGGAGTACGTTCGCAAGAATGAAACTCAAAGGAATTGACGGGGACCCGCACAAGCGGTGGAG
CATGTGGTTTAATTCGAAGCAACGCGAAGAACCTTACCTGGTCTTGACATCTAACTGACCGGTTCGTA
ATGGGACCTTTCCTTCGGGACAGTTAAGACAGGTGGTGCATGGTTGTCGTCAGCTCGTGTCGTGAGAT
GTTGGGTTAAGTCCCGCAACGAGCGCAACCCCTATCTTTAGTAGCCAGCATATAAGGTGGGCACTCTA
GAGAGACTGCCAGGGATAACCTGGAGGAAGGTGGGGACGACGTCAAATCATCATGCCCCTTATGGCCA
GGGCTACACACGTGCTACAATGGCGTAAACAAAGGGAAGCGAAGTCGTGAGGCGAAGCAAATCCCAGA
AATAACGTCTCAGTTCGGATTGTAGTCTGCAACTCGACTACATGAAGCTGGAATCGCTAGTAATCGTG
AATCAGAATGTCACGGTGAATACGTTCCCGGGTCTTGTACACACCGCCCGTCACACCATGGGAGTCAG
TAACGCCCGAAGTCAGTGACCCAACCTTATAGGAGGGAGCTGCCGAAGGTGGGACCGATAACTGGGGT
GAAGTCGTAACAAGGTAGCCGTATCGGAAGGTGCGGCTGGATCACCTCCTTTCTAAGGAAGAAGAAGT
AGGGGTTGTCTTATTGTTTAGTTATCAATGATAACAAAAAGTATCAAGCAAGGAAGTTCTATTGCACT
AAA
Strain 7 16S ribosomal RNA Anaerotruncus colihominis
SEQ ID NO: 7
ATCAAAGAGTTTGATCCTGGCTCAGGACGAACGCTGGCGGCGCGCCTAACACATGCAAGTGTGAGTAA
CACGTGAGCAACCTGCCTTTCAGAGGGGGATAACAGCCGGAAACGGCTGCTAATACCGCATGATGTTG
CGGGGGCACATGCCCCTGCAACCAAAGGAGCAATCCGCTGAAAGATGGGCTCGCGTCCGATTAGCCAG
TTGGCGGGGTAACGGCCCACCAAAGCGACGATCGGTAGCCGGACTGAGAGGTTGAACGGCCACATTGG
GACTGAGACACGGCCCAGACTCCTACGGGGGAAGACGGTCTTCGGATTGTAAACCTCTGTCTTTGGGG
AAGAAAATGACGGTACCCAAAGAGGAAGCTCCGGCTAACTACGTGCCAGCAGCCGCGGTAATACGTAG
GGAGCAAGCGTTGTCCGGAATTACTGGGTGTAAAGGGAGCGTAGGCGGGATGGCAAGTAGAATGTTAA
ATCCGAATTCCTAGTGTAGCGGTGAAATGCGTAGATATTAGGAGGAACACCAGTGGCGAAGGCGGCCT
GCTGGGCTTTAACTGACGCTGAGGCTCGAAAGCGTGGGGAGCAAACAGGATTAGATACCCTGGTAGTC
CACGCCGTAAACGATGATTACTAGGTGTGGGGGGACTGACCCCTTCCGTGCCGCAGTTAACACAATAA
GTAATCCACCTGGGGAGTACGGCCGCAAGGTTGAAACTCAAAGGAATTGACGGGGGCCCGCACAAGCA
GTGGAGTATGTGGTTTAATTCGAAGCAACGCGGGCATCCAGACAGGTGGTGCATGGTTGTCGTCAGCT
CGTGTCGTGAGATGTTGGGTTAAGTCCCGCAACGAGCGCAACCCTTATTATTAGTTGCTACGCAAGAG
CACTCTAATGAGACTGGGCTACACACGTACTACAATGGCACTAAAACAGAGGGCGGCGACACCGCGAG
GTGAAGCGAATCCCGAAAAAGTGTCTCAGTTCAGATTGCAGGCTGCAACCCGCCTGCATGAAGTCGGA
ATTGCTAGTAATCGCGGATCAGCATGCCGCGGTGAATACGTTCCCGGGCCTTGTACACACCGCTGTCG
AAGGTGGGATTGATGACTGGGGTGAAGTCGTAACAAGGTAGCCGTATCGGAAGGTGCGGCTGGATCAC
CTCCTTTCTAAGGAGCGAAGCCGGCGGAGAGCCGGCGGACTCTGGTCAGACAGGG
Strain 8 16S ribosomal RNA Drancourtella massiliensis
SEQ ID NO: 8
AACAGGAACTTTAAGTTCCACAAACCAGACAACAGTAACGGGATAGATTAGCGAGAGTTGATCTTGAC
CAGAACAAGTTCTTATTACGAGAGTTTGATCCTGGCTCAGGATGAACGCTGGCGGCGTGCCTAACACA
TGCAAGTCGAGCGAAGCGCTGTTTTCAGAATCTTCGGAGGAAGAGGACAGTGACTGAGCGGCGGACGG
GTGAGTAACGCGTGGGCAACCTGCCTCATACAGGGGGATAACAGTTAGAAATGACTGCTAATACCGCA
TAAGCGCACAGGACCGCATGGTGTAGTGTGAAAAACTCCGGTGGTATGAGATGGACCCGCGTCTGATT
AGGTAGTTGGTGGGGTAAAGGCCTACCAAGCCGACGATCAGTAGCCGACCTGAGAGGGTGACCGGCCA
CATTGGGACTGAGACACGGCCCAAACTCCTACGGGAGGCAGCAGTGGGGAATATTGCACAATGGGGGA
AACCCTGATGCAGCGACGCCGCGTGAAGGAAGAAGTATTTCGGTATGTAAACTTCTATCAGCAGGGAA
GAAGATGACGGTACCTGAGTAAGAAGCACCGGCTAAATACGTGCCAGCAGCCGCGGTAATACGTATGG
TGCAAGCGTTATCCGGATTTACTGGGTGTAAAGGGAGCGTAGACGGATAGGCAAGTCTGGAGTGAAAA
CCCAGGGCTCAACCCTGGGACTGCTTTGGAAACTGCAGATCTGGAGTGCCGGAGAGGTAAGCGGAATT
CCTAGTGTAGCGGTGAAATGCGTAGATATTAGGAGGAACACCAGTGGCGAAGGCGGCTTACTGGACGG
TGACTGACGTTGAGGCTCGAAAGCGTGGGGAGCAAACAGGATTAGATACCCTGGTAGTCCACGCCGTA
AACGATGACTACTAGGTGTCGGTGTGCAAAGCACATCGGTGCCGCAGCAAACGCAATAAGTAGTCCAC
CTGGGGAGTACGTTCGCAAGAATGAAACTCAAAGGAATTGACGGGGACCCGCACAAGCGGTGGAGCAT
GTGGTTTAATTCGAAGCAACGCGAAGAACCTTACCTGGTCTTGACATCCGGATGACGGGCGAGTAATG
TCGCCGTCCCTTCGGGGCATCCGAGACAGGTGGTGCATGGITGTCGTCAGCTCGTGTCGTGAGATGTT
GGGTTAAGTCCCGCAACGAGCGCAACCCTTATCTTCAGTAGCCAGCATATAAGGTGGGCACTCTGGAG
AGACTGCCAGGGAGAACCTGGAGGAAGGTGGGGATGACGTCAAATCATCATGCCCCTTATGGCCAGGG
CTACACACGTGCTACAATGGCGTAAACAAAGGGAAGCGAGAGGGTGACCTGGAGCGAATCCCAAAAAT
AACGTCTCAGTTCGGATTGTAGTCTGCAACTCGACTACATGAAGCTGGAATCGCTAGTAATCGCGGAT
CAGCATGCCGCGGTGAATACGTTCCCGGGTCTTGTACACACCGCCCGTCACACCATGGGAGTCAGTAA
CGCCCGAAGCCAGTGACCCAACCTTAGAGGAGGGAGCTGTCGAAGGCGGGACGGATAACTGGGGTGAA
GTCGTAACAAGGTAGCCGTATCGGAAGGTGCGGCTGGATCACCTCCTTTCTAAGGGAAGAAGTAAGGG
ACGTTGTCTATTGTTGAGCGATCGAAGAGGGGAGACCCGAAGAGGAGCTCAGAAGCAGTTGGTGGTGA
Strain 9 16S ribosomal RNA Clostridium lavalense
SEQ ID NO: 9
GAACCTTGAAAATCAAAGATTGAACAGTATGTAAAACCCTGAAAATTCTAAAAAATAAAGGTCTGGTT
TAGACCTTTGGATTTGAGAAAATTCAGAACAAAACCAAGTAATGAAGGTTAAAGATAACTAGCCAAGC
TAAGTAGTCTAAGACCAAGAATCAACTTTTAATTTGAGAGTTTGATCCTGGCTCAGGATGAACGCTGG
CGGCGTGCCTAACACATGCAAGTCGAACGAAGCATTTTAGATGAAGTTTTCGGATGGATTCTGAGATG
ACTGAGTGGCGGACGGGTGAGTAACACGTGGATAACCTGCCTCACACTGGGGGACAACAGTTAGAAAT
GACTGCTAATACCGCATAAGGATTAGCCAGTTGGCGGGGTAACGGCCCACCAAAGCGACGATCAGTAG
CCGACCTGAGAGGGTGACCGGCCACATTGGGACTGAGACACGGCCCAAACTCCTACGGGAGGCAGCAG
TGGGGAATATTGCACAATGGGCGAAAGCCTGATGCAGCGACGCCGCGTGAGTGAAGAAGTATTTCGGT
ATGTAAAGCTCTATCAGCAGGGAAGATAATGACGGTACCTGACTAAGAAGCCCCGGCTAACTACGTGC
CAGCAGCCGCGGTAATACGTAGGGGGCAAGCGTTATCCGGATTTACTGGGTGTAAAGGGAGCGTAGAC
GGCATGGCAAGTCTGAAGTGAAAACCCAGGGCTCAACCCTGGGACTGCTTTGGAAACTGTCAAGCTAG
AGTGCAGGAGAGGTAAGTGGAATTCCTAGTGTAGCGGTGAAATGCGTAGATATTAGGAGGAACACCAG
TGGCGAAGGCGGCTTACTGGACTGTAACTGACGTTGAGGCTCGAAAGCGTGGGGAGCAAACAGGATTA
GATACCCTGGTAGTCCACGCCGTAAACGATGAGTGCTAGGTGTTGGGGGGCAAAGCCCTTCGGTGCCG
TCGCAAACGCAATAAGCACTCCACCTGGGGAGTACGTTCGCAAGAATGAAACTCAAAGGAATTGACGG
GGACCCGCACAAGCGGTGGAGCATGTGGTTTAATTCGAAGCAACGCGAAGAACCTTACCAAGTCTTGA
CATCCTCTTGACCGGCGTGTAACGGCGCCTTTCCTTCGGGACAAGAGAGACAGGTGGTGCATGGTTGT
CGTCAGCTCGTGTCGTGAGATGTTGGGTTAAGTCCCGCAACGAGCGCAACCCTTATCCTTAGTAGCCA
GCATTAAGATGGGCACTCTAGGGAGACTGCCAGGGACAACCTGGAGGAAGGTGGGGATGACGTCAAAT
CATCATGCCCCTTATGATTTGGGCTACACACGTGCTACAATGGCGTAAACAAAGGGAAGCGACCCTGC
GAAGGTGAGCAAATCTCAAAAATAACGTCCCAGTTCGGACTGTAGTCTGCAACCCGACTACACGAAGC
TGGAATCGCTAGTAATCGCGAATCAGAATGTCGCGGTGAATACGTTCCCGGGTCTTGTACACACCGCC
CGTCACACCATGGGAGTCAGCAACGCCCGAAGTCAGTGACCCAACCGAAAGGAGGGAGCTGCCGAAGG
CGGGGCAGGTAACTGGGGTGAAGTCGTAACAAGGTAGCCGTATCGGAAGGTGCGGCTGGATCACCTCC
TTTCTAAGGAAGAAGAAGTAAGGGTTTTATATACTGTTGAATCTTGGATTTGCCAAGGT
Strain 10 16S ribosomal RNA Clostridium symbiosum
SEQ ID NO: 10
CAAAACAAGATTGATGAAAATATATAAAAATATCTGGAAAAACAGGCTTGACAACCCAATCTTAATAG
TTTTAAGTAATAACTGCGGAGAAATACTCAAGAGGCCGAAGAGGCGCCCCTGCTAAGGGTGTAGGTCG
GGCAACCGGCGCGAGGGTTCAAATCCCTCTTTCTCCGTTTTCTTTTGCTGCTGAGAACAAACGACAGC
GTTCAACAACAAAGAACCTTGAAAACTGAACAGTATGTAAAACCTTGAAAATTCTTAAAGAACAAAGG
CTTTTGGTTAAGCCTAAAAGTTCGAGAGTTTTGAACGGAATCAAAACTTAAACATGAGAGTTTGATCC
TAGCTCAGGATGAACGCTGGCGGCGTGCCTAACACATGCAAGTCGAACGAAGCAATTTAACGGAAGTT
TTCGGATGGAAGITGAATTGACTGAGTGGCGGACGGGTGAGTAACGCGTGGGTAACCTGCCTTGTACT
GGGGGACAACAGTTAGAAATGACTGCTAATACCGCATAAGCGCACAGTATCGCATGATACAGTGTGAA
AAACTCCGGTGGTACAAGATGGACCCGCGTCTGATTAGCTAGTTGGTAAGGTAACGGCTTACCAAGGC
GACGATCAGTAGCCGACCTGAGAGGGTGACCGGCCACATTGGGACTGAGACACGGCCCAAACTCCTAC
GGGAGGCAGCAGTGGGGAATATTGCACAATGGGCGAAAGCCTGATGCAGCGACGCCGCGTGAGTGAAG
AAGTATTTCGGTATGTAAAGCTCTATCAGCAGGGAAGAAAATGACGGTACCTGACTAAGAAGCCCCGG
CTAACTACGTGCCAGCAGCCGCGGTAATACGTAGGGGGCAAGCGTTATCCGGATTTACTGGGTGTAAA
GGGAGCGTAGACGGTAAAGCAAGTCTGAAGTGAAAGCCCGCGGCTCAACTGCGGGACTGCTTTGGAAA
CTGTTTAACTGGAGTGTCGGAGAGGTAAGTGGAATTCCTAGTGTAGCGGTGAAATGCGTAGATATTAG
GAGGAACACCAGTGGCGAAGGCGACTTACTGGACGATAACTGACGTTGAGGCTCGAAAGCGTGGGGAG
CAAACAGGATTAGATACCCTGGTAGTCCACGCCGTAAACGATGAATACTAGGTGTTGGGGAGCAAAGC
TCTTCGGTGCCGTCGCAAACGCAGTAAGTATTCCACCTGGGGAGTACGTTCGCAAGAATGAAACTCAA
AGGAATTGACGGGGACCCGCACAAGCGGTGGAGCATGTGGTTTAATTCGAAGCAACGCGAAGAACCTT
ACCAGGTCTTGACATCGATCCGACGGGGGAGTAACGTCCCCTTCCCTTCGGGGCGGAGAAGACAGGTG
GTGCATGGTTGTCGTCAGCTCGTGTCGTGAGATGTTGGGTTAAGTCCCGCAACGAGCGCAACCCTTAT
TCTAAGTAGCCAGCGGTTCGGCCGGGAACTCTTGGGAGACTGCCAGGGATAACCTGGAGGAAGGTGGG
GATGACGTCAAATCATCATGCCCCTTATGATCTGGGCTACACACGTGCTACAATGGCGTAAACAAAGA
GAAGCAAGACCGCGAGGTGGAGCAAATCTCAAAAATAACGTCTCAGTTCGGACTGCAGGCTGCAACTC
GCCTGCACGAAGCTGGAATCGCTAGTAATCGCGAATCAGAATGTCGCGGTGAATACGTTCCCGGGTCT
TGTACACACCGCCCGTCACACCATGGGAGTCAGTAACGCCCGAAGTCAGTGACCCAACCGCAAGGAGG
GAGCTGCCGAAGGCGGGACCGATAACTGGGGTGAAGTCGTAACAAGGTAGCCGTATCGGAAGGTGCGG
CTGGATCACCTCCTTTCTAAGGAAGAAGAAGTAGAGGTTTTATATACTGTTGAGTTTTCAAGCTCAGA
CACTTCCGGTGTCGATGCGCTCAGGGGTCACACCCGTTCCCATCCCGAACACGATGGTTAAGACCTGA
GCGGCCGATGATACTATATTGGAGACGATATGGGAAAGCAGGTGGATGCCGGATTTTTCTTTAAAAAA
CTCACTCGTAAGAGTGTTTTATATAGAACGGACACATCTGAATGGATGAATTCGTAGTTTCTTTAGAT
GAAAAAGACATCTGTGACGACGATGTGTTAACCCACTCCAGCAGGGAAGTGCTGTTCCATATAACTGG
TTTTTACCAGTGATTAGTGAATTTGAGTTTATCGAGTTTGCTAATGACTGATAAAGTTCAGTCAGATA
TGTACCTTGAAAACTGCATATTGAAATAATATCTAGATAGAGTTTCGAAACTCTTCCGAGTGGCTTGC
CACAAGGGAGAAAAGAGAGACAAAATCAAGACATCCGAGGTGTTACACGAAAGTGTAACCAAAACAAA
CCAAAAATTTTATTGTAAACCTAAACCAAAGATTCAACGCTATGAATCTTAGACT
Strain 11 16S ribosomal RNA Clostridium ramosum
SEQ ID NO: 11
ATTGAAAACTAAACAGAATTAAGAAACACACGTCAATATCCAGAAGGATAAAAAAGAAAAAAGAGCTA
AACAAACGAAGAGTIGTTTAAATAAACAATGGAGAGTTTGATCCTGGCTCAGGATGAACGCTGGCGGC
GTGCCTAATACATGCAAGTCGAACGCGAGCACTTGTGCTCGAGTGGCGAACGGGTGAGTAATACATAA
GTAACCTGCCCTAGACAGGGGGATAACTATTGGAAACGATAGCTAAGACCGCATAGGTACGGACACTG
CATGGTGACCGTATTAAAAGTGCCTCAAAGCACTGGTAGAGGATGGACTTATGGCGCATTAGCTGGTT
GGCGGGGTAACGGCCCACCAAGGCGACGATGCGTAGCCGACCTGAGAGGGTGACCGGCCACACTGGGA
CTGAGACACGGCCCAGACTCCTACGGGAGGCAGCAGTAGGGAATTTTCGGCAATGGGGGAAACCCTGA
CCGAGCAACGCCGCGTGAAGGAAGAAGGTTTTCGGATTGTAAACTTCTGTTATAAAGGAAGAACGGCG
GCTACAGGAAATGGTAGCCGAGTGACGGTACTTTATTAGAAAGCCACGGCTAACTACGTGCCAGCAGC
CGCGGTAATACGTAGGTGGCAAGCGTTATCCGGAATTATTGGGCGTAAAGAGGGAGCAGGCGGCAGCA
AGGGTCTGTGGTGAAAGCCTGAAGCTTAACTTCAGTAAGCCATAGAAACCAGGCAGCTAGAGTGCAGG
AGAGGATCGTGGAATTCCATGTGTAGCGGTGAAATGCGTAGATATATGGAGGAACACCAGTGGCGAAG
GCGACGATCTGGCCTGCAACTGACGCTCAGTCCCGAAAGCGTGGGGAGCAAATAGGATTAGATACCCT
AGTAGTCCACGCCGTAAACGATGAGTACTAAGTGTTGGATGTCAAAGTTCAGTGCTGCAGTTAACGCA
ATAAGTACTCCGCCTGAGTAGTACGTTCGCAAGAATGAAACTCAAAGGAATTGACGGGGGCCCGCACA
AGCGGTGGAGCATGTGGTTTAATTCGAAGCAACGCGAAGAACCTTACCAGGTCTTGACATACTCATAA
AGGCTCCAGAGATGGAGAGATAGCTATATGAGATACAGGTGGTGCATGGTTGTCGTCAGCTCGTGTCG
TGAGATGTTGGGTTAAGTCCCGCAACGAGCGCAACCCTTATCGTTAGTTACCATCATTAAGTTGGGGA
CTCTAGCGAGACTGCCAGTGACAAGCTGGAGGAAGGCGGGGATGACGTCAAATCATCATGCCCCTTAT
GACCTGGGCTACACACGTGCTACAATGGATGGTGCAGAGGGAAGCGAAGCCGCGAGGTGAAGCAAAAC
CCATAAAACCATTCTCAGTTCGGATTGTAGTCTGCAACTCGACTACATGAAGTTGGAATCGCTAGTAA
TCGCGAATCAGCATGTCGCGGTGAATACGTTCTCGGGCCTTGTACACACCGCCCGTCACACCACGAGA
GTTGATAACACCCGAAGCCGGTGGCCTAACCGCAAGGAAGGAGCTGTCTAAGGIGGGATTGATGCTAA
GGAGAAACGCATGATCGAAGATGATGCACAAGAGAAGTGATGTTTCGAAGATACTGTTTAGTTTTGAG
TGATACTCAGGT
Strain 12 16S ribosomal RNA Eubacterium contortum
SEQ ID NO: 12
ACTGAGATTAATCTCGGTGGTAAGAACAGTAAAAAGGGATAGAATTAGCTAGTAGTTAATTCTGACCC
GGATACAAACACTTTTAACGAGAGTTTGATCCTGGCTCAGGATGAACGCTGGCGGCGTGCTTAACACA
TGCAAGTCGAGCGAAGCGCTTTACTTAGATTTCTTCGGATTGAAAAGTTTTGCGACTGAGCGGCGGAC
GGGTGAGTAACGCGTGGGTAACCTGCCTCATACAGGGGGATAACAGTTAGAAATGACTGCTAATACCG
CATAAGACCACAGTACCGCATGGTACAGTGGGAAAAACTCCGGTGGTATGAGATGGACCCGCGTCTGA
TTAGCTAGTTGGTAAGGTAACGGCTTACCAAGGCGACGATCAGTAGCCGACCTGAGAGGGTGACCGGC
CACATTGGGACTGAGACACGGCCCAAACTCCTACGGGAGGCAGCAGTGGGGAATATTGCACAATGGGG
GAAACCCTGATGCAGCGACGCCGCGTGAAGGATGAAGTATTTCGGTATGTAAACTTCTATCAGCAGGG
AAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGTTATGTAAGTCTGATGTGAA
AACCCGGGGCTCAACCCCGGGACTGCATTGGAAACTATGTAACTAGAGTGTCGGAGAGGTAAGTGGAA
TTCCTAGTGTAGCGGTGAAATGCGTAGATATTAGGAGGAACACCAGTGGCGAAGGGGCTTACTGGAC
GATGACTGACGTTGAGGCTCGAAAGCGTGGGGAGCAAACAGGATTAGATACCCTGGTAGTCCACGCCG
TAAACGATGCATACTAGGTGTCGGGTGGCAAAGCCATTCGGTGCCGCAGCAAACGCAATAAGTATGCC
ACCTGGGGAGTACGTTCGCAAGAATGAAACTCAAAGGAATTGACGGGGACCCGCACAAGCGGTGGAGC
ATGTGGTTTAATTCGAAGCAACGCGAAGAACCTTACCTGCTCTTGACATCCCCCTGACCGGCGCGTAA
TGGTGCCTTTCCTTCGGGACAGGGGAGACAGGTGGTGCATGGTTGTCGTCAGCTCGTGTCGTGAGATG
TTGGGTTAAGTCCCGCAACGAGCGCAACCCTTATCTTTAGTAGCCAGCGGTTTGGCCGGGCACTCTAG
AGAGACTGCCAGGGATAACCTGGAGGAAGGTGGGGATGACGTCAAATCATCATGCCCCTTATGAGCAG
GGCTACACACGTGCTACAATGGCGTAAACAAAGGGAGGCGAAGCCGCGAGGTGGAGCAAATCCCAAAA
ATAACGTCTCAGTTCGGATTGTAGTCTGCAACTCGACTACATGAAGCTGGAATCGCTAGTAATCGCGA
ATCAGAATGTCGGTGAAGTCGTAACAAGGTAGCCGTATCGGAAGGTGCGGCTGGATCACCTCCTTTCT
AAGGAAGAAAGAAGTAAAGAGTATGTTGT
Strain 13 16S ribosomal RNA Clostridium scindens
SEQ ID NO: 13
TCAACCACAGAACCTTGATAATTGAACAATAGACAACAACCCTGAAAATTTCTTGTAAAGAGAGAATT
TCAGAACAGAACGGGACATCGAGAGATGTCCGACCTTAAAAACAGTAATAACGGGAAAGAATTAGCCA
AGAGTTGATTCTGACCGCGATCACAACTTTTAACGAGAGTTTGAGCGTGGGCAACCTGCCTTGCACTG
GGGGATAACAGCCAGAAATGGCTGCTAATACCGCATAAGACCGAAGCGCCGCATGGCGCAGCGGCCAA
AGCCCCGGCGGTGCAAGATGGGCCCGCGTCTGATTAGGTAGTTGGCGGGGTAACGGCCCACCAAGCCG
ACGATCAGTAGCCGACCTGAGAGGGTGACCGGCCACATTGGGACTGAGACACGGCCCAGACTCCTACG
GGAGGCAGCAGTGGGGAATATTGCACAATGGGGGAAACCCTGATGCAGCGACGCCGCGTGAAGGATGA
AGTATTTCGGTATGTAAACTTCTATCAGCAGGGAAGAAGATGACGGTACCTGACTAAGAAGCCCCGGC
TAACTACGTGCCAGCAGCCGCGGTAATACGTAGGGGGCAAGCGTTATCCGGATTTACTGGGTGTAAAG
GGAGCGTAGACGGCGATGCAAGCCAGATGTGAAAGCCCGGGGCTCAACCCCGGGACTGCATTTGGAAC
TGCGTGGCTGGAGTGTCGGAGAGGCAGGCGGAATTCCTAGTGTAGCGGTGAAATGCGTAGATATTAGG
AGGAACACCAGTGGCGAAGGCGGCCTGCTGGACGATGACTGACGTTGAGGCTCGAAAGCGTGGGGAGC
AAACAGGATTAGATACCCTGGTAGTCCACGCCGTAAACGATGACTACTAGGTGTCGGGTGGCAAGGCC
ATTCGGTGCCGCAGCAAACGCAATAAGTAGTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTA
CCTGATCTTGACATCCCGATGCCAAAGCGCGTAACGCGCTCTTTCTTCGGAACATCGGTGACAGGTGG
TGCATGGTTGTCGTCAGCTCGTGTCGTGAGATGTTGGGTTAAGTCCCGCAACGAGCGCAACCCCTATC
TTCAGTAGCCAGCATTCCGGATGGGCACTCTGGAGAGACTGCCAGGGACAACCTGGAGGAAGGTGGGG
ATGACGTCAAATCATCATGCCCCTTATGACCAGGGCCAAAAATAACGTCTCAGTTCGGATTGTAGTCT
GCAACTCGACTACATGAAGCTGGAATCGCTAGTAATCGCGAATCAGAATGTCGCGGTGAATACGTTCC
CGGGTCTTGTACACACCGCCCCGTCGAAGGTGGGACCGATAACTGGGGTGAAGTCGTAACAAGGTAGC
CGTATCGGAAGG
Strain 14 16S ribosomal RNA Lachnospiraceae bacterium
SEQ ID NO: 14
TCGTGTCGTGAGATGTTGGGTTAAGTCCCGCAACGAGCGCAACCCTTATCCTTAGTAGCCAGCAGGTA
AAGCTGGGCACTCTAGGGAGACTGCCGGGGATAACCCGGAGGAAGGCGGGGATGACGTCAAATCATCA
TGCCCCTTATGATTTGGGCTACACACGTGCTACAATGGCGTAAACAAAGGGAAGCGAGACAGTGATGT
TGAGCAAATCCCAGAAATAACGTCTCAGTTCGGATTGTAGTCTGCAACTCGACTACATGAAGCTGGAA
TCGCTAGTAATCGCGAATCATAACTGGGGTGAAGTCGTAACAAGGTAGCCGTATCGGAAGGTGCGGCT
GGATCACCTCCT
Strain 15 Clostridium sp.
SEQ ID NO: 15
AATTTGAGAGTTTGATCCTGGCTCAGGATGAACGCTGGCGGCGTGCCTAACACATGCAAGGGGTGAGT
AACGCGTGGATAACCTGCCTCACACTGGGGGATAACAGTTAGAAATGGCTGCTAATACCGCATAAGCG
CACAGTACCGCATGGTACGGTGTGAAAAACCCAGGTGGTGTGAGATGGATCCGCGTCTGATTAGCCAG
TTGGCGGGGTAACGGCCCACCAAAGCGACGATCAGTAGCCGACCTGAGAGGGTGACCGGCCACATTGG
GACTGAGACACGGCCCAAACTCCTACGGGAGGCAGCAGTGGGGAATATTGCACAATGGGCGAAAGCCT
GATGCAGCGACGCCGCGTGAGTGAAGAAGTATCTCGGTATGTAAAGCTCTATCAGCAGGGAAGAAAAT
GACGGTACCTGACTAAGAAGCCCCGGCTAACTACGTGCCAGCAGCCGCGGTAATACGTAGGGGGCAAG
CGTTATCCGGATTTACTGGGTGTAAAGGGAGCGTAGACGGCGATGCAAGTCTGAAGTGAAAGCCCGGG
GCTCAACCCCGGGACTGCTTTGGAAACTGTGTGGCTGGAGTGCAGGAGAGGTAAGTGGAATTCCTAGT
GTAGCGGTGAAATGCGTAGATATTAGGAGGAACACCAGTGGCGAAGGCGGCTTACTGGACTGTAACTG
ACGTTGAGGCTCGAAAGCGTGGGGAGCAAACAGGATTAGATACCCTGGTAGTCCACGCCGTAAACGAT
GAATGCTAGGTGTCGGGGGGCAAAGCCCCTCGGTGCCGCCGCTAACGCAATAAGCATTCCACCTGGGG
AGTACGTTCGCAAGAATGAAACTCAAAGGAATTGACGGGGACCCGCACAAGCGGTGGAGCATGTGGTT
TAATTCGAAGCAACGCGAAGAACCTTACCAAGTCTTGACATCCCCCTGACCGGACAGTAACGTGTCCC
TTCCTTCGGGACAGGGGAGACAGGTGGTGCATGGTTGTCGTCAGCTCGTGTCGTGAGATGTTGGGTTA
GGAGACTGCCAGGGATAACCTGGAGGAAGGTGGGGATGACGTCAAATCATCATGCCCCTTTGAGCAAA
TCCCAAAAATAACGTCCCAGTTCGGACTGTAGTCTGCAACCCGACTACACGAAGCTGGAATCGCTAGT
AATCGCGGATCAGAATGCCGCGGTGAATACGTTCCCGGGTCTTGTACACACCGCCCGTCACACCATGG
GAGTCAGCAACGCCCGAAGTCAGTGACCCAACCGAAAGGAGGGAGCTGCCGAAGGCGGGGCAGGTAAC
TGGGGTGAAGTCGTAACAAGGTAGCCGT
Strain 16 16S ribosomal RNA Lachnospiraceae bacterium
SEQ ID NO: 16
TAGAGTTAATCTTAACTGGGTTAAACACTTAAACAAGAGAGTTTGATCCTGGCTCAGGATGAACGCTG
GCGGCGTGCCTAACACATGCAAGTCGAACGGAGTTATGCAGAGGAAGTTTTCGGATGGAATCGGCGTA
ACTTAGTGGCGGACGGGTGAGTAACGCGTGGGAAACCTGCCCTGTACCGGGGGATAACACTTAGAAAT
AGGTGCTAATACCGCATAAGCGCACAGCTTCACATGAGGCAGTGTGAAAAACTCCGGTGGTACAGGAT
GGTCCCGCGTCTGATTAGCCAGTTGGCAGGGTAACGGCCTACCAAAGCGACGATCAGTAGCCGGCCTG
AGAGGGTGAACGGCCACATTGGGACTGAGACACGGCCCAAACTCCTACGGGAGGCAGCAGTGGGGAAT
ATTGCACAATGGGGGAAACCCTGATGCAGCGACGCCGCGTGAGTGAAGAAGTATTTCGGTATGTAAAG
CTCTATCAGCAGGGAAGAAAATGACGGTACCTGACTAAGAAGCCCCGGCTAACTACGTGCCAGCAGCC
GCGGTAATACGTAGGGGGCAAGCGTTATCCGGATTTACTGGGTGTAAAGGGAGCGTAGACGGCATGAC
AAGCCAGATGTGAAAACCCAGGGCTCAACCCTGGGACTGCATTTGGAACTGCCAGGCTGGAGTGCAGG
AGAGGTAAGCGGAATTCCTAGTGTAGCGGTGAAATGCGTAGATATTAGGAGGAACACCAGTGGCGAAG
GCGGCTTACTGGACTGTAACTGACGTTGAGGCTCGAAAGCGTGGGGAGCAAACAGGATTAGATACCCT
GGTAGTCCACGCGGTAAACGATGATTGCTAGGTGTAGGTGGGTATGGACCCATCGGTGCCGCAGCTAA
CGCAATAAGCAATCCACCTGGGGAGTACGTTCGCAAGAATGAAACTCAAAGGAATTGACGGGGACCCG
CACAAGCGGTGGAGCATGTGGTTTAATTCGAAGCAACGCGAAGAACCTTACCAAGTCTTGACATCCCA
ATGACGTGTCCGTAACGGGGCATTCTCTTCGGAGCATTGGAGACAGGTGGTGCATGGTTGTCGTCAGC
TGACTGGCAGAACAACATGTGATAAGATATCAACTGTTGCGGCTGAGAAAGCAGCACACAGTGAGTAA
CGCGTGGGAAACCTGCCCTGTACCGGGGGATAACACTTAGAAATAGGTGCTAGGTCCCGCGTCTGATT
AGCCAGTTGGCAGGGTAACGGCCTACCAAAGCGACGATCAGTAGCCGGCCTGAGAGGGTGAACGGCCA
CATTGGGACTGAGACACGGCCCAAACTCCTACGGGAGGCAGCAGTGGGGAATATTGCACAATGGGGGA
AACCCTGATGCAGCGACGCCGCGTGAGTGAAGAAGTATTTCGGTATGTAAAGCTCTATCAGCAGGGAA
GAAAATGACGGTACCTGACTAAGAAGCCCCGGCTAACTACGTGCCAGCAGCCGCGGTAATACGTAGGG
GGCAAGCGTTATCCGGATTTACTGGGTGTAAAGGGAGCGTAGACGGCATGGCAAGCCAGATGTGAAAA
CCCAGGGCTCAACCTTGGGATTGCATTTGGAACTGCCAGGCTGGAGTGCAGGAGAGGTAAGCGGAATT
CCTAGTGTAGCGGTGAAATGCGTAGATATTAGGAGGAACACCAGTGGCGAAGGCGGCTTACTGGACTG
TAACTGACGTTGAGGCTCGAAAGCGTGGGGAGCAAACAGGATTAGATACCCTGGTAGTCCACGCGGTA
AACGATGATTGCTAGGTGTAGGTGGGTATGGACCCATCGGTGCCGCAGCTAACGCAATAAGCAATCCA
CCTGGGGAGTACGTTCGCAAGAATGAAACTCAAAGGAATTGACGGGGACCCGCACAAGCGGTGGAGCA
TGTGGTTTAATTCGAAGCAACGCGAAGAACCTTACCAAGTCTTGACATCCCAATGACGCACCTGTAAA
GAGGTGTTCCCTTCGGGGCATTGGAGACAGGTGGTGCATGGTTGTCGTCAGCTCGTGTCGTGAGATGT
TGGGTTAAGTCCCGCAACGAGCGCAACCCTTATTCTTAGTAGCCAGCAGGTAAAGCTGGGCACTCTAA
GGAGACTGCCGGGGATAACCCGGAGGAAGGCGGGGATGACGTCAAATCATCATGCCCCTTATGATTTG
GGCTACACACGTGCTACAATGGCGTAAACAAAGGGAAGCGAGACAGTGATGTGGAGCAAATCCCAGAA
ATAACGTCTCAGTTCGGATTGTAGTCTGCAACTCGACTACATGAAGCTGGAATCGCTAGTAATCGCGA
ATCAGCATGTCGCGGTGAATACGTTCCCGGGTCTTGTACACACCGCCCGTCACACCATGGGAGTTGGA
AATGCCCGAAGTCTGTGACCTAACCGAAAGGGAGGAGCAGCCGAAGGCAGGTCTGATAACTGGGGTGA
AGTCGTAACAAGGTAGCCGTATCGGAAGGTGCGGCTGGATCACCTCCTTTCTAAGGCGAAGAAGTAGG
GGTTGTATTACTGTTTAGATGTT

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Detection, Abundance, and Colonization Kinetics of Bacterial Strains from an 11-Valent Bacterial Mixture in the Heathy Human Gut by qPCR

IBD is characterized by mucosal immune dysregulation and an altered microbiome (dysbiosis). The gut microbiome of patients with Inflammatory Bowel Disease (IBD) is characterized by decreased α-diversity, decreased levels of Clostridium clusters IV and XIVa, and increased levels of Enterobacteriacea, Ruminococcus gnavus (Sartor, Gastroenterology (2010) 139(6): 1816-1819). Without wishing to be bound by any particular theory, microbiome-based therapeutics have the potential to ameliorate inflammatory diseases by displacing pro-inflammatory bacteria, upregulate regulatory immune processes and increase the local concentration of regulatory metabolites (short chain fatty acids). Defined consortia of bacteria address the safety and reproducibility issues found with fecal transplant through controlled manufacturing and quality protocols. Such consortia eliminate concerns around donor dependence, material consistency, the presence of antibiotic resistance genes in bacterial genomes, and the presence of pathogens/uncharacterized organisms/unknown material.

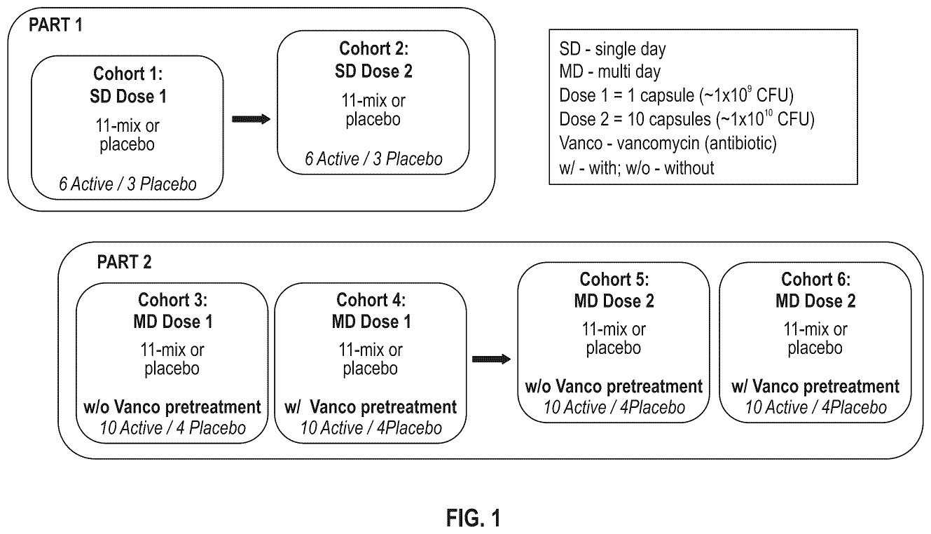

This Example describes the effects of a live biotherapeutic product (LBP) containing 11 bacterial strains (11-mix) (FIG. 1). The 11-mix is comprised of 11 commensal, nonpathogenic Clostridia strains derived from a healthy donor that induce regulatory T cells, as described in Table 1. The safety, tolerability, and pharmacokinetics of colonization (presence and abundance) of 11-mix strains following dosing of the 11-mix in healthy adult volunteers (HV) were assessed throughout the 6-month duration of a study in both single and multiple dose cohorts. Also described is the development and results of a qPCR assay developed as a pharmacokinetic (PK) readout to determine the presence and abundance of 11-mix strains in stool samples collected throughout the study.

The objectives of this study were to support the secondary endpoints of the 11-mix human study, which are to detect and determine the abundance of 11-mix strains using a quantitative polymerase chain reaction (qPCR) assay. The primary objectives of the qPCR analysis are as follows:
To assess the colonization dynamics of 11-mix in the stool following single and multiple oral study intervention administrations (Parts 1 and 2).
To assess the colonization dynamics of 11-mix following multiple oral study intervention administrations with specific pretreatments (Part 2).
To assess the effects and recovery from vancomycin treatment on the bacterial load of the healthy human gut (Part 2).

### Method Overview

This was Phase 1, randomized, double-blind, placebo-controlled, single and multiple-day dose, multicenter, interventional study in healthy male and female participants, 18 to 60 years of age, inclusive.

The study was executed in 2 Parts, as shown in FIG. 1.

In Part 1, single-day dose administration of 11-mix was evaluated in 2 cohorts (n=9 per cohort; 6 active and 3 placebo):
Cohort 1: Dose 1 (˜1×10⁹ CFU total per day; 1 capsule); and
Cohort 2: Dose 2 (˜1×10¹⁰ CFU total per day; 10 capsules each containing ˜1×10⁹ CFU).

In Part 2, multiple-day dose administrations of 11-mix were evaluated in 4 cohorts (n=14 per cohort; 10 active and 4 placebo). Two cohorts (cohorts 4 and 6) were pretreated with vancomycin (125 mg, qid) for five days prior to dosing:
Cohort 3: Dose 1 (˜1×10⁹ CFU total per day; 1 capsule; 14-day dose regimen) without vancomycin pretreatment;
Cohort 4: Dose 1 (˜1×10⁹ CFU total per day; 1 capsule; 14-day dose regimen) with vancomycin pretreatment for 5 days;
Cohort 5: Dose 2 (˜1×10¹⁰ CFU total per day; 10 capsules; 14-day dose regimen) without vancomycin pretreatment; and
Cohort 6: Dose 2 (˜1×10¹⁰ CFU total per day; 10 capsules; 14-day dose regimen) with vancomycin pretreatment for 5 days.

Stool samples were collected frequently throughout the duration of the study up to a six-month long-term follow-up.

Participants were healthy male and female individuals between 18 and 60 years of age (inclusive), who had a body mass index (BMI) between 18 and 30 kg/m² (inclusive) and body weight of not less than 50 kg. Female participants of childbearing potential were excluded if pregnant, undergoing assisted reproduction, or would be breast feeding during the study period and 3 months after last dose of study intervention.

Participants collected a small volume of stool into a tube following the manufacturer's instructions. Stool samples collected at the clinical sites were stored immediately at −75° C.±10° C. post collection. Samples collected at home were stored at room temperature (25° C.±10° C.) for up to 1 week before being stored at −75° C.±10° C.

Bacterial DNA was extracted using the different high throughput extraction kits according to manufacturer's protocol (with some modifications) and quantitated DNA libraries were constructed with the bacterial DNA from each kit for whole genome sequencing and sequenced on the Illumina NovaSeq System.

Strain specific primers (25 bp each) were designed based on data generated to detect each of the bacterial strains of the pharmaceutical compositions using qPCR. Primers used to detect each strain are shown in Table 2. The amplicons generated by each pair of qPCR primers are shown in Table 3.

TABLE 2
Sequences of primers used to detect bacterial strains

| Primer Name | Species | Sequence |
|---|---|---|
| Strain-1.1F | Clostridium | TTTTGGCGTTATCGGGCAAACATAT |
| | saccharogumia | (SEQ ID NO: 17) |
| Strain-1.1R | Clostridium | GAAAAATCATCAGCTATAGCATTAA |
| | saccharogumia | (SEQ ID NO: 33) |
| Strain-3.1F | Flavonifractor plautii | ACCCGGATCTAATTTTGGTCGAAAA |
| | | (SEQ ID NO: 18) |
| Strain-3.1R | Flavonifractor plautii | CTCCGTCACTTAAATACATATCAAC |
| | | (SEQ ID NO: 34) |
| Strain-4.1F | Clostridium hathewayi | ACAGAAATATTGAAAGCTTTGTGAT |
| | | (SEQ ID NO: 19) |
| Strain-4.1R | Clostridium hathewayi | TTATCAGCATCTAATATGTGCGTAT |
| | | (SEQ ID NO: 35) |
| Strain-6.2F | Blautia coccoides | TCCGTGGCCCCGAAGGAGGCTTCTG |
| | | (SEQ ID NO: 20) |
| Strain-6.2R | Blautia coccoides | TGCCCCAAAGACAGAATGTATACCT |
| | | (SEQ ID NO: 36) |
| Strain-7.2F | Clostridium bolteae | GAAATCTTACACACCTATTCAGACC |
| | | (SEQ ID NO: 21) |
| Strain-7.2R | Clostridium bolteae | ATCATCCATCTGCTGTTCCAGGCTC |
| | | (SEQ ID NO: 37) |
| Strain-8.1F | Dielma fastidiosa | TGTCTACGGAAAGATGCAAAAAGTG |
| | | (SEQ ID NO: 65) |
| Strain-8.1R | Dielma fastidiosa | ATTGCTGCTGCATCAGCATTCAATA |
| | | (SEQ ID NO: 66) |
| Strain-9.2F | Clostridium indolis | CAAGGGAGCTTTGGTGAGTTTACAC |
| | | (SEQ ID NO: 22) |
| Strain-9.2R | Clostridium indolis | TGAGCCATCCTGAGATGAAATATTG |
| | | (SEQ ID NO: 38) |
| Strain-13.4F | Anaerotruncus colihominis | TTGTACTGTTCCCAATGAGTATCAA |
| | | (SEQ ID NO: 23) |
| Strain-13.4R | Anaerotruncus colihominis | GAATATAATCAAGATCCTCCAGCGG |
| | | (SEQ ID NO: 39) |
| Strain-14.1F | Ruminococcus sp. | CTGATAGACGAAGAAGGCGCATACT |
| | | (SEQ ID NO: 24) |
| Strain-14.1R | Ruminococcus sp. | TTCTCCATTTTCCATCATCCTTTCA |
| | | (SEQ ID NO: 40) |
| Strain-15.1F | Clostridium lavalense | GTTGCTTCGGGAATCAATCAAATAT |
| | | (SEQ ID NO: 25) |
| Strain-15.1R | Clostridium lavalense | CTCCATGGCTCTGGCGGAATTAAAG |
| | | (SEQ ID NO: 41) |
| Strain-16.4F | Clostridium symbiosum | ATCATCCGCTACTCCAAACCCGATT |
| | | (SEQ ID NO: 26) |
| Strain-16.4R | Clostridium symbiosum | GTGCGATGGGGAGGACGATTATGCC |
| | | (SEQ ID NO: 42) |
| Strain-18.4F | Clostridium ramosum | TTACATTTATTCCTTTAATCCCCAT |
| | | (SEQ ID NO: 27) |
| Strain-18.4R | Clostridium ramosum | TCACAGTCGGCATTGGGATATACCG |
| | | (SEQ ID NO: 43) |
| Strain-21.4F | Eubacterium contortum | CGGAAGGATCAAAGTATATATGGAT |
| | | (SEQ ID NO: 28) |
| Strain-21.4R | Eubacterium contortum | TCAAATATTTCTAACTCGTTGGCTT |
| | | (SEQ ID NO: 44) |
| Strain-26.2F | Clostridium scindens | TTCGCCACTTAATATACGGATGTTA |
| | | (SEQ ID NO: 29) |
| Strain-26.2R | Clostridium scindens | GGACACGAATTATCCAAAAAAGCCG |
| | | (SEQ ID NO: 45) |
| Strain-27.2F | Lachnospiraceae | TCGATACCAATTTGTAATAATCCAG |
| | bacterium | (SEQ ID NO: 30) |
| Strain-27.2R | Lachnospiraceae | AATGAAGCGGCAGCAGCGTCCAATA |
| | bacterium | (SEQ ID NO: 46) |
| Strain-28.1F | Clostridium sp. | CATCCACCAAAAGCAGTCCCCCTAC |
| | | (SEQ ID NO: 31) |
| Strain-28.1R | Clostridium sp. | GGTTAAGATTGGTTGGCAGGCCATA |
| | | (SEQ ID NO: 47) |
| Strain-29.1F | Lachnospiraceae | GATCGTCCGGGTGCTGCAAAAAATG |
| | bacterium | (SEQ ID NO: 32) |
| Strain-29.1R | Lachnospiraceae | TCCTTCAGTGCTCCATAGTATCCTT |
| | bacterium | (SEQ ID NO: 48) |

TABLE 3
Amplicon sequences of strain-specific qPCR.

| Amplicon and | | |
|---|---|---|
| Type | Species | Sequence |
| 1.1 Sanger | Clostridium | TTTTGGCGTTATCGGGCAAACATATGATGAATATGTAAAACCGGC |
| | saccharogumia | ATTTGATAAAATAAAGCAGGGATTAACAAGTATTGTAAGCACCGT |
| | | ACTTGATGCCTATAACTCTAATATCGCTCCAGTAGTTAATGCTAT |
| | | AGCTGATGATTTTTC (SEQ ID NO: 49) |
| 1.1 | Clostridium | TTTTGGCGTTATCGGGCAAACATATGATGAATATGTAAAACCGGC |
| Computational | saccharogumia | ATTTGATAAAATAAAGCAGGGATTAACAAGTATTGTAAGCACCGT |
| | | ACTTGATGCCTATAACTCTAATATCGCTCCAGTAGTTAATGCTAT |
| | | AGCTGATGATTTTTC (SEQ ID NO: 49) |
| 3.1 Sanger | Flavonifractor | ACCCGGATCTAATTTTGGTCGAAAACAGCGAAGGAATTCTTGGAT |
| | plautii | ATGTTAAATCAGAGGATTTAGATGGTTCCAATTATCTGACGCCGG |
| | | AGGAGGCGATATTGAACCAATCACAAGGGTACAGCGTTGATATGT |
| | | ATTTAAGTGACGGAG (SEQ ID NO: 50) |
| 3.1 | Flavonifractor | ACCCGGATCTAATTTTGGTCGAAAACAGCGAAGGAATTCTTGGAT |
| Computational | plautii | ATGTTAAATCAGAGGATTTAGATGGTTCCAATTATCTGACGCCGG |
| | | AGGAGGCGATATTGAACCAATCACAAGGGTACAGCGTTGATATGT |
| | | ATTTAAGTGACGGAG (SEQ ID NO: 50) |
| 4.1 Sanger | Clostridium | ACAGAAATATTGAAAGCTTTGTGATTCCTATAGTCAAAGTTATCG |
| | hathewayi | GTAGAAAAGGAACGATTTTATAAGGATTGCAGGCTATGTTCAAAC |
| | | GCCAGCTTTAAGGAGATGCACGAAAATACGCGTAAATACGCACAT |
| | | ATTAGATGCTGATAA (SEQ ID NO: 51) |
| 4.1 | Clostridium | ACAGAAATATTGAAAGCTTTGTGATTCCTATAGTCAAAGTTATCG |
| Computational | hathewayi | GTAGAAAAGGAACGATTTTATAAGGATTGCAGGCTATGTTCAAAC |
| | | GCCAGCTTTAAGGAGATGCACGAAAATACGCGTAAATACGCACAT |
| | | ATTAGATGCTGATAA (SEQ ID NO: 51) |
| 6.2 Sanger | Blautia coccoides | TCCGTGGCCCCGAAGGAGGCTTCTGCCCGCAGAGCCACGCCGTCC |
| | | ATAGCGCTTGCCGCATAATGGGGTGCACAGATAACAGCATAAACA |
| | | GCCTTTGCAGTGACCCTCCCGCAGGCCTCCTGAACAGGTATACAT |
| | | TCTGTCTTTGGGGCA (SEQ ID NO: 52) |
| 6.2 | Blautia coccoides | TCCGTGGCCCCGAAGGAGGCTTCTGCCCGCAGAGCCACGCCGTCC |
| Computational | | ATAGCGCTTGCCGCATAATGGGGTGCACAGATAACAGCATAAACA |
| | | GCCTTTGCAGTGACCCTCCCGCAGGCCTCCTGAACAGGTATACAT |
| | | TCTGTCTTTGGGGCA (SEQ ID NO: 52) |
| 7.2 Sanger | Clostridium | GAAATCTTACACACCTATTCAGACCAGATGCCGAAGCCGCCGCCG |
| | bolteae | CTTTCCAAGCAGAAAGAATTCAAACAAATCAAGAACATAATTATC |
| | | CGCGTGGCCATGGAGTTGGAACCGGCCGGAACATGGAGCCTGGAA |
| | | CAGCAGATGGATGAT (SEQ ID NO: 53) |
| 7.2 | Clostridium | GAAATCTTACACACCTATTCAGACCAGATGCCGAAGCCGCCGCCG |
| Computational | bolteae | CTTTCCAAGCAGAAAGAATTCAAACAAATCAAGAACATAATTATC |
| | | CGCGTGGCCATGGAGTTGGAACCGGCCGGAACATGGAGCCTGGAA |
| | | CAGCAGATGGATGAT (SEQ ID NO: 53) |
| 8.1 Sanger | Dielma fastidiosa | TGTCTACGGAAAGATGCAAAAAGTGTATACAATTGAATCACAATT |
| | | WAATACAGTCGGTAACACACCTTCATTCAGCGTTATAAAAATGTC |
| | | CAATGAAGATATGAAAAAAGCTTCCTTTGCTGCGTTATTGAATGC |
| | | TGATGCAGCAGCAAT (SEQ ID NO: 67) |
| 8.1 | Dielma fastidiosa | TGTCTACGGAAAGATGCAAAAAGTGTATACAATTGAATCACAATT |
| Computational | | AAATACAGTCGGTAACACACCTTCATTCAGCGTTATAAAAATGTC |
| | | CAATGAAGATATGAAAAAAGCTTCCTTTGCTGCGTTATTGAATGC |
| | | TGATGCAGCAGCAAT (SEQ ID NO: 68) |
| 9.2 Sanger | Clostridium indolis | YAAKGGAGCTTTGGTGAGTTTACACCGACATATGGAGATTCTGGA |
| | | TCTTCTACACAAAAGAAGCGAAGAATCACCGTCCGAAAAGTCACG |
| | | GAGGAGGAACTTAGAAAGTTTGCAGAGAATAGCAGCAATATTTCA |
| | | TCTCAGGATGGCTCA (SEQ ID NO: 69) |
| 9.2 | Clostridium indolis | CAAGGGAGCTTTGGTGAGTTTACACCGACAGATGGAGATTCTGGA |
| Computational | | TCTTCTACACAAAAGAAGCGAAGAATCACCGTCCGAAAAGTCACG |
| | | GAGGAGGAACTTAGAAAGTTTGCAGAGAATAGCAGCAATATTTCA |
| | | TCTCAGGATGGCTCA (SEQ ID NO: 54) |
| 13.4 Sanger | Anaerotruncus | TTGTACTGTTCCCAATGAGTATCAAAGATGGGCGAATCGATTTTT |
| | colihominis | CCCGGTTGGAACAATATATTACACCCAAAACAAAAATATTTGGGT |
| | | TATGCAACCCGCATAACCCGCTGGGAATGTGTTATCCGCTGGAGG |
| | | ATCTTGATTATATTC (SEQ ID NO: 55) |
| 13.4 | Anaerotruncus | TTGTACTGTTCCCAATGAGTATCAAAGATGGGCGAATCGATTTTT |
| Computational | colihominis | CCCGGTTGGAACAATATATTACACCCAAAACAAAAATATTTGGGT |
| | | TATGCAACCCGCATAACCCGCTGGGAATGTGTTATCCGCTGGAGG |
| | | ATCTTGATTATATTC (SEQ ID NO: 55) |
| 14.1 Sanger | Ruminococcus sp. | CTGATAGACGAAGAAGGCGCATACTATCGGTGGAGCTGTCCGAAA |
| | | ACAGCTGTATGGAAGGGCTGTTCTGTCTCAATGCACCCGGAGATG |
| | | GAGAAAATCACAGATCCAGTCCGTTGCAGACAATGTGAAAGGATG |
| | | ATGGAAAATGGAGAA (SEQ ID NO: 56) |
| 14.1 | Ruminococcus sp. | CTGATAGACGAAGAAGGCGCATACTATCGGTGGAGCTGTCCGAAA |
| Computational | | ACAGCTGTATGGAAGGGCTGTTCTGTCTCAATGCACCCGGAGATG |
| | | GAGAAAATCACAGATCCAGTCCGTTGCAGACAATGTGAAAGGATG |
| | | ATGGAAAATGGAGAA (SEQ ID NO: 56) |
| 15.1 Sanger | Clostridium | GTTGCTTCGGGAATCAATCAAATATCATCAGCCATTCAAGGATTT |
| | lavalense | TCCGAGCGGACTAAGACCGCCGATTTTAGCCGGGTTGTTACTGGA |
| | | ATGAATAAGCTGGCCCAGGTAGATTCGCAAGGAGTCTTTAATTCC |
| | | GCCAGAGCCATGGAG (SEQ ID NO: 57) |
| 15.1 | Clostridium | GTTGCTTCGGGAATCAATCAAATATCATCAGCCATTCAAGGATTT |
| Computational | lavalense | TCCGAGCGGACTAAGACCGCCGATTTTAGCCGGGTTGTTACTGGA |
| | | ATGAATAAGCTGGCCCAGGTAGATTCGCAAGGAGTCTTTAATTCC |
| | | GCCAGAGCCATGGAG (SEQ ID NO: 57) |
| 16.4 Sanger | Clostridium | ATCATCCGCTACTCCAAACCCGATTCCTTTTCTCCTCAGCGTTTC |
| | symbiosum | CCAGAATGGCTCAAAGGTAATCATATCCTCATCTTGTCCATTGCC |
| | | CGCATAATTGCCTCAAAAGCATCATCCCAGCCTCTGGCATAATCG |
| | | TCCTCCCCATCGCAC (SEQ ID NO: 58) |
| 16.4 | Clostridium | ATCATCCGCTACTCCAAACCCGATTCCTTTTCTCCTCAGCGTTTC |
| Computational | symbiosum | CCAGAATGGCTCAAAGGTAATCATATCCTCATCTTGTCCATTGCC |
| | | CGCATAATTGCCTCAAAAGCATCATCCCAGCCTCTGGCATAATCG |
| | | TCCTCCCCATCGCAC (SEQ ID NO: 58) |
| 18.4 Sanger | Clostridium | TTACATTTATTCCTTTAATCCCCATATGTCCAGTACTCCTATCCT |
| | ramosum | CTGTCTTTTAAGTCCCAGTCTTGATAATTCACTTTTTTTAATAAA |
| | | CAGCCCTCCACCGGGTACAAGATATGTTCCTGAAGCGGTATATCC |
| | | CAATGCCGACTGTGA (SEQ ID NO: 59) |
| 18.4 | Clostridium | TTACATTTATTCCTTTAATCCCCATATGTCCAGTACTCCTATCCT |
| Computational | ramosum | CTGTCTTTTAAGTCCCAGTCTTGATAATTCACTTTTTTTAATAAA |
| | | CAGCCCTCCACCGGGTACAAGATATGTTCCTGAAGCGGTATATCC |
| | | CAATGCCGACTGTGA (SEQ ID NO: 59) |
| 21.4 Sanger | Eubacterium | CGKAAGGATCAAAGTATATATGGATGATGCCTTCTGTGGTGATAT |
| | contortum | CACTATCGCGGACGGTTTACAAAGCGGAGGAAAATTGAACGCTGA |
| | | TGCCGGAAAATATGAGCTGCGGTTGTCCTTTGAGGAAGCCAACGA |
| | | GTTAGAAATATTTGA (SEQ ID NO: 60) |
| 21.4 | Eubacterium | CGGAAGGATCAAAGTATATATGGATGATGCCTTCTGTGGTGATAT |
| Computational | contortum | CACTATCGCGGACGGTTTACAAAGCGGAGGAAAATTGAACGCTGA |
| | | TGCCGGAAAATATGAGCTGCGGTTGTCCTTTGAGGAAGCCAACGA |
| | | GTTAGAAATATTTGA (SEQ ID NO: 60) |
| 26.2 Sanger | Clostridium | TTCGCCACTTAATATACGGATGTTAGGCCGTATATGGCTATTATC |
| | scindens | TAAAATATTGCCAATGTCGTAGTTTAAGCCTTCGGCAGAAATAAC |
| | | CATGGGATAGTCTGCAAGATCCTGTGCCAGTATTGCGGCTTTTTT |
| | | GGATAATTCGTGTCC (SEQ ID NO: 61) |
| 26.2 | Clostridium | TTCGCCACTTAATATACGGATGTTAGGCCGTATATGGCTATTATC |
| Computational | scindens | TAAAATATTGCCAATGTCGTAGTTTAAGCCTTCGGCAGAAATAAC |
| | | CATGGGATAGTCTGCAAGATCCTGTGCCAGTATTGCGGCTTTTTT |
| | | GGATAATTCGTGTCC (SEQ ID NO: 61) |
| 27.2 Sanger | Lachnospiraceae | TCGATACCAATTTGTAATAATCCAGTTCACACGCTTAATCTGCCC |
| | bacterium | CATCTTGCCGCTATCCACGATTTCCTTCATCTTACGATACACACA |
| | | ATTGGTCCGCTGGTTAAACATAATTGCAAACGTCTTATTGGACGC |
| | | TGCTGCCGCTTCATT (SEQ ID NO: 62) |
| 27.2 | Lachnospiraceae | TCGATACCAATTTGTAATAATCCAGTTCACACGCTTAATCTGCCC |
| Computational | bacterium | CATCTTGCCGCTATCCACGATTTCCTTCATCTTACGATACACACA |
| | | ATTGGTCCGCTGGTTAAACATAATTGCAAACGTCTTATTGGACGC |
| | | TGCTGCCGCTTCATT (SEQ ID NO: 62) |
| 28.1 Sanger | Clostridium sp. | YATCCACCAAAAGCAGTCCCCCTACCCACTTCCCAGCAAAAACCA |
| | | CTAAACTCCACATCCCAGGCCTGCCAGCCCCTCATATTCACCAAA |
| | | CCATGGCATGGCCAATCTAAACCATTAAACTATGGTATGGCCTGC |
| | | CAACCAATCTWAACC (SEQ ID NO: 63) |
| 28.1 | Clostridium sp. | CATCCACCAAAAGCAGTCCCCCTACCCACTTCCCAGCAAAAACCA |
| Computational | | CTAAACTCCACATCCCAGGCCTGCCAGCCCCTCATATTCACCAAA |
| | | CCATGGCATGGCCAATCTAAACCATTAAACTATGGTATGGCCTGC |
| | | CAACCAATCTTAACC (SEQ ID NO: 63) |
| 29.1 Sanger | Lachnospiraceae | GATCGTCCGGGTGCTGCAAAAAATGATAAGTATCGGGGACTTTAC |
| | bacterium | GATGTACTTATCTGCAATAAACAGCTTCAGCAGTTCAATGAATGA |
| | | TTTAATGAAAAGCTGTGTTGAAATCAAACAGTTTGAAGGATACTA |
| | | TGGAGCACTGAAGGA (SEQ ID NO: 64) |
| 29.1 | Lachnospiraceae | GATCGTCCGGGTGCTGCAAAAAATGATAAGTATCGGGGACTTTAC |
| Computational | bacterium | GATGTACTTATCTGCAATAAACAGCTTCAGCAGTTCAATGAATGA |
| | | TTTAATGAAAAGCTGTGTTGAAATCAAACAGTTTGAAGGATACTA |
| | | TGGAGCACTGAAGGA (SEQ ID NO: 64) |

The 11-mix strain quantification qPCR assay reactions were prepared: 5 ng of bacterial DNA was added to a strain specific primer set (0.8 μM), mixed with PerfeCTa SYBR Green FastMix Low ROX (QuantaBio). An standard curve was also generated using strain specific gDNA for each strain of the 11-mix. qPCR amplification was performed on the QuantStudio™ 12K (ThermoFisher) using the following conditions:
Run Method: Hold Stage: 95° C.×10 min,
PCR Stage: 40 cycles (95° C.×10 sec, 60° C.×20 sec),
Melt Curve: 95° C.×15 sec, 60° C.×1 min, 95° C.×15 sec.

For abundance estimation the mean RA value was taken for multiple samples. Relative abundances (%) measures were estimated from qPCR data to help in future comparisons with metagenomic shotgun sequencing data. This was done by normalizing the qPCR output (ng) by the total input into the qPCR assay, which was assumed to be 5 ng:

ng 11-mix strains gDNA/5 ng input total DNA

3 samples were determined to have >100% RA of total 11-mix strains, for downstream analysis these samples were forced to 100%.

Data was log₁₀ transformed in figures to show small variation in baseline samples. An arbitrarily small value of 0.01 was added before log transformation.

Raw values were taken for each output even if the value was below LLOQ. An arbitrarily small value of 0.01 was added to relative abundance values before log 10 transformation.

Detection and abundance measures from qPCR were used to assess PK parameters such as Cₘₐₓ, Tₘₐₓ, and C_{first}/Cₗₐₛₜ/Tₗₐₛₜ. Non-parametric statistical tests were used unless otherwise specified.

### Results

2,247/2,449 (91.8%) scheduled stool samples were collected, and DNA was extracted from the 6 cohorts. 164 (6.7%) stool samples were not produced, and 38 (1.6%) samples failed the DNA extraction step.

Most participants demonstrated a background detection of one to three 11-mix strains in baseline samples.

There was large variation in microbial density observed between participants in the baseline samples (mean 18.96±13.80 ng/mg, minimum 0.42 ng/mg, maximum 65.58 ng/mg). A trend of lower microbial density was observed in cohort 4 screening samples which was not statistically significant (Kruskall-Wallis p=0.22). The microbial density of the screening samples did not demonstrate any significant difference when comparing the active and placebo treated arms across cohorts in the study.

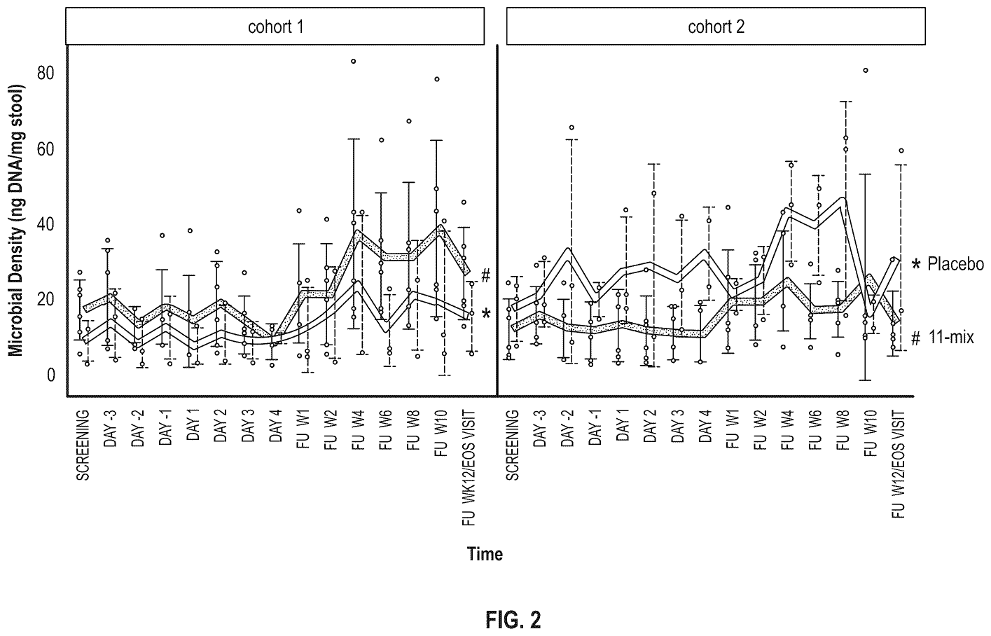

The microbial density of participants in Part 1 did not display any significant change in the duration of the trial. There was no difference between active and placebo treated arms (FIG. 2).

Participants that did not receive vancomycin pretreatment (cohorts 3 and 5) exhibited microbial density dynamics similar to participants in Part 1. There was high intra- and inter-individual variability observed, but no significant changes were observed associated with either 11-mix or placebo treatment.

Vancomycin pretreatment cohorts (Cohorts 4 and 6) were included to determine how vancomycin-mediated reduction of the abundance of bacteria in the host microbiome may augment 11-mix strain colonization into the microbiome of the subject. Th3 short course of pretreatment (5 days) was well tolerated and resulted in transient self-resolving treatment related adverse events.

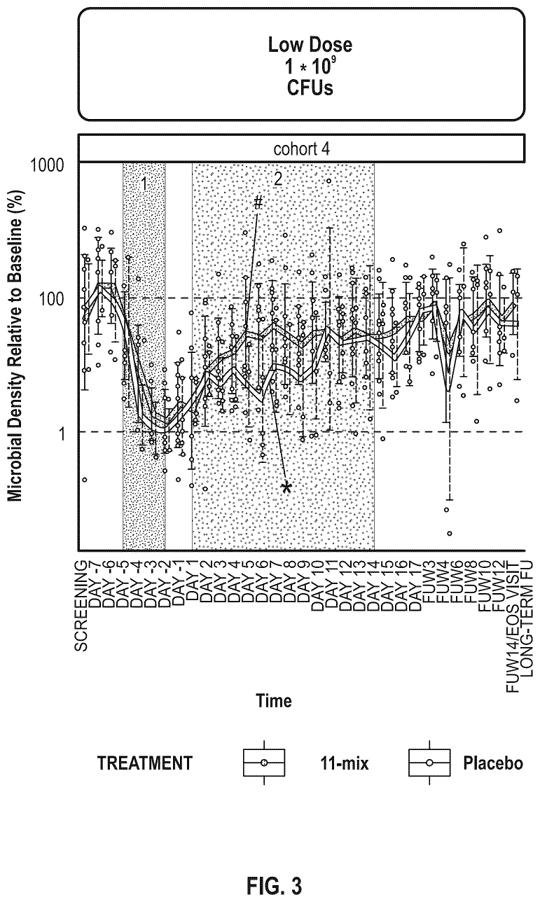

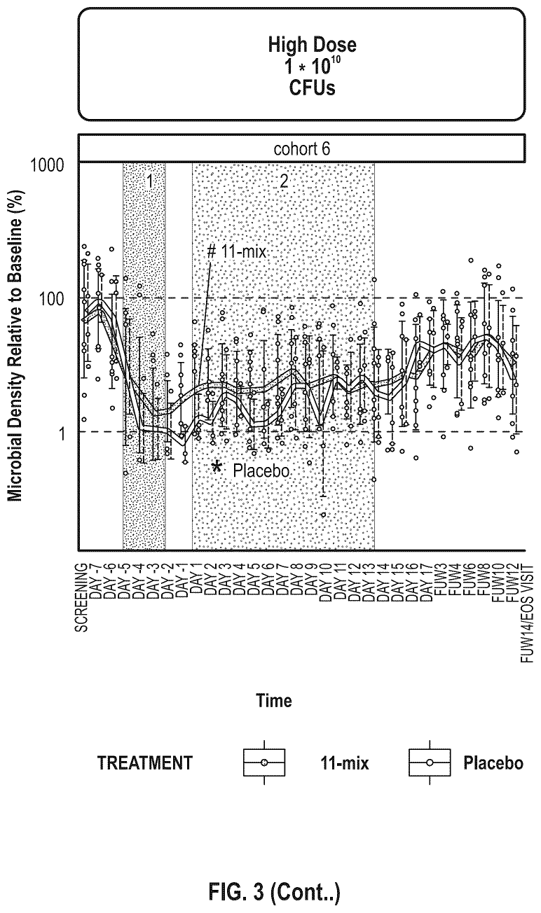

Vancomycin pretreatment resulted in a rapid and robust decrease in microbial density starting on the second day of dosing and continuing to the final day of dosing in both cohorts. A roughly 90% reduction in microbial density was observed across cohorts and treatment arms (FIG. 3).

Microbial density in cohort 4 recovered to baseline (shown as 100% in FIG. 3) after 4 weeks post cessation of vancomycin while in cohort 6 it was only observed to recover to roughly ˜50% baseline values. There was no significant difference in microbial density comparing active and placebo treatment arms at any timepoints.

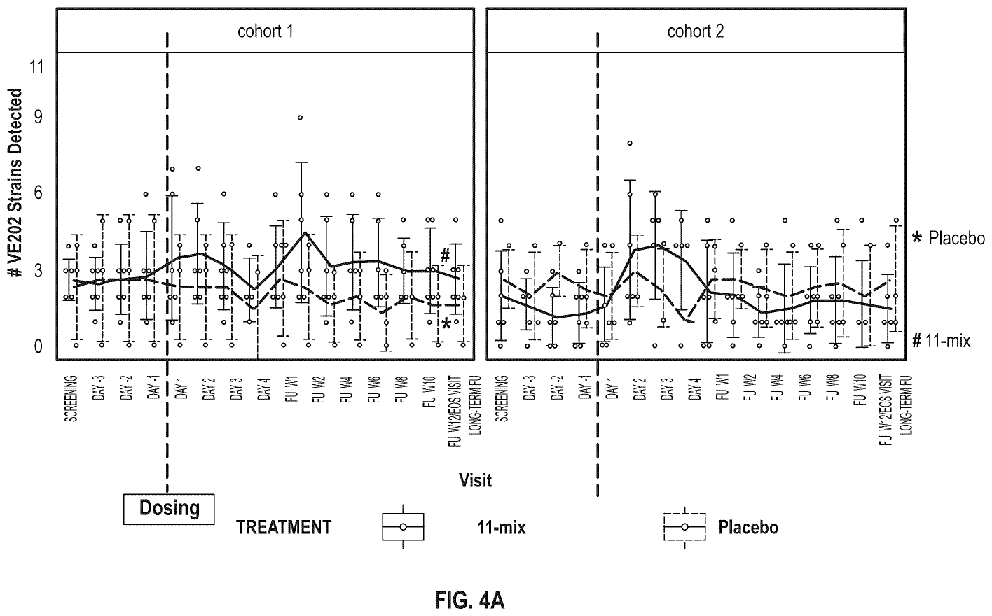

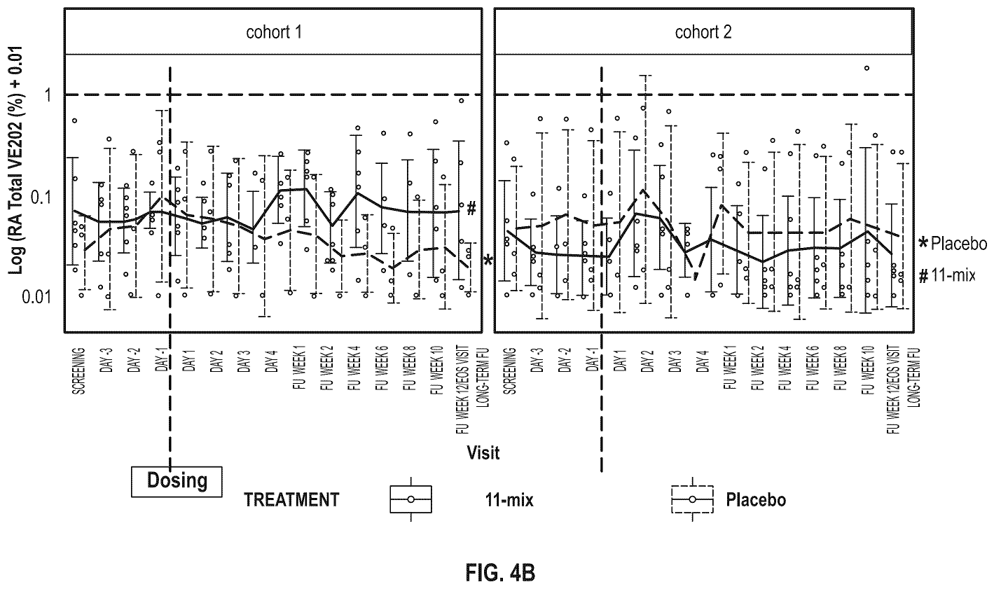

On average, a single day of dosing without vancomycin pretreatment did not increase detection of 11-mix strains or changes in relative abundance measures of 11-mix strains compared to baseline (FIG. 4A).

Tₘₐₓ for cohort 1 was observed at week 2 follow-up with Cₘₐₓ 4.5±2.7 detected strains in subjects that received the 11-mix. In cohort 2, Tₘₐₓ was observed at day 3 with 4.0±2.1 detected strains. In subjects that received the placebo for both cohorts, no variation was observed across any timepoints with a maximum mean detected strains 2.8±2.3 observed at week 1 FU for cohort 1 and 2.7±1.5 in cohort 2.

Tₘₐₓ for cohort 1 was at week 2 follow-up with mean total RA of 11-mix strains of 0.55%±0.36%. In Cohort 2, Tₘₐₓ was at the 12-week end of study (EOS) visit with mean total abundance of 11-mix strains 0.32%±0.75%. For placebo subjects. Tₘₐₓ was observed at Day −1 for cohort 1 with mean Cₘₐₓ 0.19%±0.17% and cohort 2 with mean Cₘₐₓ 0.40%±2.1 (FIG. 4B).

There was no observed difference between active treatment and placebo arms in either detection of 11-mix strains or in relative abundance of strains in either single dose cohorts (cohorts 1 and 2).

There was evidence of transient colonization of certain individuals. A single participant in cohort 1 exhibited detection of 9/11 strains of the 11-mix at the week 2 follow-up. In cohort 2, a single participant showed detection of 8/11 strains of the 11-mix at 48 hours post dosing. This detection of strains decreased to 5 strains at day 4 and returned to baseline levels by week 2. The total abundance of these strain remained low (<1% RA).

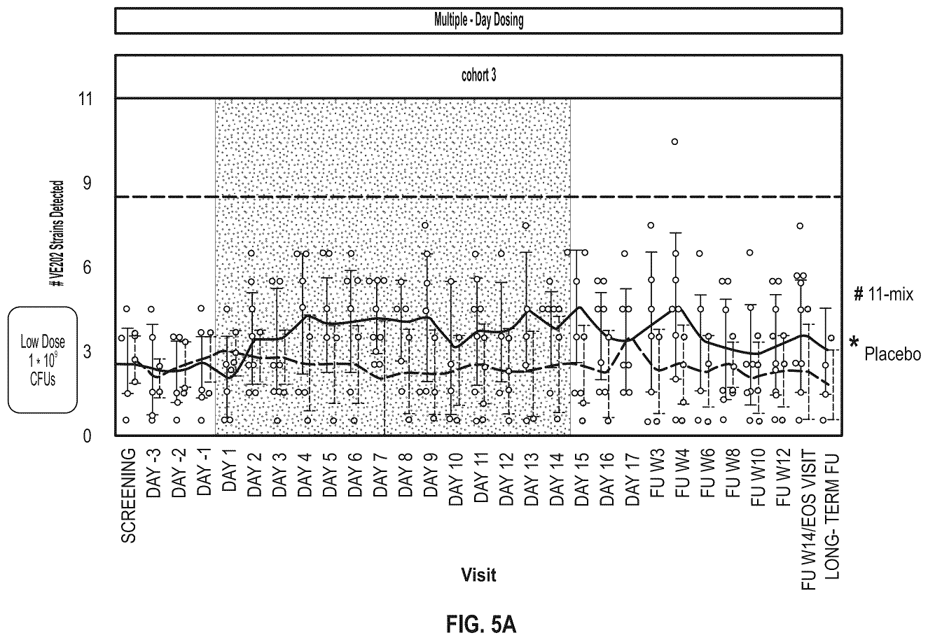

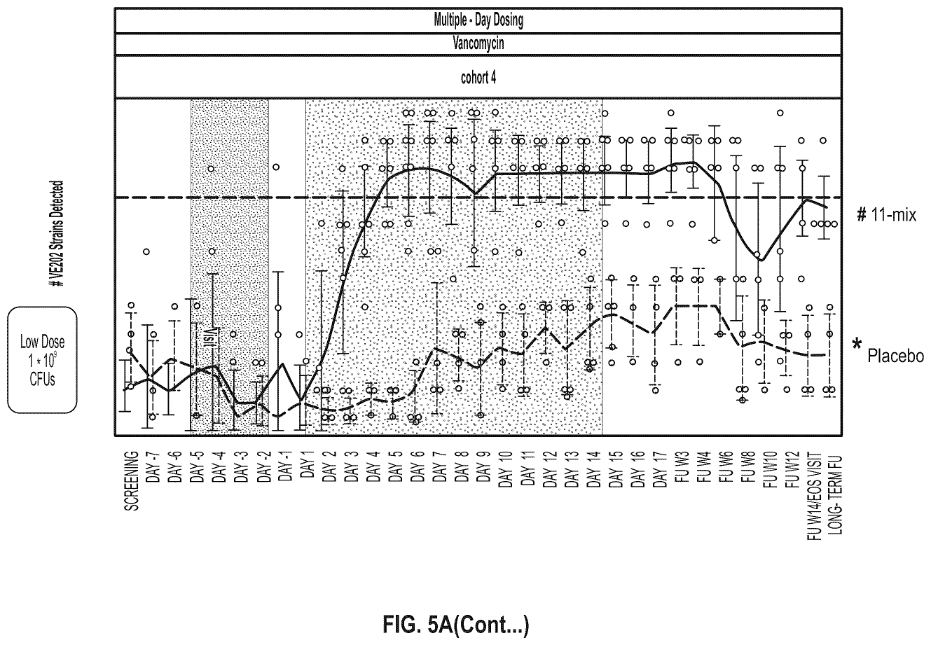

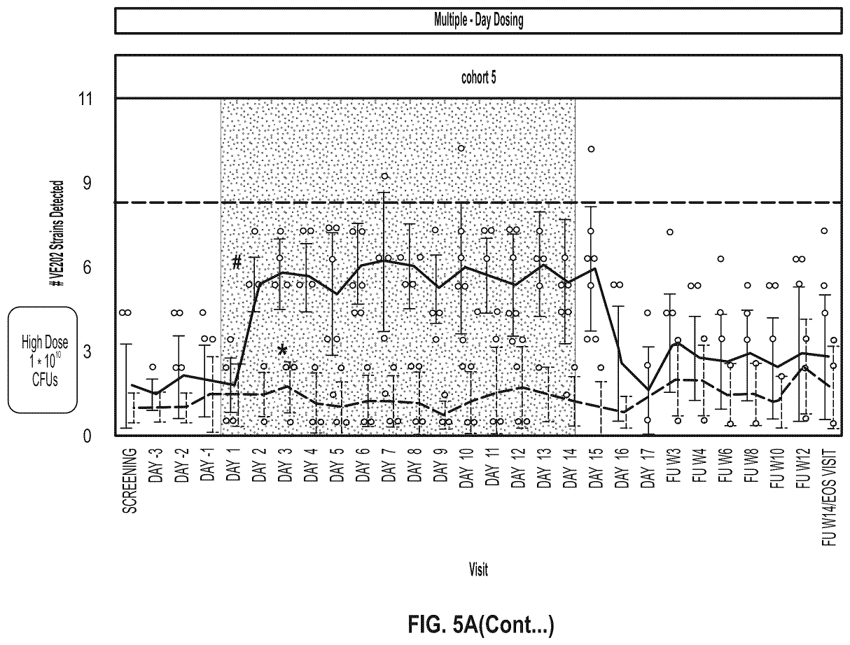

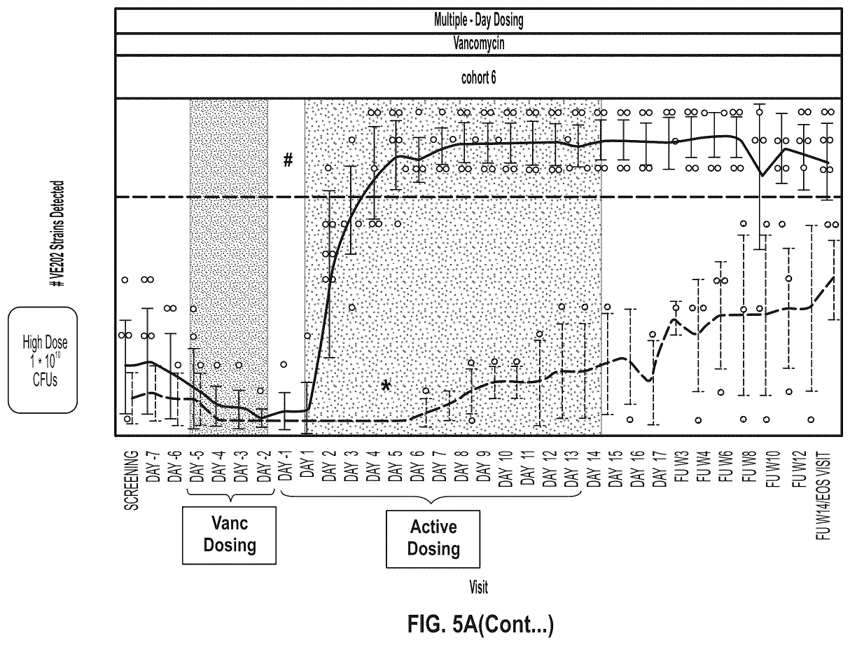

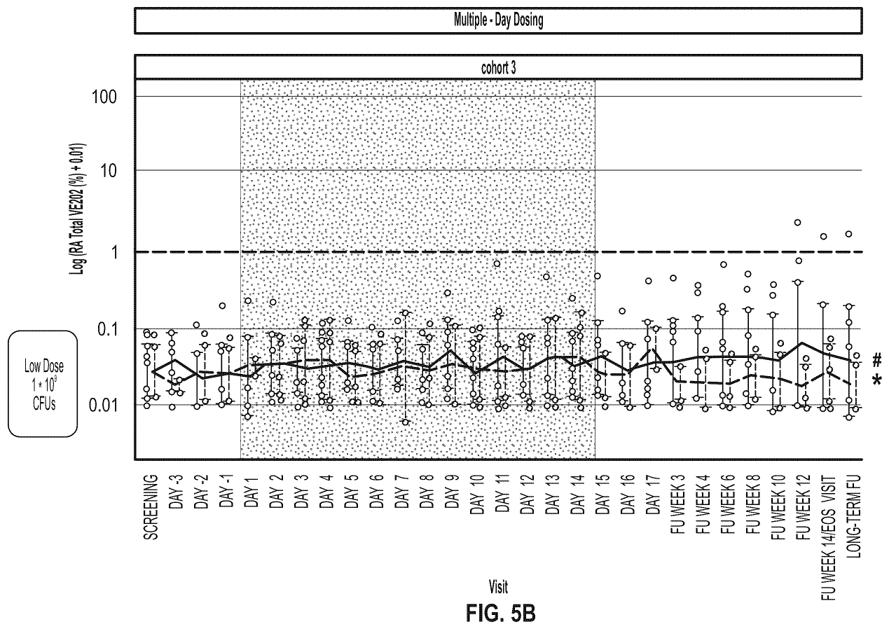

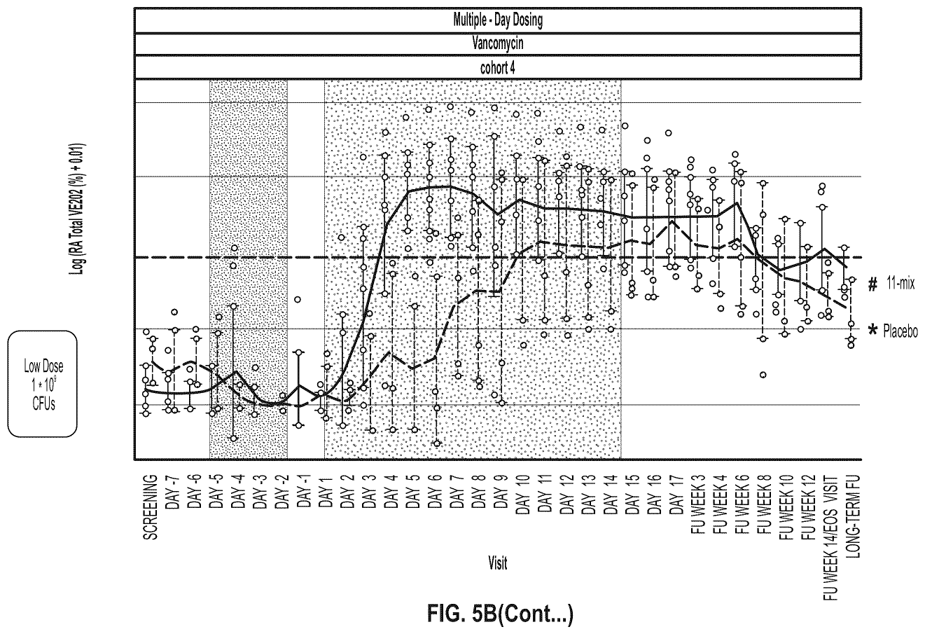

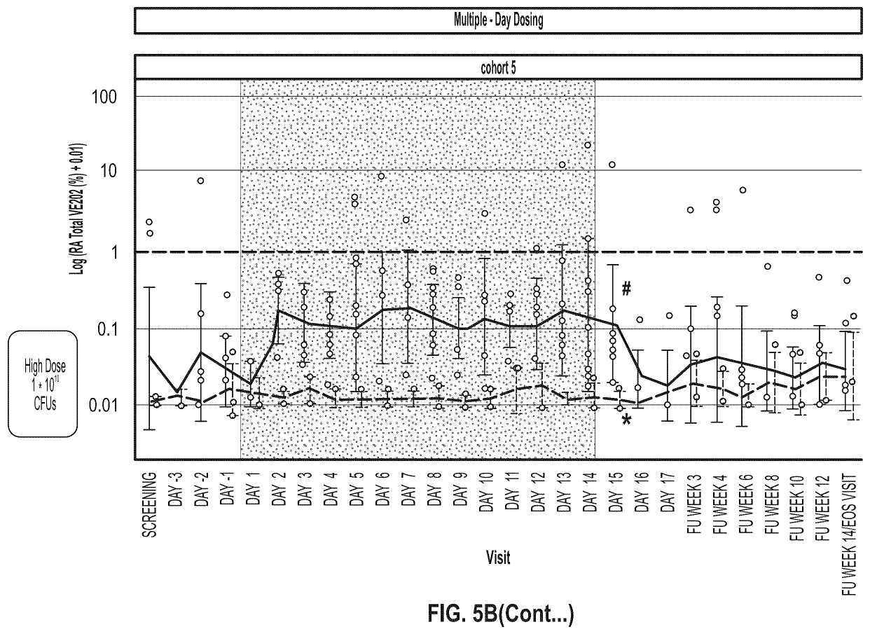

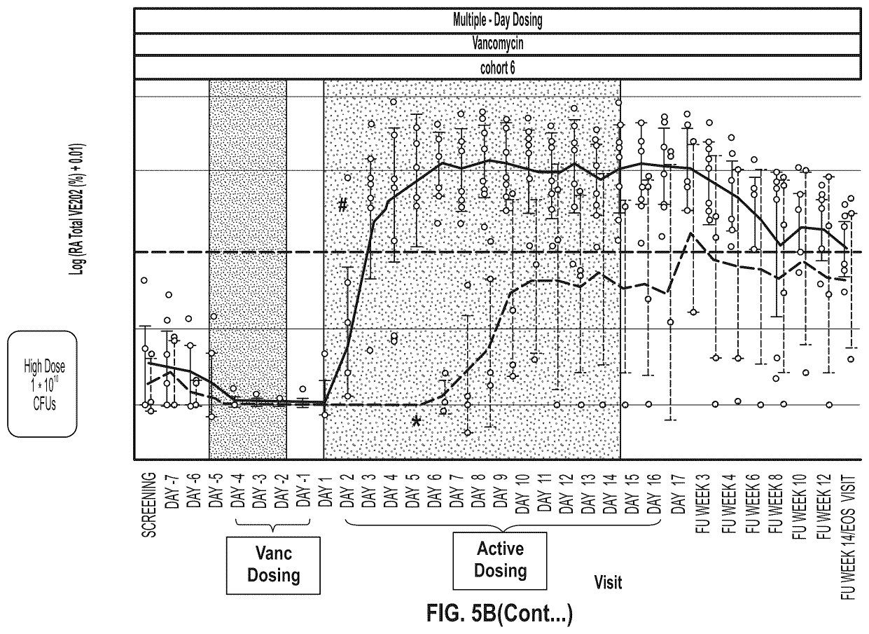

Summaries of the Detection and abundance of 11-mix strains in Part 2 are shown in FIGS. 5A and 5B.

In non-vancomycin pretreated cohorts, 11-mix strains transiently increased in detection and abundance during the active dosing period and then Strain detection and abundance returned to baseline within three days post-dosing. There was no observable increase in detection or the abundance of strains of the 11-mix strains in the placebo-treated cohorts.

Vancomycin pretreated cohorts rapidly and significantly increased in both detection and abundance of 11-mix strains within two days of the active dosing period. This was sustained throughout the active dosing period and up to two weeks post dosing. Detection and abundance of strains then slowly declined but remained substantially higher at the end of study visit when compared to baseline samples.

Placebo-treated participants who were pretreated with vancomycin were co-housed with 11-mix-treated participants through the 2-week active dosing period and 2-week follow-up. These placebo participants also exhibited an increase in detection of 11-mix strains during the second week of active dosing which was sustained to the end of study.

Population-level summary statistics for Cₘₐₓ, Tₘₐₓ, C_{first}, and Cₗₐₛₜ parameters are described below.

In low dose (cohort 3) and high dose (cohort 5) cohorts, respectively, the mean Cₘₐₓ of 4.1±2.0 and 5.9±2.5 total detected strains were observed at Tₘₐₓ of Day 9 and Day 15. No significant differences were observed comparing Cₗₐₛₜ to C_{first} in the non-vancomycin pretreated cohorts (Cₗₐₛₜ 2.4±1.6 and 2.1±2.5 compared to C_{first} of 2.1±1.2 and 1.3±1.6).

The relative abundance of all strains showed a mean Cₘₐₓ of 0.39%±0.77 and 2.96%±7.99 RA of total strains of the 11-mix at Tₘₐₓ at the week 12 follow-up and Day 14, respectively. No significant differences were observed comparing Cₗₐₛₜ to C_{first} in the cohorts that did not receive vancomycin pretreatment. There was no observed difference between the Cₗₐₛₜ (0.24%±0.61 and 0.03%±0.04) and C_{first} (0.03%±0.03 and 0.66%±1.13).

In low dose (cohort 4) and high dose (cohort 6) cohorts, respectively, detection of Cₘₐₓ of 9.00±1.58 and 10.00±1.00 were observed at Tₘₐₓ Day 15 and Day 7, respectively. The Cₗₐₛₜ was significantly higher with 7.6±1.1 and 8.9±1.6 mean total detected strains compared to C_{first} of 1.1±0.9 and 1.9±1.7 for low and high doses, respectively.

The Relative Abundance of all strains showed a mean Cₘₐₓ of 18.84%±29.40 and 39.23%±31.84 observed at Tₘₐₓ Day 7 and Week 3 follow-up. Abundance was substantially higher at Cₗₐₛₜ (0.83%±0.46 and 1.08%±1.31) compared to C_{first} (0.02%±0.04 and 0.08%±0.15).

In cohorts 4 and 6 respectively, strain detection was Cₘₐₓ/Tₘₐₓ 4.00±1.00 at Week 6 follow up and 5.33±1.53 at Week 14 follow-up. Abundance Cₘₐₓ/Tₘₐₓ was 8.8%±8.75 at Week 3 follow-up and 13.31%±18.36 at week-3 follow-up.

Detection and abundance of 11-mix strains were sustained to the end of study in the vancomycin pretreated placebo participants. Cₗₐₛₜ/C_{first} was 2.2±1.5:2.5±1.3 and 3.2±3.0:0.8±1.0 for detection while Cₗₐₛₜ/C_{first} for RA was 0.25%±0.19:0.04%±0.03 and 1.56%±1.84:0.02%±0.02.

Two participants presented with severe adverse events (SAEs) cohorts of Part 2 of the study. One subject was diagnosed with apathy and withdrew from the trial before the end of the study. The second participant was diagnosed with tinnitus and hearing loss. One SAE (hearing loss) was determined to be possibly related to vancomycin. The microbial density measures, detection and abundance of 11-mix strains were evaluated for these two participants and found to not be outliers in the analysis. Though, the participant with hearing loss had the highest microbial density measures observed in the cohort.

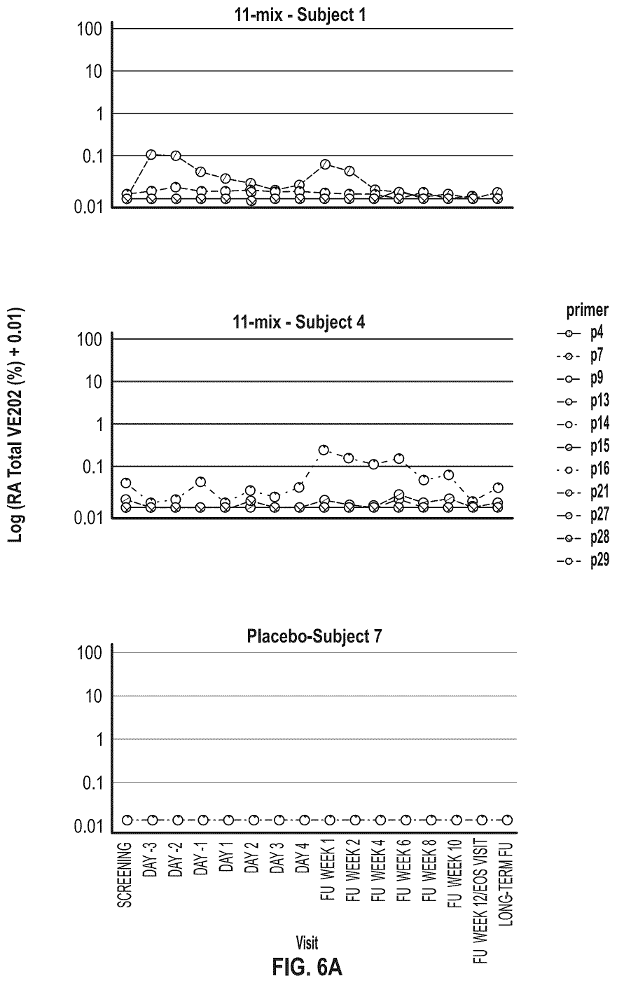

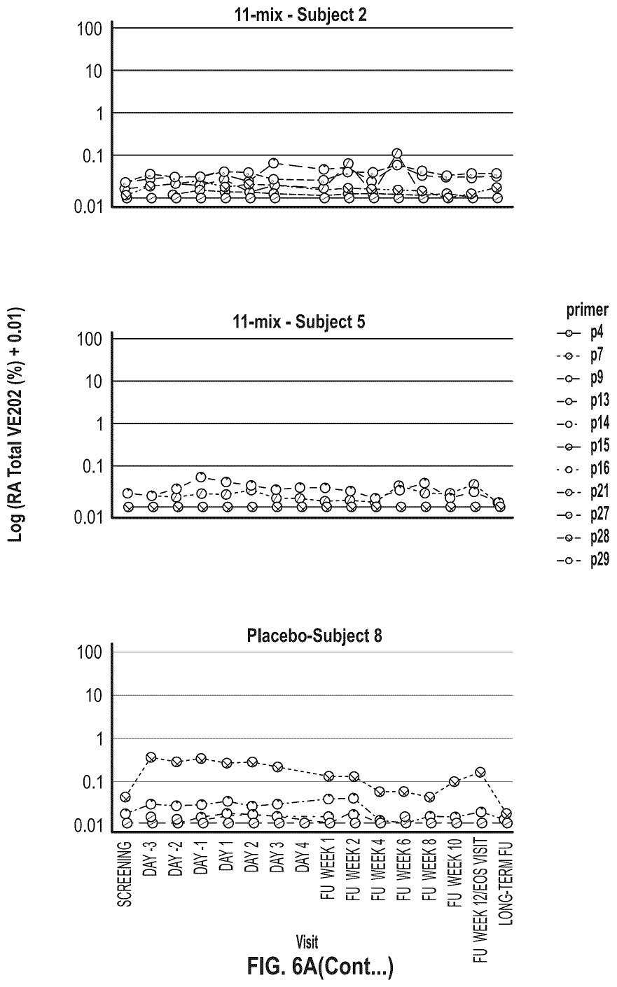

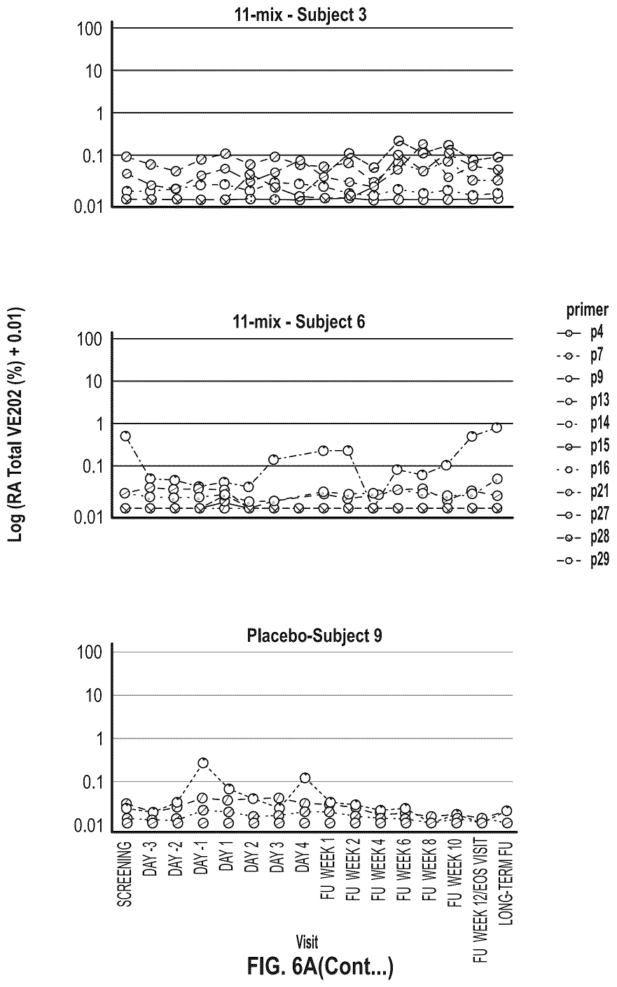

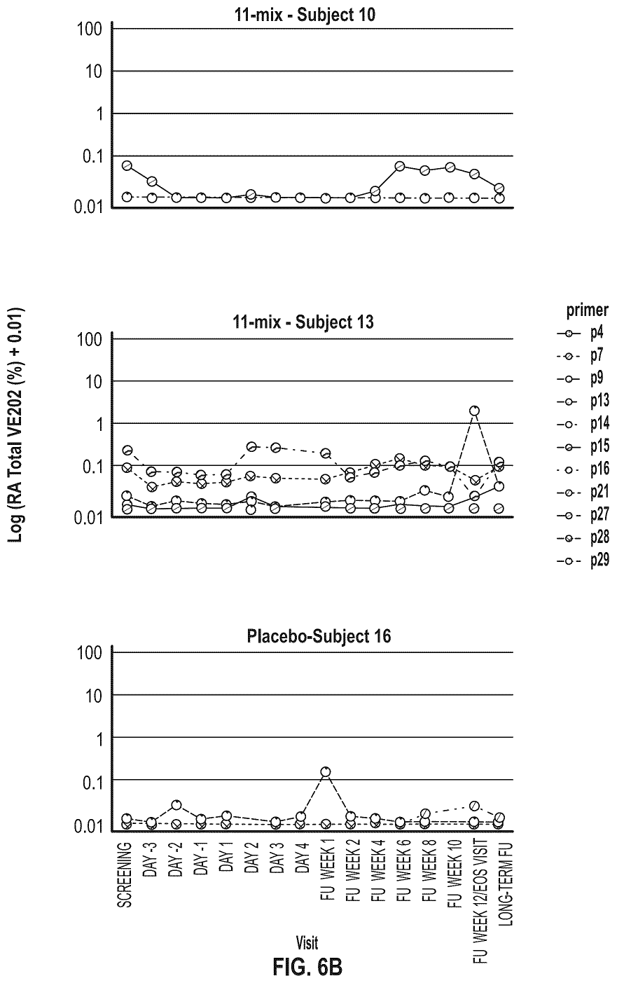

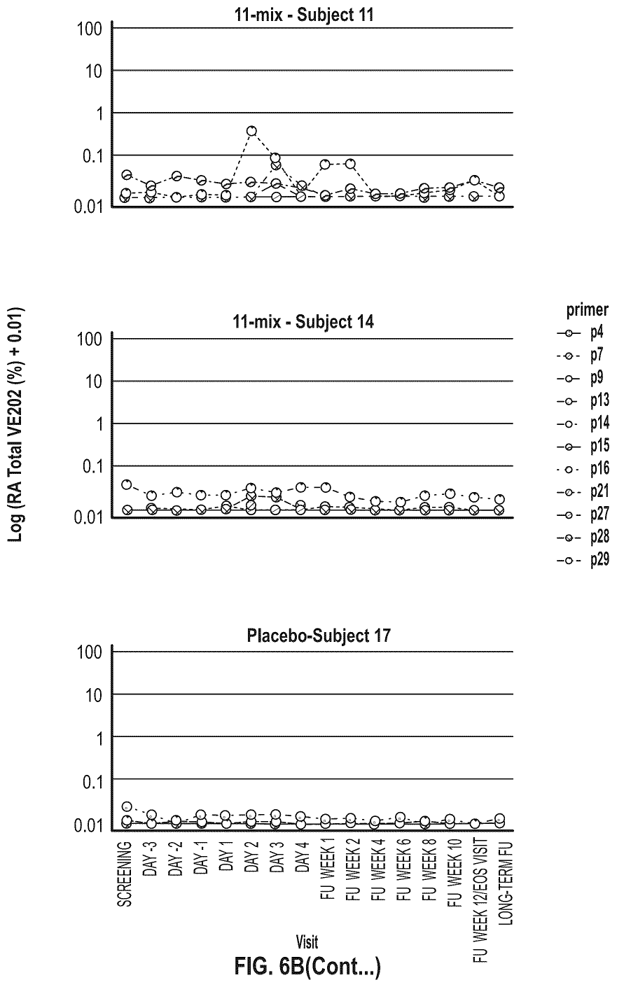

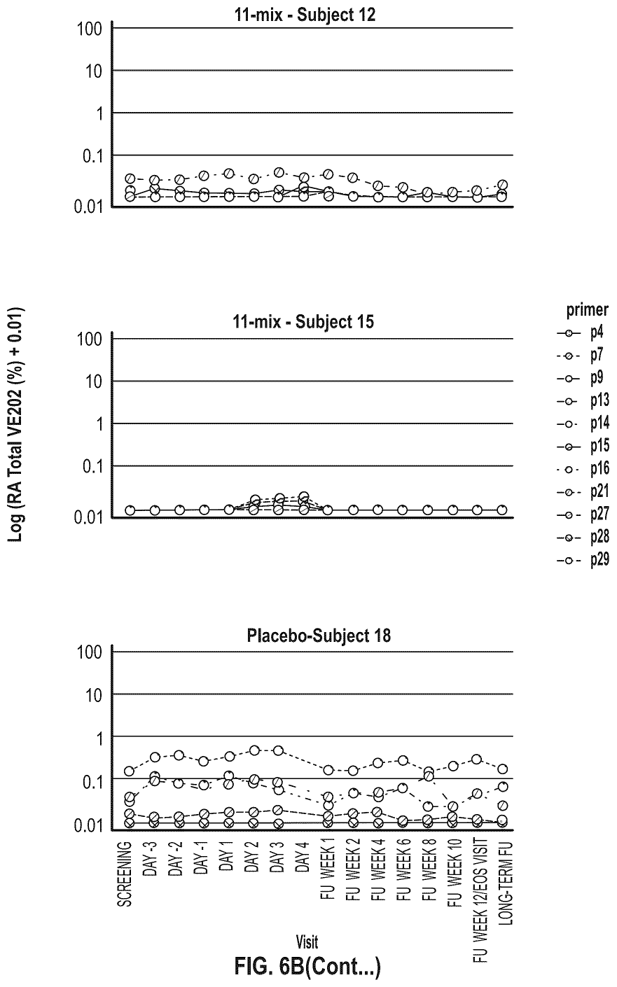

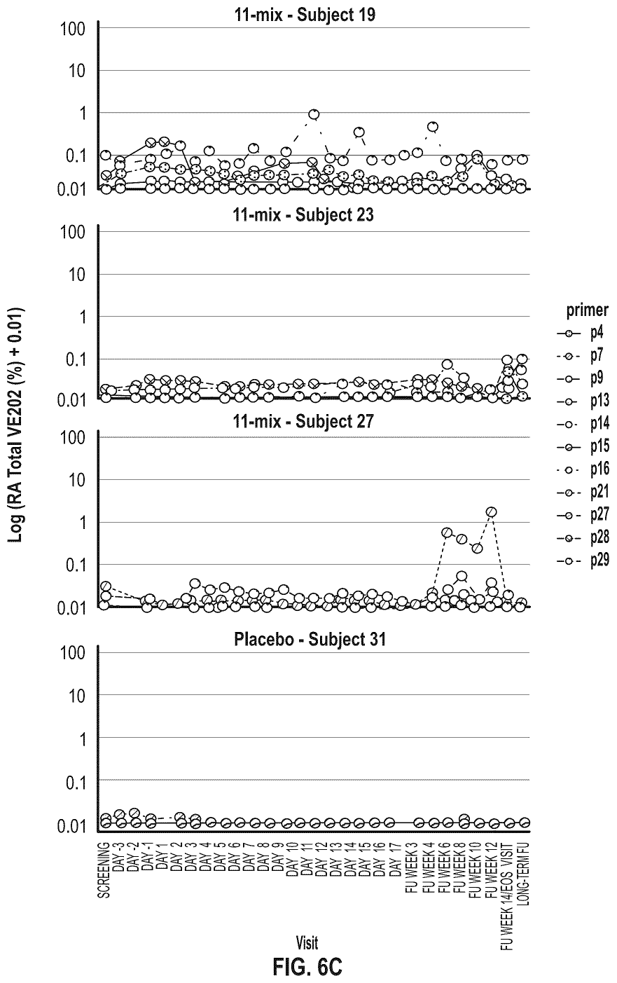

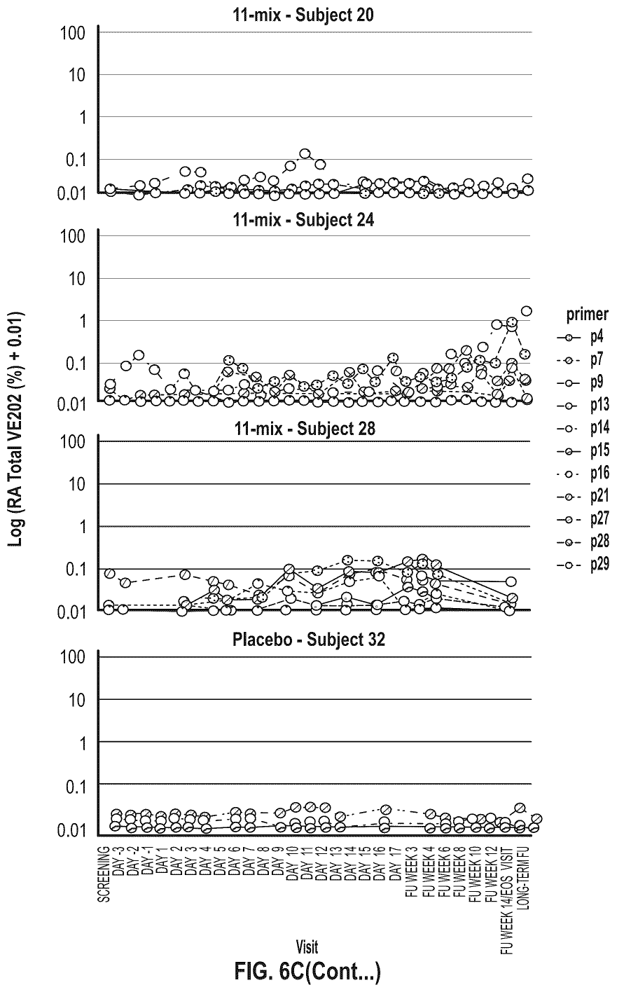

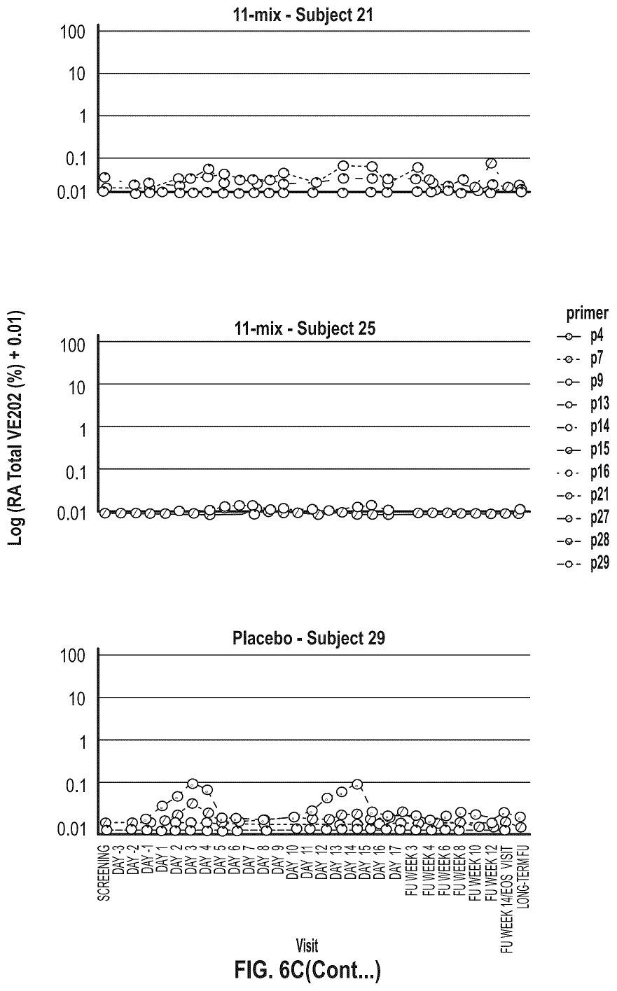

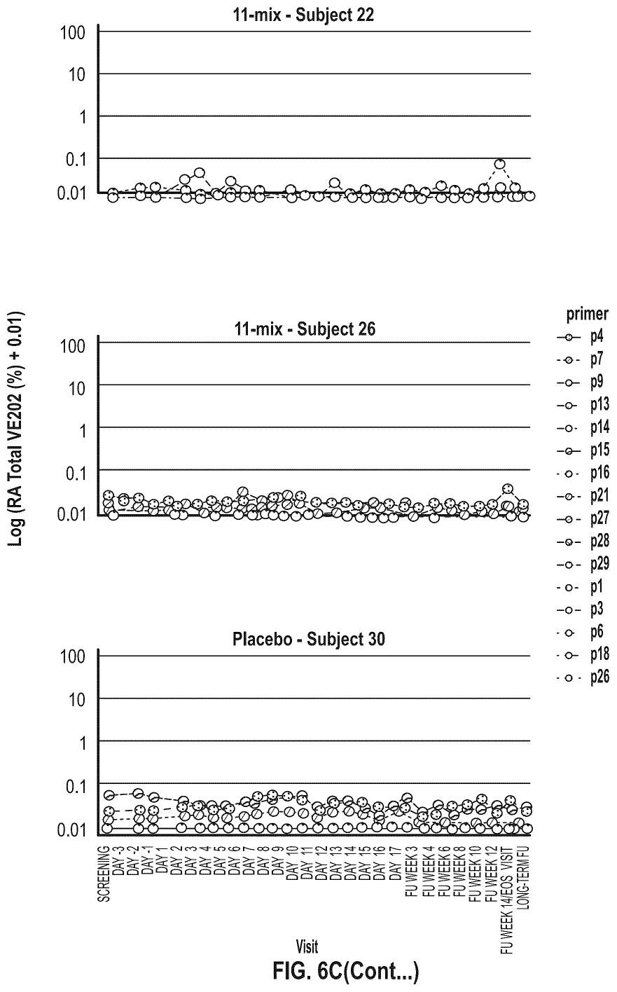

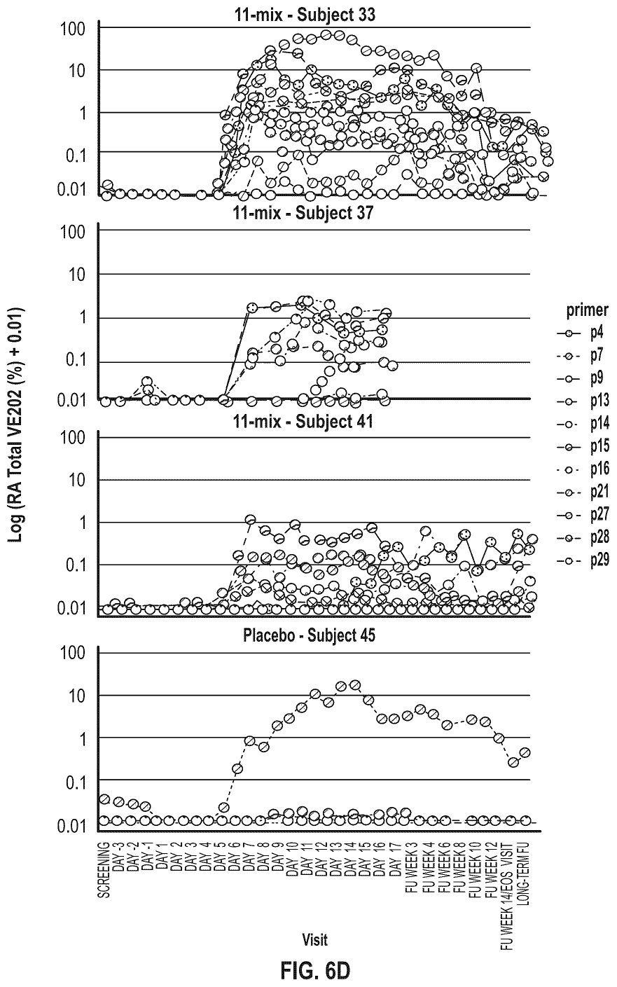

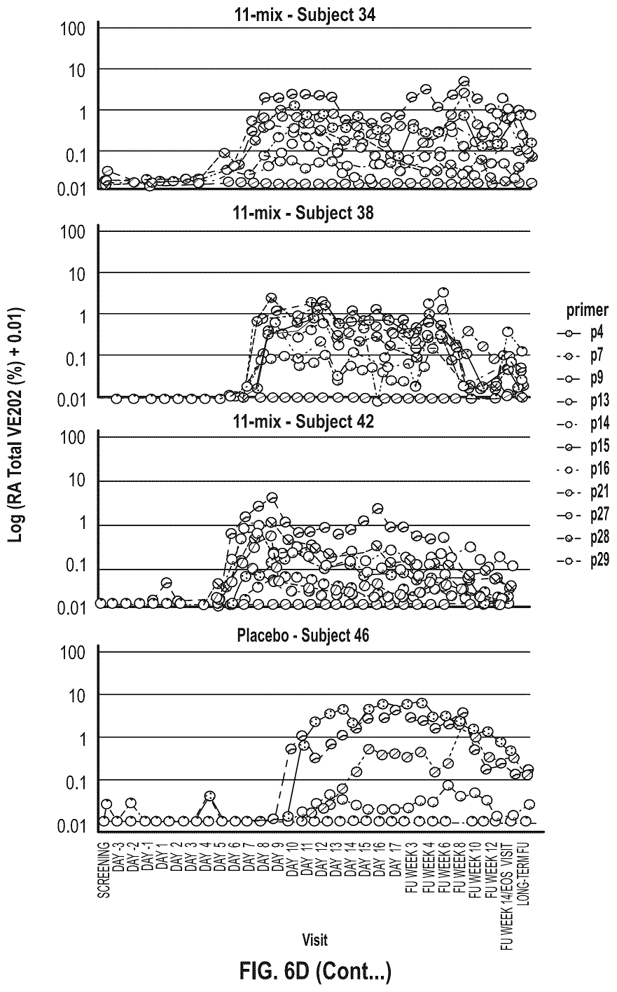

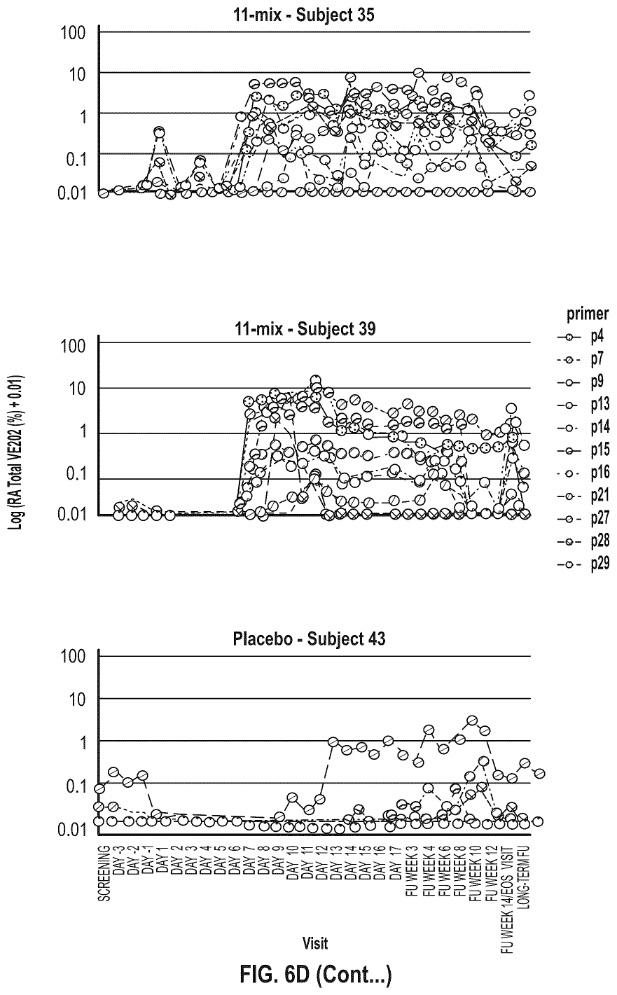

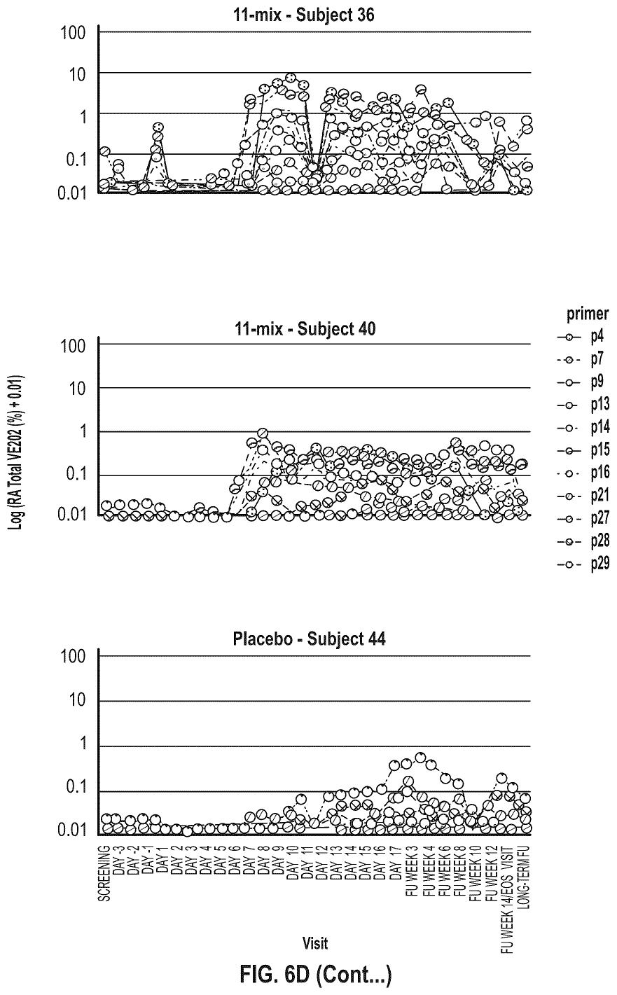

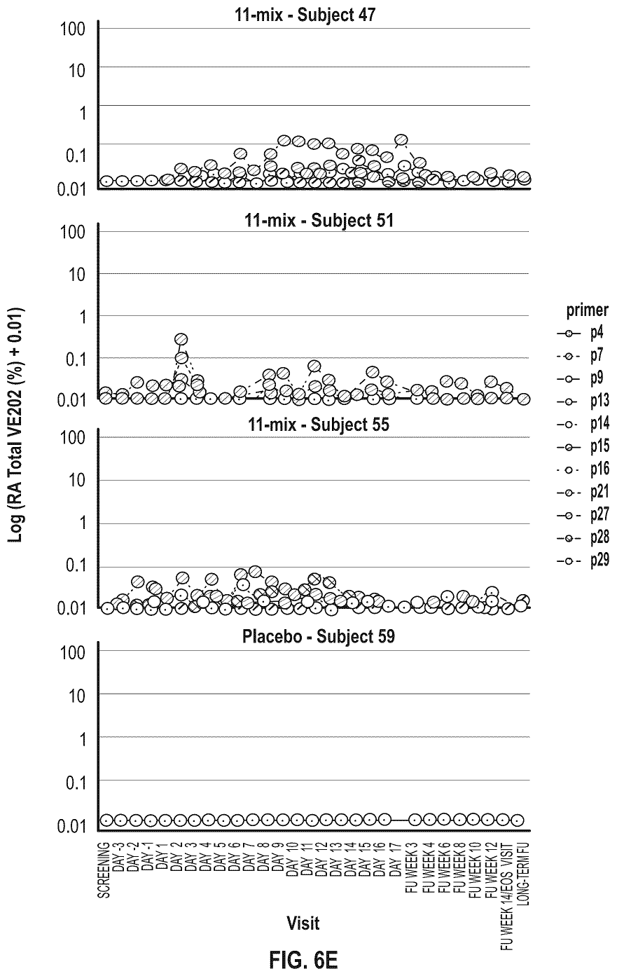

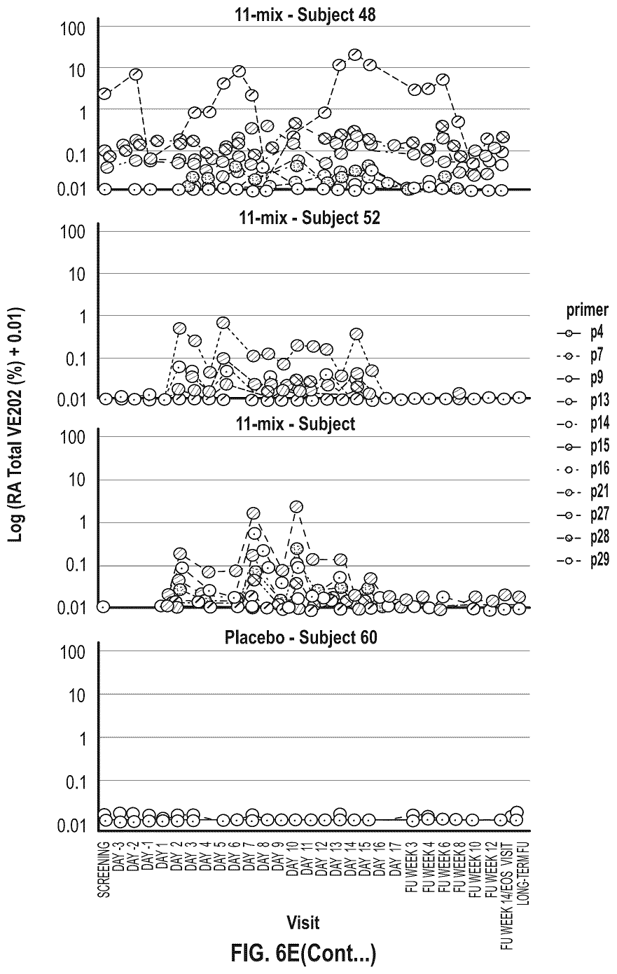

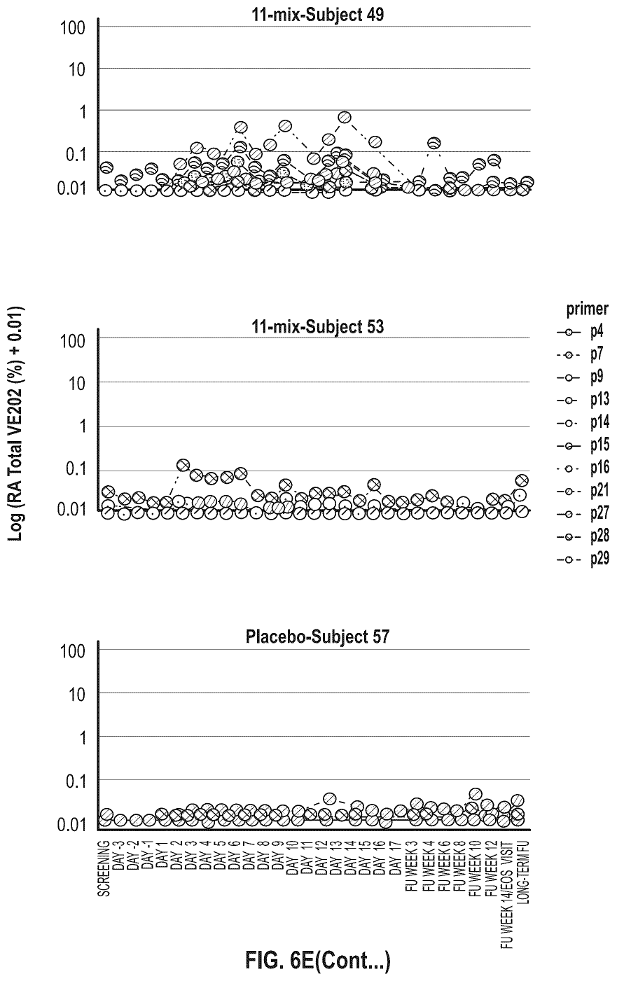

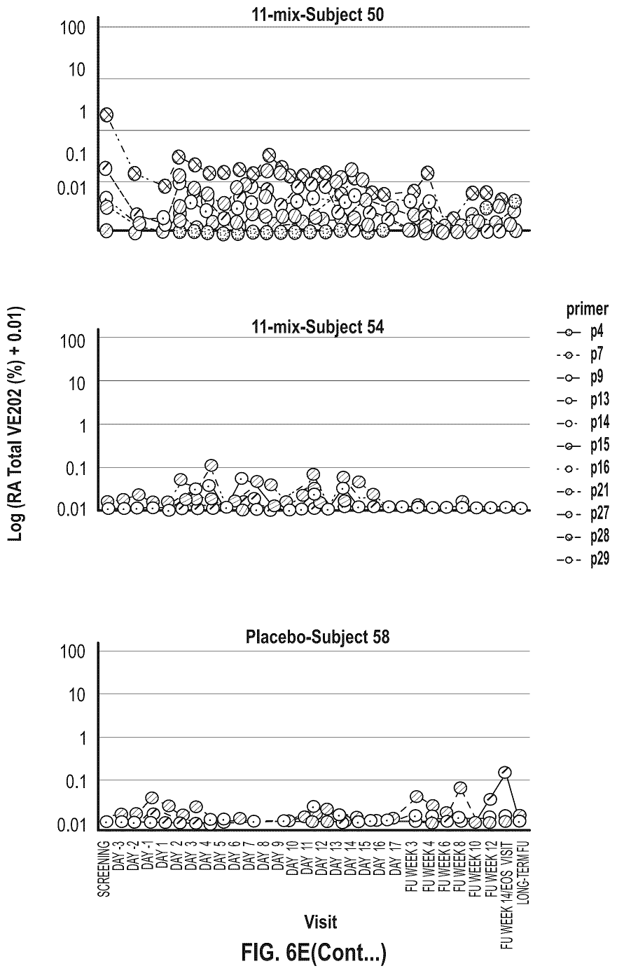

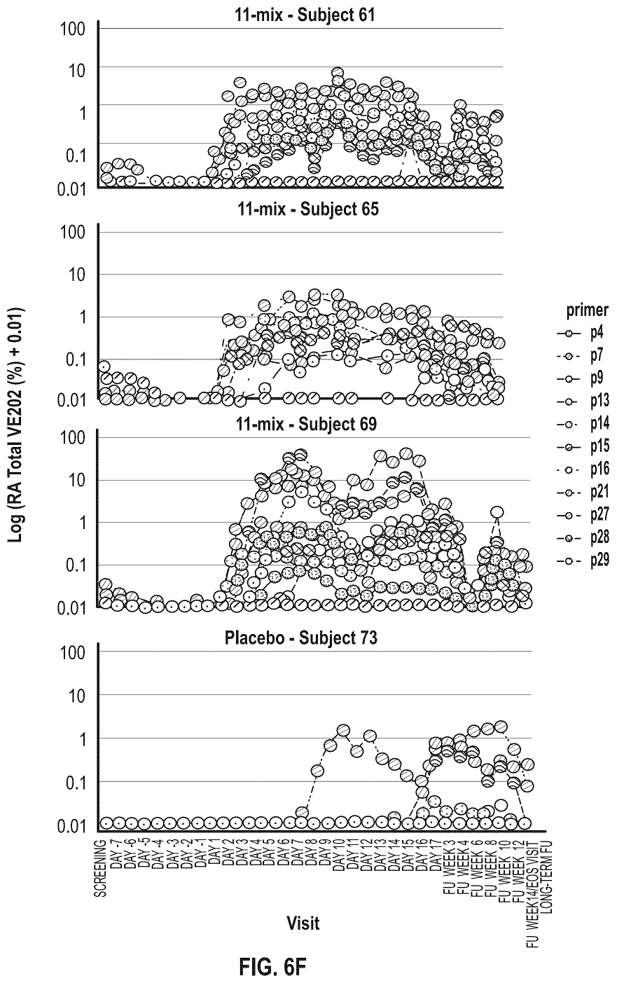

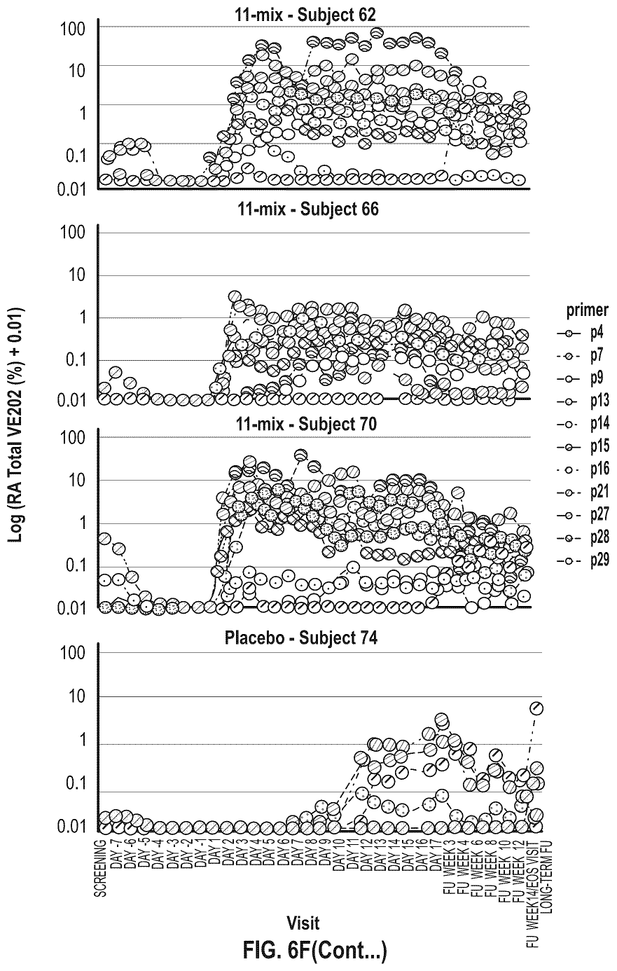

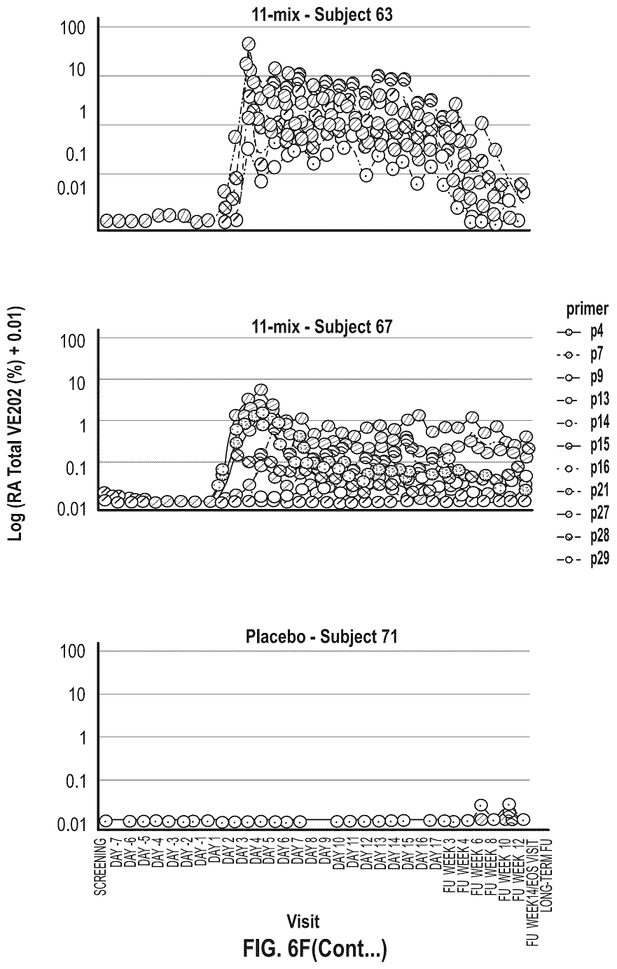

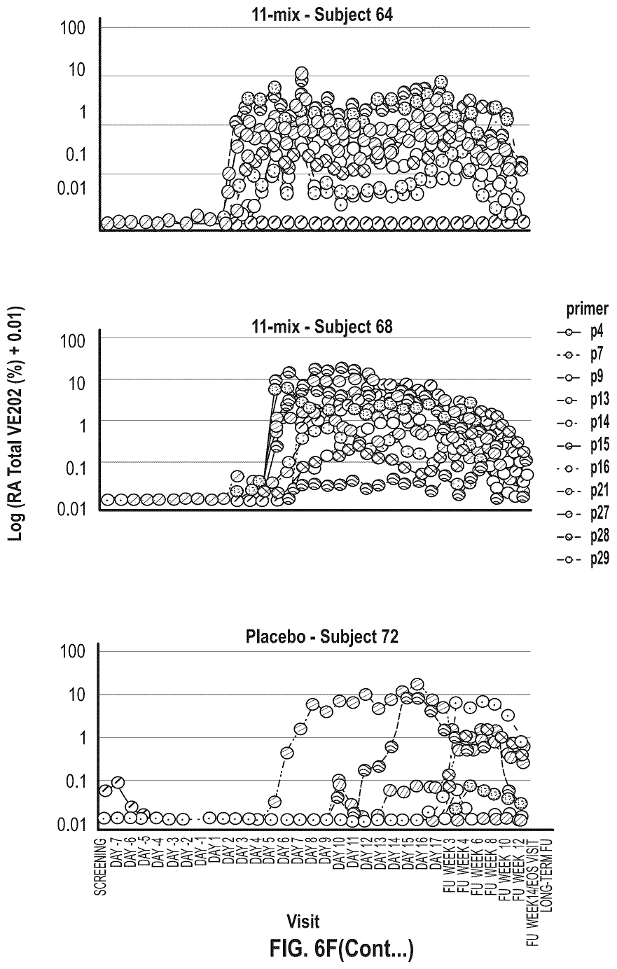

Strain level dynamics were cohort, treatment, and participant-specific. Plots showing strain level dynamics for each participant are shown in FIGS. 6A-6F.

Part 1 of the study showed few examples of increased strain detection. In cohort 1, participant 2270 showed subtle increased levels (<0.1% RA) of Clostridium lavalense post treatment which persisted to the last visit. In cohort 2, most subjects that received the 11-mix showed transient detection of several strains, but there was no evidence of strains persisting beyond the active dosing period.

In part 2, cohort 3 participants not pretreated with vancomycin showed colonization of individual strains at long term follow-up visits. For example, one subject showed durable colonization of Clostridium lavalense at low levels (<0.1% RA). Subjects in Cohort 5 showed transiently increased abundance of several strains, however there was little evidence of prolonged colonization of these strains. One subject showed high background detection of several strains (Clostridium bolteae, Ruminococcus sp., Lachnospiraceae bacterium).

Certain patterns were observed in subjects that were administered the 11-mix following pretreatment with vancomycin. Certain strains, for example Clostridium lavalense, showed rapidly increased abundance in several subjects in cohort 4 and 6. Other strains (e.g., Anaerotruncus colihominis) showed slower colonization dynamics, frequently rising in detection in the 2nd week of active dosing.

Placebo-treated subjects who were pretreated with vancomycin showed variable detection of strains. Some subjects showed high level colonization of an individual strain (Clostridium bolteae), while other participants showed colonization of several strains. Colonization dynamics differed between subjects as well. One subject showed an initial increased abundance of Lachnospiraceae bacterium during the active dosing period, while other strains (Clostridium indolis, Clostridium lavalense, Clostridium sp.) were only detected in the post dosing follow-up visits. These dynamics were also observed in two placebo-treated subject cohort 6. In these subjects, Clostridium bolteae increased in abundance early and was followed by other strains (e.g., Clostridium hathewayi, Clostridium indolis, Clostridium lavalense). There was also an example of a vancomycin pretreated placebo participant who showed no increased abundance of any of the strains of the 11-mix at any point during the study.

### Discussion

The results of the qPCR assay developed detect and quantify 11-mix strains in donor stool in the FIH trial of 11-mix are presented in this Example.

There was batch variability observed in the DNA extraction prior to the qPCR assay, which could impact downstream microbial density measurements. The results described herein highlight the variability involved with estimating total bacterial load from stool samples. Despite a level of assay variability, microbial density was observed to consistently decrease by roughly ˜90% in all participants following vancomycin pretreatment (cohorts 4 and 6). Stool samples were not homogenized prior to storage which could help explain this variation between sampling.

The qPCR assay described herein was effective in detecting small, but significant, colonization in pre-dose samples which suggest the presence of genetically similar bacterial strains in the healthy human gut. This can be expected as the 11-mix strains were initially isolated from a healthy human volunteer (Atarashi et al. Science (2013)).

The colonization kinetics (CK) of a single day of dosing without vancomycin pretreatment did not result in increased detection or abundance of strains of the 11-mix. In multiple day dosing cohorts, vancomycin pretreatment reduced gut microbial density which gradually recovered throughout the course of the study, although this was highly variable. Vancomycin pretreatment was required for sustained detection of strain of the 11-mix strains as compared to non-vancomycin pre-treated cohorts. In cohorts 4 and 6, detection of the strains of the 11-strains rapidly increased within 2 days of dosing and peaked at day 8. In these cohorts, strains of the 11-mix were detected up to 6 months post-treatment. Detection of the strains of the 11-mix were also detected in vancomycin pre-treated placebo-treated subjects.

The detection and abundance of strains of the 11-mix at long-term follow-up suggests a long-term colonization of strains of the 11-mix in the antibiotic pretreated healthy gut. Individual strain dynamics were subject-dependent and demonstrate the variability of the human microbiome and host:microbiome interactions.

Unexpectedly, placebo-treated subjects that received vancomycin pretreatment co-housed with active drug treated participants also demonstrated low level 11-mix strain-specific colonization although at significantly lower RA levels. The strains that colonized were subject specific, and one subject did not show any colonization of strains.

The qPCR assay presented in this report could be used as a rapid and cost-effective co-diagnostic method to assess the colonization of strains in participants receiving live bacterial products.

In conclusion, use of the 11-mix live bacterial product was determined to be safe and tolerable and exhibited significant and durable colonization of the healthy human gut following vancomycin pretreatment.

### Example 2: Detection, Abundance, and Colonization Kinetics of Bacterial Strains from a 16-Mix Bacterial Mixture in the Heathy Human Gut by qPCR

This example describes a randomized, double-blind, placebo-controlled, single and multiple-day, multicenter, interventional study was conducted to evaluate the colonization efficacy of the 16-mix as described in Table 1. Subjects were between 18 and 60 years of age (inclusive), who had a body mass index (BMI) between 18 and 30 kg/m² (inclusive), and body weight of not less than 50 kg. Female participants of childbearing potential were excluded if pregnant, undergoing assisted reproduction, or would be breast feeding during the study period and 3 months after last dose of study intervention.

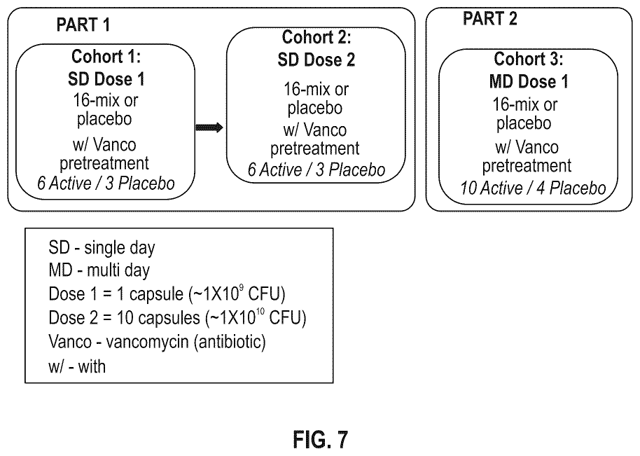

The study was executed in two parts, as shown in FIG. 7.

In Part 1, a single-day dose administration of the 16-mix was evaluated in 2 cohorts (n=9 per cohort; 6 active and 3 placebo). Subjects were pretreated with vancomycin (125 mg, administered 4 times daily (500 mg total per day)) for five days prior to dosing:
Cohort 1: Dose 1 (˜1×10⁹ colony forming units (CFU); 1 capsule); and
Cohort 2: Dose 2 (˜1×10¹⁰ CFU per day; 10 capsules).

In Part 2, multiple-day dose administrations of the 16-mix were evaluated in one cohort (n=14; 10 active and 4 placebo). Subjects were pretreated with vancomycin (125 mg, qid) for five days prior to dosing:
Cohort 3: Dose 1 (˜1×10⁹ CFU; 1 capsule; 14-day dose regimen) with vancomycin pretreatment.

Stool samples were collected frequently throughout the duration of the study up to a six-month long-term follow-up. Participants collected a small volume of stool into a tool following the manufacturer's instructions. Stool samples collected at the clinical sites were stored immediately at −75° C.±10° C. post collection. Samples collected at home were stored at room temperature (25° ° C.±10° C.) for up to 1 week before being stored at −75° C.±10° C.

Bacterial DNA was extracted using high throughput extraction kits and quantitated. DNA libraries were constructed with the bacterial DNA from each kit for whole genome sequencing and sequenced on the Illumina NovaSeq System.

Strain specific primers (25 nt each) based designed based on data generated to detect each of the 17 strains using qPCR. Primers used to detect each strain are shown in Table 2. The amplicons generated by each pair of qPCR primers are shown in Table 3.

The 16-mix strain quantification qPCR assay was prepared: 5 ng of bacterial DNA was added to a strain specific primer set (0.8 μM), mixed with PerfeCTa SYBR Green FastMix Low ROX (QuantaBio). A standard curve was also generated using strain specific gDNA for each strain of the 16-mix. qPCR amplification was performed on the QuantStudio™ 12K (ThermoFisher) using the following conditions:
Run Method: Hold Stage: 95° C.×10 min,
PCR Stage: 40 cycles (95° C.×10 sec, 60° C.×20 sec),
Melt Curve: 95° C.×15 sec, 60° C.×1 min, 95° C.×15 sec.

For abundance estimation the mean RA value was taken for multiple samples. Relative abundances (%) measures were estimated from qPCR data to help in future comparisons with metagenomic shotgun sequencing data. This was done by normalizing the qPCR output (ng) by the total input into the qPCR assay, which was assumed to be 5 ng:

ng 16-mix gDNA/5 ng input total DNA

Data was log₁₀ transformed in figures to show small variation in baseline samples. An arbitrarily small value of 0.01 was added before log transformation.

### Results

Most participants demonstrated a background detection of one to three strains of the 16-mix in baseline samples.

The highest number of detected strains in screening samples were strains 29, which was detected in 2/3 of screening samples, followed by strains 26, 27, 7, 21, 28, 6 which were detected in more than 10% of screening samples (Table 4). All other strains were detected in less than 10% of screening samples. All strains were detected at very low abundance level. The maximum total RA % of all 16 strains observed in a screening sample was 0.396% in subject 473074. The mean total RA % of the 16 strains was 0.066%+/−0.103.

There was large variation in microbial density observed between participants in the baseline samples (mean 19.48±13.99 ng/mg, minimum 1.94 ng/mg, maximum 55.87 ng/mg). No significant differences were observed in microbial density between cohorts or when stratified by treatment cohort. Three subjects from the placebo-treated group in cohort 2 exhibited a non-significant trend of lower microbial density, but this was not statistically significant.

All participants in this study received vancomycin pretreatment (125 mg administered 4 times daily for 5 days). Vancomycin pretreatment was used to determine whether antibiotic-mediated reduction of the abundance of bacteria in the host microbiome is required facilitate colonization of the recipient microbiome by bacterial strains of the 16-mix. This short course of vancomycin pretreatment was well-tolerated and resulted in transient self-resolving treatment-related adverse events.

Vancomycin pretreatment resulted in a substantial decrease in microbial density of the native host microbiome. By Day −1, a roughly 50-60% reduction in microbial density was observed across cohorts and treatment arms. Microbial density recovered to baseline levels by day 7 in the multiple day dosing cohort, while single dose cohorts exhibited a return to baseline levels at the week 2 follow-up visit.

Summaries of the detection and abundance of 16-mix strains are shown in FIGS. 8A and 8B and Tables 4 and 5. All cohorts demonstrated rapid and significantly increased detection and abundance of 16-mix strains within two days of the active dosing period. This was sustained throughout the active dosing period and up to two weeks post dosing. Detection and abundance of strains then slowly declined but remained substantially higher at the end of study visit when compared to baseline samples.

Placebo-treated subjects who were pretreated with vancomycin were co-housed with participants receiving the 16-mix through the 2-week active dosing period and 2-week follow-up. These placebo participants also exhibited an increase in detection of the 16-mix strains during the second week of active dosing, which was sustained to the end of study.

Population-level summary statistics for Cₘₐₓ, Tₘₐₓ, C_{first}, and Cₗₐₛₜ parameters are described below.

In low dose and high dose cohorts, cohorts 1 and 2, respectively, the mean Cₘₐₓ of 8.2±2.3 and 13.0±0.0 total detected strains were observed at Tₘₐₓ of Week 1 follow-up and Week 12 follow-up. There was a significant difference observed comparing Cₗₐₛₜ to C_{first} in the single dose cohorts (Cₗₐₛₜ 7.0±2.4 and 8.8±1.5 compared to C_{first} of 3.0±1.8 and 2.8±2.2).

Abundance mean Cₘₐₓ was 29.53%±17.27 and 13.31%±6.29 RA of total strains of the 16-mix at Tₘₐₓ at the Week 1 follow-up and Day 4, respectively. Cₗₐₛₜ was higher than C_{first} for both cohorts (Cₗₐₛₜ 0.83%±0.78 and 0.77%±0.46 vs. C_{first} 0.08%±0.10 and 0.12%±0.28).

In the low multiple day dosing cohort (cohort 3) detection Cₘₐₓ of 14.3±0.9 was observed at Tₘₐₓ Day 4. Cₗₐₛₜ was significantly higher with 11.3±1.8 mean total detected strains compared to C_{first} of 2.7±1.2.

The relative abundance of all strains demonstrated a mean Cₘₐₓ of 32.23%±18.97 that was observed at Tₘₐₓ of Day 7. The abundance was substantially higher at Cₗₐₛₜ (2.45%±2.13) compared to C_{first} (0.31%±0.81).

In cohorts 1 and 2, respectively, strain detection was Cₘₐₓ/Tₘₐₓ 3.3±2.1 at the Week 8 follow-up and 3.0±1.7 at the Week 2 follow-up. The abundance Cₘₐₓ/Tₘₐₓ was 0.36%±0.50 at the Week 8 follow-up and 1.78%±1.76 at the Week 1 follow-up.

The detection and abundance of the strains of the 16-mix were sustained to the end of study in the vancomycin pretreated placebo participants. The Cₗₐₛₜ/C_{first} for cohorts 1 and 2 were 3.0+/−2.0/2.0+/−1.0 and 2.7+/−1.5/2.0+/−1.0, respectively for detection, while Cₗₐₛₜ/C_{first} for RA was 0.83%+/−0.78/0.08%+/−0.10 and 0.7%+/−0.46/0.12%+/−0.28, respectively.

In the multiple day dosing cohort 3, placebo-treated subjects showed a mean detection Cₘₐₓ/Tₘₐₓ 6.0+/−2.2 at the Week 4 follow-up. The Cₗₐₛₜ/C_{first} was 6.0+/−3.2/2.5+/−1.0. The mean abundance Cₘₐₓ/Tₘₐₓ was 2.62%+/−3.87 at Day 17 and Cₗₐₛₜ/C_{first} was 0.94%+/−1.15/0.05+/−0.04.

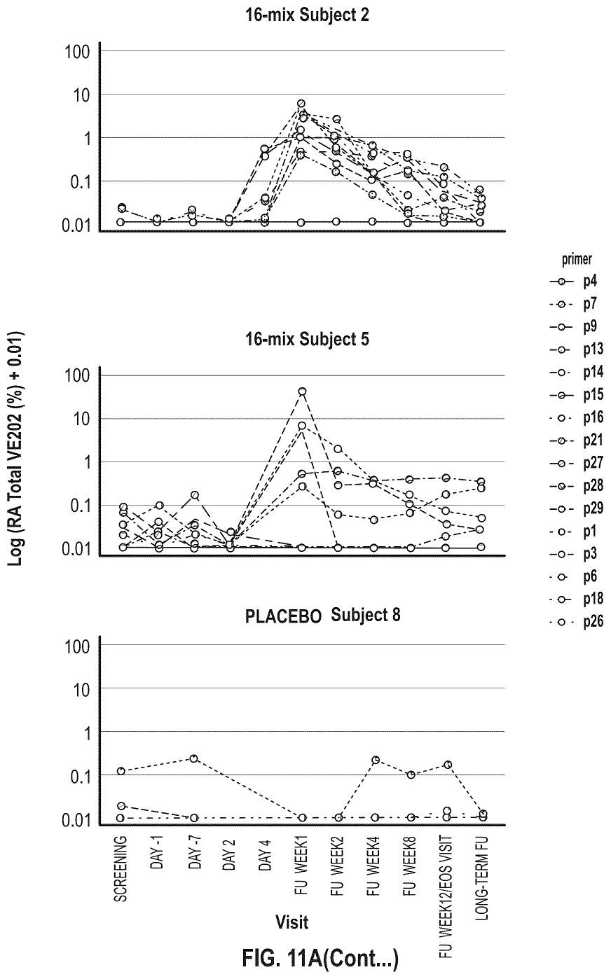

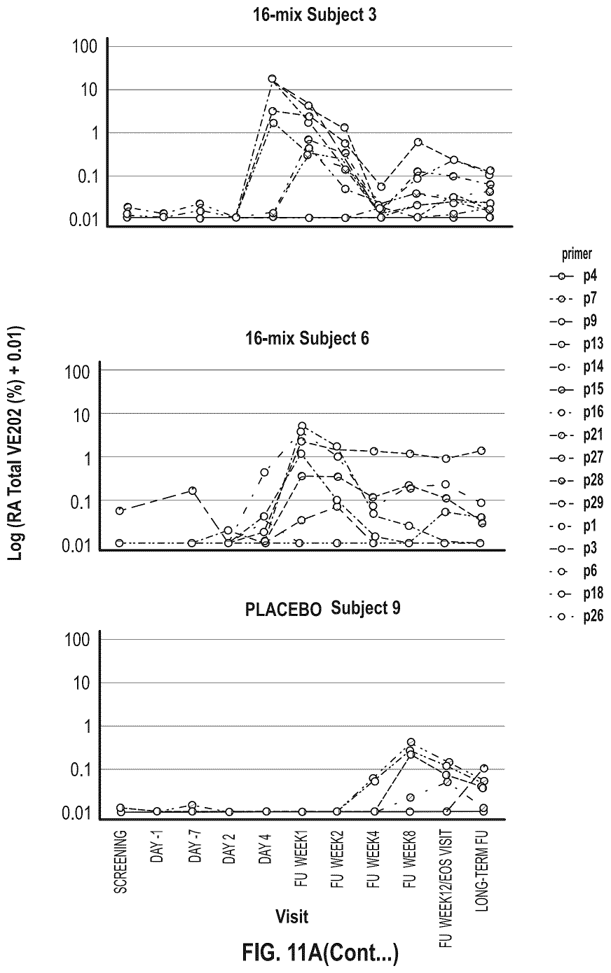

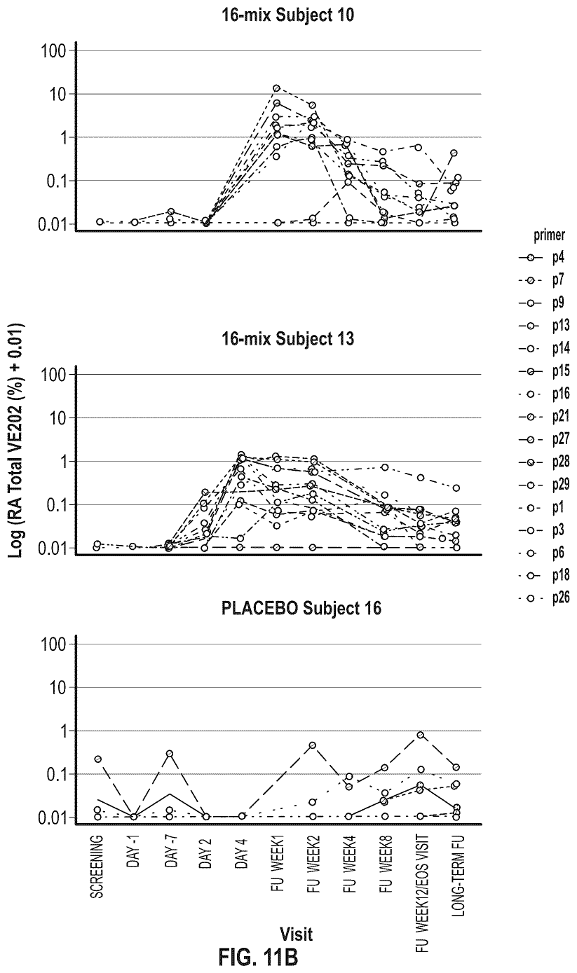

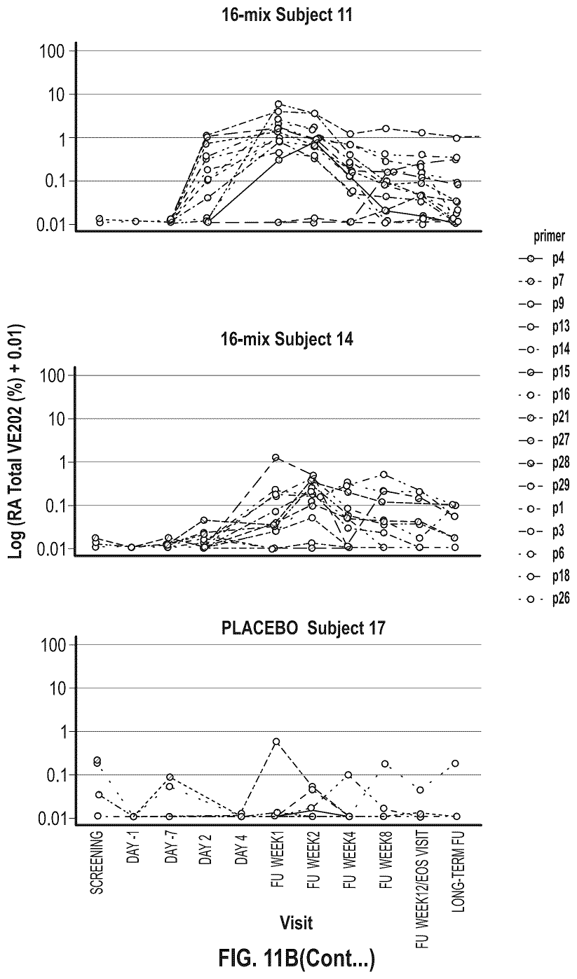

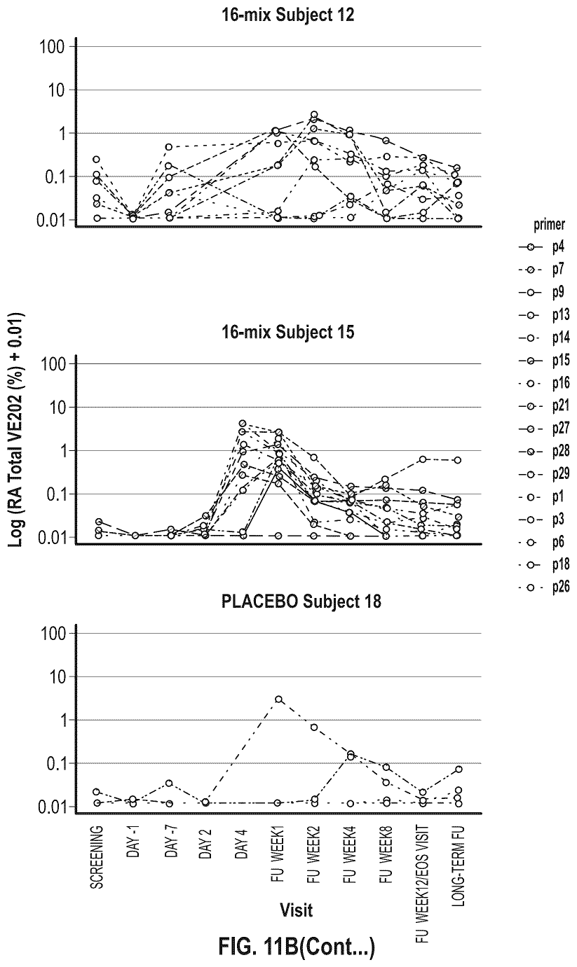

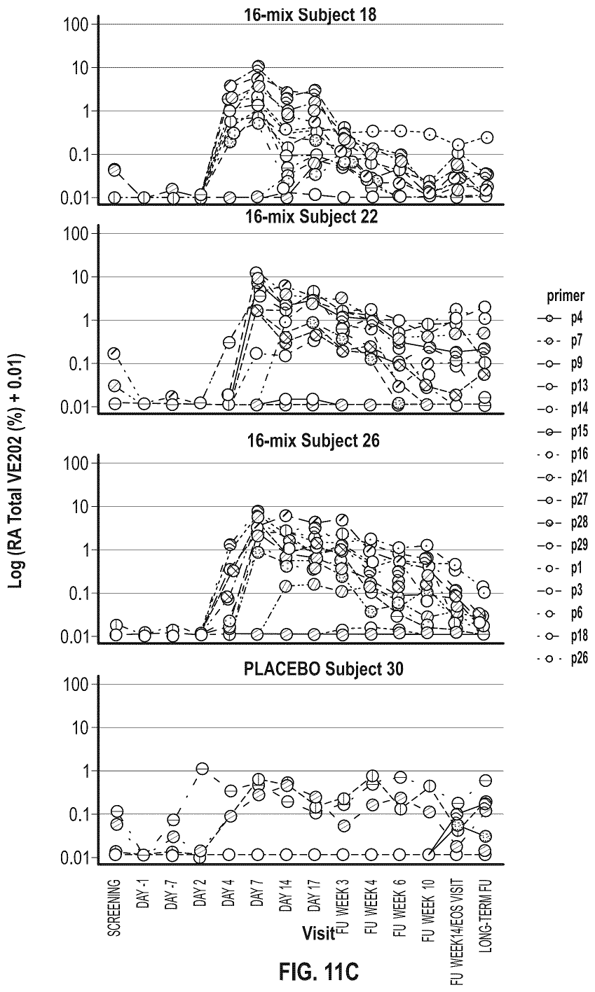

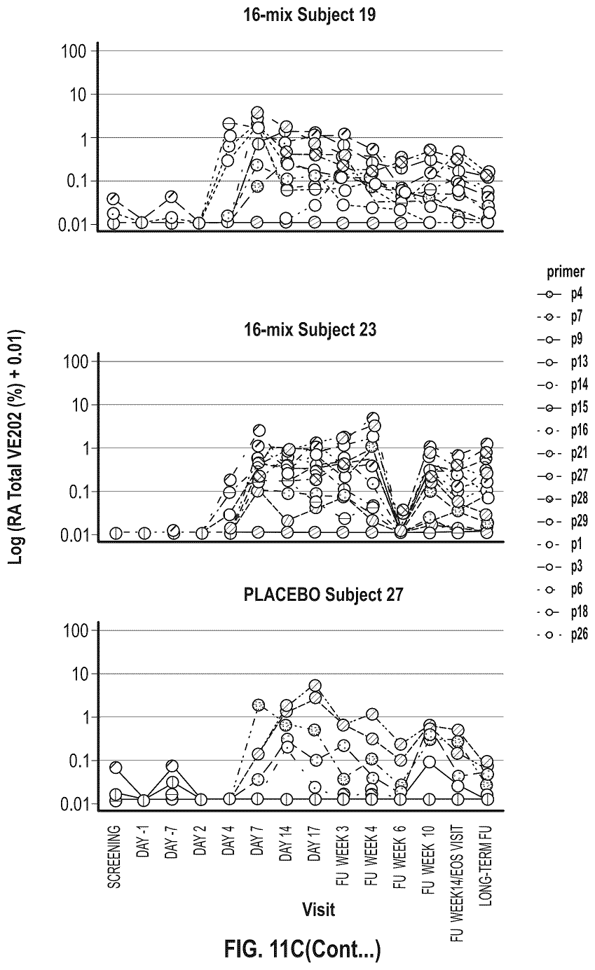

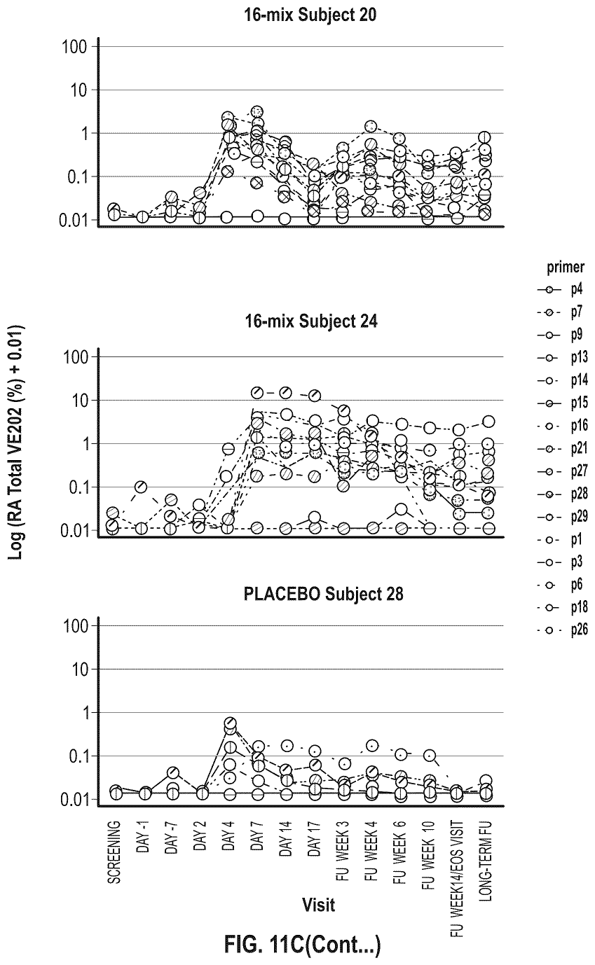

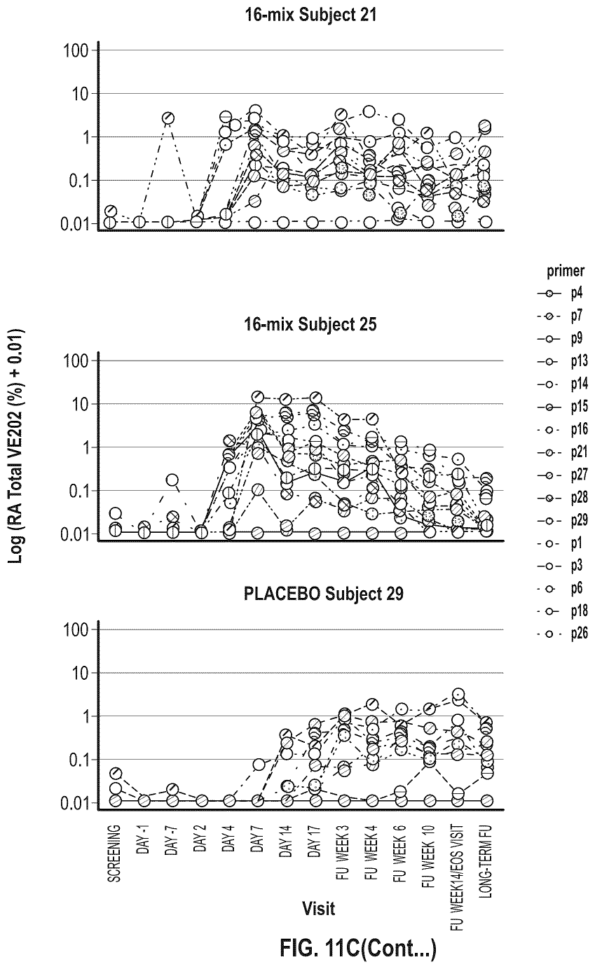

Strain level dynamics were cohort, treatment, and subject-specific. Plots showing strain level dynamics for each participant are shown in FIGS. 11A-11C.

TABLE 4
Number of strains of the 16-mix detected over time (Mean ± SD)

| | ACTIVE | | | PLACEBO | | |
|---|---|---|---|---|---|---|
| Cohort | 1 | 2 | 3 | 1 | 2 | 3 |
| SCREENING | 2.5 +/− 1.6 | 2.0 +/− 1.8 | 2.9 +/− 2.1 | 1.7 +/− 1.5 | 2.3 +/− 1.2 | 2.2 +/− 0.5 |
| DAY −7 | 3.0 +/− 1.8 | 2.8 +/− 2.2 | 2.7 +/− 1.2 | 2.0 +/− 1.0 | 2.0 +/− 1.0 | 2.5 +/− 1.0 |
| DAY −1 | 3.4 +/− 5.0 | 0.0 +/− 0.0 | 0.6 +/− 0.7 | 1.5 +/− 2.1 | 0.0 +/− 0.0 | 0.5 +/− 1.0 |
| DAY 2 | 2.7 +/− 3.8 | 6.8 +/− 4.2 | 4.1 +/− 4.3 | 0.0 +/− 0.0 | 0.0 +/− 0.0 | 0.8 +/− 1.0 |
| DAY 4 | 7.2 +/− 1.3 | 13.0 +/− 0.0 | 9.8 +/− 3.6 | 1.0 +/− 1.4 | 0.0 +/− 0.0 | 2.0 +/− 2.4 |
| DAY 7 | | | 13.4 +/− 0.9 | | | 3.5 +/− 2.1 |
| DAY 14 | | | 14.3 +/− 0.9 | | | 4.8 +/− 1.3 |
| DAY 17 | | | 14.1 +/− 1.4 | | | 5.5 +/− 2.5 |
| FU WEEK 1 | 8.2 +/− 2.3 | 11.2 +/− 2.5 | | 0.7 +/− 1.2 | 1 5 +/− 0.7 | |
| FU WEEK 2 | 8.0 +/− 2.6 | 11.5 +/− 2.5 | | 1.7 +/− 2.9 | 3.0 +/− 1.7 | |
| FU WEEK 3 | | | 14.1 +/− 0.8 | | | 5.2 +/− 2.6 |
| FU WEEK 4 | 7.0 +/− 2.9 | 10.8 +/− 2.2 | 14.1 +/− 0.8 | 3.0 +/− 2.6 | 1.7 +/− 0.6 | 6.0 +/− 2.2 |
| FU WEEK 6 | | | 12.3 +/− 4.1 | | | 5.0 +/− 2.7 |
| FU WEEK 8 | 7.3 +/− 3.3 | 10.0 +/− 1.7 | | 3.3 +/− 2.1 | 3.0 +/− 1.0 | |
| FU WEEK 10 | | | 12.3 +/− 2.5 | | | 5.2 +/− 2.6 |
| FU WEEK 12/ | 7.8 +/− 2.9 | 9.3 +/− 2.3 | | 3.3 +/− 1.2 | 2.7 +/− 1.2 | |
| EOS VISIT | | | | | | |
| FU WEEK 14/ | | | 11.6 +/− 1.6 | | | 5.5 +/− 2.5 |
| EOS VISIT | | | | | | |
| LONG-TERM FU | 7.0 +/− 2.4 | 8.8 +/− 1.5 | 11.3 +/− 1.8 | 3.0 +/− 2.0 | 2.7 +/− 1.5 | 6.0 +/− 3.2 |

TABLE 5
Estimated relative abundance of strains of the 16-mix over time (Mean % ± SD)

| | ACTIVE | | | PLACEBO | | |
|---|---|---|---|---|---|---|
| VISIT | 1 | 2 | 3 | 1 | 2 | 3 |
| SCREENING | 0.5 +/− 0.07 | 0.07 +/− 0.16 | 0.04 +/− 0.05 | 0.04 +/− 0.06 | 0.2 +/− 0.15 | 0.06 +/− 0.06 |
| DAY −7 | 0.08 +/− 0.1 | 0.12 +/− 0.28 | 0.31 +/− 0.81 | 0.08 +/− 0.13 | 0.14 +/− 0.14 | 0.05 +/− 0.04 |
| DAY −1 | 0.06 +/− 0.13 | 0 +/− 0 | 0..01 +/− 0.03 | 0.01 +/− 0.02 | 0 +/− 0 | 0 +/− 0 |
| DAY 2 | 0.12 +/− 0.27 | 0.87 +/− 1.67 | 0.02 +/− 0.02 | 0 +/− 0 | 0 +/− 0 | 0.27 +/− 0.53 |
| DAY 4 | 18.09 +/− 24.36 | 13.31 +/− 6.29 | 5.82 +/− 6.04 | 0.16 +/− 0.22 | 0 +/− 0 | 0.38 +/− 0.49 |
| DAY 7 | | | 32.23 +/− 18.97 | | | 0.96 +/− 0.96 |
| DAY 14 | | | 15.48 +/− 12.45 | | | 1.50 +/− 1.65 |
| DAY 17 | | | 15.72 +/− 12.67 | | | 2.62 +/− 3.87 |
| FU WEEK 1 | 29.53 +/− 17.27 | 12.89 +/− 10.96 | | 0.15 +/− 0.26 | 1.78 +/− 1.76 | |
| FU WEEK 2 | 10.24 +/− 12.25 | 8.77 +/− 7.31 | | 0.06 +/− 0.10 | 0.37 +/− 0.26 | |
| FU WEEK 3 | | | 9.41 +/− 5.92 | | | 1.68 +/− 2.18 |
| FU WEEK 4 | [symbol] +/− 6.86 | 2.62 +/− 1.62 | 7.15 +/− 4.55 | 0.16 +/− 0.07 | 0.16 +/− 0.09 | 1.77 +/− 1.63 |
| FU WEEK 6 | | | 3.31 +/− 2.5 | | | 1.43 +/− 1.94 |
| FU WEEK 8 | 1.65 +/− 1.48 | 1.37 +/− 0.83 | | 0.36 +/− 0.50 | 0.14 +/− 0.05 | |
| FU WEEK 10 | | | 2.79 +/− 1.56 | | | 1.78 +/− 1.75 |
| FU WEEK 12/ | 0.93 +/− 0.54 | 1.10 +/− 0.69 | | 0.19 +/− 0.15 | 0.34 +/− 0.54 | |
| EOS VISIT | | | | | | |
| FU WEEK 14/ | | | 2.27 +/− 1.54 | | | 2.32 +/− 3.85 |
| EOS VISIT | | | | | | |
| LONG-TERM FU | [symbol] +/− 0.78 | 0.77 +/− 0.46 | 2.45 +/− 2.13 | 0.07 +/− 0.10 | 0.15 +/− 0.08 | 0.94 +/− 1.15 |

[symbol]

indicates data missing or illegible when filed

### Discussion

The results of the qPCR assay described herein detects and quantifies the strains of the 16-mix in a first in human trial of the 16-mix live bacterial product. This study is complementary to a parallel study of an 11-mix described in Example 1.

All samples processed in this study were done in a single batch, to minimize concerns related to batch variability. The microbial density was observed to consistently decrease by roughly ˜50% in all participants after vancomycin pretreatment. In this study, microbial density was observed to return to baseline after a week, a shorter timeframe than what was observed in Example 1.

The qPCR assay described herein was able to detect small, but significant, colonization in pre-dose samples which suggest the presence of genetically similar bacterial strains in the healthy human gut.

Interestingly, it was found that a single dose of the 16-mix was sufficient for long-term colonization of the vancomycin pre-treated healthy human gut. Strains were detected in samples up to the 6-month follow-up visit, providing evidence of long-term colonization.

The colonization kinetics (CK) of a single day of dosing following vancomycin pretreatment resulted in substantially increased detection and abundance of strains of the 16-mix. Single dose cohorts exhibited a dose-dependent relationship in the detection of strains, but not in relative abundance. This suggests that higher doses promote the long-term colonization at low abundance levels of a wider number of strains and limits the larger expansion of an individual strain.

Individual strain dynamics were determined by inter-individual variation and demonstrate the variability of the human microbiome and host:microbiome interactions.

As observed in Example 1, placebo-treated vancomycin pre-treated participants co-housed with active 16-mix treated subjects also demonstrated low level colonization with strains of the 16-mix, although at significantly lower RA levels. The strains that were colonized were subject-specific. Additionally, colonization of strains was observed in placebo-treated subjects. Occasionally, strains were only detected by qPCR weeks after the active dosing period, suggesting that strains can remain viable in the gastrointestinal tract for weeks before being reliably detected in stool by qPCR.

The qPCR assay described herein may be used as a rapid and cost-effective co-diagnostic method to assess the colonization of strains in subjects receiving live bacterial products.

In conclusion, the 16-mix described herein was determined to be safe and tolerable. A single dose of the 16-mix was sufficient for significant and durable colonization of the vancomycin pretreated healthy human gut.

### REFERENCES

1. Sartor. RB. Genetics and environmental interactions shape the intestinal microbiome to promote inflammatory bowel disease versus mucosal homeostasis. Gastroenterology (2010) 139(6):1816-1819.
2. Atarashi, K., Tanoue, T., Oshima, K. et al. Treg induction by a rationally selected mixture of Clostridia strains from the human microbiota. Nature (2013) 500, 232-236.

### Example 3: Clinical Study to Evaluate Use of the 16-Mix for Treating Mild-to-Moderate Ulcerative Colitis

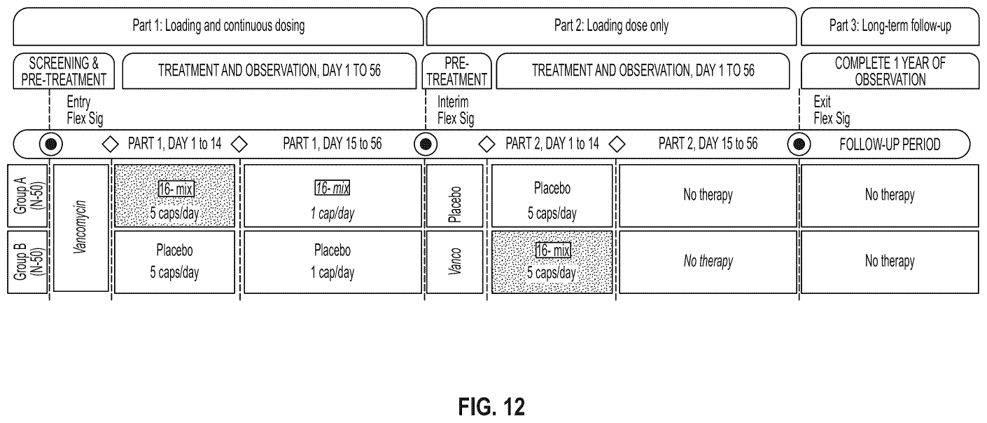

A clinical trial was developed to evaluating use of the 16-mix described herein for treating mild-to-moderate ulcerative colitis. As shown in FIG. 12, the study involves three parts: Part I, which includes a loading period and a continuous dosing (maintenance) period; Part 2, which includes a loading period only; and Part 3: long-term follow-up.

In Part 1 of the study, a first group of subjects (Group A) are pretreated with vancomycin, followed by multiple doses of the 16-mix (5 capsules per day, each capsule containing 1×10⁹ colony forming units (CFUs) (i.e., 5×10⁹ CFU total)) for a loading period of 14 days. From day 15 to day 56, subjects are administered multiple doses of the 16-mix (1 capsule per day containing 1×10⁹ colony forming units (CFUs)) for a maintenance period. In Part 2 of the study, subjects of Group A receive placebo during the pretreatment period, followed by administration of multiple doses of a placebo (5 capsules per day) for a 14 day loading period, followed by no therapy from days 15-56. In Part 1 of the study, a second group of subjects (Group B) are pretreated with vancomycin, followed by multiple doses of a placebo (5 capsules per day) for a loading period of 14 days. From day 15 to day 56, subjects are administered multiple doses of a placebo for a maintenance period. In Part 2 of the study, subjects of Group B are pretreated with vancomycin, followed by multiple doses of the 16-mix (5 capsules per day, each capsule containing 1×10⁹ colony forming units (CFUs) (i.e., 5×10⁹ CFU total)) for a 14 day loading period, followed by no therapy from days 15-56. Both groups are subjected to long-term follow-up over the course of a year.

Key inclusion criteria for subjects include being biologic-naïve and having stable doses of all medications the subject may be taking. Key exclusion criteria for subjects include limited rectal disease, primary sclerosing cholangitis, active dysplasia, or adenomatous polyps.

Subjects are evaluated based on the primary end point of endoscopic response at week 8. Subjects will further be followed to week 52 to evaluate safety, disease activity, and pharmacokinetics/pharmacodynamics of the 16-mix dosage regimen.

### Example 4: Analysis of Metabolites in the Guts of Subjects Administered with 11-Strain or 16-Strain Bacterial Mixtures

In the studies described in Examples 1 and 2, the contents of various metabolites were analyzed in the stools of participants. Raw stool samples were collected from participants receiving 5 days of vancomycin and 14 days of 1×10¹⁰ CFU per day of the 11-mix or placebo (cohort 6) on Days −7, Day −1, Day 1, Day 2, Day 4, Day 7, Day 10, Day 14, Day 27, and Day 92. Raw stool samples were collected from participants receiving 5 days of vancomycin and 14 days of 1×10⁹ CFU per day of the 16-mix or placebo (cohort 3) on Day −1, Day 2, Day 4, Day 7, Day 14, and Day 92. Samples were frozen fresh and stored at −75+/−10° C. prior to physical homogenization in liquid nitrogen and quantification.

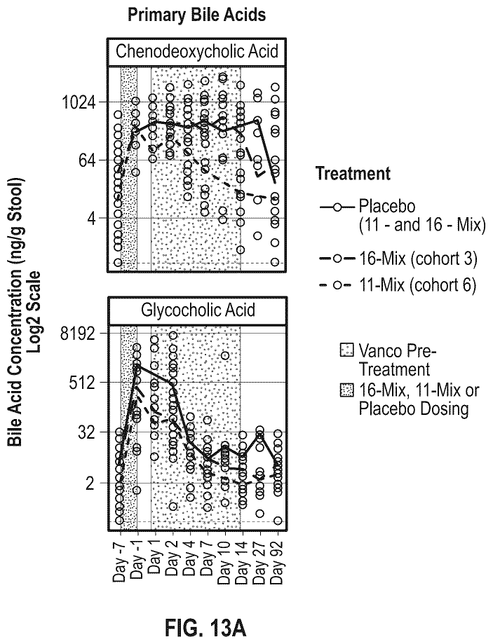

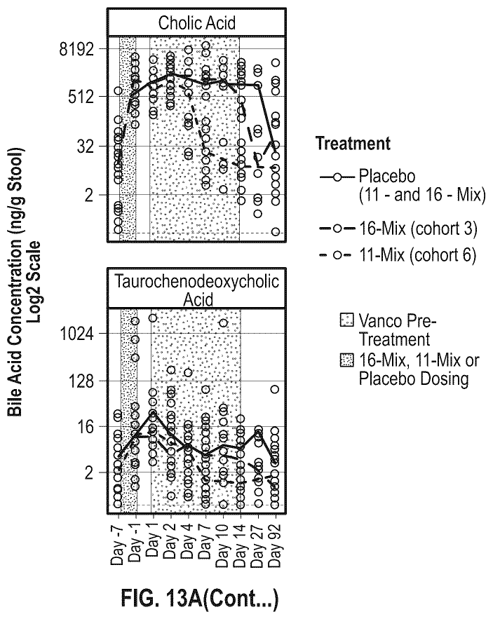

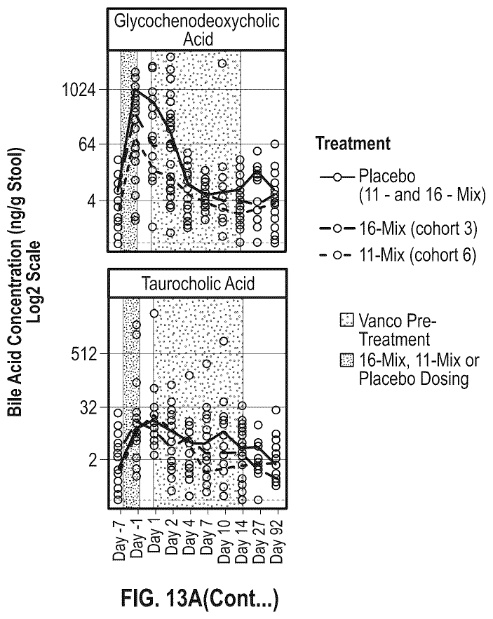

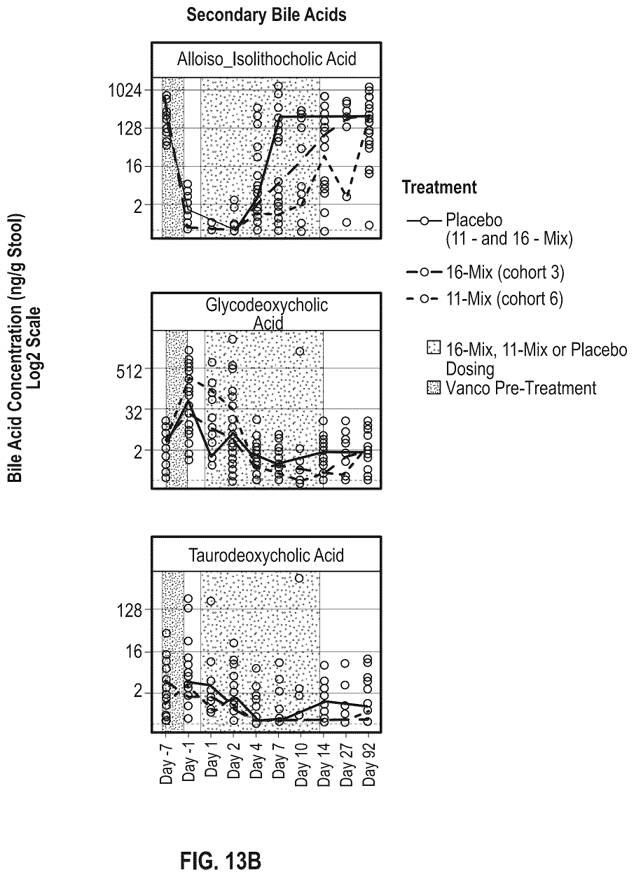

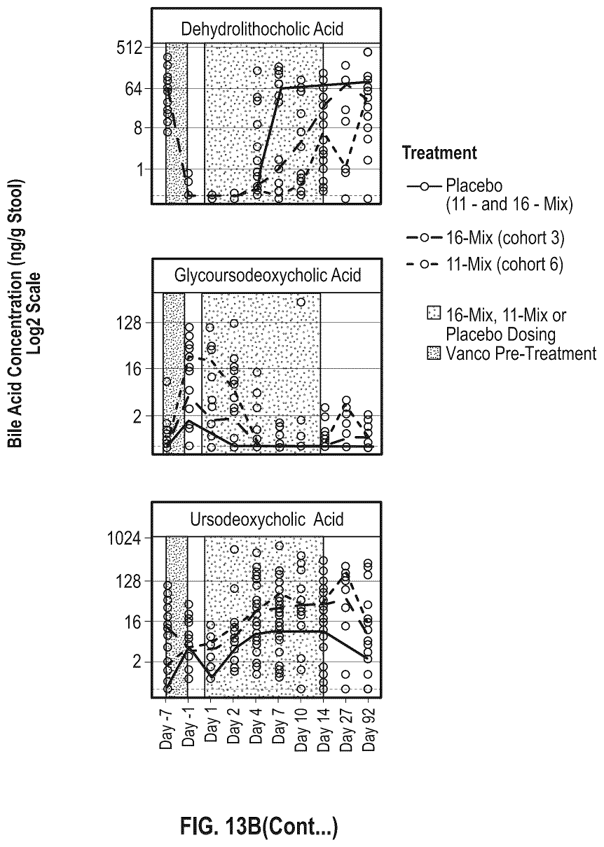

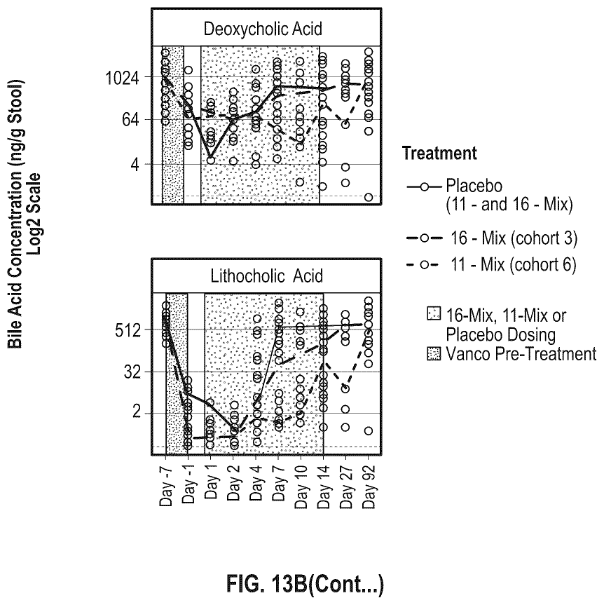

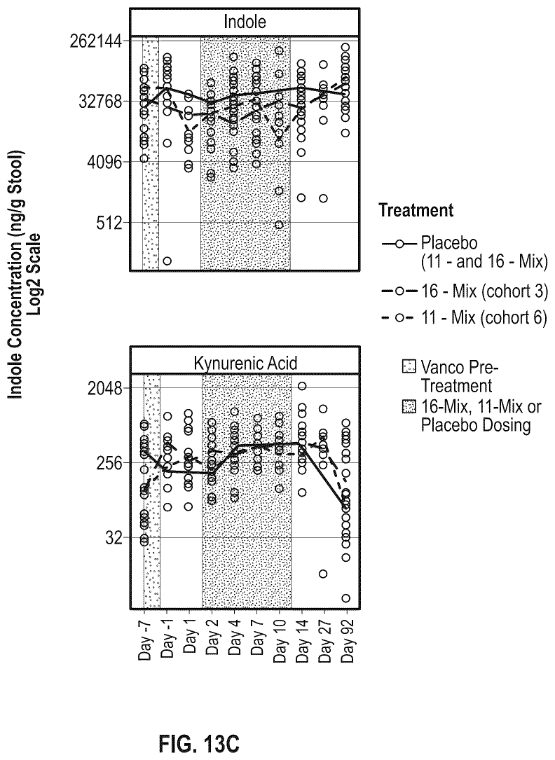

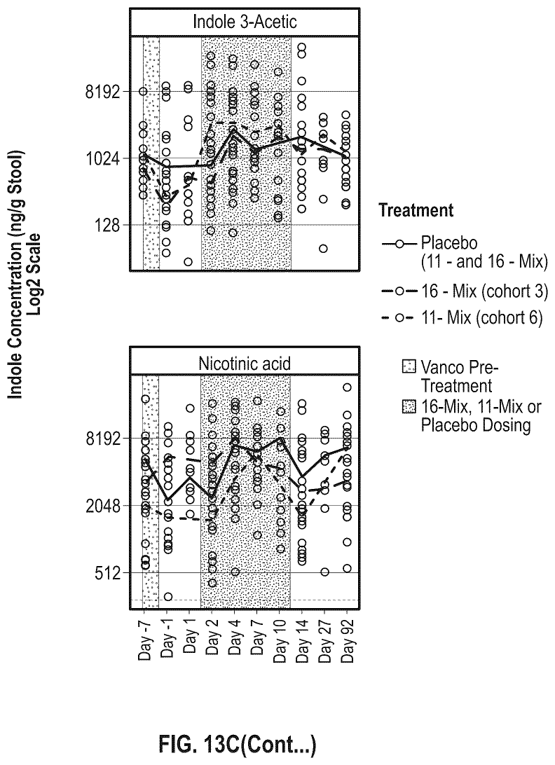

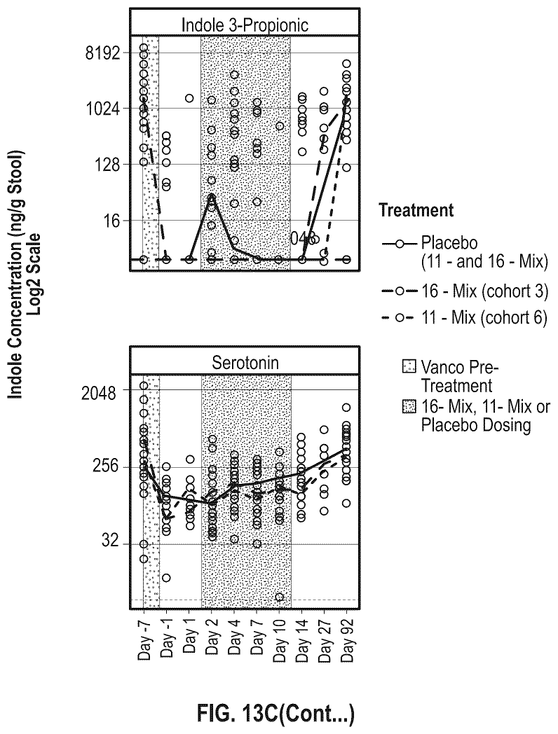

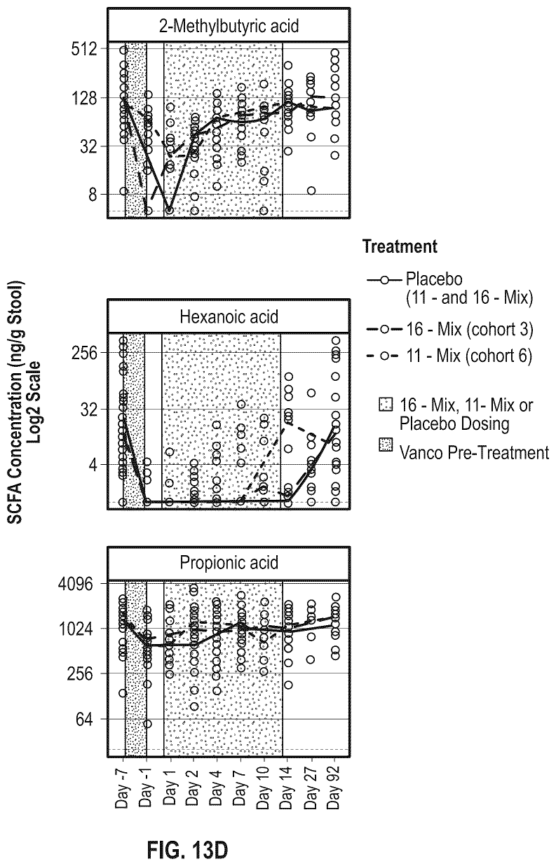

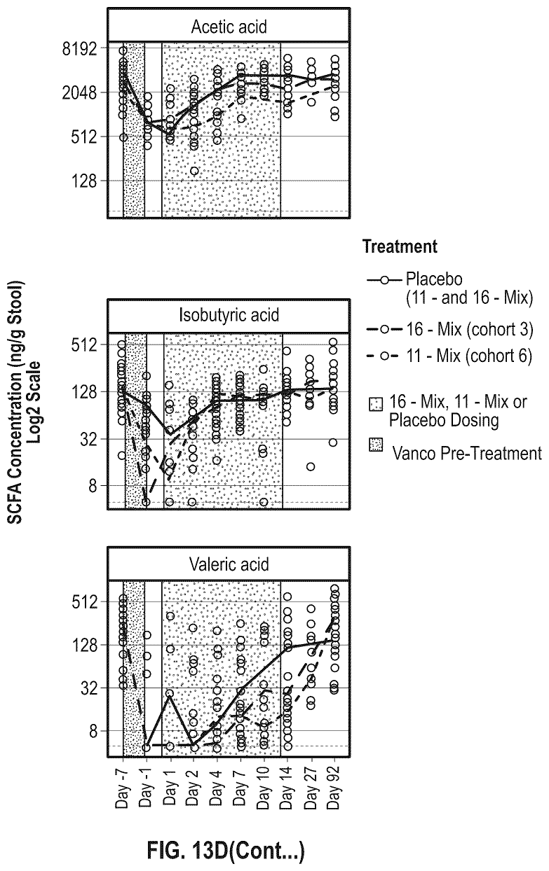

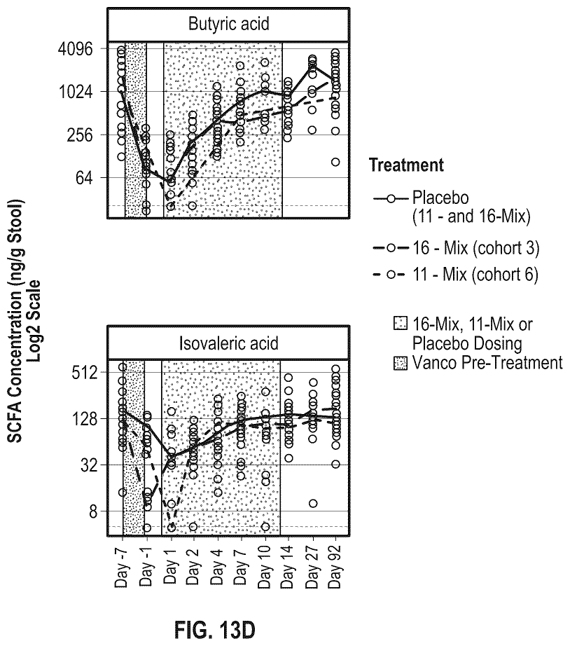

Primary and secondary bile acids were quantified using liquid chromatography with tandem mass spectrometry (LC-MS-MS) (FIGS. 13A-13B). The panel of bile acid panel quantified included of allo-iso isolithocholic acid, chenodeoxycholic acid, cholic acid, dehydrolithocholic acid, deoxycholic acid, isodeoxycholic acid, lithocholic acid, and ursodeoxycholic acid. Additionally, the glycine and taurine conjugated forms were also quantified including glycochenodeoxycholic acid, glycocholic acid, glycodeoxycholic acid, glycolithocholic acid, glycoursodeoxycholic acid, taurochenodeoxycholic acid, taurocholic acid, taurodeoxycholic acid, taurolithocholic acid, and tauroursodeoxycholic acid.

Short Chain Fatty Acids (SCFAs) were quantified using liquid chromatography with tandem mass spectrometry (LC-MS-MS) (FIG. 13C). Targeted SCFAs included 2-methylbutyric acid, acetic acid, butyric acid, hexanoic acid, isobutyric acid, isovaleric acid, propionic acid, succinic acid, and valeric acid.

Indoles were quantified with two methods:hydrophilic interaction liquid chromatography (HILIC) and reverse phase chromatography (FIG. 13D). Indoles quantified by these methods included kynurenic acid, serotonin, nicotinic acid, indole, indole 3-acetic, and indole 3-propionic acid.

Data from placebo subject in the 11- and 16-mix studies were pooled for data analysis. The 11-mix and the 16-mix promoted recovery of bile acids to pre-vancomycin concentrations during the 14-day treatment window. Specifically, by Day 7 the subjects receiving the 16-mix had significantly lower cholic acid, a primary bile acid, than the pooled placebo subjects (FIG. 13A). Additionally, several secondary bile acids with anti-inflammatory and antibacterial properties were found to be at significantly higher concentrations in the subjects treated with the 16-mix than pooled placebo subjects on Day 7 (FIG. 13B). These results indicate that both the 11-strain mixture of Example 1 and the 16-strain mixture of Example 2 promoted the reduction of primary bile acid concentrations and increase of secondary bile acid concentrations in treated subjects.

1. A method for colonizing a microbiome in a subject, the method comprising administering to the subject a therapeutically effective amount of a pharmaceutical composition comprising a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

2. The method of claim 1, wherein the purified bacterial mixture comprises at least three, four, five, six, seven, eight, nine, ten, eleven, twelve, thirteen, fourteen, fifteen, or sixteen, bacterial strains.

3. The method of claim 1 or 2, wherein the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

4. The method of claim 3, wherein the subject has, is suspected of having, or is at risk of having inflammatory bowel disorder (IBD).

5. The method of claim 1 or 2, wherein the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

6. The method of claim 5, wherein the subject has, is suspected of having, or is at risk of having graft versus host disease (GvHD).

7. A method for colonizing a microbiome in a subject, the method comprising administering to the subject a therapeutically effective of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16.

8. The method of claim 7, wherein the purified bacterial mixture comprises at least three, four, five, six, seven, eight, nine, ten, eleven, twelve, thirteen, fourteen, fifteen, or sixteen bacterial strains.

9. The method of claim 7 or 8, wherein the pharmaceutical composition comprises a purified bacterial mixture consisting of bacterial strains that comprise 16S rDNA sequences of at least 97% sequence identity to the nucleic acid sequences set forth as SEQ ID NOs:1-16.

10. The method of claim 9, wherein the subject has, is suspected of having, or is at risk of having inflammatory bowel disorder (IBD).

11. The method of claim 10, wherein the IBD is ulcerative colitis or Crohn's disease.

12. The method of claim 10 or 11, wherein the subject has one or more risk factors associated with IBD.

13. The method of claim 7 or 8, wherein the pharmaceutical composition comprises a purified bacterial mixture consisting of bacterial strains that comprise 16S rDNA sequences of at least 97% sequence identity to the nucleic acid sequences set forth as SEQ ID NOs:3, 5-10, 12, and 14-16.

14. The method of claim 13, wherein the subject has, is suspected of having, or is at risk of having graft versus host disease (GvHD).

15. The method of any one of claims 1-14, wherein each of the bacterial strains of the pharmaceutical composition colonizes the microbiome.

16. The method of any one of claims 1-15, wherein the bacterial strains of the pharmaceutical composition colonize the microbiome over an extended period of time.

17. The method of any one of claims 1-16, wherein the pharmaceutical composition reduces an amount of one or more primary bile acids in the subject.

18. The method of claim 17, wherein the primary bile acid is chenodeoxycholic acid, cholic acid, glycochenodeoxycholic acid, glycocholic acid, taurochenodeoxycholic acid, or taurocholic acid.

19. The method of any one of claims 1-18, wherein the pharmaceutical composition reduces levels of primary bile acids in the subject by 2-fold to 100,000-fold.

20. The method of any one of claims 1-19, wherein the pharmaceutical composition increases an amount of one or more secondary bile acids in the subject.

21. The method of claim 20, wherein the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid.

22. The method of any one of claims 1-21, wherein the pharmaceutical composition increases levels of secondary bile acids in the subject by 2-fold to 100,000-fold.

23. The method of any one of claims 1-22, wherein the pharmaceutical composition increases an amount of one or more short-chain fatty acids (SCFAs) in the subject.

24. The method of claim 23, wherein the SCFA is 2-methylbutyric acid, acetic acid, butyric acid, hexanoic acid, isobutyric acid, isovaleric acid, propionic acid, or valeric acid.

25. The method of any one of claims 1-24, wherein the pharmaceutical composition increases levels of short-chain fatty acids (SCFAs) in the subject by 2-fold to 100,000-fold.

26. The method of any one of claims 1-25, wherein the pharmaceutical composition increases an amount of one or more indoles in the subject.

27. The method of claim 26, wherein the indole is kynurenic acid, serotonin, nicotinic acid, indole, indole 3-acetic, or indole 3-propionic acid.

28. The method of any one of claims 1-27, wherein the pharmaceutical composition increases levels of indoles in the subject by 2-fold to 100,000-fold.

29. The method of any one of claims 1-28, further comprising administering to the subject an antibiotic.

30. The method of any one of claims 1-29, wherein the subject was administered an antibiotic prior to administration of the pharmaceutical composition.

31. The method of any one of claims 1-28, wherein administration of the pharmaceutical composition is not preceded by administration of an antibiotic.

32. The method of any one of claims 29-31, wherein the antibiotic is vancomycin, fidaxomycin, or ridinilazole.

33. The method of claim 32, wherein the antibiotic is vancomycin.

34. The method of claim 33, wherein the vancomycin is administered at a dose sufficient to allow for colonization of one or more of the bacterial strains of the pharmaceutical composition.

35. The method of claim 33 or 34, wherein the vancomycin is administered in 4 doses of 125 mg per day.

36. The method of any one of claims 33-35, wherein the vancomycin is administered for five consecutive days.

37. The method of claim 35 or 36, wherein the vancomycin is administered on five consecutive days immediately prior to the day of the administration of the pharmaceutical composition.

38. The method of claim 35 or 36, wherein the vancomycin is administered on five consecutive days up to two days prior to the day of the administration of the pharmaceutical composition, and wherein the method includes a washout day one day prior to the day of the administration of the pharmaceutical composition.

39. The method of any one of claims 1-38, wherein the pharmaceutical composition is administered as a single dose.

40. The method of any one of claims 1-38, wherein the pharmaceutical composition is administered in multiple doses.

41. The method of any one of claims 1-40, wherein a dose of the pharmaceutical composition comprises between 10⁸ to 10¹¹ total Colony Forming Units (CFUs).

42. The method of any one of claims 1-41, wherein a dose of the pharmaceutical composition comprises about 10⁹ total CFUs.

43. The method of any one of claims 1-42, wherein a dose of the pharmaceutical composition comprises about 10¹⁰ total CFUs.

44. The method of any one of claims 39-43, wherein each dose comprises the administration of multiple capsules.

45. The method of claim 44, wherein each capsule comprises about 10⁹ total CFUs.

46. The method of claim 44 or 45, wherein each dose comprises administration of 10 capsules each comprising about 10⁹ total CFUs.

47. The method of any one of claims 40-46, wherein the multiple doses are administered on consecutive days.

48. The method of any one of claims 40-47, wherein the multiple doses are administered on 7-14 consecutive days.

49. The method of any one of claims 1-48, wherein the method further comprises administering a therapeutic agent.

50. The method of claim 49, wherein the therapeutic agent is a therapeutic agent for treating IBD or GvHD.

51. The method of any one of claims 1-50, wherein the two or more bacterial strains are lyophilized.

52. The method of any one of claims 1-51, wherein the two or more bacterial strains are spray-dried.

53. The method of any one of claims 1-52, wherein one or more of the two or more bacterial strains are in spore form.

54. The method of any one of claims 1-53, wherein each of the two or more bacterial strains are in spore form.

55. The method of any one of claims 1-53, wherein one or more of the two or more bacterial strains are in vegetative form.

56. The method of any one of claim 1-52 or 55, wherein each of the two or more bacterial strains are in vegetative form.

57. The method of any one of claims 1-56, wherein the pharmaceutical composition further comprises one or more enteric polymers.

58. The method of any one of claims 1-57, wherein the administration is oral administration.

59. The method of any one of claims 1-58, wherein the pharmaceutical composition is formulated for oral delivery.

60. The method of any one of claims 1-59, wherein the pharmaceutical composition is formulated for rectal delivery.

61. The method of any one of claims 1-60, wherein the pharmaceutical composition is formulated for delivery to the intestine.

62. The method of any one of claims 1-61, wherein the pharmaceutical composition is formulated for delivery to the colon.

63. A method for treating and/or preventing inflammatory bowel disease (IBD) in a subject, the method comprising administering to the subject a therapeutically effective amount of a pharmaceutical composition comprising a purified bacterial mixture comprising two or more bacterial strains of species elected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

64. The method of claim 63, wherein the purified bacterial mixture comprises at least three, four, five, six, seven, eight, nine, ten, eleven, twelve, thirteen, fourteen, fifteen, or sixteen, bacterial strains.

65. The method of claim 63 or 64, wherein the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

66. The method of claim 63 or 64, wherein the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

67. A method for treating and/or preventing inflammatory bowel disease (IBD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16.

68. The method of claim 67, wherein the purified bacterial mixture comprises at least three, four, five, six, seven, eight, nine, ten, eleven, twelve, thirteen, fourteen, fifteen, or sixteen bacterial strains.

69. The method of claim 67 or 68, wherein the pharmaceutical composition comprises a purified bacterial mixture consisting of bacterial strains that comprise 16S rDNA sequences of at least 97% sequence identity to the nucleic acid sequences set forth as SEQ ID NOs: 1-16.

70. The method of claim 67 or 68, wherein the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

71. The method of any one of claims 63-70, wherein the subject is at risk of developing IBD.

72. The method of claim 71, wherein the IBD is ulcerative colitis or Crohn's disease.

73. The method of any one of claims 63-72, wherein the pharmaceutical composition is administered after a first therapeutic agent for treating IBD.

74. The method of claim 73, wherein the first therapeutic for treating IBD is an antibiotic or aminosalicylate (5-ASA) agent.

75. The method of any one of claims 63-74, wherein the subject has one or more risk factors associated with IBD.

76. The method of any one of claims 63-75, wherein each of the bacterial strains of the pharmaceutical composition colonizes the microbiome.

77. The method of any one of claims 63-76, wherein the bacterial strains of the pharmaceutical composition colonize the microbiome over an extended period of time.

78. The method of any one of claims 63-77, wherein the pharmaceutical composition reduces an amount of one or more primary bile acids in the subject.

79. The method of claim 78, wherein the primary bile acid is chenodeoxycholic acid, cholic acid, glycochenodeoxycholic acid, glycocholic acid, taurochenodeoxycholic acid, or taurocholic acid.

80. The method of any one of claims 63-79, wherein the pharmaceutical composition reduces levels of primary bile acids in the subject by 2-fold to 100,000-fold.

81. The method of any one of claims 63-80, wherein the pharmaceutical composition increases an amount of one or more secondary bile acids in the subject.

82. The method of claim 81, wherein the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid.

83. The method of any one of claims 63-82, wherein the pharmaceutical composition increases levels of secondary bile acids in the subject by 2-fold to 100,000-fold.

84. The method of any one of claims 63-83, wherein the pharmaceutical composition increases an amount of one or more short-chain fatty acids (SCFAs) in the subject.

85. The method of claim 84, wherein the SCFA is 2-methylbutyric acid, acetic acid, butyric acid, hexanoic acid, isobutyric acid, isovaleric acid, propionic acid, or valeric acid.

86. The method of any one of claims 63-85, wherein the pharmaceutical composition increases levels of short-chain fatty acids (SCFAs) in the subject by 2-fold to 100,000-fold.

87. The method of any one of claims 63-86, wherein the pharmaceutical composition increases an amount of one or more indoles in the subject.

88. The method of claim 87, wherein the indole is kynurenic acid, serotonin, nicotinic acid, indole, indole 3-acetic, or indole 3-propionic acid.

89. The method of any one of claims 63-88, wherein the pharmaceutical composition increases levels of indoles in the subject by 2-fold to 100,000-fold.

90. The method of any one of claims 63-89, further comprising administering to the subject an antibiotic.

91. The method of any one of claims 63-90, wherein the subject was administered an antibiotic prior to administration of the pharmaceutical composition.

92. The method of any one of claims 63-90, wherein administration of the pharmaceutical composition is not preceded by administration of an antibiotic.

93. The method of any one of claims 90-92, wherein the antibiotic is vancomycin, fidaxomycin, or ridinilazole.

94. The method of claim 93, wherein the antibiotic is vancomycin.

95. The method of claim 94, wherein the vancomycin is administered at a dose sufficient to allow for colonization of one or more of the bacterial strains of the pharmaceutical composition.

96. The method of claim 94 or 95, wherein the vancomycin is administered in 4 doses of 125 mg per day.

97. The method of any one of claims 93-96, wherein the vancomycin is administered for five consecutive days.

98. The method of any one of claims 93-97, wherein the vancomycin is administered on five consecutive days immediately prior to the day of the administration of the pharmaceutical composition.

99. The method of any one of claims 93-97, wherein the vancomycin is administered on the same day as administration of a first dose of a pharmaceutical composition.

100. The method of any one of claims 93-97, wherein the vancomycin is administered on five consecutive days up to two days prior to the day of the administration of the pharmaceutical composition, and wherein the method includes a washout day one day prior to the day of the administration of the pharmaceutical composition.

101. The method of any one of claims 63-100, wherein the pharmaceutical composition is administered as a single dose.

102. The method of any one of claims 63-100, wherein the pharmaceutical composition is administered in multiple doses.

103. The method of any one of claims 63-102, wherein a dose of the pharmaceutical composition comprises between 10⁸ to 10¹¹ total colony forming units (CFUs).

104. The method of any one of claims 63-103, wherein a dose of the pharmaceutical composition comprises about 10⁹ total CFUs.

105. The method of any one of claims 63-103, wherein a dose of the pharmaceutical composition comprises about 10¹⁰ total CFUs.

106. The method of any one of claims 101-105, wherein each dose comprises the administration of multiple capsules.

107. The method of claim 106, wherein each capsule comprises about 10⁹ total CFUs.

108. The method of claim 106 or 107, wherein each dose comprises administration of 10 capsules each comprising about 10⁹ total CFUs.

109. The method of any one of claims 102-108, wherein the multiple doses are administered on consecutive days.

110. The method of any one of claims 102-109, wherein the multiple doses are administered on 7-14 consecutive days.

111. The method of any one of claims 63-110, wherein the method further comprises administering a therapeutic agent.

112. The method of claim 11, wherein the therapeutic agent is a therapeutic agent for treating IBD.

113. The method of any one of claims 63-112, wherein the two or more bacterial strains are lyophilized.

114. The method of any one of claims 63-112, wherein the two or more bacterial strains are spray-dried.

115. The method of any one of claims 63-114, wherein one or more of the two or more bacterial strains are in spore form.

116. The method of any one of claims 63-115, wherein each of the two or more bacterial strains are in spore form.

117. The method of any one of claims 63-115, wherein one or more of the two or more bacterial strains are in vegetative form.

118. The method of any one of claims 63-114 and 117, wherein each of the two or more bacterial strains are in vegetative form.

119. The method of any one of claims 63-118, wherein the pharmaceutical composition further comprises one or more enteric polymers.

120. The method of any one of claims 63-119, wherein the administration is oral administration.

121. The method of any one of claims 63-120, wherein the pharmaceutical composition is formulated for oral delivery.

122. The method of any one of claims 63-120, wherein the pharmaceutical composition is formulated for rectal delivery.

123. The method of any one of claims 63-122, wherein the pharmaceutical composition is formulated for delivery to the intestine.

124. The method of any one of claims 63-123, wherein the pharmaceutical composition is formulated for delivery to the colon.

125. A method for reducing risk and/or occurrence of inflammatory bowel disease (IBD) in a subject, the method comprising administering to the subject a therapeutically effective amount of a pharmaceutical composition comprising a purified bacterial mixture comprising two or more bacterial strains of species elected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

126. The method of claim 125, wherein the purified bacterial mixture comprises at least three, four, five, six, seven, eight, nine, ten, eleven, twelve, thirteen, fourteen, fifteen, or sixteen, bacterial strains.

127. The method of claim 125 or 126, wherein the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

128. The method of claim 125 or 126, wherein the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

129. A method for reducing risk and/or occurrence of inflammatory bowel disease (IBD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16.

130. The method of claim 129, wherein the purified bacterial mixture comprises at least three, four, five, six, seven, eight, nine, ten, eleven, twelve, thirteen, fourteen, fifteen, or sixteen bacterial strains.

131. The method of claim 129 or 130, wherein the pharmaceutical composition comprises a purified bacterial mixture consisting of bacterial strains that comprise 16S rDNA sequences of at least 97% sequence identity to the nucleic acid sequences set forth as SEQ ID NOs: 1-16.

132. The method of claim 129 or 130, wherein the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

133. The method of any one of claims 125-132, wherein the IBD is ulcerative colitis or Crohn's disease.

134. The method of any one of claims 125-133, wherein the pharmaceutical composition is administered after a first therapeutic agent for treating IBD.

135. The method of claim 134, wherein the first therapeutic for treating IBD is an antibiotic or aminosalicylate (5-ASA) agent.

136. The method of any one of claims 125-135, wherein the subject has one or more risk factors associated with IBD.

137. The method of any one of claims 125-136, wherein each of the bacterial strains of the pharmaceutical composition colonizes the microbiome.

138. The method of any one of claims 125-137, wherein the bacterial strains of the pharmaceutical composition colonize the microbiome over an extended period of time.

139. The method of any one of claims 125-138, wherein the pharmaceutical composition reduces an amount of one or more primary bile acids in the subject.

140. The method of claim 139, wherein the primary bile acid is chenodeoxycholic acid, cholic acid, glycochenodeoxycholic acid, glycocholic acid, taurochenodeoxycholic acid, or taurocholic acid.

141. The method of any one of claims 125-140, wherein the pharmaceutical composition reduces levels of primary bile acids in the subject by 2-fold to 100,000-fold.

142. The method of any one of claims 125-141, wherein the pharmaceutical composition increases an amount of one or more secondary bile acids in the subject.

143. The method of claim 142, wherein the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid.

144. The method of any one of claims 125-143, wherein the pharmaceutical composition increases levels of secondary bile acids in the subject by 2-fold to 100,000-fold.

145. The method of any one of claims 125-144, wherein the pharmaceutical composition increases an amount of one or more short-chain fatty acids (SCFAs) in the subject.

146. The method of claim 145, wherein the SCFA is 2-methylbutyric acid, acetic acid, butyric acid, hexanoic acid, isobutyric acid, isovaleric acid, propionic acid, or valeric acid.

147. The method of any one of claims 125-146, wherein the pharmaceutical composition increases levels of short-chain fatty acids (SCFAs) in the subject by 2-fold to 100,000-fold.

148. The method of any one of claims 125-147, wherein the pharmaceutical composition increases an amount of one or more indoles in the subject.

149. The method of claim 148, wherein the indole is kynurenic acid, serotonin, nicotinic acid, indole, indole 3-acetic, or indole 3-propionic acid.

150. The method of any one of claims 125-149, wherein the pharmaceutical composition increases levels of indoles in the subject by 2-fold to 100,000-fold.

151. The method of any one of claims 125-150, further comprising administering to the subject an antibiotic.

152. The method of any one of claims 125-151, wherein the subject was administered an antibiotic prior to administration of the pharmaceutical composition.

153. The method of any one of claims 125-151, wherein administration of the pharmaceutical composition is not preceded by administration of an antibiotic.

154. The method of any one of claims 151-153, wherein the antibiotic is vancomycin, fidaxomycin, or ridinilazole.

155. The method of claim 154, wherein the antibiotic is vancomycin.

156. The method of claim 155, wherein the vancomycin is administered at a dose sufficient to allow for colonization of one or more of the bacterial strains of the pharmaceutical composition.

157. The method of claim 155 or 156, wherein the vancomycin is administered in 4 doses of 125 mg per day.

158. The method of any one of claims 155-157, wherein the vancomycin is administered for five consecutive days.

159. The method of any one of claims 153-158, wherein the vancomycin is administered on five consecutive days immediately prior to the day of the administration of the pharmaceutical composition.

160. The method of any one of claims 153-158, wherein the vancomycin is administered on the same day as administration of a first dose of a pharmaceutical composition.

161. The method of any one of claims 153-158, wherein the vancomycin is administered on five consecutive days up to two days prior to the day of the administration of the pharmaceutical composition, and wherein the method includes a washout day one day prior to the day of the administration of the pharmaceutical composition.

162. The method of any one of claims 125-161, wherein the pharmaceutical composition is administered as a single dose.

163. The method of any one of claims 125-161, wherein the pharmaceutical composition is administered in multiple doses.

164. The method of any one of claims 125-161, wherein a dose of the pharmaceutical composition comprises between 10⁸ to 10¹¹ total colony forming units (CFUs).

165. The method of any one of claims 162-164, wherein a dose of the pharmaceutical composition comprises about 10⁹ total CFUs.

166. The method of any one of claims 162-164, wherein a dose of the pharmaceutical composition comprises about 10¹⁰ total CFUs.

167. The method of any one of claims 162-166, wherein each dose comprises the administration of multiple capsules.

168. The method of claim 167, wherein each capsule comprises about 10⁹ total CFUs.

169. The method of claim 167 or 168, wherein each dose comprises administration of 10 capsules each comprising about 10⁹ total CFUs.

170. The method of any one of claims 163-169, wherein the multiple doses are administered on consecutive days.

171. The method of any one of claims 163-170, wherein the multiple doses are administered on 7-14 consecutive days.

172. The method of any one of claims 125-171, wherein the method further comprises administering a therapeutic agent.

173. The method of claim 171, wherein the therapeutic agent is a therapeutic agent for treating IBD.

174. The method of any one of claims 125-173, wherein the two or more bacterial strains are lyophilized.

175. The method of any one of claims 125-173, wherein the two or more bacterial strains are spray-dried.

176. The method of any one of claims 125-175, wherein one or more of the two or more bacterial strains are in spore form.

177. The method of any one of claims 125-176, wherein each of the two or more bacterial strains are in spore form.

178. The method of any one of claims 125-176, wherein one or more of the two or more bacterial strains are in vegetative form.

179. The method of any one of claims 125-177 and 178, wherein each of the two or more bacterial strains are in vegetative form.

180. The method of any one of claims 125-179, wherein the pharmaceutical composition further comprises one or more enteric polymers.

181. The method of any one of claims 125-180, wherein the administration is oral administration.

182. The method of any one of claims 125-181, wherein the pharmaceutical composition is formulated for oral delivery.

183. The method of any one of claims 125-182, wherein the pharmaceutical composition is formulated for rectal delivery.

184. The method of any one of claims 125-183, wherein the pharmaceutical composition is formulated for delivery to the intestine.

185. The method of any one of claims 125-184, wherein the pharmaceutical composition is formulated for delivery to the colon.

186. A method for treating and/or preventing graft versus host disease (GvHD) in a subject, the method comprising administering to the subject a therapeutically effective amount of a pharmaceutical composition comprising a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

187. The method of claim 186, wherein the purified bacterial mixture comprises at least three, four, five, six, seven, eight, nine, ten, eleven, twelve, thirteen, fourteen, fifteen, or sixteen, bacterial strains.

188. The method of claim 186 or 187, wherein the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

189. The method of claim 186 or 187, wherein the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

190. A method for treating and/or preventing graft versus host disease (GvHD) in a subject, the method comprising administering to the subject a therapeutically effective amount of a pharmaceutical composition comprising a purified bacterial mixture comprising two or more bacterial strains that comprise 16S rDNA sequences of at least 97% sequence identity to the nucleic acid sequences set forth as SEQ ID NOs: 1-16.

191. The method of claim 190, wherein the purified bacterial mixture comprises at least three, four, five, six, seven, eight, nine, ten, eleven, twelve, thirteen, fourteen, fifteen, or sixteen bacterial strains.

192. The method of claim 190 or 191, wherein the pharmaceutical composition comprises a purified bacterial mixture consisting of bacterial strains that comprise 16S rDNA sequences of at least 97% sequence identity to the nucleic acid sequences set forth as SEQ ID NOs: 1-16.

193. The method of claim 190 or 191, wherein the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

194. The method of any one of claims 186-193, wherein the subject is at risk of developing GvHD.

195. The method of any one of claims 186-194, wherein the pharmaceutical composition is administered after a first therapeutic agent for treating GvHD.

196. The method of any one of claims 186-195, wherein each of the bacterial strains of the pharmaceutical composition colonizes the microbiome.

197. The method of any one of claims 186-196, wherein the bacterial strains of the pharmaceutical composition colonize the microbiome over an extended period of time.

198. The method of any one of claims 186-197, wherein the pharmaceutical composition reduces an amount of one or more primary bile acids in the subject.

199. The method of claim 198, wherein the primary bile acid is chenodeoxycholic acid, cholic acid, glycochenodeoxycholic acid, glycocholic acid, taurochenodeoxycholic acid, or taurocholic acid.

200. The method of any one of claims 186-199, wherein the pharmaceutical composition reduces levels of primary bile acids in the subject by 2-fold to 100,000-fold.

201. The method of any one of claims 186-200, wherein the pharmaceutical composition increases an amount of one or more secondary bile acids in the subject.

202. The method of claim 201, wherein the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid.

203. The method of any one of claims 186-202, wherein the pharmaceutical composition increases levels of secondary bile acids in the subject by 2-fold to 100,000-fold.

204. The method of any one of claims 186-203, wherein the pharmaceutical composition increases an amount of one or more short-chain fatty acids (SCFAs) in the subject.

205. The method of claim 204, wherein the SCFA is 2-methylbutyric acid, acetic acid, butyric acid, hexanoic acid, isobutyric acid, isovaleric acid, propionic acid, or valeric acid.

206. The method of any one of claims 186-205, wherein the pharmaceutical composition increases levels of short-chain fatty acids (SCFAs) in the subject by 2-fold to 100,000-fold.

207. The method of any one of claims 186-206, wherein the pharmaceutical composition increases an amount of one or more indoles in the subject.

208. The method of claim 207, wherein the indole is kynurenic acid, serotonin, nicotinic acid, indole, indole 3-acetic, or indole 3-propionic acid.

209. The method of any one of claims 186-208, wherein the pharmaceutical composition increases levels of indoles in the subject by 2-fold to 100,000-fold.

210. The method of any one of claims 186-209, further comprising administering to the subject an antibiotic.

211. The method of any one of claims 186-210, wherein the subject was administered an antibiotic prior to administration of the pharmaceutical composition.

212. The method of any one of claims 186-210, wherein administration of the pharmaceutical composition is not preceded by administration of an antibiotic.

213. The method of any one of claims 210-212, wherein the antibiotic is vancomycin, fidaxomycin, or ridinilazole.

214. The method of claim 213, wherein the antibiotic is vancomycin.

215. The method of claim 214, wherein the vancomycin is administered at a dose sufficient to allow for colonization of one or more of the bacterial strains of the pharmaceutical composition.

216. The method of claim 214 or 215, wherein the vancomycin is administered in 4 doses of 125 mg per day.

217. The method of any one of claims 214-216, wherein the vancomycin is administered for five consecutive days.

218. The method of any one of claims 214-217, wherein the vancomycin is administered on five consecutive days immediately prior to the day of the administration of the pharmaceutical composition.

219. The method of any one of claims 214-218, wherein the vancomycin is administered on the same day as administration of a first dose of a pharmaceutical composition.

220. The method of any one of claims 214-218, wherein the vancomycin is administered on five consecutive days up to two days prior to the day of the administration of the pharmaceutical composition, and wherein the method includes a washout day one day prior to the day of the administration of the pharmaceutical composition.

221. The method of any one of claims 186-220, wherein the pharmaceutical composition is administered as a single dose.

222. The method of any one of claims 186-220, wherein the pharmaceutical composition is administered in multiple doses.

223. The method of any one of claims 186-2223, wherein a dose of the pharmaceutical composition comprises between 10⁸ to 10¹¹ total colony forming units (CFUs).

224. The method of any one of claims 186-223, wherein a dose of the pharmaceutical composition comprises about 10⁹ total CFUs.

225. The method of any one of claims 186-224, wherein a dose of the pharmaceutical composition comprises about 10¹⁰ total CFUs.

226. The method of any one of claims 221-225, wherein each dose comprises the administration of multiple capsules.

227. The method of claim 226, wherein each capsule comprises about 10⁹ total CFUs.

228. The method of claim 226 or 227, wherein each dose comprises administration of 10 capsules each comprising about 10⁹ total CFUs.

229. The method of any one of claims 222-228, wherein the multiple doses are administered on consecutive days.

230. The method of any one of claims 222-229, wherein the multiple doses are administered on 7-14 consecutive days.

231. The method of any one of claims 186-230, wherein the method further comprises administering a therapeutic agent.

232. The method of claim 231, wherein the therapeutic agent is a therapeutic agent for treating GvHD.

233. The method of any one of claims 186-232, wherein the two or more bacterial strains are lyophilized.

234. The method of any one of claims 186-232, wherein the two or more bacterial strains are spray-dried.

235. The method of any one of claims 186-234, wherein one or more of the two or more bacterial strains are in spore form.

236. The method of any one of claims 186-235, wherein each of the two or more bacterial strains are in spore form.

237. The method of any one of claims 186-235, wherein one or more of the two or more bacterial strains are in vegetative form.

238. The method of any one of claims 186-234 and 237, wherein each of the two or more bacterial strains are in vegetative form.

239. The method of any one of claims 186-238, wherein the pharmaceutical composition further comprises one or more enteric polymers.

240. The method of any one of claims 186-239, wherein the administration is oral administration.

241. The method of any one of claims 186-240, wherein the pharmaceutical composition is formulated for oral delivery.

242. The method of any one of claims 186-240, wherein the pharmaceutical composition is formulated for rectal delivery.

243. The method of any one of claims 186-242, wherein the pharmaceutical composition is formulated for delivery to the intestine.

244. The method of any one of claims 186-243, wherein the pharmaceutical composition is formulated for delivery to the colon.

245. A method, comprising administering to a subject a therapeutically effective amount of a pharmaceutical composition in a loading period followed by administering to the subject a therapeutically effective amount of the pharmaceutical composition a maintenance period, wherein the pharmaceutical composition comprises a purified bacterial mixture comprising two or more bacterial strains of species selected from the group consisting of Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

246. The method of claim 245, wherein the purified bacterial mixture comprises at least three, four, five, six, seven, eight, nine, ten, eleven, twelve, thirteen, fourteen, fifteen, or sixteen, bacterial strains.

247. The method of claim 245 or 246, wherein the purified bacterial mixture consists of bacterial strains of species Clostridium saccharogumia, Flavonifractor plautii, Clostridium hathewayi, Blautia coccoides, Clostridium bolteae, Clostridium indolis, Anaerotruncus colihominis, Ruminococcus sp., Clostridium lavalense, Clostridium symbiosum, Clostridium ramosum, Eubacterium contortum, Clostridium scindens, Lachnospiraceae bacterium, Clostridium sp., and Lachnospiraceae bacterium.

248. The method of claim 245 or 246, wherein the purified bacterial mixture consists of bacterial strains of species Clostridium hathewayi, Clostridium bolteae, Clostridium indolis, Anaerotruncus colihominis, Ruminococcus sp., Clostridium lavalense, Clostridium symbiosum, Eubacterium contortum, Lachnospiraceae bacterium, Clostridium sp., and Lachnospiraceae bacterium.

249. A method, comprising administering to a subject a therapeutically effective amount of a pharmaceutical composition in a loading period followed by administering to the subject a therapeutically effective amount of the pharmaceutical composition a maintenance period, wherein the pharmaceutical composition comprises a purified bacterial mixture comprising two or more bacterial strains that comprise 16S rDNA sequences of at least 97% sequence identity to the nucleic acid sequences set forth as SEQ ID NOs: 1-16.

250. The method of claim 249, wherein the purified bacterial mixture comprises at least three, four, five, six, seven, eight, nine, ten, eleven, twelve, thirteen, fourteen, fifteen, or sixteen bacterial strains.

251. The method of claim 249 or 250, wherein the pharmaceutical composition comprises a purified bacterial mixture consisting of bacterial strains that comprise 16S rDNA sequences of at least 97% sequence identity to the nucleic acid sequences set forth as SEQ ID NOs: 1-16.

252. The method of claim 249 or 250, wherein the pharmaceutical composition comprises a purified bacterial mixture consisting of bacterial strains that comprise 16S rDNA sequences of at least 97% sequence identity to the nucleic acid sequences set forth as SEQ ID NOs: 3, 5-10, 12, and 14-16.

253. The method of any one of claims 245-252, wherein the pharmaceutical composition reduces an amount of one or more primary bile acids in the subject.

254. The method of claim 253, wherein the primary bile acid is chenodeoxycholic acid, cholic acid, glycochenodeoxycholic acid, glycocholic acid, taurochenodeoxycholic acid, or taurocholic acid.

255. The method of any one of claims 245-254, wherein the pharmaceutical composition reduces levels of primary bile acids in the subject by 2-fold to 100,000-fold.

256. The method of any one of claims 245-255, wherein the pharmaceutical composition increases an amount of one or more secondary bile acids in the subject.

257. The method of claim 256, wherein the secondary bile acid is alloiso isolithocholic acid, dehydrolithocholic acid, deoxycholic acid, glycodeoxycholic acid, glycoursodeoxycholic acid, lithocholic acid, taurodeoxcycholic acid, or ursodeoxycholic acid.

258. The method of any one of claims 245-257, wherein the pharmaceutical composition increases levels of secondary bile acids in the subject by 2-fold to 100,000-fold.

259. The method of any one of claims 245-258, wherein the pharmaceutical composition increases an amount of one or more short-chain fatty acids (SCFAs) in the subject.

260. The method of claim 259, wherein the SCFA is 2-methylbutyric acid, acetic acid, butyric acid, hexanoic acid, isobutyric acid, isovaleric acid, propionic acid, or valeric acid.

261. The method of any one of claims 249-260, wherein the pharmaceutical composition increases levels of short-chain fatty acids (SCFAs) in the subject by 2-fold to 100,000-fold.

262. The method of any one of claims 245-261, wherein the pharmaceutical composition increases an amount of one or more indoles in the subject.

263. The method of claim 262, wherein the indole is kynurenic acid, serotonin, nicotinic acid, indole, indole 3-acetic, or indole 3-propionic acid.

264. The method of any one of claims 245-263, wherein the pharmaceutical composition increases levels of indoles in the subject by 2-fold to 100,000-fold.

265. The method of any one of claims 245-264, further comprising administering to the subject an antibiotic prior to the loading period.

266. The method of claim 265, wherein the loading period is not preceded by administration of an antibiotic.

267. The method of claim 265 or 266, wherein the antibiotic is vancomycin, fidaxomycin, or ridinilazole.

268. The method of claim 267, wherein the antibiotic is vancomycin.

269. The method of any one of claims 245-268, wherein the loading period is at least 7 days and a loading dose of the pharmaceutical composition is administered to the subject at least every 3 days for the loading period.

270. The method of claim 269, wherein the loading period is between 7 and 14 days.

271. The method of claim 269 or 270, wherein the loading dose of the pharmaceutical composition is administered to the subject daily for the loading period.

272. The method of any one of claims 245-271, wherein the maintenance period is 6 weeks, and a maintenance dose of the pharmaceutical composition is administered to the subject daily for the maintenance period.

273. The method of any one of claim 272, wherein the maintenance dose comprises fewer total colony forming units as compared to the loading dose.

274. The method of any one of claims 245-273, wherein the loading period and maintenance period are repeated every 2 months, 3 months, 4 months, 5 months, 6 months, 12 months, 18 months, or 24 months.

275. The method of any one of claims 245-274, wherein the two or more bacterial strains are lyophilized.

276. The method of any one of claims 245-275, wherein the two or more bacterial strains are spray-dried.

277. The method of any one of claims 245-276, wherein one or more of the two or more bacterial strains are in spore form.

278. The method of any one of claims 245-277, wherein each of the two or more bacterial strains are in spore form.

279. The method of any one of claims 245-277, wherein one or more of the two or more bacterial strains are in vegetative form.

280. The method of any one of claims 245-276 and 279, wherein each of the two or more bacterial strains are in vegetative form.

281. The method of any one of claims 245-280, wherein the pharmaceutical composition further comprises one or more enteric polymers.

282. The method of any one of claims 245-281, wherein the administration is oral administration.

283. The method of any one of claims 245-282, wherein the pharmaceutical composition is formulated for oral delivery.

284. The method of any one of claims 245-282, wherein the pharmaceutical composition is formulated for rectal delivery.

285. The method of any one of claims 245-284, wherein the pharmaceutical composition is formulated for delivery to the intestine.

286. The method of any one of claims 245-285, wherein the pharmaceutical composition is formulated for delivery to the colon.

287. A method for assessing colonization of one or more bacterial strains of a bacterial composition in a microbiome of a subject, the method comprising
isolating nucleic acid from a sample of the microbiome of the subject; and
determining the presence of at least one bacterial strain of the bacterial composition by amplifying a nucleotide sequence of a genomic marker for the at least one the bacterial strains in the isolated nucleic acid;
wherein if a genomic marker for a bacterial strain is present in the amplified nucleotide sequences, the microbiome is colonized with the bacterial strain.

288. The method of claim 287, wherein amplifying comprises performing one or more quantitative polymerase chain reactions (qPCR).

289. The method of claim 287 or 288, wherein the qPCR is performed using one or more pair of primers, wherein each pair of primers comprises a forward primer and a reverse primer for amplifying the nucleotide sequence of the genomic marker of bacterial strain.

290. The method of claim 289, wherein the pair of primers for amplifying the nucleotide sequence of the genomic marker of comprises the forward primer set forth in any one of SEQ ID NOs: 17-32 and the reverse primer set forth in any one of SEQ ID NOs: 33-48.

291. The method of any one of claims 288-290, wherein the qPCR reaction further comprises a DNA probe.

292. The method of claim 291, wherein the DNA probe comprises a fluorophore and at least one quencher.

293. The method of claim 291 or 292, wherein the DNA probe comprises a sequence that is present in any one of the sequences set forth in SEQ ID NOs: 49-64.

294. The method of any one of claims 287-293, wherein if a genomic marker for a bacterial strain is absent in the amplified nucleotide sequences, the method further comprises administering one or more additional doses of the bacterial composition to the subject.
